(19) United States
(12) Patent Application Publication
(10) Pub. No.: US 2007/0099232 A1
(43) Pub. Date: May 3, 2007

(54) Ecdysone receptor ligand-binding domain structure

(76) Inventors: Michael C. Lawrence, Newport (AU); Patricia Pilling, North Fitzroy (AU); George O. Lovrecz, North Balwyn (AU); Vidana C. Epa, Coburg (AU); Jennifer A. Camichael, East Brunswick (AU); Leonie Noyce, Marrickville (AU); Lloyd Graham, North Ryde (AU); Garry N. Hannan, Hunters Hill (AU); David Winkler, Belgrave (AU); Ronald J. Hill, Forestville (AU)

Correspondence Address: GREENLEE WINNER AND SULLIVAN P C, 4875 PEARL EAST CIRCLE, SUITE 200, BOULDER, CO, 80301, US

(21) Appl. No.: 10/558,231

(22) PCT Filed: May 27, 2004
(86) PCT No.: PCT/AU04/00713

§ 371 Date: Jun. 21, 2006

(30) Foreign Application Priority Data
May 27, 2003 (AU) 2003902621

(51) Int. Cl. G01N 33/53

(52) U.S. Cl. 435/7.1

(57) ABSTRACT

The present invention relates to structural studies of the functional insect ecdysone receptor. More particularly, the invention relates to the crystal structure of the whitefly ecdysone receptor ligand-binding domain, specifically that of Bemisia tabaci, and uses of the crystal and related structural information to select and screen for compounds that interact with the receptor.

### FIELD OF THE INVENTION

The present invention relates to structural studies of the functional insect ecdysone receptor. More particularly, the invention relates to the crystal structure of the whitefly ecdysone receptor ligand-binding domain, specifically that of Bemisia tabaci, and uses of the crystal and related structural information to select and screen for compounds that interact with the receptor. Moreover, the crystal structure of the present invention can be used to predict the structure of the ligand-binding pocket of functional ecdysone receptors from related species and to guide site-directed mutagenesis of amino acid residues influencing discrimination between different ligands.

### BACKGROUND OF THE INVENTION

Carroll Williams in the 1960s pointed out that over 99% of insect species are either innocuous or beneficial from the human point of view. Some are even indispensable, e.g. bees via their role in pollination. Approximately 0.1% of insects are actually pests. Williams suggested that a new generation of safer insecticides exhibiting specificity for particular pests might be developed based on the chemistry of the insect's own hormones (Williams, 1967; Williams, 1967). The levels of the non-peptide hormones controlling growth and development, 20-hydroxyecdysone and juvenile hormone, are precisely controlled. Inappropriate levels of compounds with ecdysteroid or juvenoid activity lead to major perturbation of insect development and subsequent lethality.

A problem with this approach, not initially appreciated, stems from the efficient mechanisms insects possess for clearing these hormones by metabolic degradation during normal development. This problem might be overcome by the discovery of compounds exhibiting high receptor affinities but with different chemistry to the native hormones and thus not subject to the host's catabolic pathways.

The two non-peptide hormones known to play key roles in regulating insect growth and development are the steroid moulting hormone, 20-hydroxyecdysone, hereafter referred to as ecdysone, and the sesquiterpenoid juvenile hormone, hereafter referred to as JH. JH is responsible for maintaining larval or nymphal states in moulting insects in addition to a role in adults in the regulation of reproductive processes. The titre of ecdysone may rise and fall as many as six or more times during the life cycle of insects, regulating, for example, the moulting process between larval instars, the synthesis of new cuticle, the onset of metamorphosis (after a decline in JH titre) and aspects of vitellogenesis in the adult ovary. The giant polytene chromosomes seen in the dipteran Drosophila melanogaster, have given insights into the complexity of the response to a rise in ecdysone titre at the level of changes in gene expression. It was postulated by Ashburner and co-workers (Ashburner et al., 1974) that ecdysone exerts its action in regulating gene expression via a protein receptor. A few early responding genes produce further gene transcription regulatory proteins that transmit the response to a whole bank of late responding genes; these regulatory proteins can be detected in action at the late-responding chromosomal loci (Hill et al., 1993).

Over the past decade much progress has been made in understanding the molecular mechanisms underlying the key role of ecdysone in controlling insect development. This research has been led by studies involving the combined power of genetics and molecular biology employing the fly D. melanogaster. Of particular importance to the present application has been the elucidation of the nature of the ecdysone receptor. It has been shown to be a heterodimer made up of the products of two genes called ecr and usp (Yao et al., 1993). The protein products of these genes, EcR and USP, are members of the nuclear receptor superfamily. This family is characterised by an overall structural plan in which a series of domains impart, in order from the N-terminus: transcriptional activation (A/B), DNA binding (C), nuclear localisation (so-called “linker”, D) and ligand binding (E/F). The ligand-binding domain also imparts transactivation in response to the binding of agonist ligands.

Both the EcR and USP subunits of ecdysone receptors have been cloned from a number of insects—see for example (Koelle et al., 1991; Hannan & Hill, 1997; Hannan & Hill, 2001; Oro et al., 1990, WO 99/36520, WO 01/02436).

Despite the considerable interest in JH since the 1960's, the nature of its receptor has proven more elusive. Research reported late in 1997 and subsequent publications suggests that this long-sought-after receptor may also involve USP (Jones & Sharp, 1997; Sasorith et al., 2002) but this remains controversial. Even in the absence of precise knowledge of its receptor, JH has served as a model for intensive programs of chemical synthesis over three decades leading to a host of diverse molecules that mimic the activity of the hormone. Some of these have been registered as insecticides. Recently, three-dimensional structures have been solved for monomeric ligand-binding domains of USPs from the lepidopteran Heliothis virescens (Billas et al., 2001) and the dipteran D. melanogaster (Clayton et al., 2001).

Until the 1980's, chemical approaches to the development of ecdysone mimics were hampered by the structural complexity and synthetic inaccessibility of the steroids for commercial-scale field applications. However in 1988, Rohm and Haas Company scientists (Wing et al., 1988; Wing, 1988) reported that a class of bisacylhydrazine insecticides, which the company had discovered serendipitously, were acting primarily via interaction with ecdysone receptors. Members of this class display remarkable selectivity at the level of orders within the Insecta, for example RH-5992 is some two to three orders of magnitude more effective against Lepidotera than it is against Diptera. This difference correlates with different dissociation constants for interaction of the compound with ecdysone receptors from the two insect orders (Dhadialla et al., 1998). Although subsequent studies (Sundaram et al., 1998) have demonstrated a contribution in some cases by active transport clearance, there is little doubt that variation in the structure of the ecdysone receptors per se between different orders plays a very significant role in underlying the selectivity of extant insecticides in this class.

The selectivity of the bisacylhydrazines for the Lepidoptera and some Coleoptera has both positive and negative connotations. On the positive side, we see a harbinger of safer, more environmentally-friendly insecticides targeting a receptor not only absent from vertebrates but also exhibiting sufficient variation across the Insecta to allow discrimination between pests and friendly or innocuous species. On the negative side, the present relatively narrow spectrum of activity limits sales and also leaves a significant number of insect orders that cannot be controlled by safe ecdysone receptor targeting chemistries. Industry has been trying to extend the spectrum of activity of agents with this mode of action but with relatively little success.

Since much of the selectivity of current agents stems from variations in the structure of the ecdysone receptor, there is a need in the art for knowledge of the three-dimensional structures of the ligand-binding domain of ecdysone receptors, not only to guide the design of new ligand chemistries, but also to introduce into this design process an understanding of receptor atomic features underlying selectivity of action.

Homology modelling based on the known three-dimensional structures of other nuclear receptor ligand-binding domains, including those of retinoic acid receptor and vitamin D receptor (Wurtz et al., 2000) and those of human thyroid hormone receptor β, human estrogen receptor a and human progesterone receptor (Kasuya et al., 2003), has been employed to predict the structure of the corresponding domains of ecdysone receptors for ligand docking studies. Such approaches do not distinguish between alternative potential three-dimensional protein structures and alternative orientations for ligand docking.

Furthermore, ecdysone receptors and their functional domains are employed as components of ecdysone switches for the control of therapeutic genes in mammalian cells and for control of transgenes more generally in agriculturally important species, both animal and plant. Knowledge of the three-dimensional structure of the ligand-binding domain of ecdysone receptors should aid in the design of safer more effective ligands to act as effectors for such switches and to guide site-directed mutagenesis to change ligand preferences of the receptors.

Accordingly, knowledge of the three-dimensional structure co-ordinates of the ecdysone receptor, and in particular the ligand-binding pocket of the receptor, would be useful in facilitating the design of potential selective agonists/antagonists which, in turn, are expected to have insecticidal activity and to include potential safe effectors for ecdysone switches.

### SUMMARY OF THE INVENTION

The present inventors have now obtained three-dimensional structural information concerning the functional ligand-binding domain of the ecdysone receptor of Bemisia tabaci (silverleaf whitefly). The functional B. tabaci ecdysone receptor is a heterodimeric receptor comprising ecdysone receptor subunit protein (BtEcR) and ultraspiracle subunit protein (BtUSP). The BtUSP partner protein associates with the BtEcR receptor protein to confer greatly enhanced affinity for insect steroids or analogues thereof. Compounds that typically modulate the activity of the ecdysone receptor include 20-hydroxyecdysone (henceforth referred to as “ecdysone”), ponasterone A, muristerone A, analogues of an ecdysteroid or certain non-steroidal ecdysone receptor agonists or antagonists, including for example those having dibenzoyl hydrazine chemistries.

The information presented in this application can be used to predict the structure of related members of the ecdysone receptor family from other species as well as to select and/or design compounds which interact with the B. tabaci ecdysone receptor and other ecdysone receptors for use as insecticidally-active agents.

In the remainder of this application the term “ecdysone receptor” is used to denote the functional EcR/USP heterodimer receptor and the subunits are referred to as EcR and USP. Specifically the subunits from B. tabaci are referred to as BtEcR and BtUSP. The term ligand-binding domain will be abbreviated to LBD.

Accordingly, in a first aspect the present invention consists in a crystalline composition comprising BtEcR/BtUSP heterodimer LBD or portion thereof, or a crystalline composition comprising BtEcR/BtUSP heterodimer LBD or portion thereof co-crystallized with a ligand.

In a second aspect the present invention provides a method of selecting or designing a compound that interacts with an ecdysone receptor and modulates an activity mediated by the receptor, the method comprising the step of assessing the stereochemical complementarity between the compound and a topographic region of the BtEcR/BtUSP heterodimer LBD, wherein the heterodimer LBD is characterised by
(a) amino acids 179-415 of the BtEcR monomer and amino adds 300-492 of the BtUSP monomer positioned at atomic coordinates as shown in Appendix I, or structural coordinates wherein the backbone atoms of each monomer has a root mean square deviation from the backbone atoms of their corresponding partners in either amino adds 179-415 of the BtEcR monomer or amino adds 300-492 of the BtUSP monomer of not more than 1.5 Å; or
(b) one or more subsets of said amino adds related to the coordinates of the monomers shown in Appendix I by whole body translations and/or rotations.

By “stereochemical complementarity” we mean that the compound or a portion thereof makes a sufficient number of energetically favourable contacts with the receptor, or topographic region thereof, as to have a net reduction of free energy on binding to the receptor, or topographic region thereof.

Stereochemical complementarity or how well a given chemical compound structure binds or fits to a specified site or cavity in the protein structure can be measured by using one or more of the scoring functions available for this purpose. (See for example P. Ferrara, H. Gohlke, D. J. Price, G. Klebe, and C. L. Brooks III, Assessing scoring functions for protein-ligand interactions, J. Med. Chem., vol. 47, 3032-3047(2004).) A specific example of such a scoring function is X-SCORE (R. Wang, L. Lai, S. Wang, Further development and validation of empirical scoring functions for structure-based binding affinity prediction, J. Comput.-Aided Mol. Des., vol. 16, 11-26(2002)), which is a scoring function that calculates the dissociation constant of a given protein-ligand complex, and was constructed by calibrating to experimental data on a set of 200 protein-ligand complexes.

By “topographic region” is meant a subset of the molecular surface (Connolly, 1983) of the BtEcR LBD alone, the BtUSP LBD alone or the BtEcR/BtUSP heterodimer LBD. This subset may consist of either a single region or multiple disjoint regions. In this context the surface of enclosed cavities within the BtEcR/BtUSP heterodimer LBD or its constituent partners is also treated as part of the molecular surface.

In a third aspect the present invention provides a computer-assisted method for identifying potential compounds able to interact with an ecdysone receptor and thereby modulate an activity mediated by the receptor, using a programmed computer comprising a processor, an input device, and an output device, comprising the steps of:
(a) inputting into the programmed computer, through the input device, data comprising the atomic coordinates of amino adds 179-415 of the BtEcR monomer and amino adds 300-492 of the BtUSP monomer and ponasterone A positioned at atomic coordinates as shown in Appendix I, or structural coordinates wherein the backbone atoms of each monomer has a root mean square deviation from the backbone atoms of their corresponding partners in either amino acids 179-415 of the BtEcR monomer or amino acids 300-492 of the BtUSP monomer of not more than 1.5 Å, or one or more subsets of said amino acids, or one or more subsets of said amino acids related to the coordinates shown in Appendix I by whole body translations and/or rotations;
(b) generating, using computer methods, a set of atomic coordinates of a structure that possesses stereochemical complementarity to the atomic coordinates of amino acids 179-415 of the BtEcR monomer and/or amino acids 300-492 of the BtUSP monomer positioned at atomic coordinates as shown in Appendix I, or structural coordinates having a root mean square deviation from the backbone atoms of their corresponding partners in either amino acids 179-415 of the BtEcR monomer or amino acids 300-492 of the BtUSP monomer of not more than 1.5 Å, or one or more subsets of said amino adds, or one or more subsets of said amino adds related to the coordinates shown in Appendix I by whole body translations and/or rotations, thereby generating a criteria data set;
(c) comparing, using the processor, the criteria data set to a computer database of chemical structures;
(d) selecting from the database, using computer methods, chemical structures which are similar to a portion of said criteria data set; and
(e) outputting, to the output device, the selected chemical structures which are complementary to or similar to a portion of the criteria data set.

In a fourth aspect the present invention provides a computer for producing a three-dimensional representation of a molecule or molecular complex, wherein the computer comprises:
(a) a machine-readable data storage medium comprising a data storage material encoded with machine-readable data, wherein the machine readable data comprises the atomic coordinates of amino acids 179-415 of the BtEcR monomer and amino acids 300-492 of the BtUSP monomer and ponasterone A as shown in Appendix I, or structural coordinates wherein the backbone atoms of each monomer has a root mean square deviation from the backbone atoms of their corresponding partners in either amino acids 179-415 of the BtEcR monomer or amino acids 300-492 of the BtUSP monomer of not more than 1.5 Å, or one or more subsets of said amino acids, or one or more subsets of said amino acids related to the coordinates shown in Appendix I by whole body translations and/or rotations;
(b) a working memory for storing instructions for processing the machine-readable data;
(c) a central-processing unit coupled to the working memory and to the machine-readable data storage medium, for processing the machine-readable data into the three dimensional representation; and
(d) an output hardware coupled to the central processing unit, for receiving the three-dimensional representation.

In a fifth aspect the present invention provides a compound able to modulate an activity mediated by an ecdysone receptor, the compound being obtained by a method according to the present invention.

In a sixth aspect the present invention provides a compound which possesses stereochemical complementarity to a topographic region of the BtEcR/BtUSP heterodimer LBD and which modulates an activity mediated by the receptor, wherein the heterodimer is characterised by
(a) amino acids 179-415 of the BtEcR monomer and amino acids 300-492 of the BtUSP monomer positioned at atomic coordinates as shown in Appendix I, or structural coordinates wherein the backbone atoms of each monomer has a root mean square deviation from the backbone atoms of their corresponding partners in either amino acids 179-415 of the BtEcR monomer or amino acids 300-415 of the BtUSP monomer of not more than 1.5 Å; or
(b) one or more subsets of said amino acids related to the coordinates of the monomers shown in Appendix I by whole body translations and/or rotations;
   with the proviso that the compound is not a naturally occurring ligand of a molecule of the B. tabaci ecdysone receptor.

In a seventh aspect, the present invention provides an insecticidal composition for control of insects which comprises a compound according to the fifth or sixth aspects of the present invention and a pharmaceutically acceptable carrier or diluent.

In yet another aspect, the present invention provides a method for evaluating the ability of a chemical entity to interact with an ecdysone receptor LBD, said method comprising the steps of:
(a) creating a computer model of at least one region of the BtEcR/BtUSP heterodimer LBD using structure coordinates wherein the root mean square deviation between the backbone atoms of the (i) the BtEcR component of the model and the corresponding structure coordinates of amino acids 179-415 of the BtEcR monomer or (ii) the BtUSP component of the model and the corresponding structure coordinates of amino acids 300-492 of the BtUSP monomer as set forth in Appendix I, are not more than 1.5 Å;
(b) employing computational means to perform a fitting operation between the chemical entity and said computer model of at least one region of the monomers of the BtEcR/BtUSP heterodimer LBD; and
(c) analysing the results of said fitting operation to quantify the association between the chemical entity and at least one region of the BtEcR/BtUSP heterodimer LBD model.

In another aspect the present invention consists in a method of assessing the interaction between a compound and the BtEcR/BtUSP heterodimer LBD, the method comprising exposing a crystalline composition comprising BtEcR/BtUSP heterodimer LBD or portion thereof or variant of these to the compound and measuring the level of binding of the compound to the crystal.

As will be readily understood by persons skilled in this field, the methods of the present invention provide a rational method for designing and selecting compounds which interact with an ecdysone receptor and specifically that of B. tabaci. In the majority of cases these compounds will require further development in order to increase activity. Such further development is routine in this field and will be assisted by the structural information provided in this application and screens employing EcR and optionally USP nucleotide and/or polypeptide sequences. In vitro competitive binding screens compete unlabelled test compounds against a labelled ligand (tracer) to observe if they inhibit the binding of the latter to functional receptor LBDs. In vitro competition binding screens may utilise LBD sequences or D (linker) domain sequences linked to LBD sequences. In vivo cell-based screens employ full-length EcR and optionally full-length USP nucleotide sequences functionally linked to suitable promoters for expression in mammalian, insect or yeast cells containing a suitable reporter gene construct. Alternatively, in vivo cell-based screens may employ the EF or DEF domain encoding regions of EcR and optionally of USP nucleotide sequences functionally linked to nucleotide sequences encoding domains from other transcription factors. In particular, the BtEcR nucleotide sequence (SEQ ID NO 1) and/or polypeptide sequence (SEQ ID NO 2) and optionally BtUSP nucleotide sequence and/or polypeptide sequence or the corresponding EF or DEF domains may be utilised in screens to develop improved compounds derived by rational design employing the B. tabaci EcR/USP crystal structure. It is intended that in particular embodiments the methods of the present invention includes such further developmental steps.

In yet a further aspect, the invention provides a method of utilizing molecular replacement to obtain structural information about a molecule or a molecular complex of unknown structure, comprising the steps of:
(a) crystallising said molecule or molecular complex;
(b) collecting an X-ray diffraction data set from said crystallized molecule or molecular complex;
(c) applying at least a portion of the structure coordinates set forth in Appendix I to the X-ray diffraction data set to generate a three-dimensional electron density map of at least a portion of the molecule or molecular complex whose structure is unknown. The term “molecular replacement” refers to a method that involves generating a preliminary model of an ecdysone receptor crystal whose structure coordinates are unknown, by orienting and positioning a molecule whose structure coordinates are known (e.g., BtEcR/BtUSP heterodimer LBD coordinates from Appendix I) within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal (Lattman, 1985; Rossmann, 1990).

As will be clear from the following discussion the present inventors have also isolated a nucleic add molecule encoding the BtEcR.

Accordingly, in a further aspect the present invention provides an isolated nucleic acid molecule comprising a nucleotide sequence which encodes at least the LBD of BtEcR, wherein the nucleotide sequence is selected from the group consisting of:
(i) a nucleotide sequence comprising a sequence having at least 90% identity to the sequence from nucleotide 535 to nucleotide 1248 of SEQ ID NO: 1 or the complementary nucleotide sequence;
(ii) a nucleotide sequence comprising a sequence that hybridises under high stringency conditions to the sequence from nucleotide 535 to nucleotide 1248 of SEQ ID NO: 1 or the complementary nucleotide sequence; and
(iii) a nucleotide sequence which encodes a polypeptide comprising the sequence from amino acid P179 to amino acid S416 of SEQ ID NO: 2.

### BRIEF DESCRIPTION OF THE FIGURES

FIG. 1 Schematic diagram of the structure of the BtEcR/BtUSP heterodimer LBD with bound ponasterone A shown in the binding pocket. The BtEcR LBD is shown in grey, whilst the BtUSP LBD is in black. Individual helices are shown as cylinders and individual β-strands as arrows. The N- and C-terminii of each molecule are labelled. Ponasterone A is shown in black with its oxygen atoms in white. Helix 3 of the BtEcR LBD is rendered transparent in order to enable viewing of the ponasterone A moeity. The surface of the binding pocket itself is shown in transparent grey.

FIG. 2 View of the extended ecdysteroid binding pocket, showing the surface of the pocket, bound ponasterone A and all residues that form the walls of the pocket. The pocket is separated into two parts for clarity—the entire pocket can be re-generated by rotating the lower image about a vertical axis running in the plane of the paper and placing it on top of the upper image. Ponasterone A is shown in black as a “thick” stick representation in both images with its oxygen atoms represented by black balls. Surrounding residues that form the cavity are labelled and are shown as “thin” grey sticks if they are totally conserved across all species, else they are shown as “thin” black sticks. Individual atoms within residues are rendered as follows—nitrogen: small balls, oxygen: medium balls and sulphur: large balls. Again for clarity, side chain or alternatively backbone atoms are omitted for individual residues if these groups of atoms do not form any part of the cavity wall. Hydrogen bonds between individual protein residues and ponasterone A are shown as black dotted lines. The molecular surface of the binding pocket is shown in transparent grey.

FIG. 3 Stick/CPK diagram of the BtEcR LBD co-activator/co-repressor binding groove (without H12) with individual residues labelled. All atoms from residues 231 to 265 are rendered as transparent CPK and a Cα trace to delineate the groove. Individual residues with putatively capable of interaction with co-activator/co-repressor proteins are rendered in black stick format, with nitrogen atoms as small grey balls and oxygen atoms as large grey balls.

FIG. 4 An analysis of freshly-prepared recombinant BtEcR LBD samples by 12% SDS-PAGE, with staining by Coomassie Blue. Samples were boiled in the presence of 5% (v/v) 2-mercaptoethanol before loading. M: marker proteins, with molecular masses shown in kilodaltons (kDa). Lane 1: Immobilised metal-ion affinity chromatography (IMAC) eluate, showing recombinant BtEcR and BtUSP LBDs (major doublet) and many additional bands (contaminating proteins). Lane 2: Concentrated gel filtration eluate, showing BtEcR and BtUSP LBD's (main doublet) with relatively few contaminating proteins.

FIG. 5 Residues defining the terminal end of the major pocket. The Van der Waals surface for the binding pocket in this region is shown as a smooth grey surface to the right. Space-filling representation of ponasterone A in the X-ray structure of the ligand-receptor complex. The smooth grey surface to the right represents the Van der Waals surface of the binding pocket in the region near the alkyl chain of the ecdysteroid.

FIG. 6 Highly-ranked FlexX docking of ponasterone A superimposed on the X-ray structure of ponasterone A bound into the EcR. The dark grey structure represents the x-ray orientation of ponasterone A and the light grey structure represents one of the FlexX poses for ponasterone A. The ability of FlexX to dock a ligand into the receptor can be assessed by the high similarity of the ponasterone A orientation from docking to that in the X-ray model.

FIG. 7 Highly-ranked FlexX docking pose for compound III. The thiophene ring extends into the lipophilic end of the receptor pocket, in the region where the C25 end of ponasterone A binds. The cyclic ester is able to make hydrogen bonds with Asn390 and the ring nitrogen form a hydrogen bond with Thr231. The phenyl ring lies in the same position as that of the C/D rings of ponasterone A.

FIG. 8 Overlay of the hemipteran B. tabaci and lepidopteran H virescens ecdysone receptor LBD ponasterone A bound pockets and superposition of ligands. The HvEcR 1R1K (ponasterone A containing) and HvEcR 1R20 (synthetic agonist BYI06830 containing) LBDs were aligned to the BtEcR (ponasterone A containing) LBD by least squares alignment of the protein Cα backbone atoms. The ligand agonists are shown in ball and stick format with the BTECR ponasterone A in “thick” black sticks, the HvEcR 1R1K ponasterone A in “thick” grey sticks and, the HvEcR 1R20 BYI06830 in “thin” grey sticks. The carbon atoms are rendered in black, the oxygen atoms in grey and the nitrogen atoms in white. The BtEcR ponasterone A bound pocket is shown as a transparent pale grey surface and the HvEcR 1R1K ponasterone A bound pocket surface shown in transparent dark grey. (For clarity, the surface of the BY106830 bound pocket is not depicted in the figure.) A pronounced bulge, absent from the BtEcR ponasterone A bound pocket, is apparent in the HvEcR ponasterone A bound pocket at top left.

### DETAILED DESCRIPTION

The present inventors have doned BtEcR and BtUSP and expressed, crystallised and determined the three-dimensional structure of the BtEcR/BtUSP heterodimer LBD of the ecdysone receptor from Bemisia tabaci.

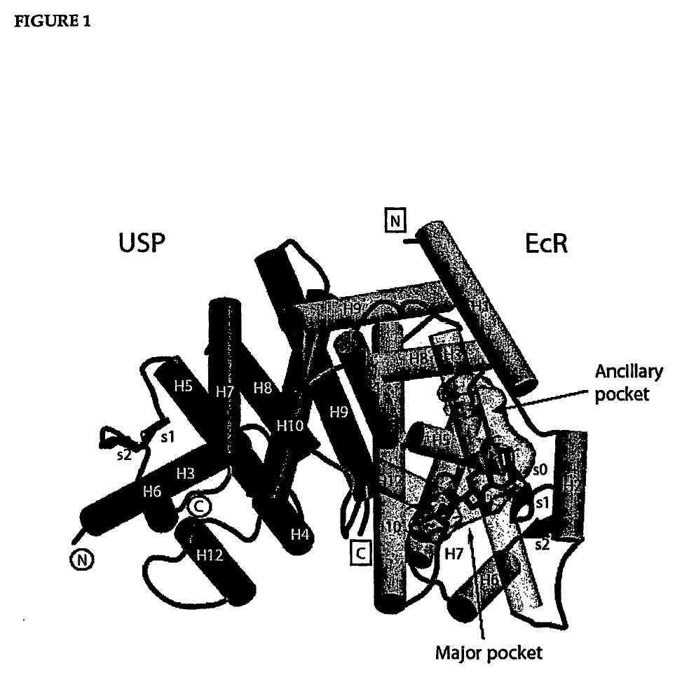

The fold of BtEcR LBD is that of a canonical nuclear hormone receptor. The secondary structure elements of BtUSP/BtEcR LBD discerned in this structure are located within the BTECR sequence as follows: helix H1—residues 182 to 198, helix H2—residues 202 to 211, helix H3—residues 220 to 244, helix H4—residues 252 to 264, helix H5—residues 267 to 275, strand s0—residues 275 to 277, strand s1—residues 282 to 285, strand s2—residues 288 to 291, helix H6—residues 292 to 300, helix H7—residues 304 to 319, helix H8—residues 321 to 334, helix H9—residues 342 to 364, helix H10—residues 368 to 400 and helix H12—residues 405 to 413. Thus, the structure of BtEcR LBD comprises α-helices H1 to H10 and H12, and β-strands s1 and s2 located between helices H5 and H6, as shown in FIG. 1. An additional short β-strand (labelled here as s0) lies between helix H5 and strand s1. Helix H12 in BtEcR is observed in the so-called agonist conformation (Renaud & Moras, 2000).

The structure of the BtEcR LBD was compared with those available for other nuclear receptors. The closest structural neighbour was the LBD of retinoic acid receptor (RAR). The root-mean-square deviation of 206 (out of 237) corresponding backbone C^{α} atoms between the BtEcR structure and that of RAR-γ2 (RCSB id: 1EXA) is 1.29 Å. The major difference between these structures lies in the conformation of the loop between helices H1 and H3. In RAR this loop has a random coil conformation and lies across the outer surface of the s1-s2 β-sheet loop. In EcR the segment contains an intact helix H2 which packs anti-parallel on the N-terminal portion of helix H3 and interacts with the opposite surface of the s1-s2 β-sheet loop.

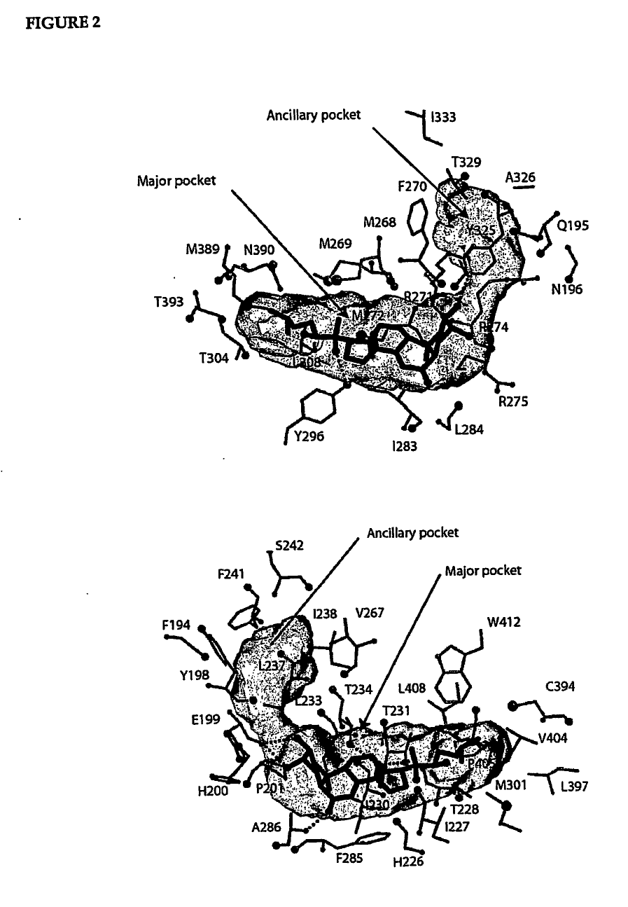

The ligand ponasterone A was observed to lie in a totally-enclosed pocket formed by residues F194, Q195, N196, Y198, E199, H200, P201, H226, I227, T228, I230, T231, L233, T234, L237, I238, F241, S242, V267, M268, M269, F270, R271, M272, R274, R275, I283, L284, F285, A286, Y296, M301, T304, L308, Y325, A326, T329, 1333, M389, N390, T393, C394, L397, V404, P405, L408 and W412 (FIG. 2). The pocket has a “J-shaped” architecture, with the major part (the leg of the “J”) accommodating the ligand, plus an ancillary part (the curved tail of the “J”) existing as an extension of the major part via a narrow channel. The inner wall of the channel linking the major and ancillary parts of the pocket is formed by the side chain of residue R271. The accessible volume of the entire cavity is approximately 766 Å³, whilst the volume of the ponasterone A itself is 434 Å³, both figures calculated using VOIDOO (Kleywegt & Jones, 1994). The ancillary cavity appears unoccupied in the structure presented here. The narrowness of channel connecting the major and ancillary parts of the pocket suggests that it in some dynamic states of the protein these two parts may become disjoint rather than forming a single topological entity.

Potential hydrogen bonds between individual protein atoms and ligand are as follows: A286 N to the ponasterone A hydroxyl at C-6, T234 O^{γ1} to the ponasterone A hydroxyl at C-14, T231 O^{γ1} to the ponasterone A hydroxyl at C-14, R271 NH1 to the ponasterone A hydroxyl at C-2, E199 O to the ponasterone A hydroxyl at C-2, E199 O to the ponasterone A hydroxyl at C-3, Y296 OH to the ponasterone A hydroxyl at C-20 (FIG. 2). The remainder of the contacts between ligands and protein are overwhelmingly hydrophobic in nature and formed by contacts between the side chains of residues P201, I227, T228, I230, M268, M269, R271, M272, R275, 1283, F285, A286, M301, L308, M389, L397, P405, L408 and W412 and the ligand.

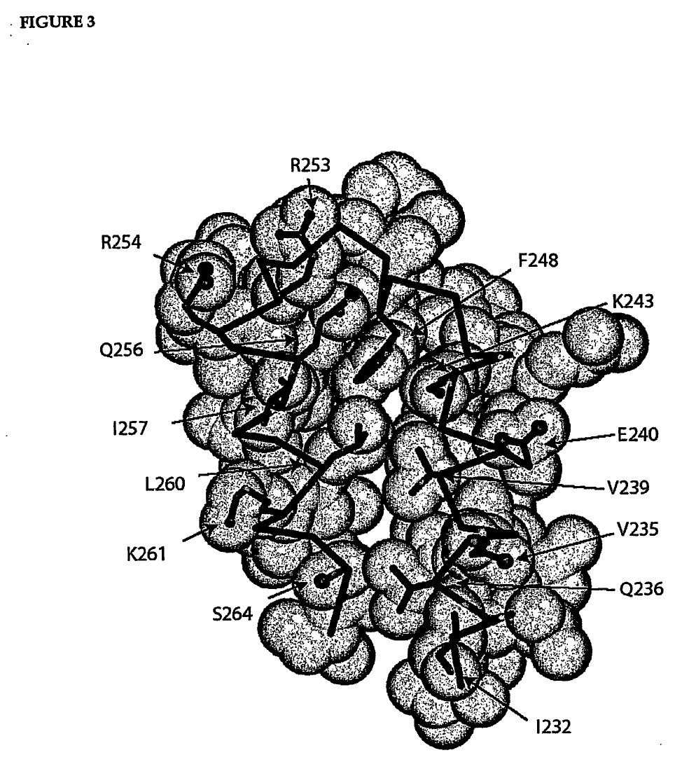

Helix H12 was observed to lie in the so-called agonistic conformation (Renaud & Moras, 2000) possibly locking the ligand into the site via the side chain of W412 which hangs into the ligand-binding site. A salt bridge between BtEcR residues D413 and K261 appears to stabilize the C-terminus of H12. In this conformation a co-activator can bind to a site that includes H12 and the surface of the hydrophobic cleft between helices H3 and H4. The molecular detail of this cleft is presented in FIG. 3. Side chains forming the cleft and its immediate surrounds include those of residues I232, V235, Q236, V239, E240, K243, F248, R253, E254, Q256, I257, L260, K261, S264, and residues S406, F407, L408, E410, I411 and D413 of H12. Excluding H12, this groove is totally conserved across all ecdysone receptor sequences except for R253. This residue lies at the distal end of the binding groove (with respect to the position of H12 shown in this structure) and it is unclear at this stage whether or not its side chain interacts directly with the co-repressor or co-activator upon binding of these elements.

The structure of the BtUSP protein resembles that of other published USP structures (Billas et al., 2001; Clayton et al., 2001), but with the following major difference. No electron density was visible for residues prior to V300, i.e. helix H1, and part of the loop connecting H1 to H3 are totally unobserved. Part of the volume occupied by these structural elements in other USP structures is now occupied by the H10-H12 loop. H12 lies in the so-called antagonistic conformation (Renaud & Moras, 2000) whilst the helix H11 appears not to be formed. No ligand was observed in the site corresponding to that occupied by phospholipid in the two above published structures, and indeed part of that binding site is now occluded by a repositioning of the H10-H12 loop, and by a repositioning of helix H6 and residues immediately adjacent to this element (residues 371 to 384). The repositioning of the H10-H12 loop likely arises from the absence of residues prior to H3 in our structure, allowing this element to collapse into the region normally occupied by the H1-H3 loop in the intact USP ligand-binding domains. Part of the movement of the H10-H12 loop may be caused by the involvement of that loop in a crystal contact with a neighbouring molecule in our structure. Alternatively the observed conformation of the H10-H12 loop may be adopted in solution as well in view of the absence of H1. The secondary structure elements of BtUSP/BtEcR LBD discerned in this structure are located within the BtUSP sequence as follows: helix H3—residues 301 to 321, helix H4—residues 328 to 339, helix H5—residues 340 to 353, strand s1 residues 359 to 361, strand s2—residues 365 to 367, helix H6—residues 371 to 376, helix H7—residues 380 to 396, helix H8—residues 399 to 411, helix H9—residues 420 to 443, helix H10—residues 448 to 466 and helix H12—residues 481 to 491.

The dimeric association between BtEcR and BtUSP ligand-binding domains resembles that of the corresponding RAR-RXR complex. These two heterodimeric structures can be overlaid with an root-mean-square deviation of 1.37 Å for 339 matched C^{α} atoms. The interface is formed by EcR residues contained in H9, H10 and the loop between H8 and H9 on one hand and USP residues contained in H7, H9, H10 and the loop between H6 and H7 on the other (see Table 5). Residues involved in the interface include BtEcR residues H314, M315, I331, S335, E336, R337, P338, E347, Q350, E351, I354, E355, K358, T370, T371, F373, A374, K375, L377, S378, L380, T381, E382, R384, T385 and N388 on one hand and BtUSP residues E342, R383, T386, E387, K391, E414, E425, E429, Y432, A433, E436, S447, G448, F450, A451, K452, L454, L455, R456, L457, P458, A459, R461, S462 and L465 on the other. The interface was estimated by computing all residues with any atom's van der Waals surface within 1.4 Å of that of any atom of the opposite chain followed by visual inspection.

Potential inter-chain salt bridges include those from USP E429 to EcR K375, USP K391 to EcR E336, USP K391 to EcR E347, USP K452 to EcR E351 and USP E425 to EcR K375. Out of these, only the salt bridge between EcR E347 and USP K391 is conserved across all species (although the Dipteran, Chironomus tentans EcR has Asp at the position corresponding to E347 in BtEcR), and compounds which bind to the interface and disrupt a particular salt bridge could be the basis of specific antagonists.

Hydrogen bonds occur between the side chains of USP S447 and the side chain of EcR E355A, between the backbone carbonyl of USP S447 and the side chain of EcR K358 and between the side chains of EcR R384 and USP S462. The remainder of the contacts are hydrophobic in nature. A single phosphate ion is included in the interface, coordinated by the side chains of the EcR residue R384, the carbonyl oxygen of EcR residue E336 and the side chains of USP residues R383, B387 and R456.

PASS (Brady & Stouten, 2000) shows the existence of a pocket on the BtEcR surface on the edge of the heterodimeric interface bounded by residues including A262, S265, E266, R337, R384, G387, N388 and S391 of BtEcR. PASS also shows the existence of a pocket on the BtUSP surface on the edge of the heterodimeric interface bounded by residues including K337, S338, N341, E342, K416, G464, L465, C467 and H470 of BtUSP.

Clearly the information provided in this application will enable rational design/selection of compounds which will interact with the ecdysone receptor.

Accordingly, in a first aspect the present invention consists in a crystalline composition comprising BtEcR/BtUSP heterodirner LBD or portion thereof, or a crystalline composition comprising BtEcR/BtUSP heterodimer LBD or portion thereof co-crystallized with a ligand.

In a second aspect the present invention provides a method of selecting or designing a compound that interacts with an ecdysone receptor and modulates an activity mediated by the receptor, the method comprising the step of assessing the stereochemical complementarity between the compound and a topographic region of the BtEcR/BtUSP heterodimer LBD, wherein the heterodimer is characterised by
(a) amino acids 179-415 of the BtEcR monomer and amino adds 300-492 of the BtUSP monomer positioned at atomic coordinates as shown in Appendix I, or structural coordinates wherein the backbone atoms of each monomer has a root mean square deviation from the backbone atoms of their corresponding partners in either amino acids 179-415 of the BtEcR monomer or amino adds 300-492 of the BtUSP monomer of not more than 1.5 Å; or
(b) one or more subsets of said amino adds related to the coordinates of the monomers shown in Appendix I by whole body translations and/or rotations.

In a preferred embodiment of this aspect of the invention the structural coordinates have a root mean square deviation from the backbone atoms of said amino acids of not more than 1.0 Å, and more preferably not more than 0.7 Å.

As discussed above the ligand ponasterone A was observed to lie in a totally-enclosed pocket formed by residues F194, Q195, N196, Y198, E199, H200, P201, H226, I227, T228, I230, T231, L233, T234, L237, I238, F241, S242, V267, M268, M269, F270, R271, M272, R274, R275, I283, L284, F285, A286, Y296, M301, T304, L308, Y325, A326, T329, I333, M389, N390, T393, C394, L397, V404, P405, L408 and W412. Accordingly, in one embodiment of the second aspect, the topographic region of the ecdysone receptor to which the compound, or a portion thereof has stereochemical complementarity is the ligand-binding pocket of the BtEcR subunit defined by amino acids F194, Q195, N196, Y198, E199, H200, P201, H226, I227, T228, I230, T231, L233, T234, L237, I238, F241, S242, V267, M268, M269, F270, R271, M272, R274, R275, I283, L284, F285, A286, Y296, M301, T304, L308, Y325, A326, T329, I333, M389, N390, T393, C394, L397, V404, P405, L408 and W412.

In another embodiment of the second aspect, the topographic region of the ecdysone receptor to which the compound, or a portion thereof has stereochemical complementarity is the interface between the BtEcR and BtUSP subunits defined by BtEcR residues H314, M315, I331, S335, E336, R337, P338, E347, Q350, E351, I354, E355, K358, T370, T371, P373, A374, K375, L377, S378, L380, T381, E382, R384, T385 and N388 on one hand and BtUSP residues E342, R383, T386, E387, K391, E414, E425, E429, Y432, A433, E436, S447, G448, F450, A451, K452, L454, L455, R456, L457, P458, A459, R461, S462 and L465 on the other. in a still further embodiment of the second aspect, the topographic region of the ecdysone receptor to which the compound, or a portion thereof has stereochemical complementarity is the co-activator/ co-repressor binding groove formed by helices H3 and H4 on the surface of BtEcR defined by residues I232, V235, Q236, V239, E240, K243, F248, R253, E254, Q256, I257, L260, K261, S264, S265, M268, S406, P407, L408, E410, I411 and D413. By “stereochemical complementarity” we mean that the compound or a portion thereof makes a sufficient number of energetically favourable contacts with the receptor, or topographic region thereof, as to have a net reduction of free energy on binding to the receptor, or topographic region thereof.

In the preferred embodiment of the second aspect of the present invention, the method comprises selecting a compound which has portions that match residues positioned in the topographic region of the receptor defined by the specified amino add residues.

By “match” we mean that the identified portions interact with the surface residues, for example, via hydrogen bonding or by enthalpy-reducing Van der Waals interactions which promote desolvation of the biologically active compound with the receptor, in such a way that retention of the compound by the receptor is favoured energetically.

Preferably, the method comprises selecting a compound which forms hydrogen bonds with at least one amino acid residue selected from the group consisting of E199, I227, T231, T234, R271, A286, Y296, T304, N390 and C394 of the ligand-binding pocket of the BtEcR LBD, wherein the compound is not a naturally-occurring ecdysteroid ligand of the ligand-binding pocket of the receptor.

Still more preferably, the method comprises selecting a compound which further forms hydrophobic contacts with the side chains of at least one amino acid residue selected from the group consisting of P201, I227, T228, I230, M268, M269, R271, M272, R275, I283, F285, A286, M301, L308, M389, L397, P405, L408 and W412 of the ligand-binding pocket of the BtEcR subunit, wherein the compound is not the natural ligand of the ligand-binding pocket of the receptor.

In another embodiment crystals of the unliganded EcR/USP heterodimer are exposed to libraries of compounds according to the method of (Nienaber et al., 2000). The most potent ligand will bind preferentially to the crystal and can be identified by difference electron density maps.

In a still further embodiment of the second aspect, the method comprises selecting a compound which is an antagonist of the B. tabaci ecdysone receptor.

Alternatively, the method comprises selecting a compound which is an agonist of the B. tabaci ecdysone receptor.

It is anticipated that modulation of the activity of the B. tabaci ecdysone receptor may be achieved by a number of different means.

The compound may bind to the receptor so as to interfere sterically or allosterically with natural steroid ligand binding. For example.
(a) The compound may bind to the BtEcR ligand-binding pocket of the receptor such as to decrease the size of the ligand-binding pocket thereby preventing access of the ligand to one or more of the specified residues critical for receptor activity.
(b) The compound may bind at or near the interface between the BtEcR and BtUSP association interface to thereby perturb the subunit association for the signalling competent ligand-receptor complex.
(c) The compound may bind at a site remote from the BtEcR ligand-binding pocket but disturb the receptor structure so as to modulate the affinity of ligand-binding.

The compound may interfere with association of the BtEcR and BtUSP subunits of the ecdysone receptor in a number of ways. For example, the compound may bind to the B. tabaci ecdysone receptor at or near one or more of the specified residues of the association interface and by steric overlap and/or electrostatic repulsion prevent association. Alternatively, the compound may bind so as to interfere allosterically with association of the subunits. In addition, the compound may bind to the BtUSP subunit so as to alter the association of the subunits and thereby modulate the affinity of the BtEcR subunit for the natural ligand.

In the preferred form, the compound is selected or designed to interact with the B. tabaci ecdysone receptor in a manner such as to interfere with the association of the BtEcR and BtUSP subunits by inhibiting the association of BtEcR residues H314, M315, I331, S335, E336, R337, P338, E347, Q350, E351, I354, E355, K358, T370, T371, F373, A374, K375, L377, S378, V379, L380, T381, E382, R384, T385 and N388 on one hand and BtUSP residues E342, R383, T386, E387, K391, I408, V409, E414, E425, R428, E429, Y432, A433, E436, S447, G448, F450, A451, K452, L454, L455, R456, L457, A459, R461, S462 and L465 on the other.

In another preferred form the compound may bind to the receptor so as to interfere with signalling of the receptor. For example, the compound may be selected or modified from a known compound (such as the natural ligand), or identified from a data base. It would be expected that such a variant would compete with binding of the natural ligand to the receptor.

In another preferred embodiment the compound is selected or designed based on the natural ligand, the compound being designed or selected such that it interacts with at least one amino add selected from the group consisting of F194, Q195, N196, Y198, E199, H200, P201, H226, I227, T228, I230, T231, L233, T234, L237, I238, F241, S242, V267, M268, M269, F270, R271, M272, R274, R275, I283, L284, F285, A286, Y296, M301, T304, L308, Y325, A326, T329, I333, M389, N390, T393, C394, L397, V404, P405, L408 and W412. In a preferred embodiment, the compound is selected or designed such that the interaction between the compound and the B. tabaci ecdysone receptor is preferred over the interaction of the natural ligand with the B. tabaci ecdysone receptor. Such compounds may be agonists or antagonists of receptor activity.

In a preferred embodiment of the second aspect the method further comprises the step of obtaining a compound which possesses stereochemical complementarity to a topographic region of the BtEcR/BtUSP heterodimer LBD and testing the compound for insecticidal activity.

In a third aspect the present invention provides a computer-assisted method for identifying potential compounds able to interact with an ecdysone receptor and thereby modulate an activity mediated by the receptor, using a programmed computer comprising a processor, an input device, and an output device, comprising the steps of:
(a) inputting into the programmed computer, through the input device, data comprising the atomic coordinates of amino acids 179-415 of the BtEcR monomer and amino acids 300-492 of the BtUSP monomer and ponasterone A positioned at atomic coordinates as shown in Appendix I, or structural coordinates wherein the backbone atoms of each monomer has a root mean square deviation from the backbone atoms of their corresponding partners in either amino adds 179-415 of the BtEcR monomer or amino acids 300-492 of the BtUSP monomer of not more than 1.5 Å, or one or more subsets of said amino acids, or one or more subsets of said amino acids related to the coordinates shown in Appendix I by whole body translations and/or rotations;
(b) generating, using computer methods, a set of atomic coordinates of a structure that possesses stereochemical complementarity to the atomic coordinates of amino acids 179-415 of the BtEcR monomer and/or amino acids 300-492 of the BtUSP monomer positioned at atomic coordinates as shown in Appendix I, or structural coordinates having a root mean square deviation from the backbone atoms of their corresponding partners in either amino adds 179-415 of the BtEcR monomer or amino adds 300-492 of the BtUSP monomer of not more than 1.5 Å, or one or more subsets of said amino acids, or one or more subsets of said amino acids related to the coordinates shown in Appendix I by whole body translations and/or rotations, thereby generating a criteria data set;
(c) comparing, using the processor, the criteria data set to a computer database of chemical structures;
(d) selecting from the database, using computer methods, chemical structures which are similar to a portion of said criteria data set; and
(e) outputting, to the output device, the selected chemical structures which are complementary to or similar to a portion of the criteria data set.

In a preferred embodiment of this aspect of the invention the structural coordinates have a root mean square deviation from the backbone atoms of said amino adds of not more than 1.0 Å, and more preferably not more than 0.7 Å.

Preferably, the method is used to identify potential compounds which are insecticidally active agents or safe effectors for ecdysone switches.

In a further preferred embodiment of the third aspect the method further comprises the step of obtaining a compound with a chemical structure selected in steps (d) and (e), and testing the compound for insecticidal activity.

In a preferred embodiment the subset of amino acids is that defining the ligand-binding pocket of the BtEcR subunit, namely P194, Q195, N196, Y198, E199, H200, P201, H226, I227, T228, I230, T231, L233, T234, L237, I238, F241, S242, V267, M268, M269, F270, R271, M272, R274, R275, I283, L284, F285, A286, Y296, M301, T304, L308, Y325, A326, T329, I333, M389, N390, T393, C394, L397, V404, P405, L408 and W412.

In another embodiment the subset of amino acids is that defining the interface between the BtEcR and BtUSP subunits defined by BtEcR residues H314, M315, I331, S335, E336, R337, P338, E347, Q350, E351, I354, E355, K358, T370, T371, F373, A374, K375, L377, S378, L380, T381, E382, R384, T385 and N388 on one hand and BtUSP residues E342, R383, T386, E387, K391, E414, E425, E429, Y432, A433, E436, S447, G448, F450, A451, K452, L454, L455, R456, L457, P458, A459, R461, S462 and L465 on the other.

In a still further embodiment the subset of amino acids is that defining the co-activator/co-repressor binding groove formed by helices H3 and H4 on the surface of BtEcR defined by residues I232, V235, Q236, V239, E240, K243, F248, R253, E254, Q256, I257, L260, K261, S264, S265, M268, S406, F407, L408, E410, I411 and D413.

The present invention also provides a method of screening of a putative compound having the ability to modulate the activity of the B. tabaci ecdysone receptor (BtEcR/BtUSP) or a heterodimer comprising BtEcR (SEQ ID NO 1) paired with another functional partner protein such as RXR, comprising the steps of identifying a putative compound according to the second or third aspects, and testing the compound for activity. In one embodiment, testing is carried out in vitro. Preferably, the in vitro test is a high throughput assay. In another embodiment, the test is carried out in vivo employing cell-based or whole organism-based screens.

In a fourth aspect the present invention provides a computer for producing a three-dimensional representation of a molecule or molecular complex, wherein the computer comprises:
(a) a machine-readable data storage medium comprising a data storage material encoded with machine-readable data, wherein the machine readable data comprises the atomic coordinates of amino acids 179-415 of the BtEcR monomer and amino adds 300-492 of the BtUSP monomer and ponasterone A as shown in Appendix I, or structural coordinates wherein the backbone atoms of each monomer has a root mean square deviation from the backbone atoms of their corresponding partners in either amino acids 179-415 of the BtEcR monomer or amino acids 300-492 of the BtUSP monomer of not more than 1.5 Å, or one or more subsets of said amino acids, or one or more subsets of said amino acids related to the coordinates shown in Appendix I by whole body translations and/or rotations;
(b) a working memory for storing instructions for processing the machine-readable data;
(c) a central-processing unit coupled to the working memory and to the machine-readable data storage medium, for processing the machine-readable data into the three dimensional representation; and
(d) an output hardware coupled to the central processing unit, for receiving the three-dimensional representation.

In a preferred embodiment of this aspect of the invention the structural coordinates have a root mean square deviation from the backbone atoms of said amino acids of not more than 1.0 Å, and more preferably not more than 0.7 Å.

In a preferred embodiment the subset of amino adds is that defining the ligand-binding pocket of the BtEcR subunit, namely F194, Q195, N196, Y198, E199, H200, P201, H226, I227, T228, I230, T231, L233, T234, L237, I238, F241, S242, V267, M268, M269, F270, R271, M272, R274, R275, I283, L284, F285, A286, Y296, M301, T304, L308, Y325, A326, T329, I333, M389, N390, T393, C394, L397, V404, P405, L408 and W412.

In another embodiment the subset of amino adds is that defining the interface between the BtEcR and BtUSP subunits defined by BtEcR residues H314, M315, I331, S335, E336, R337, P338, E347, Q350, E351, I354, E355, K358, T370, T371, F373, A374, K375, L377, S378, L380, T381, E382, R384, T385 and N388 on one hand and BtUSP residues E342, R383, T386, E387, K391, E414, E425, E429, Y432, A433, E436, S447, G448, F450, A451, K452, L454, L455, R456, L457, P458, A459, R461, S462 and L465 on the other.

In a still further embodiment the subset of amino acids is that defining the co-activator/co-repressor binding groove formed by helices H3 and H4 on the surface of BtEcR defined by residues I232, V235, Q236, V239, E240, K243, F248, R253, E254, Q256, I257, L260, K261, S264, S265, M268, S406, F407, L408, E410, I411 and D413.

In a fifth aspect the present invention provides a compound able to modulate an activity mediated by an ecdysone receptor, the compound being obtained by a method according to the present invention.

In a sixth aspect the present invention provides a compound which possesses stereochemical complementarity to a topographic region of the BtEcR/BtUSP heterodimer LBD and which modulates an activity mediated by the receptor, wherein the heterodimer LBD is characterised by (
a) amino acids 179-415 of the BtEcR monomer and amino acids 300-492 of the BtUSP monomer positioned at atomic coordinates as shown in Appendix I, or structural coordinates wherein the backbone atoms of each monomer has a root mean square deviation from the backbone atoms of their corresponding partners in either amino adds 179-415 of the BtEcR monomer or amino acids 300-492 of the BtUSP monomer of not more than 1.5 Å; or
(b) one or more subsets of said amino acids related to the coordinates of the monomers shown in Appendix I by whole body translations and/or rotations;
   with the proviso that the compound is not a naturally occurring ligand of a molecule of the receptor.

In a preferred embodiment of this aspect of the invention the structural coordinates have a root mean square deviation from the backbone atoms of said amino acids of not more than 1.0 Å, and more preferably not more than 0.7 Å.

In one embodiment the sixth aspect, the topographic region of the ecdysone receptor to which the compound, or a portion thereof has stereochemical complementarity is the ligand-binding pocket of the BtEcR subunit defined by amino adds F194, Q195, N196, Y198, E199, H200, P201, H226, I227, T228, I230, T231, L233, T234, L237, I238, F241, S242, V267, M268, M269, F270, R271, M272, R274, R275, I283, L284, F285, A286, Y296, M301, T304, L308, Y325, A326, T329, I333, M389, N390, T393, C394, L397, V404, P405, L408 and W412.

In another embodiment of the sixth aspect, the topographic region of the ecdysone receptor to which the compound, or a portion thereof has stereochemical complementarity is the interface between the BtEcR and BtUSP subunits, defined by BtEcR residues H314, M315, I331, S335, E336, R337, P338, E347, Q350, E351, I354, E355, K358, T370, T371, F373, A374, K375, L377, S378, L380, T381, E382, R384, T385 and N388 on one hand and BtUSP residues E342, R383, T386, E387, K391, E414, E425, E429, Y432, A433, E436, S447, G448, F450, A451, K452, L454, L455, R456, L457, P458, A459, R461, S462 and L465 on the other.

In still a further embodiment of the sixth aspect the topographic region of the ecdysone receptor to which the compound, or a portion thereof has stereochemical complementarity is the co-activator/co-repressor binding groove formed by helices H3 and H4 on the surface of BtEcR defined by residues I232, V235, Q236, V239, E240, K243, F248, R253, E254, Q256, I257, L260, K261, S264, S265, M268, S406, F407, L408, E410, I411 and D413.

In a seventh aspect, the present invention provides an insecticidal composition for control of insects which comprises a compound according to the fifth or sixth aspects of the present invention and a pharmaceutically acceptable carrier or diluent.

In yet another aspect, the present invention provides a method for evaluating the ability of a chemical entity to interact with the BtEcR/BtUSP heterodimer LBD, said method comprising the steps of:
(a) creating a computer model of at least one region of the BtEcR/BtUSP heterodimer LBD using structure coordinates wherein the root mean square deviation between the backbone atoms of (i) the BtEcR component of the model and the corresponding structure coordinates of amino acids 179-415 of the BtEcR monomer or (ii) the BtUSP component of the model and the corresponding structure coordinates of amino adds 300-492 of the BtUSP monomer, as set forth in Appendix I is not more than 1.5 Å;
(b) employing computational means to perform a fitting operation between the chemical entity and said computer model of at least one region of the monomers of the BtEcR/BtUSP heterodimer LBD; and
(c) analysing the results of said fitting operation to quantify the association between the chemical entity and at least one region of the BtEcR/BtUSP heterodimer LBD model.

In a preferred embodiment of this aspect of the invention the structural coordinates have a root mean square deviation from the backbone atoms of said amino acids of not more than 1.0 Å, and more preferably not more than 0.7 Å.

In a preferred embodiment the region is the ligand-binding pocket of the BtEcR subunit defined by amino adds F194, Q195, N196, Y198, E199, H200, P201, H226, I227, T228, I230, T231, L233, T234, L237, I238, F241, S242, V267, M268, M269, F270, R271, M272, R274, R275, I283, L284, F285, A286, Y296, M301, T304, L308, Y325, A326, T329, I333, M389, N390, T393, C394, L397, V404, P405, L408 and W412.

In another embodiment the region is the interface between the BtEcR and BtUSP subunits defined by BtEcR residues H314, M315, I331, S335, E336, R337, P338, E347, Q350, E351, I354, E355, K358, T370, T371, F373, A374, K375, L377, S378, L380, T381, E382, R384, T385 and N388 on one hand and BtUSP residues E342, R383, T386, E387, K391, E414, E425, E429, Y432, A433, E436, S447, G448, F450, A451, K452, L454, L455, R456, L457, P458, A459, R461, S462 and L465 on the other.

In a still further embodiment the region is the co-activator/co-repressor binding groove formed by helices H3 and H4 on the surface of BtEcR defined by residues I232, V235, Q236, V239, E240, K243, F248, R253, E254, Q256, I257, L260, K261, S264, S265, M268, S406, F407, L408, E410, I411 and D413.

As will be readily understood by persons skilled in this field the methods of the present invention provide a rational method for designing and selecting compounds which interact with the ecdysone receptor. In the majority of cases these compounds will require further development in order to increase activity. Such further development is routine in this field and will be assisted by the structural information provided in this application. It is intended that in particular embodiments the methods of the present invention includes such further developmental steps.

In another aspect the present invention consists in a method of assessing the interaction between a compound and the BtEcR/BtUSP heterodimer LBD, the method comprising exposing a crystalline composition comprising BtEcR/BtUSP heterodimer LBD or portion thereof or variant of these to the compound and measuring the level of binding of the compound to the crystal.

Accordingly, in another aspect the present invention consists in a method of designing or selecting a compound which modulates ecdysone receptor signalling, the method comprising subjecting a compound obtained by a method according to any one of the previous aspects of the present invention to biological screens and assessing the ability of the compound to modulate ecdysone receptor signalling. These screens employ cloned EcR sequences. In particular they may employ BtEcR nucleic acid sequence (SEQ ID No 1).

Another aspect of the present invention provides a method to guide site-directed mutagenesis of the ecdysone receptor ligand-binding domain to change residues in the ligand-binding domain and at the dimerisation interface in order to change ligand preferences.

For other nuclear receptor LBD's, 3D structural information has been used to identify residues involved in specific contacts with ligands. This information has been employed to guide site-directed mutagenesis of residues leading to directed changes in ligand specificity. For example, on this basis, a single E353Q replacement was made in the human estrogen receptor-α and found to causes a 9-fold reduction in transactivation potency of estradiol and a concomitant 10-140-fold increase in potency of androgens (Ekena et al., 1998).

Homology modelling has been previously employed to predict the structure of ecdysone receptor LBDs complexed with ecdysteroids and dibenzoyl hydrazines. The resultant models have been used to guide site-directed mutagenesis or interpret its outcomes (Kumar et al., 2002; Grebe & Spindler-Barth, 2002). However, the X-ray structure for the B. tabaci ecdysone receptor LBD bound to ponasterone A described in the present invention reveals the previous models to inaccurately reflect important aspects of the LBD protein structures and contacts with ligands. For example, while one single point mutation, A110P, was observed to decrease responsiveness of Choristoneura fumiferana EcR to ecdysteroids without affecting response to non-steroidal ligands RG-102240 and RG-102317 (Kumar et al., 2002), the molecular interpretation for the underlying protein-ligand interactions postulated was incorrect. For example, their model places the alkyl chain of the bound ecdysteroid ligand close to this key residue, whereas our X-ray structure shows that it is the A/B rings of the steroid core that are located close to the corresponding residue in BtEcR (A286). The B. tabaci crystal structure shows that this A286 lies in the deepest (i.e. the s1-s2 β-sheet loop) part of the ligand binding pocket. The line of reasoning advanced by (Kumar et al., 2002) to interpret the insensitivity of the binding of dibenzoyl hydrazines to replacement of this Ala residue with larger residues thus actually suggests that dibenzoyl hydrazine ligands bind

more centrally in the cavity of the ligand binding pocket or closer to its H12 end, rather than occupying the bottom part of the pocket as suggested by these workers.

An understanding of the effects involved based on the X-ray structure of the ecdysone receptor (for B. tabaci) and homology models derived therefrom (for ecdysone receptors from other species) will facilitate future site-directed mutagenesis to achieve desirable changes in ligand selectivity. The actual residues contacting steroid in the binding site and those involved in the dimerisation interface in the X-ray structure are set out in the Results Section below under the sub-heading “Structure description”.

In yet a further aspect, the invention provides a method of utilizing molecular replacement to obtain structural information about a molecule or a molecular complex of unknown structure, comprising the steps of:
(i) crystallising said molecule or molecular complex;
(ii) collecting an X-ray diffraction data set from said crystallized molecule or molecular complex;
(iii) applying at least a portion of the structure coordinates set forth in Appendix I to the X-ray diffraction data set to generate a three-dimensional electron density map of at least a portion of the molecule or molecular complex whose structure is unknown.

The term “molecular replacement” refers to a method that involves generating a preliminary model of an ecdysone receptor crystal whose structure coordinates are unknown, by orienting and positioning a molecule whose structure coordinates are known (e.g. BtEcR/BtUSP LBD heterodimer coordinates from Appendix I) within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal (Lattman, 1985; Rossmann, 1990).

The present inventors have now obtained three dimensional structural information about the ligand-binding domain of the ecdysone receptor which enables a more accurate understanding of how the binding of ligand leads to signal transduction. Such information provides a rational basis for the development of ligands for specific applications, something that heretofore could not have been predicted de novo from available sequence data.

The precise mechanisms underlying the binding of agonists and antagonists to the ecdysone receptor are not fully clarified. However, the binding of good ligands to the receptor site, for example those with a dissociation constant in the order of 10⁻⁸M or lower, is understood to arise from enhanced stereochemical complementarity relative to naturally occurring ecdysone receptor ligands.

Such stereochemical complementarity, pursuant to the present invention, is characteristic of a molecule that matches surface residues the ligand binding pocket of EcR as enumerated by the coordinates set out in Appendix I. By “match” we mean that the identified portions interact with the surface residues, for example, via hydrogen bonding or by non-covalent Van der Waals and Coulomb interactions which promote desolvation of the biologically active compound within the site, in such a way that retention of the biologically active compound within the ligand binding pocket is favoured energetically.

Substances which are complementary to the shape and electrostatics or chemistry of the ligand binding site characterised by amino acids positioned at atomic coordinates set out in Appendix I will be able to bind to the receptor, and when the binding is sufficiently strong, substantially prohibit binding of the naturally occurring ligands to the site. The substance bound to the receptor may also, of its own accord and in the absence of any natural ligand, promote either the agonist or antagonist conformation of the receptor, and thereby determine the biological outcomes effected by the receptor.

It will be appreciated that it is not necessary that the complementarity between ligands and the receptor site extend over all residues lining the pocket in order to modulate binding of the natural ligand.

In general, the design of a molecule possessing stereochemical complementarity can be accomplished by means of techniques that optimise, chemically and/or geometrically, the “fit” between a molecule and a target receptor. Known techniques of this sort are reviewed by (Goodford, 1984; Beddell, 1984; Hol, 1986; Sheridan & Venkataraghavan, 1987; Walters et al., 1998; Verlinde & Hol, 1994; Gane & Dean, 2000; Good, 2001; Langer & Hoffnann, 2001); the respective contents of which are hereby incorporated by reference. See also (Blundell et al., 1987) (drug development based on information regarding receptor structure) and (Loughney et al., 1999) (database mining application on the growth hormone receptor).

There are two preferred approaches to designing a molecule, according to the present invention, that complements the stereochemistry of the ecdysone receptor. The first approach is to dock directly in silico molecules from a three-dimensional structural database to the receptor site, using mostly, but not exclusively, geometric criteria to assess the goodness-of-fit of a particular molecule to the site. In this approach, the number of internal degrees of freedom (and the corresponding local minima in the molecular conformation space) is reduced by considering only the geometric (hard-sphere) interactions of two rigid bodies, where one body (the active site) contains “pockets” or “grooves” that form binding sites for the second body (the complementing molecule, as ligand).

This approach is illustrated by (Kuntz et al., 1982), and (Ewing et al., 2001), the contents of which are hereby incorporated by reference, whose algorithm for ligand design is implemented in a commercial software package, DOCK version 4.0, distributed by the Regents of the University of California and further described in a document, provided by the distributor, which is entitled “Overview of the DOCK program suite” the contents of which are hereby incorporated by reference. Pursuant to the Kuntz algorithm, the shape of the cavity represented by the ecdysone receptor site is defined as a series of overlapping spheres of different radii. One or more extant databases of crystallographic data, such as the Cambridge Structural Database System maintained by Cambridge University (University Chemical Laboratory, Lensfield Road, Cambridge CB2 1EW, UK.), the Protein Data Bank maintained by the Research Collaboratory for Structural Bioinformatics (Rutgers University, N.J., U.S.A.), LeadQuest (Tripos Associates, Inc., St. Louis, Mo.), Available Chemicals Directory (Molecular Design Ltd., San Leandro, Calif.), and the NCI database (National Cancer Institute, U.S.A.) is then searched for molecules which approximate the shape thus defined.

Molecules identified in this way, on the basis of geometric parameters, can then be modified to satisfy criteria associated with chemical complementarity, such as hydrogen bonding, ionic interactions and Van der Waals interactions. Different scoring functions can be employed to rank and select the best molecule from a database. See for example (Bohm & Stahl, 1999). The software package FlexX, marketed by Tripos Associates, Inc. (St. Louis, Mo.) is another program that can be used in this direct docking approach (Rarey et al., 1996).

The second preferred approach entails an assessment of the interaction of respective chemical groups (“probes”) with the active site at sample positions within and around the site, resulting in an array of energy values from which three-dimensional contour surfaces at selected energy levels can be generated. The chemical-probe approach to ligand design is described, for example, by (Goodford, 1984), the contents of which are hereby incorporated by reference, and is implemented in several commercial software packages, such as GRID (product of Molecular Discovery Ltd., West Way House, Elms Parade, Oxford OX2 9LL, U.K.). Pursuant to this approach, the chemical prerequisites for a site-complementing molecule are identified at the outset, by probing the active site with different chemical probes, e.g. water, a methyl group, an amine nitrogen, a carboxyl oxygen, and a hydroxyl. Favoured sites for interaction between the active site and each probe are thus determined, and from the resulting three-dimensional pattern of such sites a putative complementary molecule can be generated. This may be done either by programs that can search three-dimensional databases to identify molecules incorporating desired pharmacophore patterns or by programs which using the favoured sites and probes as input perform de novo design.

Programs suitable for searching three-dimensional databases to identify molecules bearing a desired pharmacophore include MACCS-3D and ISIS/3D (Molecular Design Ltd., San Leandro, Calif.) and Sybyl/3DB Unity (Tripos Associates, Inc., St. Louis, Mo.).

Programs suitable for pharmacophore selection and design include DISCO (Abbott Laboratories, Abbott Park, Ill.) and Catalyst (Accelrys, San Diego, Calif.).

Databases of chemical structures are available from a number of sources including Cambridge Crystallographic Data Centre (Cambridge, U.K.), Molecular Design, Ltd., (San Leandro, Calif.), Tripos Associates, Inc. (St. Louis, Mo.) and Chemical Abstracts Service (Columbus, Ohio).

De novo design programs include Ludi (Biosym Technologies Inc., San Diego, Calif.), LeapFrog (Tripos Associates, Inc.), Aladdin (Daylight Chemical Information Systems, Irvine, Calif.) and LigBuilder (Peking University, China).

Those skilled in the art will recognize that the design of a mimetic may require slight structural alteration or adjustment of a chemical structure designed or identified using the methods of the invention.

The invention may be implemented in hardware or software, or a combination of both. However, preferably, the invention is implemented in computer programs executing on programmable computers each comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer or workstation of conventional design.

Each program is preferably implemented in a high level procedural or object-oriented programming language to communicate with a computer system. However, the programs can be implemented in assembly or machine language, if desired. In any case, the language may be compiled or interpreted language.

Each such computer program is preferably stored on a storage medium or device (e.g. ROM or magnetic diskette) readable by a general or special purpose programmable computer, for configuring and operating the computer when the storage media or device is read by the computer to perform the procedures described herein. The inventive system may also be considered to be implemented as a computer-readable storage medium, configured with a computer program, where the storage medium so configured causes a computer to operate in a specific and predefined manner to perform the functions described herein.

Biological assays to measure the activity of ecdysone receptor agonists and antagonists are well known in the field. Traditional screens for ecdysone receptor agonists examine candidate compounds for an ability to induce the moulting or pupation of whole insect larvae (Becker, 1941; Cymborowski, 1989), the evagination of imaginal discs (Fristrom J. W. & Yund, 1976) or morphological transformation of the Drosophila BII cell line (Clément et al., 1993). More recent assays use mammalian or other eukaryotic cells that have been co-transfected with a recombinant ecdysone receptor and a reporter gene linked to an appropriate response element Both types of screen can also be reformatted to detect non-agonist ligands (antagonists), which can be recognised by their ability to inhibit the activation the receptor by an agonist provided as a standard component of the assay (Yang et al., 1986; Oberdorster et al., 2001)(Oberdorster et al, 2001). In addition, there are in vitro binding assays in which intact insect cells, cell extracts or purified recombinant ecdysone receptors are incubated with a radioactive ecdysone receptor ligand such as ³H-ponasterone A. These assays detect both agonists and antagonists indiscriminately because both types of ligand compete with the radioactive tracer for binding to the ecdysone receptor (Yund et al., 1978; Cherbas et al., 1988). In addition, potential agonists and antagonists may be screened for their ability to inhibit the binding of europium-labelled ecdysone receptor ligands to soluble, recombinant ecdysone receptor in a microplate-based format Europium is a lanthanide fluorophore, the presence of which can be measured using time-resolved fluorometry. The sensitivity of this assay matches that achieved by radioisotopes, measurement is rapid and is performed in a microplate format to allow high-sample throughput, and the approach is gaining wide acceptance as the method of choice in the development of screens for receptor agonists/antagonists (Appell et al., 1998; Inglese et al., 1998). Binding affinity and inhibitor potency may also be measured for candidate inhibitors using biosensor technology.

The three-dimensional structure ligand-binding pocket of the B. tabadecdysone receptor provided in the present application makes it possible to predict, by homology modelling methods, the three-dimensional structure of the ligand-binding pockets of ecdysone receptors from other organisms. For example, the program Modeler (Sali & Blundell, 1993) builds homology models from the satisfaction of spatial restraints derived from the alignment of the target (i.e. an EcR LBD from other species) with the template (which would be three-dimensional structure of the BtEcR LBD this case). Differences in the ligand-binding pockets of different species can thus be modelled.

In a further aspect the present invention provides an isolated nucleic acid molecule comprising a nucleotide sequence which encodes at least the ligand binding domain of BtEcR, wherein the nucleotide sequence is selected from the group consisting of:
(i) a nucleotide sequence comprising a sequence having at least 90% identity to the sequence from nucleotide 535 to nucleotide 1248 of SEQ ID NO: 1 or the complementary nucleotide sequence;
(ii) a nucleotide sequence comprising a sequence that hybridises under high stringency conditions to the sequence from nucleotide 535 to nucleotide 1248 of SEQ ID NO: 1 or the complementary nucleotide sequence; and
(iii) a nucleotide sequence which encodes a polypeptide comprising the sequence from amino acid P179 to amino acid S416 of SEQ ID NO: 2.

In a preferred embodiment the nucleic acid molecule further comprises a nucleotide sequence selected from the group consisting of:
(i) a nucleotide sequence comprising a sequence having at least 90% identity to the sequence from nucleotide 355 to nucleotide 1248 of SEQ ID NO: 1 or the complementary nucleotide sequence;
(ii) a nucleotide sequence comprising a sequence that hybridises under high stringency conditions to the sequence from nucleotide 355 to nucleotide 1248 of SEQ ID NO: 1 or the complementary nucleotide sequence; and
(iii) a nucleotide sequence which encodes a polypeptide comprising the sequence from amino acid R119 to amino acid S416 of SEQ ID NO: 2.

In a further preferred embodiment the nucleic acid molecule comprises a nucleotide sequence selected from the group consisting of:
(i) a nucleotide sequence comprising a sequence having at least 90% identity to SEQ ID NO: 1 or the complementary nucleotide sequence;
(ii) a nucleotide sequence comprising a sequence that hybridises under high stringency conditions to SEQ ID NO: 1 or the complementary nucleotide sequence; and
(iii) a nucleotide sequence which encodes the polypeptide of SEQ ID NO: 2.

In a further preferred embodiment the level of identity is at least 95%, more preferably at least 97% and most preferably at least 99%.

In a further preferred embodiment the nucleic acid molecule comprises the sequence set out in SEQ ID NO. 1 or comprises a nucleotide sequence which encodes the polypeptide of SEQ ID NO. 2.

In determining whether or not two nucleotide sequences fall within these percentage limits, those skilled in the art will be aware that it is necessary to conduct a side-by-side comparison or multiple alignment of sequences. In such comparisons or alignments, differences may arise in the positioning of non-identical residues, depending upon the algorithm used to perform the alignment. In the present context, reference to a percentage identity between two or more nucleotide sequences shall be taken to refer to the number of identical residues between said sequences as determined using any standard algorithm known to those skilled in the art. For example, nucleotide sequences may be aligned and their identity calculated using the BESTFIT programme or other appropriate programme of the Genetics Computer Group, Inc., University Research Park, Madison, Wis., United States of America (Devereux et al., 1984)

The concept of hybridisation under high stringency conditions is a concept well understood in the art. For the purposes of defining the level of stringency as used herein “high stringency” comprises a hybridisation and/or a wash carried out in 0.1×SSC−0.2×SSC buffer, 0.1% (w/v) SDS at a temperature of at least 55° C. Conditions for hybridisations and washes are well understood by one normally skilled in the art. For the purposes of further clarification only, reference to the parameters affecting hybridisation between nucleic acid molecules is found in (Ausubel, 1992), which is herein incorporated by reference.

Throughout this specification, the word “comprise”, or variations such as “comprises” or “comprising” will be understood to imply the inclusion of a stated element, integer or step, or groups of elements, integers or steps, but not the exclusion of any other element, integer or step, or groups of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of the application.

In order that the nature of the present invention may be more dearly understood, preferred forms thereof will now be described with reference to the following non-limiting examples.

### Experimental

### Methods

Cloning and Characterizing the EcR and USP Subunits of the B. tabaci Ecdysone Receptor

Experimental Animals and RNA Isolation

Animals were reared and maintained by the CSIRO Division of Entomology, Canberra. Fourth instar nymphs, collected in our laboratory in Sydney from the under-side of hibiscus leaves, were rapidly subjected to total RNA isolation using the guanidine isothiocynate-CsTFA method (Okayama et al., 1987). mRNA was subsequently purified using the PolyATract mRNA isolation kit (Promega) and quantitated in aqueous ethidium bromide under UV light.

Screening Probe Preparations by PCR

Animals were collected as described above and B. tabaci genomic DNA was isolated according to methods described in (Sambrook et al., 1989). To amplify a homologous B. tabaci EcR screening probe from the genomic DNA, two degenerate primers were employed to obtain a 165 bp product encompassing sequence encoding most of the DNA binding domain (DBD), as described by (Hannan & Hill, 1997). A product of the correct size was obtained, cloned into Bluescript SK + (Stratagene), cycle sequenced (ABI Prism, Perkin-Elmer with gel separation by Australian Genome Research Facility) in both directions and subjected to database analyses by BlastA via the Australian National Genomic Information Service. The information obtained indicated that product encoded the DBD of a steroid nuclear receptor, with highest identity to L. migratoria EcR (Genbank accession number AF049136). Similar efforts, using degenerate primers (Tzertzinis et al., 1994), were unsuccessful in doning a USP screening probe from the B. tabaci genomic DNA. However, the use of library cDNA as a template yielded a product of the correct size (147 bp), and BlastA analysis of the TOPO TA (Invitrogen) cloned product revealed that this fragment had highest identity to the USP/RXR family.

cDNA Library Construction and Screening

A B. tabaci cDNA library was constructed from cDNA that had been oligo-dT primed (Hannan & Hill, 1997) from 5 μg of high quality mRNA and cloned into a Lambda ZapII vector employing a (Stratagene) kit. This primary library, consisting of 1.9×10⁶ plaque forming units (pfu), was amplified once to give a titre of 1.5×10⁹ pfu/ml. Screening for EcR required the plating of 2.5×10⁶ pfu on an E. coli XL1 Blue (Stratagene) lawn and screening for USP required 1.5×10⁶ pfu. Plaques were lifted onto Hybond N (Amersham) membranes, denatured and fixed according to the manufacturer's instructions. Probes were labelled and hybridised as described (Hannan & Hill, 1997). Lambda plaques were converted to pBK-CMV phagemid vector by the excision method (Stratagene) and ORF's were cycle sequenced in both directions using multiple primers and compilation employing the GCG Wisconsin package.

Library screening with the EcR DBD probe identified four pBK-CMV clones, three of which (pBK-CMV4, 6 and 8) were truncated in the LBD at position 1078 bp (methionine is +1). The fourth clone (pBK-CMV7) was identical at the nucleotide level to the first three but had a complete LBD (an extra 173 bp) and a 3′ UTR with polyA tail. In total, pBK-CMV7 contained a 2291 bp cDNA insert with an ORF of 1251 bp encoding a 416 aa protein. BlastN and BlastP analysis of the ORF/putative peptide revealed similarity to ecdysone receptor analogues. Specifically, highest identity was to Locusta migratoria EcR, which was 73% identical at the DNA level and 79% identical at the peptide level. Alignment of the encoded peptide (BtEcR) along with that of other arthropods (data not shown), reveals conservation of the nuclear receptor domains. Specifically, BtEcR exhibits the characteristic five-domain structure (A/B, C, D, E, F) with highest conservation (88% and 48% amino acid sequence identity) observed in the DBD and LBD regions, respectively. Additionally, we observe the conservation of the P and D-boxes, the regions thought to mediate hormone response element (HRE) binding (Zilliacus et al., 1995). We also observed conservation of the AF-2 ligand dependent activation region (Durand et al., 1994) with the invariant Glu410 (numbers are from BtEcR) located in the last helix of the E domain. This Glu along with Lys261 have been suggested to be implicated in salt bridge formation on the basis of homology modelling (Wurtz et al., 2000). The B. tabaci crystal structure does indicate a salt bridge in this vicinity but it actually involves Lys261 and Asp413 (which is also highly conserved).

The library was also screened with the USP DBD probe for the USP cDNA. In this screen, three positive dones were identified and after preliminary data base analysis (BlastN and BlastP) one clone, pBK-CMV21(a), revealed high sequence identity to the USP/RXR receptor family members (WO 01/02436). Of the remaining two clones, one showed no significant identity to lodged sequences and the other corresponded to the Drosophilia Thr3 gene. Thr3 is an orphan nuclear receptor. pBK-CMV21(a) contains a 4.2 kb cDNA insert, cloned in the reverse orientation, with a 1491 bp ORF encoding a 496 aa protein. BlastN and BlastP analysis revealed the ORF and encoded protein had highest similarity to Locusta migratoria RXR, the two species being 62% and 72% identical at the DNA and protein level, respectively. Interestingly, the region corresponding to the USP screening probe does not exactly match done pBK-CMV21(a), the two only being 72% identical at the DNA level (data not shown). Amino acid alignment of the putative peptide (BtUSP) along with USP/RXR from related species (data not shown) revealed the canonical domain structure (A/B, C, D, E/F) and sequence conservation strongest in the DNA binding region. We note that for this region BtUSP retains a perfect P-box and imperfect D-box, the regions implicated in DNA sequence specificity (Danielsen et al., 1989; Umesono & Evans, 1989) and a perfect T-box, a region also thought to direct DNA binding (Chung et al, 1998). Additionally, the ninth heptad repeat of the LBD, a region thought to direct heterodimer formation and the selection of HRE's (Perlmann et al., 1996), is well conserved. Also present is a putative AF-2 site, a region involved in coactivator binding and transactivation (Le Douarin et al., 1995). As with EcR, the highest sequence conservation is observed in the C domain but in contrast to EcR the E/F domain was less conserved.

In-vitro Translation and Gel Retardation Assays

Gene integrity was confirmed by coupled reticulocyte lysate transcription and translation (TNT, Promega). For EcR, transcription/translation was performed with 1.0 μg of done pBK-CMV 7 utilizing the T3 promoter of the vector and ³⁵S methionine incorporation. For USP, however, a 1.7 kb SspI fragment encompassing the ORF was cloned into pBluescript SK (Stratagene) and then transcribed from this vector's T3 promoter. This re-cloning of BtUSP was performed to remove the lengthy (1.3 kb) 5′UTR from the pBK-CMV 21(a) insert. After these reactions, 2 μl of the mix was electrophoresed in an SDS-polyacrylamide gel according to the manufacturer's (Promega) instructions and product was detected by the Molecular Dynamics Phosphorimaging system. As anticipated, the BtEcR recombinant plasmid produced a 50 kDa protein (expected size, 47.5 kDa) and BtUSPplasmid produced a 62 kDa protein (expected size, 55.6 kDa).

DNA binding function was assessed by electrophoretic mobility shift assay (EMSA). For these experiments, EcR and USP proteins were translated as above but using unlabelled methionine. The EcRE probe (hsp27 response element) was prepared by α-³²P labelling 5 pmol of annealed oligo (5′AGCTTCAAGGGTTCAATGCACTTGTCCATCG3′ and 5′AGCTCGATGGACAAGTGCATTGAACCCTTGA3′) with Klenow (GIGAPRIME Labelling Kit, Geneworks). This mix was then phenol/chloroform extracted, ethanol precipitated and resuspended in 100 μl of TE. Binding and electrophoresis were performed as described by (Molloy, 2000). For a 20 μl reaction mix, 2.5 μl of each translated extract was incubated with 8.0 μl of BufferA (20 mM HEPES pH 7.9, 100 mM KCI, 2 mM dithiothreitol (DTT), 1 mM EDTA, 20% (v/v) glycerol), 2 μl of 2% (v/v) NP-40, 1 μl BSA (10 mg/ml), 0.5 μl 2 mg/ml poly (dI-dC).poly(dI-dC), 6 μg single stranded DNA plus or minus 1 μl of 100 mM MgCl₂. After 20 minutes at room temperature, 0.05 pmoles of labelled probe was added and the mix was incubated again for 20 minutes. One sample also had the addition of excess (1.25 pmoles) unlabelled EcRE DNA. 10 μl of the mix was analysed by electrophoresis at 4° C., 80 V in a 0.25×TBE, 5% polyacrylamide gel. After fixing and drying, radioactive species were visualized by the Molecular Dynamics Phosphorimaging system. Once DNA binding conditions had been established, the reactions were repeated (in the presence of 5mM MgCl₂) +/−ponasterone A. As before, probe was added after 20 minutes and electrophoresis was at 40 minutes. In one case, the order of probe and ponasterone A additions were reversed, i.e. probe was included from the beginning and ponasterone A was added after 20 minutes.

The results indicated that BtEcR and BtUSP bind EcRE DNA only as a heterdodimer and not as homodimers. We observed binding to be greatly enhanced in the presence of Mg²⁺. Binding specificity also was confirmed by the by successful competition with unlabelled competitor DNA. The presence of hormone (ponasterone A) clearly enhanced binding of the receptor heterodimer to EcRE when compared to binding without hormone. Again, the binding was specific and the amount of receptor-EcRE complex was reduced by the inclusion of unlabelled EcRE DNA. Adding EcRE 20 minutes before the addition of hormone did not increase the binding of receptor to EcRE.

The nucleotide and amino acid sequence of BtEcR are set out in SEQ ID NO. 1 and SEQ ID NO. 2, respectively.

The conceptually-translated amino add sequence of BtEcR is 416 residues long and displays the five domains typical of a nuclear receptor. The BtUSP protein is 496 residues in length and also displays all domains typical of a nuclear receptor. In functional assays, we demonstrated specific and co-operative binding of the BtEcR and BtUSP subunits to an ecdysone response element and showed that this phenomenon was enhanced by the addition of an ecdysteroid ligand, ponasterone A.

Construction of a Baculovirus for Co-expression of the Ligand-binding Domains of BtEcR and BtUSP

Step 1: Cloning pFastBacDual metHis₆EcR

pBK-CMV7 was digested with HaeIII and PstI to excise a 1.3 kb DNA fragment (Fragment A) which encodes the BtEcR D and E domains.

Two oligonucleotides were synthesised (1) ncoI metHis₆ upper (CATGGGTATGAGAGGATCGCATCACCATCACCATCACAGG) and (2) ncoI metHis₆ lower (CCTGTGATGGTGATGGTGATGCGATCCTCTCATACC) treated with kinase and annealed to construct a DNA duplex (Linker A) which encodes a hexahistidine tag at the amino terminus of the BtEcR D domain.

pFastbac Dual (Invitrogen) was digested with NcoI and NsiI and treated with phosphatase by standard methods (Sambrook et al., 1989).

Fragment A and Linker A were ligated into the NcoI and NsiA treated pFastBacDual to construct pFastbac metHis₆ EcR.

Step 2: Cloning pFastBacDual His₆EcR FLAG USP

pBK-CMV21(a) was used as template in a PCR (TdIDNA polymerase, Promega) with primers (1) avaIIusp5 (TGTCTCGCTATGGGACCGAAAAGAGAAGCC) and (2) pstusp3 (GATAATGCTGCAGATGGTGATAATT) to produce a 1370 bp DNA fragment (Fragment B) encoding the BtUSP D and E domains. A PsfI site exists in the 3′UTR of BtUSP but a 5′ AvaII site is introduced by primer avaIIusp5.

Two oligonucleotides were synthesised (1) BssHuspFLAGupper (CGCGCTTAACTATGGACTACAAGGACGACGATGACAAGG) and (2) avauspFLAGlower (GGTCCCTTGTCATCGTCGTCCTTGTAGTCCATAGTTAAG) treated with kinase and annealed to construct a DNA duplex (Linker B) which encodes a FLAG tag at the amino terminus of the BtUSP D domain.

pFastbac metHis₆EcR was digested with BssHI(PauI) and PstI. Fragment B and Linker B were ligated into the BssHI(PauI) and PstI treated pFastBacDual to construct pFastbac His₆EcR FLAG USP.

Step 3. Transposition From pFastbac His₆EcR FLAG USP Into a Bacmid and Baculovirus Construction.

The mini-Tn7 expression cassette in the donor plasmid pFastbac His₆EcR FLAG USP was transposed into a baculovirus genome by transformation into DH10Bac competent cells and selection of white colonies. White colonies were colony purified and grown up in liquid culture. Mini-preparations of Bacmid DNA were made using a alkaline lysis procedure in which attention was payed to minimisation of shear forces. The resultant DNA was monitored for the presence of high molecular weight bacmid DNA by electophoresis through a 0.5% agarose gel.

Mid-log phase Sf9 insect cells were transfected with bacmid DNA using Cellfectin (Invitrogen) and standard procedures and grown for 72 hours at 27° C. Virus was harvested from the culture supernatant and titrated by plaque assay.

Expression and Purification of Recombinant Heterodimeric EcR-USP Ligand-binding Domain

Pilot-scale expression of recombinant heterodimeric BtEcR-BtUSP LBD was achieved by infection of suspension cultures of Sf9, Sf21 and or Hi-5 insect cells in spinner flasks or Schott bottles on a shaker platform maintained at 27° C. Insect cells infected with the virus engineered to express BtEcR/BtUSP ligand-binding domain were shown by gel electrophoresis to contain the expressed polypeptides corresponding to the two tagged domains. The recombinant cell lysates had a greatly enhanced ability to bind the radiolabelled ecdysteroid, [³H]-ponasterone A, compared to control cell lysates. These results indicated that the recombinant virus was expressing functional LBDs that were able to heterodimerise and form a recombinant B. tabaci receptor LBD that bound ecdysteroids with high affinity. Equilibrium binding studies with [₃H]-ponasterone A as ligand gave (by direct curve fitting) a K_{d} value of 1.21±0.17 nM.

Large-scale recombinant protein production was carried out in a Celligen (New Brunswick Scientific) stirred bioreactor under controlled conditions (27° C., 35 r.p.m.). Successful 5-6 L cultures yielded 70-100 g wet cells, which typically contained about 0.2 mg recombinant LBD protein per gram cells. Heterodimer could be affinity-purified from cell extracts by using a nickel chelate resin to capture the His₆-tag of the recombinant EcR ligand-binding domain. Further purification could be achieved by subjecting the affinity-purified material to ion exchange chromatography (Pharmacia Mono-Q) or gel filtration (Pharmacia Superdex-200). All three chromatography steps were efficient (>60% yield) and inexpensive. Yields were estimated from measurements of protein concentration and from binding of [³H]-ponasterone A. Identity, integrity and purity were monitored by SDS-polyacrylamide gel electrophoresis (PAGE; Coomassie-stained or immunoblotted), non-denaturing PAGE, non-denaturing isoelectric focussing gels, and mass spectrometry.

To purify the recombinant heterodimeric LBD (with bound ecdysteroid ligand) for crystallization trials, 60-70 g recombinant cells were lysed by sonication in the presence of excess ligand (ponasterone A) and the receptor LBD-ligand complex was purified from the clarified lysate using affinity purification followed by at least one other chromatography step (see above). In the absence of reducing conditions, such as prevails in conventional crystallisation trials, disulphide bonds rapidly form within and/or between some of the recombinant LBD molecules. Fortunately, the undesirable disulphide-mediated oligomerisation could be suppressed by using thiol-specific reagents (iodoacetic acid or iodoacetamide) to modify the surface-accessible cysteine residues. The chemical modification was preferably done between the first and second chromatography steps. However, mass spectrometry suggested that such modification was introducing chemical microheterogeneity into the recombinant proteins. A way was therefore found to conduct crystallisation trials under reducing conditions in a nitrogen atmosphere (see below), which obviated the need for chemical modification. Amplified recombinant baculovirus engineered to express the heterodimeric B. tabaci ligand-binding domain, prepared as described above, was used to infect a 5-litre culture of Hi-5 insect cells in the a Celligen Bioreactor with a multiplicity of infection of approximately 1. Harvested at 49 h post-infection, this culture yielded 65 g wet weight of recombinant insect cells, which were snap-frozen in liquid nitrogen and stored at −70° C. The entire batch of cells was later thawed and suspended in 130 ml HEPES buffer containing sufficient ponasterone A to saturate the anticipated number of ligand-binding sites (100 mM HEPES, 40 mM KCI, 10% glycerol, 1 M EDTA, 3 mM sodium azide, 52 μM ponasterone A, 8.9 μM leupeptin, 2.7 μM pepstatin, 1.3 mM phenylmethanesulphonyl fluoride, 26 mM Na₂S₂O₅, 13 mM 2-mercaptoethanol, pH 7.0, 4° C.) and sonicated to break open the cells (4 batches of equal volume, each treated with 13×5 sec pulses, with 25 sec cooling in salted ice between each pulse, on a MSE Type 11 74.MK2 sonicator fitted with a 19 mm diameter probe). The sonicates were recombined (210 ml total volume) and the ionic strength was then raised by addition of 20.8 ml 4M KCI. This sample was ultracentrifuged to pellet cellular debris (Beckman 60Ti rotor in Beckman L8-80M Ultracentrifuge: 100 000 g, 2 h, 4° C.). The supernatant was dialysed (Spectrum Spectra/Por 1 tubing, 40 cm long×5 cm diameter) for 3 h at 4° C. against 1100 ml HEPES buffer (25 mM HEPES, 40 mM KCI, 10% glycerol, 1 mM EDTA, 3 mM sodium azide, 10 mM 2-mercaptoethanol, 0.1 μM ponasterone A, pH 7.0) to lower the ionic strength. The dialysate (which had become cloudy) was clarified by centrifugation (Beckman JA14 rotor in Beckman J2-21 centrifuge, 12 000 rpm, 30 min, 4° C.). The pH was found to have dropped below pH 7, so 20 ml 0.5M HEPES pH 7.0 was added dropwise with stirring (on ice) to elevate it before snap-freezing the sample in liquid nitrogen and storing it at −70° C. To resume the purification, the sample was thawed rapidly (by shaking in a 37° C. water bath) and dialysed (Spectrum Spectra/Por 1 tubing, 40 cm long×5 cm diameter) twice for 3 h at 4° C. against 1100 ml phosphate buffer (50 mM sodium phosphate, 10% glycerol, 0.3M NaCl, 10 mM mercaptoethanol, 0.1 μM ponasterone A, 3 mM sodium azide, pH 7.4). The dialysate (200 ml total) was then snap-frozen in liquid nitrogen and stored at −70° C.

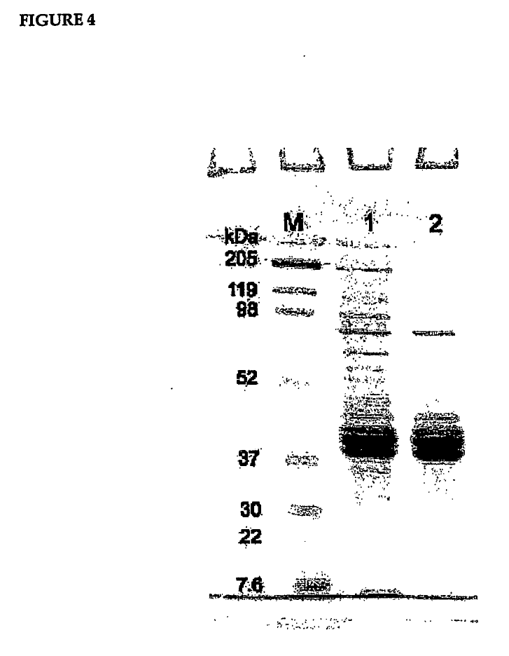

In the immobilized metal-ion affinity chromatography (IMAC) step, Ni-NTA-agarose was used to capture the recombinant heterodimer by way of the His₆-tag on the BtEcR LBD. Capture, wash and elution were performed in the presence of 2-mercaptoethanol and ponasterone A, as follows. The frozen dialysate was thawed rapidly (by shaking in a 37° C. water bath) and re-clarified (Beckman JA14 rotor in Beckman J2-21 centrifuge, 12 000 rpm, 20 min, 4° C.). To the clarified protein sample was added 2 ml 2M imidazole, pH 7.4, containing 3 mM sodium azide. A 12 ml portion of a 50% slurry of Ni-NTA agarose beads (Qiagen, Cat. 30210) was washed twice with 20 ml phosphate buffer (50 mM sodium phosphate, 10% glycerol, 0.3M NaCl, 10 nM 2-mercaptoethanol, 3 mM sodium azide, pH 7.4). The washed beads were combined with the protein sample and the suspension was rotated slowly (RotoTorque: 10 rpm, 3 h, 4° C.). The beads were then pelleted by centrifugation (Beckman JA14 rotor in Beckman J2-21 centrifuge, 10 000 rpm, 20 min, 4° C.). The supernatant was removed carefully, after which the beads were transferred to a mini-column (a 20 ml syringe body clamped upright, with a disc of Whatman filter-paper serving as a frit at the base) at 4° C. Unbound proteins were removed by washing the column of beads with 120 ml phosphate buffer (50 mM sodium phosphate, 10% glycerol, 0.3M NaCl, 10 mM 2-mercaptoethanol, 20 mM imidazole, 0.5 μM ponasterone A, 3 mM sodium azide, pH 7.4) at 4° C. Specifically-bound proteins were eluted with a buffer containing a high imidazole concentration (50 mM sodium phosphate, 10% glycerol, 0.3M NaCl, 10 mM 2-mercaptoethanol, 250 mM imidazole, 3 μM ponasterone A, 3 mM sodium azide, pH 7.4). To maximise recovery, the elution buffer was applied to the column as 2×4.5 ml aliquots with a 20 min interval between each application. The eluates were combined and a portion was assayed for protein content (Pierce Coomassie Plus assay, calibrated using bovine serum albumin). The IMAC step yielded a total of 41 mg of purified receptor. This procedure typically yields a preparation in which the recombinant EcR and USP LBDs are present in approximately equal amounts. An analysis of the IMAC eluate by reducing SDS-PAGE is shown (FIG. 4, lane 1). The MAC eluate was snap-frozen in liquid nitrogen and stored at −70° C.

To resume the purification, the IMAC eluate was thawed rapidly by shaking in a 37° C. water bath. Since we had evidence that the non-denaturing detergent 3-[(3-cholamidopropyl)dimethylammonio]-1-propanesulphonate (CHAPS) could maximise the extent of high-affinity receptor-ecdysteroid binding, the IMAC eluate was dialysed (Spectrum Spectra/Por 1 tubing, 150 mm long×15 mm diam.) twice for 3 h at 4° C. against 500 ml CHAPS-containing Tris buffer (50 mM Tris, 230 mM NaCl, 10% glycerol, 10 mM 2-mercaptoethanol, 0.5 μM ponasterone A, 2 mM CHAPS, 3 mM sodium azide, pH 7.5). Following this, any additional ligand-binding capacity was satisfied by incubating the sample overnight at 4° C. in the presence of CHAPS and a large excess of ponasterone A; this was done by transferring the dialysis bag to a 100 ml graduated cylinder containing 100 ml 50 mM Tris, 230 mM NaCl, 10% glycerol, 10 mM 2-mercaptoethanol, 61 μM ponasterone A, 2 mM CHAPS, 3 mM sodium azide, pH 7.5, and dialysing overnight at 4° C. However, since it was also feared that CHAPS might interfere with crystallisation, this additive was removed by a subsequent dialysis step into a CHAPS-free Tris buffer, and CHAPS was omitted from later stages of the purification. (It was expected that any improvements in ligand-binding stoichiometry would persist after the removal of the CHAPS so long as free ponasterone A was maintained at saturating concentrations). Thus, CHAPS was removed from the sample by dialysing it (Spectrum Spectra/Por 1 tubing, 150 mm long×15 mm diam) twice for 3 h at 4° C. against 1000 ml 50 mM Tris, 230 mM NaCl, 10% glycerol, 2 mM dithiothreitol, 0.5μM ponasterone A, 3 mM sodium azide, pH 7.5. The dialysate was supplemented to a final concentration of 3 μM ponasterone A, snap-frozen in liquid nitrogen, and stored at −70° C. To resume the purification, the sample was thawed rapidly by shaking in a 37° C. water bath. The heterodimer sample was then concentrated by ultrafiltration (Pall MicroSep-10, spun in Beckman JA-20 rotor in Beckman J2-21 centrifuge, 7500 rpm, 4° C.) until the volume of retentate was about 0.7 ml. The retentate was then supplemented with 0.1 ml fresh 16 mM dithiothreitol solution and incubated on ice, 2 h, to ensure the reduction of any disulphide bonds that might have formed during the concentration step. The sample (38 mg protein) was then split into two aliquots (so as not to overload the column) and each aliquot was purified identically by high-performance gel filtration chromatography (Pharmacia Superdex-200 HR 10/30 column, equilibrated at room temperature in 50 mM Tris, 230 mM NaCl, 10% glycerol, 2 mM dithiothreitol, 1 μM ponasterone A, 3 mM sodium azide, pH 7.5, flow rate 0.5 ml/min). The UV absorbance of the column eluate (monitored at 280 nm) indicated that a significant amount of material with molecular masses above that expected for the recombinant heterodimer complex was resolved by the column in each case. In each case, the absorbance peak for the recombinant heterodimer itself was sharp and symmetrical, and the eluate fractions (from both column runs) that corresponded to this dominant peak were pooled to provide a single sample of purified heterodimer for further processing. The pooled eluate was concentrated by ultrafiltration (Pall NanoSep-10, spun in Sigma 1K15 minifuge, 14 000 g, 4° C.). The retentate was retrieved, combined with washings of the ultrafiltration membrane, and supplemented to a final concentration of 3 μM ponasterone A. The concentrated sample was sterilized by spin-filtration (Costar Spin-X 0.22 μm cellulose acetate filter) and stored at 4° C. under nitrogen. At this stage, the recombinant heterodimer sample contained 13.2 mg protein in 0.33 ml buffer (50 mM Tris, 230 mM NaCl, 10% glycerol, 2 mM dithiothreitol, 3 μM ponasterone A, 3 mM sodium azide, pH 7.5). Analysis by SDS-PAGE confirmed the presence of the purified recombinant BtEcR and BtUSP LBDs, (FIG. 4, lane 2). Their gene-predicted molecular masses are 35.8 and 30 kDa, respectively, but we find that recombinant LBDs from the ecdysone receptors of many insects typically run more slowly than expected on SDS-PAGE.

Samples of the purified receptor complex were tested in crystallisation trials, as described below.

Crystallisation

Crystals of the BtEcR/BtUSP heterodimer ligand-binding domain were grown using the hanging drop vapour diffusion method (McPherson, 1982). The well solution contained 0.1M sodium HEPES (pH 7.5), 1.0 M ammonium dihydrogen phosphate, 4.5% trehalose and 10 mM dithiothreitol, while the drop solution contained 1 μl of protein (40 mg/ml) in 50 mM Tris HCI (pH 7.5), 0.23 M sodium chloride, 10% glycerol, 10 mM dithiothreitol, 3 mM sodium azide, and 3 μM ponasterone A, mixed with 1 μl of well solution. Crystals were also found to grow in an alternate well solution containing 0.1M Citrate (pH 5.2), 7-8.5% PEG 3350, 67 mM KH₂PO₄ and 10 nM TCEP HCI (Tris(2-carboxyethyl)phosphine hydrochloride). The drops were set up under a nitrogen atmosphere and the plates stored at room temperature (20° C.) in a nitrogen incubator. Crystals appeared after 3 months and had a maximum dimension of 0.5 mm.

Data Collection

Crystals were transferred to a solution containing 0.1M sodium HEPES (pH 7.5), 1.0 M ammonium dihydrogen phosphate, 4.5% trehalose, 10 mM dithiothreitol and 30% glycerol, mounted in a cryoloop (Teng, 1990) and frozen in a stream of nitrogen gas at −160° C. X-ray diffraction data from the crystal were then collected on a MacSdence X-ray generator equipped with focusing mirrors, a helium path and a Rigaku R-Axis IV detector. Data processing was conducted using the HKL suite of software (Otwinowski & Minor, 1997). Data statistics are presented in Table 1. The crystal had unit cell dimensions 143.01 Å×143.01 Å×84.01 Å and belonged either to space group P4₁2₁2 or P4₃2₁2.

Homology Modelling

A homology model of the BtEcR/BtUSP ligand-binding domains heterodimer was constructed using as the template, the crystal structure of the heterodimeric complex between the ligand-binding domains of human RAR-α and mouse RXR-α (RCSB id: 1DKF). The A-chain of the structure (mRXR-α) was the structural template for USP while the B-chain (hRAR-α) was the template for EcR. The fold recognition module of the program ProCeryon (ProCeryon Biosciences GmbH, Salzburg, Austria) was used to thread the respective sequences on to the structural templates, and these alignments, after some manual adjustments, were used as the input to the program Modeller (Sali & Blundell, 1993) as implemented within InsightII v. 98.0 (Accelrys, Inc., San Diego, USA) to generate several three-dimensional models of the target protein complex. The model with the lowest objective function value was chosen as the best model, and its quality was checked with the programs Profiles-3D (Lüthy et al., 1992), ProsaII (Sippl, 1993) and ProCheck (Laskowski et al., 1993). It should be noted that in this model helix H12 of both EcR and of USP was in the antagonist conformation, i.e. lying in the groove between helices H3 and H4 of the respective LBD's (Renaud & Moras, 2000).

Structure Determination

Structure solution proceeded via molecular replacement using the program MOLREP (Vagin & Teplyakov, 1997) within the CCP4 software suite (Collaborative Computing Project No. 4, 1994). Molecular replacement employed all data to a resolution of 4.0 Å within the above homology model as the search structure. The correct solution exhibited a correlation coefficient of 0.319, convincingly above the next highest value of 0.278. The space group was verified to be P43212 and the solution demonstrated viable crystal packing of the heterodimer model. Crystallographic refinement then proceeded via simulated annealing within X-PLOR (Brünger, 1992) which reduced the crystallographic R-factor to 0.331 (Rfree=0.441), confirming that the molecular replacement solution was substantially correct. Iterative rounds of model building using O (Jones et al., 1991) and crystallographic refinement using CNS (Brünger et al., 1998) yielded a model encompassing residues P179 to V415 of BtEcR and V300 to S492 of BtUSP (employing the single-letter amino acid code for naming residues here and throughout). Electron density that could readily be interpreted as the ligand ponasterone A, was visible in the anticipated site within the BtEcR LBD (Renaud & Moras, 2000). The non-planarity of the four-ring moiety allowed unambiguous assignment of ligand orientation and position. Details of the final refinement statistics are presented in Table 2. Stereochemical analysis of the structure showed that only BtEcR residue I180 and BtUSP residue T363 lay in the disallowed regions of the Ramachandran plot. Electron density associated with these residues was poor and their backbone conformation could not be modelled accurately. However, neither of these residues lay in the vicinity of the ponasterone A binding site and the accuracy of their conformation was thus highly unlikely to be of any consequence to the structural details and implications of the ponasterone A binding site. Also included in the model are three phosphate ions, presumably arising from the solution used for crystallization of the heterodimer.

The crystallographic R-factors in Table 2 suggested that the structure was essentially correct to the resolution determined (viz. 3.07 Å). The observed absence of residues N-terminal of BtEcR P179 and N-terminal of BtUSP V300 could be due to their being totally disordered in the crystal or due to their prior removal via contaminating proteases, or both. Analysis of SDS PAGE gels of crystals (run under reducing conditions) indicated the presence of bands at 31 kDa, 26 kDa, 23 kDa and 22 kDa, all of these being smaller than the apparent molecular weights of the freshly purified LBDs (FIG. 4, lane 2) and also smaller than the gene-predicted molecular masses of the intact ligand-binding domains (35.8 and 30 kDa). We thus conclude that partial proteolysis may have contributed in whole or in part to the absence of these residues.

### RESULTS

Structure Description

The fold of the BtEcR LBD is that of a canonical nuclear hormone receptor (FIG. 1). The secondary structure elements of BtUSP/BtEcR LBD discerned in this structure are located within the BTECR sequence as follows: helix H1—residues 182 to 198, helix H2—residues 202 to 211, helix H3—residues 220 to 244, helix H4—residues 252 to 264, helix H5—residues 267 to 275, strand s0—residues 275 to 277, strand s1—residues 282 to 285, strand s2—residues 288 to 291, helix H6—residues 292 to 300, helix H7—residues 304 to 319, helix H8—residues 321 to 334, helix H9—residues 342 to 364, helix H10—residues 368 to 400 and helix H12—residues 405 to 413. Thus it comprises α-helices H1 to H10 and H12, and β-strands s1 and s2 located between helices H5 and H6. An additional short β-strand (labelled here as s0) lies between helix H5 and strand s1.

Helix H12 in BtEcR is observed in the so-called agonist conformation (Renaud & Moras, 2000). The structure of BtEcR was compared with those available for other nuclear receptors. The closest structural neighbour was the retinoic add receptor (RAR). The root-mean-square deviation of 206 (out of 237) corresponding backbone C^{α} atoms between the BtEcR structure and that of RAR-γ2 (RCSB id: 1EXA, in the agonist conformation) is 1.29 Å. The major difference between these structures lies in the conformation of the loop between helices H1 and H3. In RAR this loop has a random coil conformation and lies across the outer surface of the s1-s2 β-sheet loop. In EcR the segment contains an intact helix H2 which packs anti-parallel on the N-terminal portion of helix H3 and interacts with the opposite surface of the s1-s2 β-sheet loop.

The ligand ponasterone A was observed to lie in a totally-enclosed pocket formed by residues F194, Q195, N196, Y198, E199, H200, P201, H226, I227, T228, I230, T231, L233, T234, L237, I238, F241, S242, V267, M268, M269, F270, R271, M272, R274, R275, I283, L284, F285, A286, Y296, M301, T304, L308, Y325, A326, T329, I333, M389, N390, T393, C394, L397, V404, P405, L408 and W412 (FIG. 2). The pocket has a “J-shaped” architecture, with the major part (the leg of the “J”) accommodating the ligand, plus an ancillary part (the curved tail of the “J”) existing as an extension of the major part via a narrow channel. The inner wall of the channel linking the major and ancillary parts of the pocket is formed by the side chain of residue R271. The accessible volume of the entire cavity is approximately 766 Å³, whilst the volume of the ponasterone A itself is 434 Å³, both figures calculated using VOIDOO (Kleywegt & Jones, 1994). The ancillary cavity appears unoccupied in the structure presented here. The narrowness of channel connecting the major and ancillary parts of the pocket suggests that it in some dynamic states of the protein these two parts may become disjoint rather than forming a single topological entity.

Potential hydrogen bonds between individual protein atoms and ligand are as follows: A286 N to the ponasterone A hydroxyl at C-6, T234 O^{γ1} to the ponasterone A hydroxyl at C-14, T231 O^{γ1} to the ponasterone A hydroxyl at C-14, R271 NH1 to the ponasterone A hydroxyl at C-2, E199 O to the ponasterone A hydroxyl at C-2, E199 O to the ponasterone A hydroxyl at C-3, Y296 OH to the ponasterone A hydroxyl at C-20 (FIG. 2). The remainder of the contacts between ligands and protein are overwhelmingly hydrophobic in nature and formed by contacts between the side chains of residues P201, I227, T228, I230, M268, R271, M272, R275, I283, F285, A286, M301 and W412 and the ligand. The hydrogen bond between the side-chain of Y296 and the C-20 hydroxyl of ponasterone A probably explains the importance for high-affinity binding of having a C-20 hydroxyl group in the ecdysteroid. The Tyr at position 296 (of BtEcR) is completely conserved across insect orders, suggesting that this hydrogen bond may be a general feature of high-affinity ecdysteroid binding by EcR. The significance of this interaction was not apparent from earlier homology models of EcR (Wurtz et al., 2000; Kasuya et al., 2003).

Helix H12 was observed to lie in the so-called agonistic conformation (Renaud & Moras, 2000) possibly locking the ligand into the site via the side chain of W412 which hangs into the ligand-binding site. A salt bridge between BtEcR residues D413 and K261 appears to stabilize the C-terminus of H12. In this conformation a co-activator can bind to a site that includes H12 and the surface of the hydrophobic cleft between helices H3 and H4. The molecular detail of this cleft is presented in FIG. 3. Side chains forming the deft and its immediate surrounds include those of residues V235, Q236, V239, E240, K243, F248, R253, Q256, I257, L260, K261, S264, S265 and M268. This groove is totally conserved across all ecdysone receptor sequences displayed in Table 5, apart from the residue R253. This residue lies at the distal end of the binding groove (with respect to the position of H12 shown in this structure) and it is unclear at this stage whether or not its side chain interacts with the co-repressor or co-activator upon binding of these elements.

The structure of the BtUSP protein closely resembles that of other published USP structures (Billas et al., 2001; Clayton et al., 2001) but with the following major difference. The secondary structure elements of BtUSP/BtEcR LBD discerned in this structure are located within the BtUSP sequence as follows: helix H3—residues 301 to 321, helix H4—residues 328 to 339, helix H5—residues 340 to 353, strand s1 residues 359 to 361, strand s2—residues 365 to 367, helix H6—residues 371 to 376, helix H7—residues 380 to 396, helix H8—residues 399 to 411, helix H9—residues 420 to 443, helix H10—residues 448 to 466 and helix H12—residues 481 to 491. No electron density was visible for residues prior to V300, i.e. helix H1, and part of the loop connecting H1 to H3 are totally unobserved. Part of the volume occupied by these structural elements in other USP structures is now occupied by the H10-H12 loop. H12 lies in the so-called antagonistic conformation (Renaud & Moras, 2000). The helix H11 appears not to be formed. No ligand was observed in the site corresponding to that occupied by phospholipid in the two above published structures, and indeed part of that binding site is now occluded by a repositioning of the H10-H12 loop, and by a repositioning of helix H6 and residues immediately adjacent to this element (residues 371 to 384). The repositioning of the H10-H12 loop likely arises from the absence of residues prior to H3 in our structure, allowing this element to collapse into the region normally occupied by the H1-H3 loop in the intact USP ligand-binding domains. Part of the movement of the H10-H12 loop may be caused by the involvement of that loop in a crystal contact with a neighbouring molecule in our structure.

The dimeric association between BtEcR and BtUSP ligand-binding domains resembles that of the corresponding RAR-RXR complex. These two heterodimeric structures can be overlaid with an root-mean-square deviation of 1.37 Å for 339 matched Cα atoms. The interface is formed by EcR residues contained in H9, H10 and the loop between H8 and H9 on one hand and USP residues contained in H7, H9, H10 and the loop between H6 and H7 on the other (see Table 5). Residues involved in the interface include BtEcR residues H314, M315, I331, S335, E336, R337, P338, E347, Q350, E351, I354, E355, K358, T370, T371, F373, A374, K375, L377, S378, L380, T381, E382, R384, T385 and N388 on one hand and BtUSP residues E342, R383, T386, E387, K391, E414, E425, E429, Y432, A433, E436, S447, G448, F450, A451, K452, L454, L455, R456, L457, P458, A459, R461, S462 and L465 on the other. The interface was estimated by computing all residues with any atom's van der Waals surface within 1.4 Å of that of any atom of the opposite chain followed by visual inspection.

Potential inter-chain salt bridges include those from USP E429 to EcR K375, USP K391 to EcR E336, USP K391 to EcR E347, USP K452 to EcR E351 and USP E425 to EcR K375. Out of these, only the salt bridge between EcR E347 and USP K391 is conserved across all species (although the Dipteran Chiromus tentans EcR has Asp instead of Glu at the position corresponding to residue 347 in BtEcR), and compounds which bind to the interface and disrupt a particular salt bridge could be the basis of specific antagonists.

Hydrogen bonds occur between the side chains of USP S447 and the side chain of EcR E355A, between the backbone carbonyl of USP S447 and the side chain of EcR K358 and between the side chains of EcR R384 and USP S462. The remainder of the contacts are hydrophobic in nature. A single phosphate ion is included in the interface, coordinated by the side chains of the EcR residue R384, the carbonyl oxygen of EcR residue E336 and the side chains of USP residues R383, E387 and R456.

PASS (Brady & Stouten, 2000) shows the existence of a pocket on the BtEcR surface on the edge of the heterodimeric interface bounded by residues including A262, S265, E266, R337, R384, G387, N388 and S391 of BtEcR. PASS also shows the existence of a pocket on the BtUSP surface on the edge of the heterodimeric interface bounded by residues including K337, S338, N341, E342, K416, G464, L465, C467 and H470 of BtUSP.

Designing Species-specific Agonists in the EcR Pocket

Table 3 presents the inter-order variation apparent across a variety of Insecta EcR LBDs for those residues that line the ecdysteroid binding pocket observed in the B. tabaci structure. Analysis of Table 3 indicates that there are differences in the residues in the ligand binding pocket of EcRs between insect species. For example, in the hemipteran B. tabaci (resistant to the bisacylhydrazine compounds) residue 272 is methionine, whereas in lepidopteran species (susceptible to bisacylhydrazines) the residue at this position is a smaller valine. Attention has also been drawn to the potential importance of the residue at this position in relation to the control spectrum of bisacylhydrazine insecticides in the communication by Billas et al. (2003) reporting the crystal structure of the lepidopteran Heliothis virescens EcR/USP heterodimeric LBD. It is apparent from Table 3 of the present application that the methionine at position 272 is present in the Hemiptera, Diptera, Orthoptera and Coleoptera while the residue in this position of the Lepidoptera is valine. The Lepidoptera, Diptera and Coleoptera have been shown to be susceptible to bisacylhydrazines in varying degrees generally correlating with the binding affinities of their ecdysone receptors for the agonists (Dhadialla et al, 1998). Furthermore our laboratory has carried out in vitro binding studies employing purified recombinant LBDs to demonstrate significant affinity of a dipteran (Lucilia cuprina) receptor and only very low affinity of the whitefly (B. tabaci) receptor for RH5992 (unpublished results). Clearly the response to bisacylhydrazines is not simply dependent on the residue at the position corresponding to 272 in B. tabaci.

We propose that the methionine residue at position 272 in B. tabaci does not act as a single determinant but that it has a synergistic effect with leucine 308 and methionine 389, and that the collective length, bulk and charge state of these side chains may lead to changes in the shape and affinity of the binding pocket for various agonists/antagonists. A methionine at position 389 is only found in the Hemiptera and Arachnida. Using this triplet of residues as an example, and assuming the binding pocket remains essentially the same in gross topography, the overall reduction in side-chain bulk at residues 272, 308 and 389 in the lepidopteran EcR creates an additional bulge in the lepidopteran pocket helping to accommodate the bisacylhydrazines.

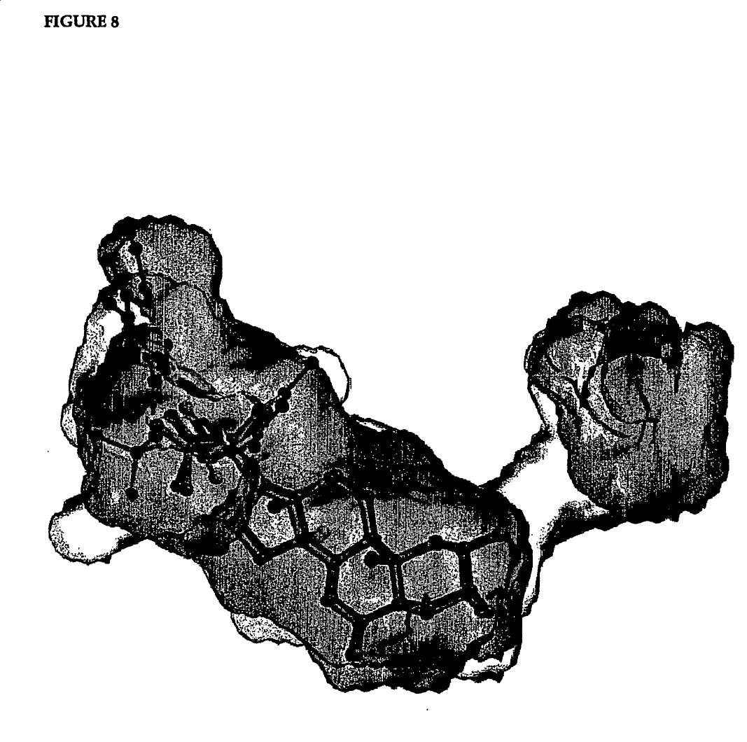

A comparison of the ecdysteroid binding pockets of the hemipteran BtEcR and lepidopteran HvEcR shows that the triptych of residues discussed in the previous paragraph is largely responsible for differences in the pocket shape near the unoccupied region adjacent to C22-OH of ponasterone A in BtEcR. In HvEcR this unoccupied region is extended into a pronounced bulge in the ecdysteroid bound pocket (see distinct bulge in the HvEcR pocket at top left of FIG. 8). Least squares alignments of the protein backbone C-alpha atoms of the EcR domains of all three structures, BtEcR (ponasterone A bound) HvEcR 1R1K (ponasterone A bound) and HvEcR 1R20 (synthetic agonist BYI06830 bound) places the A and B rings of the agonist BYI06830 in the vicinity of, but not enclosed by, this extra bulge in the HvEcR 1R1K ecdysteroid bound pocket. In the lepidopteran HvEcR 1R20 structure this bulge is further extended, probably by induced fit, to accommodate the synthetic agonist. We propose that in the hemipteran BtEcR structure, the absence of the bulge in this region of the potential binding pocket conformation would prevent initial binding of many of the bisacylhydrazines and subsequent expansion of the bulge by an induced-fit mechanism.

Clearly other changes in binding site residues which occur between orders, as detailed in Table 3, would alter the topography of the binding site, allowing for taxon-specific design of steroids or small molecule mimetics, which exploit these differences. Such design would be implemented using tools available to those skilled in the art as described above. M389 is found towards the C-terminus of H10/11 and the pocket opening that is closed by H12 on agonist binding. M389 makes minimal contact with the ponasterone A ligand; however, mutation of this residue to a smaller side chain such as valine, found in the Lepidoptera, or glycine as found in the Arachnida, could weaken the interaction between H11 and H7. This weakening appears to open up the binding site towards the C-terminus of H10/11 revealing the conserved L308 and highly conserved L386 as forming a hydrophobic indentation, potentially capable of accommodating ligand antagonist/agonists with bulky substituents such as a t-butyl group or even a benzene ring as found in some of the bisacylhydrazines.

The X-ray structure provides a precise description of the relative positions in three-dimensional space of the residues lining the binding pocket of BtEcR. The ecdysteroid ligand, ponasterone A, fits snugly into the major part of the binding pocket, with almost all receptor-free volume over the rigid steroid framework being occupied (FIG. 2).

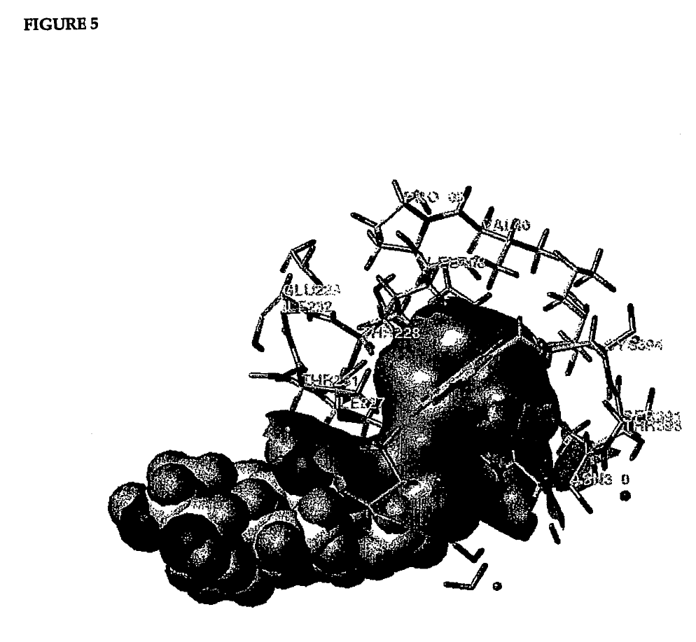

However, certain sites of extension are available as follows. There is a small pocket near the C20/C21 region of the ecdysteroid which is not fully occupied (as described two paragraphs above), and a larger volume beyond the terminus of the steroid alkyl chain which is also unfilled. There is significant receptor pocket volume not occupied by the ligand below and at the terminus of the alkyl chain. This larger, partially filled region is bounded by L408, V404, N390, C394, L408, I227, T228, T231, T393 and P405 as FIG. 5 shows. The region denoted as the ancillary part of the binding pocket (the curved part of the ‘J’) may also be available for occupation by ligands, depending on the accessibility of this pocket

Clearly the BtEcR LBD X-ray structure could be used, together with molecular modelling methods well known to those skilled in the art, to design modifications of the steroid which better fill the receptor volume.

Alternatively, synthetic organic molecules could be designed by taking account of the properties of the residues lining the binding site, and using methods such as GRID (Goodford, 1984) to locate regions favourable for binding of particular substituents. Such substituents could be linked together by a scaffold or other molecular framework to present the ligand binding groups in optimum three-dimensional orientation to interact with complementary binding groups in the binding site. This can be done manually by a person skilled in the art, or in an automated fashion using programs such as LeapFrog (Tripos Associates, Inc., St. Louis, Mo.).

Another alternative would be that the three-dimensional orientation of complementary binding groups in the protein (derived from knowledge of the X-ray structure of the receptor) could be used as a pharmacophore query for database searching. This would identify molecules with correctly oriented functional groups which would be putative ligands for the receptor.

Another alternative would be using the shape and properties of the binding site obtained from the X-ray structure of the receptor as a database query directly. Programs such as DOCK (Ewing et al., 2001; Kuntz et al., 1982) and FlexX (Rarey et al., 1996) can use this type of information to search through databases of real or hypothetical molecules to find ones with the correct properties to bind to the receptor.

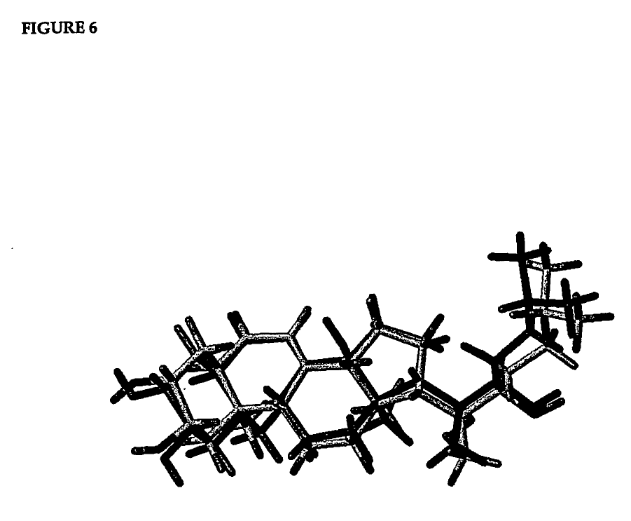

An example of how this can be done uses the program FlexX to dock known and putative ligands into the binding site of the EcR. The receptor structure was pre-processed to add all hydrogen atoms to the amino acids, and charges were applied using standard rules. A region within 6.5 Å of the ponasterone A ligand bound into the site was used for the FlexX calculations. To assess that the program was able to use the X-ray data to correctly dock ligands, the ponasterone A ligand was extracted from the X-ray structure, energy minimized and re-docked into the binding site. The FlexX program docked the ponasterone A ligand into a binding pose essentially identical to that in the X-ray structure (RMS 0.79 Å) with a very favourable docking score (−23.92). The quality of the docking results can be seen in FIG. 6.

In another docking experiment with FlexX, the score for the ponasterone A ligand was −28.4. In the same experiment a number of other potent EcR steroidal ligands gave the following scores:- muristerone A −27.6, 20-hydroxyecdysone −29.0, inokosterone −31.7. The highest ranked poses bound to the EcR in a similar mode to that of ponasterone A, and they exhibited similar binding scores to that computed for ponasterone A.

As a further example, several small synthetic molecules were docked into the EcR X-ray structure using FlexX. These were: bisacylhydrazines I and RH5992 (which show negligible binding in BtEcR competitive binding assay with [³H]-ponasterone A as tracer); an oxadiazole derivative II (also negligible binding); an oxazolidinone derivative III (weak-moderate binding in assay); thiotetrahydroimidazole derivative IV (weak-moderate binding in assay).

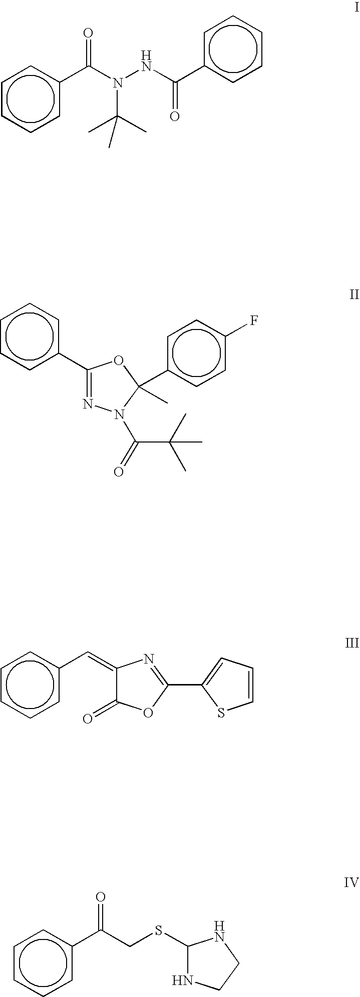

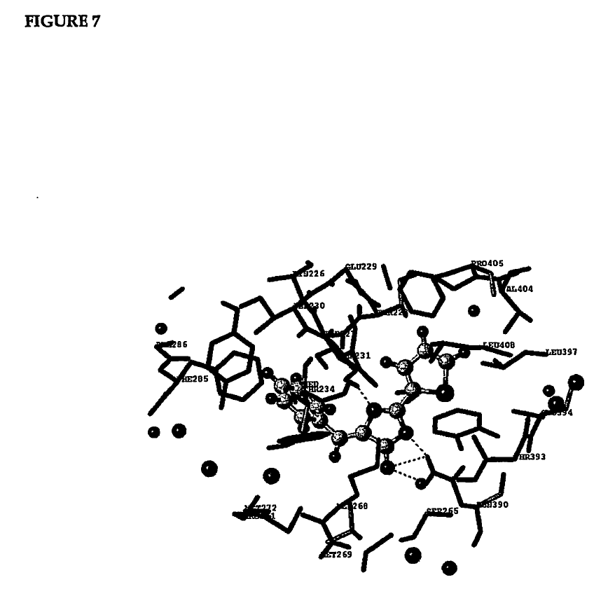

FlexX docking calculations were unable to find any binding poses in the BtEcR pocket which scored well with relatively low internal energy for the bisacylhydrazines (I and RH5992) and the oxadiazole derivative (II). However the two weak-moderate binding ligands, III and IV, were successfully docked into the EcR X-ray structure with relatively low internal strain and favourable docking scores (−14.9 and −15.8 respectively). Both of these compounds had FlexX binding poses which oriented their structures over the C/D rings of ponasterone A X-ray structure, and the alkyl chain of the steroid. An example of a successful docking pose for one of these small, synthetic ligands, the oxazolidinone derivative III, is given in FIG. 7.

Designing Compounds that Target the BtEcR/BtUSP Interface and Alter the Quaternary Association of these Molecules.

In a further aspect of this invention compounds (non-peptidic, peptidic or peptidomimetic) can be designed that mimic the USP component of the heterodimer interface. Details of residues forming this interface and their variation across orders are given in Table 4. Such compounds may bind to the EcR monomer and prevent the formation of a functional EcR USP heterodimer. Such design would utilize the conformational detail of the EcR/USP interface revealed in this application. Such design would also utilize the detail of the ligand binding interactions to identify ligand derivatization sites that could be used to disrupt the conformations and hence the interactions of the EcR helices involved in dimerization. Similarly compounds can be designed that mimic the EcR component of the heterodimer interface and so bind to the USP component, again preventing formation of the functional EcR/USP heterodimer.

Design of such compounds is feasible for the estrogen receptor, see for example (Yudt & Koide, 2001). Rational interface peptide design has been demonstrated in a variety of other protein-protein interactions (Singh et al., 2001; Berezov et al., 2002).In addition, compounds can be designed/selected so as to bind into either of the two ‘pockets’ associated with the interface and form the basis of platforms to create steric hindrance to the process of heterodimerization and thus inhibit the function of the ecdysone receptor.

Designing Compounds that Target the BtEcR Co-activator/co-repressor Binding Cleft

In a further aspect of this invention compounds can be designed based on the BtEcR structure to target the co-activator/co-repressor binding cleft, and thereby be capable of acting as agents that modulate transactivation (Tran et al., 2001; Westin et al., 1998). This site is formed by two antiparallel helices, H3 and H4 and presents a groove into which the co-activator or co-repressor would bind. Co-activators have a conserved LXXLL motif (the “NR” box) which has been shown in studies of other nuclear receptors to form part of an amphipathic helix which interacts with the H3/H4 cleft via the leucines. Also involved in this interaction are the highly conserved glutamate in H12 and lysine in H3. On this basis it becomes possible to those skilled in the art to design peptides or peptidominmetics that mimic the binding of the co-activator NR box to the deft, utilizing the conformational detail of the EcR H3/H4 cleft presented here. Such compounds would have the potential to modulate the transactivational state of the receptor. This groove is substantially conserved across all known EcR sequences (Table 5).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### TABLES

TABLE 1
X-ray data collection statistics

| | |
|---|---|
| No. frames | 302 |
| Oscillation angle (°) | 0.5 |
| No. measurements | 191046 |
| No. reflections | 16725 |
| Multiplicity | 11.8 (10.5)¹ |
| Resolution range (Å) | 30.0-3.07 |
| Completeness (%) | 99.9 (100.0) |
| <I/σ(I)> | 19.3 (5.6) |

¹Numbers in parenthesis refer to the statistic in the highest resolution shell.

TABLE 2
Crystallographic refinement statistics

| | |
|---|---|
| Resolution range (Å) | 100-3.07 |
| Total no. of reflections used | 16756 |
| Crystallographic R-factor | 0.203 |
| Free R-factor (5% of total reflections) | 0.275 |
| No. of protein atoms | 3475 |
| No. of ligand + solvent atoms | 53 |
| Root-mean-square deviation of bond | 0.012 |
| lengths from ideality (Å) | |
| Root-mean-square deviation of bond | 1.56 |
| angles from ideality (°) | |

TABLE 3
Residues lining the ponasterone A binding pocket in BtEcR and their
inter-order variation across the sequences

| | lining atoms | Hemiptera | Diptera | Lepidoptera | Orthoptera | Coleoptera | Arachnida | Crustacea |
|---|---|---|---|---|---|---|---|---|
| F194 | 2M | F | Y | Y | F | F | Y | Y |
| Q195* | 4M, 5S | Q | Q | Q | Q | Q | Q | Q |
| N196 | 1M | N, D | D | D, E | N | N | Q | E |
| Y198 | 4M, 1S | Y | Y | Y | Y | Y | F | F |
| E199 | 4M, 5S | E | E | E, D | E | E | E | E |
| H200 | 3M, 4S | H, A | Q | Q | S | H | S | Q |
| P201* | 2M, 3S | P | P | P | P | P | P | P |
| H226 | 2M | H, I | H, Y | Q | H | H | H | H |
| I227* | 4M, 4S | I | I | I | I | I | I | I |
| T228* | 4M, 3S | T | T | T | T | T | T | T |
| I230 | 4M, 4S | I, M | I, V | M | I | I | M | I |
| T231* | 2M, 3S | T | T | T | T | T | T | T |
| L233* | 3M, 3S | L | L | L | L | L | L | L |
| T234* | 3M, 3S | T | T | T | T | T | T | T |
| L237* | 2M, 3S | L | L | L | L | L | L | L |
| I238* | 4M, 4S | I | I | I | I | I | I | I |
| F241* | 2M, 7S | F | F | F | F | F | F | F |
| S242 | 2M, 2S | S, A | A | A | A | A | S | S |
| V267 | 3M, 3S | V, A | V | V | V | V | V | V |
| M268* | 3M, 3S | M | M | M | M | M | M | M |
| M269* | 2M, 4S | M | M | M | M | M | M | M |
| F270 | 3M, 3S | F | L | L | F | F | L | L |
| R271* | 4M, 7S | R | R | R | R | R | R | R |
| M272 | 4M, 4S | M, V | M | V | M | M | G | A |
| R274* | 2M, 6S | R | R | R | R | R | R | R |
| R275 | 1M, 6S | R, K | R | R | R | R | K | R |
| I283 | 1M, 4S | I | I | V | I | I | I | I |
| L284 | 4M | L, V | F | L, M | L | L | V | V |
| F285* | 4M, 7S | F | F | F | F | F | F | F |
| A286 | 2M, 1S | A | A | A | A | V | A | G |
| Y296* | 5S | Y | Y | Y | Y | Y | Y | Y |
| M301 | 4S | M, L | M, V | M, F | M | M | V | L |
| T304 | 1M, 3S | T, A | N, T | V | T | T | S | S |
| L308 | 4S | L, Q | L | L | M | L | L | L |
| Y325 | 1M, 4S | Y | Y | Y, F | Y | Y | Y | Y |
| A326* | 1M, 1S | A | A | A | A | A | A | A |
| T329 | 3S | T | T | T | T | T | T | A |
| I333* | 2S | I | I | I | I | I | I | I |
| M389 | 2M, 4S | M, E | Q K | Q | Q | Q | M | I |
| N390* | 4M 4S | N | N | N | N | N | N | N |
| T393 | 3M, 3S | T, L | M | M | M | M | M | M |
| C394* | 2M, 2S | C | C | C | C | C | C | C |
| L397* | 2S | L | L | L | L | L | L | L |
| V404 | 1M, 3S | V | L | L | L | L | L | L |
| P405* | 1M, 2S | P | P | P | P | P | P | P |
| L408* | 3S | L | L | L | L | L | L | L |
| W412* | 4S | W | W | W | W | W | W | W |

“*” indicates total conservation across all sequences considered.
The column headed “lining atoms” indicates the number of side chain atoms (S) and the number of main chain atoms (M) involved in forming the cavity wall.
Underlined residues are those judged to form the major part of the ponasterone A binding cavity, the remainder forming the walls of the ancillary part of the ponasterone A binding cavity.

TABLE 4
Residues forming the BtEcR/BtUSP LBD interface and their
inter-order variation across the sequences
(a) BtEcR residues

| | Hemiptera | Diptera | Lepidoptera | Orthoptera | Coleoptera | Arachnida | Crustacea |
|---|---|---|---|---|---|---|---|
| H314 | H, F | Q | C | Q | T | K | S |
| M315 | M | M | M | M | M | M | L |
| I331 | I | I | I, V | I | I | I | I |
| S335 | S | S | S | S | S | S | S |
| E336 | E, S | D | D | E | E | E | E |
| R337 | R | R | R | R | R | R | R |
| P338 | P | P | P | P | P | P | P |
| E347 | E | E | E | E | E | E | E |
| Q350 | Q | Q | Q | Q | Q | Q | Q |
| E351 | E | S | R | E | E | E | E |
| I354 | I, L | T, I | L | L | L | I | L |
| E355 | E | D | N | E | E | E | E |
| K358 | K | K, R | R | K | R | R | K |
| T370 | T | S, L | S, A, P | G | G | K | N |
| T371 | T, V | V | V | T | T | N | M |
| F373 | F, Y | F, Y | F, Y | F | F | F | F |
| A374 | A | A | G, A | A | A | A | A |
| K375 | K, R | K | K, R | K | K | R | K |
| L377 | L | L | L | L | L | L | L |
| S378 | S | G, S | G, S | S | S | S | N |
| L380 | L | L | L | L | L | L | L |
| T381 | T | T | S, T | T | T | T | T |
| E382 | E | E | E | E | E | E | E |
| R384 | R | R | R | R | R | R | R |
| T385 | T | T | T, S | T | T | T | T |
| N388 | N | N | M, T | N | N | N | N |

(b) BtUSP residues

| | Hemiptera | Diptera | Lepidoptera | Orthoptera | Hymenoptera | Coleoptera |
|---|---|---|---|---|---|---|
| E342 | E | E | E | E | E | E |
| R383 | R | R | R | R | R | R |
| T386 | T | S, C | S | T | S | S |
| E387 | E | E | E | E | E | E |
| K391 | K | K | K | K | K | K |
| E414 | E, G | D | D | E | E | T |
| E425 | E, Q | E, D | D, E, V | E | T | E |
| E429 | E, D | E, S | E | E | E | E |
| Y432 | Y | Y | Y, F | Y | Y | Y |
| A433 | A, V | A | L, S | A | G | G |
| E436 | E | D | D | E | E | E |
| S447 | S, P | D | E | P | A | P |
| G448 | G | G | G | G | G | G |
| F450 | F | F | F | F | F | F |
| A451 | A | A | A | A | A | A |
| K452 | K | Q | A, S | K | K | K |
| L454 | L | L | L | L | L | L |
| L455 | L | L | L | L | L | L |
| R456 | R | R | R | R | R | R |
| L457 | L | L | L | L | L | L |
| P458 | P | P | P | P | P | P |
| A459 | A, S | S | S | A, S | S | S |
| R461 | R | R | R | R | R | R |
| S462 | S | S | S | S | S | S |
| L465 | L | L | L | L | L | L |

TABLE 5
Residues forming the BtEcR LBD co-activator/co-repressor binding groove and their
inter-order variation across the sequences.

| | Hemiptera | Diptera | Lepidoptera | Orthoptera | Coleoptera | Arachnida | Crustacea |
|---|---|---|---|---|---|---|---|
| I232 | I | I | I | I | I | I | I |
| V235 | V | V | V | V | V | V | V |
| Q236 | Q | Q | Q | Q | Q | Q | Q |
| V239 | V | V | V | V | V | V | V |
| E240 | E | E | E | E | E | E | E |
| K243 | K | K | K | K | K | K | K |
| F248 | F | F | F | F | F | F | F |
| R253 | R | Q | Q | R | Q | R | R |
| E254 | E | E | P, S | E | E | E | E |
| Q256 | Q | Q | Q | Q | Q | Q | Q |
| I257 | I | I | I | I | I | I | I |
| L260 | L | L | L | L | L | L | L |
| K261 | K | K | K | K | K | K | K |
| S264 | S | S | S | S | S | S | S |
| S265 | S | S | S | S | S | S | S |
| M268 | M | M | M | M | M | M | M |
| S406* | S, P | R, K | P | P | P | P | P |
| F407* | F | F | F | F | F | F | F |
| L408* | L | L | L | L | L | L | L |
| E410* | E | E | E | E | E | E | E |
| I411* | I | V, I | I | I | I | I | I |
| D413* | D | D | D | D | D | D | D |

The * identifies residues in H12 that would be expected to interact with co-activators but their involvement in co-repressor interactions is unknown.

### REFERENCES

1. Appell, K. C., Chung, T. D. Y., Solly, K. J. & Chelsky, D. (1998). Biological characterization of neurokinin antagonists discovered through screening of a combinatorial library. J. Biomol. Screen. 3, 19-27.
2. Ashburner, M., Chihara, C., Meltzer, P. & Richards, G. (1974). Temporal control of puffing activity in polytene chromosomes. Cold Spring Harb. Symp. Quant Biol. 38, 655-62.
3. Ausubel, F. M., eds. (1992). Current protocols in molecular biology Short protocols in molecular biology: a compendium of methods from Current protocols in molecular biology/edited by Fredezick M Ausubel . . . [et al.]., 2nd ed. edit., Greene Pub. Associates; Wiley, Brooklyn, N.Y. New York, N.Y.
4. Becker, E. (1941). Uber Versuche zur Anreicherung und physiologische Charakterisierung und Wirkstoffes der Puparisierung. Biol. Zbl. 61, 360-388.
5. Beddell, C. R. (1984). Designing drugs to a fit a macromolecular receptor. Chem. Soc. Rev. 13, 279-314.
6. Berezov, A., Chen, J., Liu, Q., Zhang, H. T., Greene, M. I. & Murali, R. (2002). Disabling receptor ensembles with rationally designed interface peptidomimetics. J. Biol. Chem. 277, 28330-9.
7. Bernstein, F. C., Koetzle, T. F., Williams, G. J., Meyer, E. E. Jr, Brice, M. D., Rodgers, J. R., Kennard, O., Shimanouchi, T. & Tasumi, M. (1977). The Protein Data Bank: a computer-based archival file for macromolecular structures. J. Mol. Biol. 112, 535-542.
8. Billas, I. M., Moulinier, L., Rochel, N. & Moras, D. (2001). Crystal structure of the ligand-binding domain of the ultraspiracle protein USP, the ortholog of retinoid X receptors in insects. J. Biol Chem. 276, 7465-74.
9. Billas, I. M., Iwema, T., Garnier, J. M., Mitschler, A., Rochel, N., and Moras, D. (2003). “Structural adaptability in the ligand-binding pocket of the ecdysone hormone receptor.” Nature, 426(6962), 91-96.
10. Blundell, T. L., Sibanda, B. L., Stemberg, M. J. & Thornton, J. M. (1987). Knowledge-based prediction of protein structures and the design of novel molecules. Nature 326, 347-352.
11. Bohm, H. J. & Stahl, M. (1999). Rapid empirical scoring functions in virtual screening applications. Med. Chem. Res. 9, 445-462.
12. Brady, G. P. Jr & Stouten, P. F. (2000). Fast prediction and visualization of protein binding pockets with PASS. J. Comput. Aided Mol. Des. 14, 383-401.
13. Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T. & Warren, G. L. (1998). Crystallography & NMR system: A new software suite for macromolecular structure determination. Acta Crystallogr. D 54, 905-921.
14. Brunger, A. T. (1992). X-PLOR, Version 3.1: A system for X-ray and NMR. Yale University Press, New Haven.
15. Cherbas, P., Cherbas, L., Lee, S. S. & Nakanishi, K. (1988). 26-[125I]iodoponasterone A is a potent ecdysone and a sensitive radioligand for ecdysone receptors. Proc. Natl. Acad. Sci. USA 85, 2096-100.
16. Chung, A. C., Durica, D. S., Clifton, S. W., Roe, B. A. & Hopkins, P. M. (1998). Cloning of crustacean ecdysteroid receptor and retinoid-X receptor gene homologs and elevation of retinoid-X receptor mRNA by retinoic acid. Mol Cell Endocrinol 139, 209-27.
17. Clayton, G. M., Peak-Chew, S. Y., Evans, R. M. & Schwabe, J. W. (2001). The structure of the ultraspirade ligand-binding domain reveals a nuclear receptor locked in an inactive conformation. Proc. Natl. Acad. Sci. USA 98, 1549-1554.
18. Clément, C. Y., Bradbrook, D. A., Lafont, R. & Dinan, L. (1993). Assessment of a microplate-based bioassay for the detection of ecdysteroid-like or antiecdysteroid activities. Insect Biochem. Mol. Biol. 23, 187-192.
19. Collaborative Computing Project No. 4. (1994). The CCP4 suite: programs for protein crystallography. Acta Crystallogr. D 50, 760-763.
20. Connolly, M. L. (1983). Solvent-accessible surfaces of proteins and nucleic acids. Science 221, 709-713.
21. Cymborowski, B. (1989). Bioassays for ecdysteroids. In Ecdysone: from Chemistry to Mode of Action (J. Koolman, ed.), pp. 144-149, Thieme Medical Publishers, New York.
22. Danielsen, M., Hinck, L. & Ringold, G. M. (1989). Two amino acids within the knuckle of the first zinc finger specify DNA response element activation by the glucocorticoid receptor. Cell 57, 1131-1138.
23. Devereux, J., Haeberli, P. & Smithies, O. (1984). A comprehensive set of sequence analysis programs for the VAX. Nucleic Acids Res. 12, 387-395.
24. Dhadialla, T. S., Carlsor, G. R. & Le, D. P. (1998). New insecticides with ecdysteroidal and juvenile hormone activity. Annu. Rev. Entomol. 43, 545-69.
25. Dinan, L., Bourne, P., Whiting, P., Tsiteksi, A., Saatov, Z., Dhadialla, T. S., Hormann, R. E., Lafont, R. & Coll, J. (2002). Synthesis and biological activities of turkesterone 11α-acyl derivatives. J. Insect Sci. 3:6.
26. Durand, B., Saunders, M., Gaudon, C., Roy, B., Losson, R. & Chambon, P. (1994).

Activation function 2 (AF-2) of retinoic acid receptor and 9-cis retinoic acid receptor: presence of a conserved autonomous constitutive activating domain and influence of the nature of the response element on AF-2 activity. EMBO J. 13, 5370-5382.
27. Ekena, K., Katzenellenbogen, J. A. & Katzenellenbogen, B. S. (1998). Determinants of ligand specificity of estrogen receptor-alpha: estrogen versus androgen discrimination. j Biol. Chem. 273, 693-699.
28. Ewing, T. J., Makino, S., Skillman, A. G. & Kuntz, I. D. (2001). DOCK 4.0: search strategies for automated molecular docking of flexible molecule databases. J. Comput. Aided Mol. Des. 15, 411-428.
29. Fristrom J. W. & Yund, M. A. (1976). Characteristics of the action of ecdysones on Drosophila imaginal discs cultured in vitro. In Invertebrate Tissue Culture Research Applications (K Maramorosch (Ed.), ed.), pp. 161-178, Academic Press, New York.
30. Gane, P. J. & Dean, P. M. (2000). Recent advances in structure-based rational drug design. Curr. Opin. Struct. Biol. 10, 401-404.
31. Good, A. (2001). Structure-based virtual screening protocols. Curr. Opin. Drug Discov. Devel. 4, 301-307.
32. Goodford, P. J. (1984). Drug design by the method of receptor fit. J. Med. Chem. 27, 558-564.
33. Grebe, M. & Spindler-Barth, M. (2002). Expression of ecdysteroid receptor and ultraspirade from Chironomus tentans (Insecta, Diptera) in E. coli and purification in a functional state. Insect Biochem. Mol. Biol. 32, 167-74.
34. Hannan, G. N. & Hill, R. J. (1997). Cloning and characterization of LcEcR: a functional ecdysone receptor from the sheep blowfly Lucilia cuprina. Insect Biochem. Mol. Biol. 27, 479-88.
35. Hannan, G. N. & Hill, R. J. (2001). LcUSP, an ultraspiracle gene from the sheep blowfly, Lucilia cuprina: cDNA doning, developmental expression of RNA and confirmation of function. Insect Biochem. Mol. Biol. 31, 771-81.
36. Hill, R. J., Segraves, W. A., Choi, D., Underwood, P. A. & Macavoy, E. (1993). The reaction with polytene chromosomes of antibodies raised against Drosophila E75A protein. Insect Biochem Mol Biol 23, 99-104.
37. Hol, W. G. J. (1986). Protein crystallography and computer graphics—on the path to systematic drug design. Angewandte Chemie 98, 765-777.
38. Inglese, J., Samama, P., Patel, S., Burbaum, J., Stroke, I. L. & Appell, K. C. (1998). Chemokine receptor-ligand interactions measured using time-resolved fluorescence. Biochemistry 37, 2372-2377.
39. Jones, G. & Sharp, P. A. (1997). Ultraspiracle: an invertebrate nuclear receptor for juvenile hormones. Proc. Natl. Acad. Sci. USA 94, 13499-503.
40. Jones, T. A., Zou, J.-Y., Cowan, S. W. & Kjeldgaard, M. (1991). Improved methods for building protein models in electron density maps and the location of errors in these models. Acta Crystallogr. A 47, 110-119.
41. Kasuya, A., Sawada, Y., Tsukamoto, Y., Tanaka, K., Toya, T. & Yanagi, M. (2003).

Binding mode of ecdysone agonists to the receptor: comparative modeling and docking studies. J. Mol. Model (Online) 9, 58-65.
42. Kleywegt, G. J. & Jones, T. A. (1994). Detection, delineation, measurement and display of cavities in macromolecular structures. Acta Crystallogr. D 50, 178-185.
43. Koelle, M. R., Talbot, W. S., Segraves, W. A., Bender, M. T., Cherbas, P. & Hogness, D. S. (1991). The Drosophila EcR gene encodes an ecdysone receptor, a new member of the steroid receptor superfamily. Cell 67, 59-77.
44. Kumar, M. B., Fujimoto, T., Potter, D. W., Deng, Q. & Palli, S. R. (2002). A single point mutation in ecdysone receptor leads to increased ligand specificity: Implications for gene switch applications. Proc. Natl. Acad. Sci. USA 99, 14710-14715.
45. Kuntz, I. D., Blaney, J. M., Oatley, S. J., Langridge, R. & Ferrin, T. E. (1982). A geometric approach to macromolecule-ligand interactions. J. Mol. Biol. 161, 269-288.
46. Langer, T. & Hoffiann, R. D. (2001). Virtual Screening: An Effective Tool for Lead Structure Discovery? Currrent Pharmaceutical Design 7, 509-527.
47. Laskowski, R. A., MacArthur, M. W., Moss, D. S. & Thornton, J. M. (1993). PROCHECK: a program to check the stereochemical quality of protein structures. J. Appl Crystallogr. 26, 283-291.
48. Lattman, E. (1985). Diffraction methods for biological macromolecules. Use of the rotation and translation functions. Methods Enzymol. 115, 55-77.
49. Le Douarin, B., Zechel, C., Garnier, J. M., Lutz, Y., Tora, L., Pierrat, P., Heery, D., Gronemeyer, H., Chambon, P. & Losson, R. (1995). The N-terminal part of TIF1, a putative mediator of the ligand- dependent activation function (AF-2) of nuclear receptors, is fused to B-raf in the oncogenic protein T18. EMBO J. 14, 2020-2033.
50. Loughney, D. A., Murray, W. V. & Jolliffe, L. K. (1999). Application of virtual screening tols to a protein-protein interaction: database mining studies on the growth hormone receptor. Med. Chem. Res. 9, 579-591.
51. Lüthy, R., Bowie, J. U. & Eisenberg, D. (1992). Assessment of protein models with three-dimensional profiles. Nature 356, 83-85.
52. McPherson, A. (1982). Preparation and analysis of protein crystals. Wiley, New York.
53. Molloy, P. L. (2000). Electrophoretic mobility shift assays. Methods Mol. Biol. 130, 235-246.
54. Nienaber, V. L., Richardson, P. L., Klighofer, V., Bouska, J. J., Giranda, V. L. & Greer, J. (2000). Discovering novel ligands for macromolecules using X-ray crystallographic screening. Nat Biotechnol. 18, 1105-1108.
55. Oberdorster, E., Clay, M. A., Cottam, D. M., Wilmot, F. A., McLachlan, J. A. & Milner, M. J. (2001). Common phytochemicals are ecdysteroid agonists and antagonists: a possible evolutionary link between vertebrate and invertebrate steroid hormones. J. Steroid Biochem. Mol. Biol. 77, 229-238.
56. Okayama, H., Kawaichi, M., Brownstein M., Lee, F., Yokota, T. & Arai, K. (1987). High-efficiency cloning of full-length cDNA; construction and screening of cDNA expression libraries for mammalian cells. Methods Enzymol. 154,3-28.
57. Oro, A. E., McKeown, M. & Evans, R. M. (1990). Relationship between the product of the Drosophila ultraspiracle locus and the vertebrate retinoid X receptor. Nature 347, 298-301.
58. Otwinowski, Z. & Minor, W. (1997). Processing of X-ray diffraction data collected in oscillation mode. Methods Enzymol. 276,307-326.
59. Perlmann, T., Umesono, K., Rangarajan, P. N., Forman, B. M. & Evans, R. M. (1996). Two distinct dimerization interfaces differentially modulate target gene specificity of nuclear hormone receptors. Mol. Endocrinol. 10, 958-966.
60. Rarey, M., Kramer, B., Lengauer, T. & Klebe, G. (1996). A fast flexible docking method using an incremental construction algorithm. J. Mol. Biol. 261, 470-489.
61. Renaud, J. P. & Moras, D. (2000). Structural studies on nuclear receptors. Cell Mol. Life Sci. 57, 1748-1769.
62. Rossmann, M. G. (1990). The molecular replacement method. Acta Cyrstallogr. A 46, 73-82.
63. Sali, A. & Blundell, T. L. (1993). Comparative protein modelling by satisfaction of spatial restraints. J. Mol. Biol. 234, 779-815.
64. Sambrook, J., Fritsch, E. F. & Maniatis, T. (1989). Molecular Cloning (2nd ed.). Cold Spring Harbor Laboratory Press, Cold Spring Harbor.
65. Sasorith, S., Billas, I. M. L., Iwema, T., Moras, D. & Wurtz, J. M. (2002). Structure-based analysis of the ultraspiracle protein and docling studies of putative ligands. J. Insect Sci. 2, 1-11.
66. Sheridan, R. P. & Venkataraghavan, R. (1987). New methods in computer-aided drug design. Acc. Chem. Res. 20, 322-329.
67. Singh, S. K., Maithal, K., Balaram, H. & Balaram, P. (2001). Synthetic peptides as inactivators of multimeric enzymes: inhibition of Plasmodium falciparum triosephosphate isomerase by interface peptides. FEBS Lett. 501, 19-23.
68. Sippl, M. J. (1993). Recognition of errors in three-dimensional structures of proteins. Proteins 17, 355-362.
69. Sundaram, M., Palli, S. R., Krell, P. J., Sohi, S. S., Dhadialla, T. S. & Retnakaran, A. (1998). Basis for selective action of a synthetic molting hormone agonist, RH-5992 on lepidopteran insects. Insect Biochem. Mol. Biol. 28, 693-704.
70. Teng, T. Y. (1990). Mounting of crystal for macromolecular crystallography in a free-standing thin film. J. Appl. Crystallogr. 23, 387-391.
71. Tran, H. T., Shaaban, S., Askari, H. B., Walfish, P. G., Raikhel, A. S. & Butt, T. R. (2001). Requirement of co-factors for the ligand-mediated activity of the insect ecdysteroid receptor in yeast. J. Mol. Endocrinol. 27, 191-209.
72. Tzertzinis, G., Malecki, A. & Kafatos, F. C. (1994). BmCF1, a Bombyx mori RXR-type receptor related to the Drosophila ultraspiracle. J. Mol. Biol. 238, 479-486.
73. Umesono, K. & Evans, R. M. (1989). Determinants of target gene specificity for steroid/thyroid hormone receptors. Cell 157, 1139-1146.
74. Vagin, A. & Teplyakov, A. (1997). MOLREP: an automated program for molecular replacement. J. Appl. Cryst. 30, 1022-1025.
75. Verlinde, C. L. & Hol, W. G. (1994). Structure-based drug design: progress, results and challenges. Structure 2, 577-57.
76. Walters, W. P., Stahl, M. T. & Murcko, M. A. (1998). Virtual screening—an overview. Drug Discovery Today 3, 160-178.
77. Westin, S., Kurokawa, R., Nolte, R. T., Wisely, G. B., McInerney, E. M., Rose, D. W., Milburn, M. V., Rosenfeld, M. G. & Glass, C. K. (1998). Interactions controlling the assembly of nuclear-receptor heterodimers and co-activators. Nature 395, 199-202.
78. Williams, C. D. (1967). The juvenile hormone II. Its role in the endocrine control of molting, pupation, and adult development of the cecropia silkworm. Biol. Bull. Woods Hole 121, 572-585.
79. Williams, C. M. (1967). Third-generation pesticides. Sci. Am. 217, 13-7.
80. Wing, K. D. (1988). RH5849, a nonsteroidal ecdysone agonist: effects on a Drosophila cell line. Science 241, 467-9.
81. Wing, K. D., Slawecki, R. A. & Carlson, G. R. (1988). RH5849, a nonsteroidal ecdysone agonist, effects on larval lepidoptera. Science 241, 470-472.
82. Wurtz, J. M., Guillot, B., Fagart, J., Moras, D., Tietjen, K. & Schindler, M. (2000). A new model for 20-hydroxyecdysone and dibenzoylhydrazine binding: a homology modeling and docking approach. Protein Sci. 9, 1073-1084.
83. Yang, G., Hannan, G. N., Lockett, T. J. & Hill, R. J. (1986). Functional transfer of an elementary ecdysone gene regulatory system to mammalian cells: transient transfection and stable cell lines. Eur. J. Entomol. 92, 379-389.
84. Yao, T. P., Forman, B. M., Jiang, Z., Cherbas, L., Chen, J. D., McKeown, M., Cherbas, P. & Evans, R. M. (1993). Functional ecdysone receptor is the product of EcR and Ultraspiracle genes. Nature 366, 476-479.
85. Yudt, M. R. & Koide, S. (2001). Preventing estrogen receptor action with dimer-interface peptides. Steroids 66, 549-58.
86. Yund, M. A., King, D. S. & Fristrom, J. W. (1978). Ecdysteroid receptors in imaginal discs of Drosophila melanogaster. Proc. Natl. Acad. Sci. USA 75, 6039-43.

87. Zilliacus J., Wright, A. P., Carlstedt-Duke, J. & Gustafsson, J. A. (1995. Structural determinants of DNA-binding specificity be steroid receptors. Mol Endocrinol 9, 389-400.

APPENDIX I
The three-dimensional coordinates of the BtEcr/BtUSP LBD heterodimer
in Protein Databank format (Bernstein et al., 1977).

| | | | | | | | | | | | |
|---|---|---|---|---|---|---|---|---|---|---|---|
| ATOM | 1 | CB | VAL | U | 300 | 12.479 | 120.374 | 11.780 | 1.00 | 96.45 | U |
| ATOM | 2 | CG1 | VAL | U | 300 | 13.280 | 120.902 | 12.976 | 1.00 | 94.22 | U |
| ATOM | 3 | CG2 | VAL | U | 300 | 12.769 | 118.881 | 11.524 | 1.00 | 92.13 | U |
| ATOM | 4 | C | VAL | U | 300 | 10.448 | 119.789 | 13.212 | 1.00 | 91.04 | U |
| ATOM | 5 | O | VAL | U | 300 | 10.653 | 120.175 | 14.370 | 1.00 | 88.24 | U |
| ATOM | 6 | N | VAL | U | 300 | 10.637 | 122.069 | 12.175 | 1.00 | 91.77 | U |
| ATOM | 7 | CA | VAL | U | 300 | 10.938 | 120.609 | 12.006 | 1.00 | 93.66 | U |
| ATOM | 8 | N | SER | U | 301 | 9.797 | 118.659 | 12.928 | 1.00 | 87.56 | U |
| ATOM | 9 | CA | SER | U | 301 | 9.261 | 117.778 | 13.969 | 1.00 | 83.83 | U |
| ATOM | 10 | CB | SER | U | 301 | 8.702 | 116.493 | 13.349 | 1.00 | 82.36 | U |
| ATOM | 11 | OG | SER | U | 301 | 8.349 | 115.553 | 14.352 | 1.00 | 74.68 | U |
| ATOM | 12 | C | SER | U | 301 | 10.290 | 117.407 | 15.028 | 1.00 | 84.26 | U |
| ATOM | 13 | O | SER | U | 301 | 11.489 | 117.600 | 14.847 | 1.00 | 87.37 | U |
| ATOM | 14 | N | ASP | U | 302 | 9.812 | 116.854 | 16.134 | 1.00 | 80.44 | U |
| ATOM | 15 | CA | ASP | U | 302 | 10.692 | 116.461 | 17.223 | 1.00 | 75.43 | U |
| ATOM | 16 | CB | ASP | U | 302 | 9.860 | 116.236 | 18.489 | 1.00 | 76.98 | U |
| ATOM | 17 | CG | ASP | U | 302 | 10.571 | 116.702 | 19.741 | 1.00 | 78.13 | U |
| ATOM | 18 | OD1 | ASP | U | 302 | 11.038 | 117.865 | 19.764 | 1.00 | 77.43 | U |
| ATOM | 19 | OD2 | ASP | U | 302 | 10.656 | 115.912 | 20.705 | 1.00 | 77.91 | U |
| ATOM | 20 | C | ASP | U | 302 | 11.445 | 115.185 | 16.839 | 1.00 | 71.80 | U |
| ATOM | 21 | O | ASP | U | 302 | 12.676 | 115.121 | 16.897 | 1.00 | 68.49 | U |
| ATOM | 22 | N | ILE | U | 303 | 10.688 | 114.170 | 16.439 | 1.00 | 67.67 | U |
| ATOM | 23 | CA | ILE | U | 303 | 11.266 | 112.904 | 16.035 | 1.00 | 61.72 | U |
| ATOM | 24 | CB | ILE | U | 303 | 10.193 | 111.814 | 15.922 | 1.00 | 56.36 | U |
| ATOM | 25 | CG2 | ILE | U | 303 | 9.165 | 112.226 | 14.886 | 1.00 | 50.29 | U |
| ATOM | 26 | CG1 | ILE | U | 303 | 10.818 | 110.494 | 15.476 | 1.00 | 53.55 | U |
| ATOM | 27 | CD1 | ILE | U | 303 | 12.092 | 110.141 | 16.184 | 1.00 | 49.74 | U |
| ATOM | 28 | C | ILE | U | 303 | 11.935 | 113.064 | 14.679 | 1.00 | 64.96 | U |
| ATOM | 29 | O | ILE | U | 303 | 12.902 | 112.376 | 14.373 | 1.00 | 68.02 | U |
| ATOM | 30 | N | CYS | U | 304 | 11.422 | 113.973 | 13.860 | 1.00 | 66.56 | U |
| ATOM | 31 | CA | CYS | U | 304 | 12.003 | 114.175 | 12.548 | 1.00 | 68.23 | U |
| ATOM | 32 | CB | CYS | U | 304 | 11.138 | 115.090 | 11.694 | 1.00 | 62.87 | U |
| ATOM | 33 | SG | CYS | U | 304 | 10.279 | 114.150 | 10.444 | 1.00 | 73.57 | U |
| ATOM | 34 | C | CYS | U | 304 | 13.410 | 114.717 | 12.600 | 1.00 | 71.44 | U |
| ATOM | 35 | O | CYS | U | 304 | 14.295 | 114.194 | 11.920 | 1.00 | 75.51 | U |
| ATOM | 36 | N | GLN | U | 305 | 13.635 | 115.756 | 13.399 | 1.00 | 71.15 | U |
| ATOM | 37 | CA | GLN | U | 305 | 14.978 | 116.312 | 13.464 | 1.00 | 66.48 | U |
| ATOM | 38 | CB | GLN | U | 305 | 14.992 | 117.701 | 14.120 | 1.00 | 74.21 | U |
| ATOM | 39 | CG | GLN | U | 305 | 14.420 | 117.759 | 15.524 | 1.00 | 83.63 | U |
| ATOM | 40 | CD | GLN | U | 305 | 14.512 | 119.150 | 16.138 | 1.00 | 85.41 | U |
| ATOM | 41 | OE1 | GLN | U | 305 | 13.922 | 120.114 | 15.628 | 1.00 | 84.23 | U |
| ATOM | 42 | NE2 | GLN | U | 305 | 15.250 | 119.259 | 17.245 | 1.00 | 83.08 | U |
| ATOM | 43 | C | GLN | U | 305 | 15.886 | 115.353 | 14.199 | 1.00 | 57.19 | U |
| ATOM | 44 | O | GLN | U | 305 | 17.106 | 115.417 | 14.037 | 1.00 | 56.19 | U |
| ATOM | 45 | N | ALA | U | 306 | 15.298 | 114.457 | 14.997 | 1.00 | 48.54 | U |
| ATOM | 46 | CA | ALA | U | 306 | 16.101 | 113.458 | 15.708 | 1.00 | 45.61 | U |
| ATOM | 47 | CB | ALA | U | 306 | 15.261 | 112.698 | 16.717 | 1.00 | 28.22 | U |
| ATOM | 48 | C | ALA | U | 306 | 16.651 | 112.502 | 14.647 | 1.00 | 47.44 | U |
| ATOM | 49 | O | ALA | U | 306 | 17.826 | 112.159 | 14.660 | 1.00 | 55.69 | U |
| ATOM | 50 | N | ALA | U | 307 | 15.791 | 112.100 | 13.716 | 1.00 | 42.85 | U |
| ATOM | 51 | CA | ALA | U | 307 | 16.157 | 111.210 | 12.632 | 1.00 | 41.52 | U |
| ATOM | 52 | CB | ALA | U | 307 | 14.924 | 110.873 | 11.825 | 1.00 | 43.36 | U |
| ATOM | 53 | C | ALA | U | 307 | 17.188 | 111.885 | 11.740 | 1.00 | 42.82 | U |
| ATOM | 54 | O | ALA | U | 307 | 18.173 | 111.277 | 11.325 | 1.00 | 42.93 | U |
| ATOM | 55 | N | ASP | U | 308 | 16.947 | 113.151 | 11.448 | 1.00 | 43.61 | U |
| ATOM | 56 | CA | ASP | U | 308 | 17.849 | 113.911 | 10.605 | 1.00 | 50.98 | U |
| ATOM | 57 | CB | ASP | U | 308 | 17.300 | 115.332 | 10.383 | 1.00 | 59.56 | U |
| ATOM | 58 | CG | ASP | U | 308 | 16.155 | 115.378 | 9.349 | 1.00 | 77.63 | U |
| ATOM | 59 | OD1 | ASP | U | 308 | 16.431 | 115.189 | 8.136 | 1.00 | 77.42 | U |
| ATOM | 60 | OD2 | ASP | U | 308 | 14.980 | 115.602 | 9.745 | 1.00 | 83.75 | U |
| ATOM | 61 | C | ASP | U | 308 | 19.253 | 113.961 | 11.193 | 1.00 | 50.06 | U |
| ATOM | 62 | O | ASP | U | 308 | 20.232 | 113.759 | 10.470 | 1.00 | 48.52 | U |
| ATOM | 63 | N | ARG | U | 309 | 19.343 | 114.215 | 12.501 | 1.00 | 50.01 | U |
| ATOM | 64 | CA | ARG | U | 309 | 20.626 | 114.306 | 13.212 | 1.00 | 46.81 | U |
| ATOM | 65 | CB | ARG | U | 309 | 20.416 | 114.928 | 14.611 | 1.00 | 52.16 | U |
| ATOM | 66 | CG | ARG | U | 309 | 21.719 | 115.269 | 15.356 | 1.00 | 64.05 | U |
| ATOM | 67 | CD | ARG | U | 309 | 21.480 | 115.849 | 16.754 | 1.00 | 72.60 | U |
| ATOM | 68 | NE | ARG | U | 309 | 22.329 | 115.193 | 17.760 | 1.00 | 85.46 | U |
| ATOM | 69 | CZ | ARG | U | 309 | 22.274 | 115.409 | 19.078 | 1.00 | 83.67 | U |
| ATOM | 70 | NH1 | ARG | U | 309 | 21.406 | 116.281 | 19.585 | 1.00 | 76.09 | U |
| ATOM | 71 | NH2 | ARG | U | 309 | 23.078 | 114.731 | 19.895 | 1.00 | 78.11 | U |
| ATOM | 72 | C | ARG | U | 309 | 21.261 | 112.918 | 13.339 | 1.00 | 43.26 | U |
| ATOM | 73 | O | ARG | U | 309 | 22.461 | 112.740 | 13.156 | 1.00 | 38.45 | U |
| ATOM | 74 | N | GLN | U | 310 | 20.429 | 111.933 | 13.626 | 1.00 | 40.97 | U |
| ATOM | 75 | CA | GLN | U | 310 | 20.876 | 110.561 | 13.793 | 1.00 | 41.56 | U |
| ATOM | 76 | CB | GLN | U | 310 | 19.677 | 109.664 | 14.090 | 1.00 | 41.72 | U |
| ATOM | 77 | CG | GLN | U | 310 | 20.043 | 108.269 | 14.527 | 1.00 | 42.82 | U |
| ATOM | 78 | CD | GLN | U | 310 | 19.845 | 108.037 | 16.008 | 1.00 | 43.14 | U |
| ATOM | 79 | OE1 | GLN | U | 310 | 20.101 | 106.941 | 16.498 | 1.00 | 51.10 | U |
| ATOM | 80 | NE2 | GLN | U | 310 | 19.390 | 109.058 | 16.730 | 1.00 | 41.54 | U |
| ATOM | 81 | C | GLN | U | 310 | 21.632 | 110.016 | 12.589 | 1.00 | 41.73 | U |
| ATOM | 82 | O | GLN | U | 310 | 22.583 | 109.243 | 12.747 | 1.00 | 41.77 | U |
| ATOM | 83 | N | LEU | U | 311 | 21.197 | 110.398 | 11.391 | 1.00 | 39.72 | U |
| ATOM | 84 | CA | LEU | U | 311 | 21.866 | 109.940 | 10.182 | 1.00 | 42.01 | U |
| ATOM | 85 | CB | LEU | U | 311 | 20.929 | 110.010 | 8.990 | 1.00 | 33.72 | U |
| ATOM | 86 | CG | LEU | U | 311 | 19.901 | 108.911 | 9.253 | 1.00 | 37.21 | U |
| ATOM | 87 | CD1 | LEU | U | 311 | 18.722 | 109.006 | 8.305 | 1.00 | 42.07 | U |
| ATOM | 88 | CD2 | LEU | U | 311 | 20.620 | 107.581 | 9.118 | 1.00 | 28.89 | U |
| ATOM | 89 | C | LEU | U | 311 | 23.073 | 110.792 | 9.958 | 1.00 | 42.61 | U |
| ATOM | 90 | O | LEU | U | 311 | 24.155 | 110.282 | 9.701 | 1.00 | 47.17 | U |
| ATOM | 91 | N | TYR | U | 312 | 22.893 | 112.099 | 10.075 | 1.00 | 44.31 | U |
| ATOM | 92 | CA | TYR | U | 312 | 24.008 | 113.006 | 9.915 | 1.00 | 43.52 | U |
| ATOM | 93 | CB | TYR | U | 312 | 23.604 | 114.426 | 10.302 | 1.00 | 42.27 | U |
| ATOM | 94 | CG | TYR | U | 312 | 24.792 | 115.357 | 10.330 | 1.00 | 51.55 | U |
| ATOM | 95 | CD1 | TYR | U | 312 | 25.523 | 115.604 | 9.166 | 1.00 | 54.42 | U |
| ATOM | 96 | CE1 | TYR | U | 312 | 26.673 | 116.375 | 9.190 | 1.00 | 58.59 | U |
| ATOM | 97 | CD2 | TYR | U | 312 | 25.244 | 115.920 | 11.532 | 1.00 | 51.32 | U |
| ATOM | 98 | CE2 | TYR | U | 312 | 26.399 | 116.696 | 11.572 | 1.00 | 52.75 | U |
| ATOM | 99 | CZ | TYR | U | 312 | 27.110 | 116.923 | 10.393 | 1.00 | 60.94 | U |
| ATOM | 100 | OH | TYR | U | 312 | 28.250 | 117.704 | 10.392 | 1.00 | 62.51 | U |
| ATOM | 101 | C | TYR | U | 312 | 25.162 | 112.540 | 10.818 | 1.00 | 47.20 | U |
| ATOM | 102 | O | TYR | U | 312 | 26.327 | 112.512 | 10.401 | 1.00 | 48.75 | U |
| ATOM | 103 | N | GLN | U | 313 | 24.847 | 112.178 | 12.060 | 1.00 | 45.21 | U |
| ATOM | 104 | CA | GLN | U | 313 | 25.890 | 111.721 | 12.967 | 1.00 | 44.40 | U |
| ATOM | 105 | CB | GLN | U | 313 | 25.327 | 111.573 | 14.384 | 1.00 | 43.42 | U |
| ATOM | 106 | CG | GLN | U | 313 | 25.057 | 112.909 | 15.071 | 1.00 | 53.09 | U |
| ATOM | 107 | CD | GLN | U | 313 | 24.580 | 112.759 | 16.512 | 1.00 | 62.29 | U |
| ATOM | 108 | OE1 | GLN | U | 313 | 23.510 | 112.193 | 16.786 | 1.00 | 71.08 | U |
| ATOM | 109 | NE2 | GLN | U | 313 | 25.371 | 113.273 | 17.442 | 1.00 | 58.49 | U |
| ATOM | 110 | C | GLN | U | 313 | 26.522 | 110.403 | 12.483 | 1.00 | 46.69 | U |
| ATOM | 111 | O | GLN | U | 313 | 27.740 | 110.199 | 12.583 | 1.00 | 42.76 | U |
| ATOM | 112 | N | LEU | U | 314 | 25.700 | 109.515 | 11.935 | 1.00 | 44.18 | U |
| ATOM | 113 | CA | LEU | U | 314 | 26.205 | 108.241 | 11.455 | 1.00 | 40.49 | U |
| ATOM | 114 | CB | LEU | U | 314 | 25.078 | 107.378 | 10.894 | 1.00 | 37.07 | U |
| ATOM | 115 | CG | LEU | U | 314 | 25.487 | 106.015 | 10.333 | 1.00 | 26.82 | U |
| ATOM | 116 | CD1 | LEU | U | 314 | 25.942 | 105.112 | 11.450 | 1.00 | 20.92 | U |
| ATOM | 117 | CD2 | LEU | U | 314 | 24.305 | 105.419 | 9.604 | 1.00 | 27.59 | U |
| ATOM | 118 | C | LEU | U | 314 | 27.211 | 108.457 | 10.359 | 1.00 | 41.48 | U |
| ATOM | 119 | O | LEU | U | 314 | 28.371 | 108.067 | 10.471 | 1.00 | 48.20 | U |
| ATOM | 120 | N | ILE | U | 315 | 26.752 | 109.075 | 9.283 | 1.00 | 40.04 | U |
| ATOM | 121 | CA | ILE | U | 315 | 27.601 | 109.321 | 8.141 | 1.00 | 36.91 | U |
| ATOM | 122 | CB | ILE | U | 315 | 26.784 | 110.052 | 7.060 | 1.00 | 30.15 | U |
| ATOM | 123 | CG2 | ILE | U | 315 | 27.582 | 110.195 | 5.774 | 1.00 | 30.46 | U |
| ATOM | 124 | CG1 | ILE | U | 315 | 25.569 | 109.182 | 6.721 | 1.00 | 34.16 | U |
| ATOM | 125 | CD1 | ILE | U | 315 | 24.361 | 109.924 | 6.166 | 1.00 | 33.94 | U |
| ATOM | 126 | C | ILE | U | 315 | 28.866 | 110.078 | 8.566 | 1.00 | 43.49 | U |
| ATOM | 127 | O | ILE | U | 315 | 29.968 | 109.764 | 8.120 | 1.00 | 37.56 | U |
| ATOM | 128 | N | GLU | U | 316 | 28.731 | 111.041 | 9.470 | 1.00 | 47.27 | U |
| ATOM | 129 | CA | GLU | U | 316 | 29.905 | 111.782 | 9.902 | 1.00 | 43.86 | U |
| ATOM | 130 | CB | GLU | U | 316 | 29.477 | 113.020 | 10.704 | 1.00 | 50.83 | U |
| ATOM | 131 | CG | GLU | U | 316 | 30.378 | 114.251 | 10.478 | 1.00 | 60.61 | U |
| ATOM | 132 | CD | GLU | U | 316 | 30.496 | 114.645 | 8.997 | 1.00 | 70.18 | U |
| ATOM | 133 | OE1 | GLU | U | 316 | 29.452 | 114.676 | 8.297 | 1.00 | 69.12 | U |
| ATOM | 134 | OE2 | GLU | U | 316 | 31.633 | 114.931 | 8.536 | 1.00 | 72.57 | U |
| ATOM | 135 | C | GLU | U | 316 | 30.824 | 110.863 | 10.723 | 1.00 | 43.27 | U |
| ATOM | 136 | O | GLU | U | 316 | 32.046 | 110.987 | 10.673 | 1.00 | 47.56 | U |
| ATOM | 137 | N | TRP | U | 317 | 30.240 | 109.932 | 11.470 | 1.00 | 37.75 | U |
| ATOM | 138 | CA | TRP | U | 317 | 31.026 | 108.998 | 12.267 | 1.00 | 34.00 | U |
| ATOM | 139 | CB | TRP | U | 317 | 30.098 | 108.038 | 13.010 | 1.00 | 31.02 | U |
| ATOM | 140 | CG | TRP | U | 317 | 30.770 | 106.786 | 13.514 | 1.00 | 30.93 | U |
| ATOM | 141 | CD2 | TRP | U | 317 | 30.677 | 105.464 | 12.939 | 1.00 | 36.63 | U |
| ATOM | 142 | CE2 | TRP | U | 317 | 31.489 | 104.612 | 13.723 | 1.00 | 31.60 | U |
| ATOM | 143 | CE3 | TRP | U | 317 | 29.984 | 104.921 | 11.837 | 1.00 | 34.52 | U |
| ATOM | 144 | CD1 | TRP | U | 317 | 31.603 | 106.682 | 14.575 | 1.00 | 27.55 | U |
| ATOM | 145 | NE1 | TRP | U | 317 | 32.042 | 105.381 | 14.712 | 1.00 | 32.28 | U |
| ATOM | 146 | CZ2 | TRP | U | 317 | 31.641 | 103.245 | 13.442 | 1.00 | 34.19 | U |
| ATOM | 147 | CZ3 | TRP | U | 317 | 30.134 | 103.563 | 11.556 | 1.00 | 37.31 | U |
| ATOM | 148 | CH2 | TRP | U | 317 | 30.958 | 102.741 | 12.363 | 1.00 | 38.29 | U |
| ATOM | 149 | C | TRP | U | 317 | 31.947 | 108.198 | 11.362 | 1.00 | 39.77 | U |
| ATOM | 150 | O | TRP | U | 317 | 33.158 | 108.126 | 11.601 | 1.00 | 41.36 | U |
| ATOM | 151 | N | ALA | U | 318 | 31.353 | 107.595 | 10.328 | 1.00 | 37.36 | U |
| ATOM | 152 | CA | ALA | U | 318 | 32.080 | 106.776 | 9.367 | 1.00 | 37.84 | U |
| ATOM | 153 | CB | ALA | U | 318 | 31.113 | 106.097 | 8.414 | 1.00 | 32.85 | U |
| ATOM | 154 | C | ALA | U | 318 | 33.100 | 107.568 | 8.578 | 1.00 | 37.92 | U |
| ATOM | 155 | O | ALA | U | 318 | 34.186 | 107.062 | 8.285 | 1.00 | 35.70 | U |
| ATOM | 156 | N | LYS | U | 319 | 32.750 | 108.803 | 8.223 | 1.00 | 38.77 | U |
| ATOM | 157 | CA | LYS | U | 319 | 33.664 | 109.648 | 7.463 | 1.00 | 38.43 | U |
| ATOM | 158 | CB | LYS | U | 319 | 33.133 | 111.080 | 7.305 | 1.00 | 45.25 | U |
| ATOM | 159 | CG | LYS | U | 319 | 32.079 | 111.337 | 6.210 | 1.00 | 37.31 | U |
| ATOM | 160 | CD | LYS | U | 319 | 31.811 | 112.825 | 6.122 | 1.00 | 35.09 | U |
| ATOM | 161 | CE | LYS | U | 319 | 30.710 | 113.157 | 5.148 | 1.00 | 50.28 | U |
| ATOM | 162 | NZ | LYS | U | 319 | 30.362 | 114.621 | 5.151 | 1.00 | 53.41 | U |
| ATOM | 163 | C | LYS | U | 319 | 34.961 | 109.683 | 8.230 | 1.00 | 39.01 | U |
| ATOM | 164 | O | LYS | U | 319 | 36.032 | 109.652 | 7.622 | 1.00 | 43.09 | U |
| ATOM | 165 | N | HIS | U | 320 | 34.863 | 109.700 | 9.562 | 1.00 | 34.70 | U |
| ATOM | 166 | CA | HIS | U | 320 | 36.051 | 109.746 | 10.416 | 1.00 | 33.00 | U |
| ATOM | 167 | CB | HIS | U | 320 | 35.813 | 110.644 | 11.619 | 1.00 | 29.91 | U |
| ATOM | 168 | CG | HIS | U | 320 | 35.554 | 112.066 | 11.252 | 1.00 | 44.90 | U |
| ATOM | 169 | CD2 | HIS | U | 320 | 36.398 | 113.109 | 11.075 | 1.00 | 51.89 | U |
| ATOM | 170 | ND1 | HIS | U | 320 | 34.296 | 112.533 | 10.940 | 1.00 | 51.08 | U |
| ATOM | 171 | CE1 | HIS | U | 320 | 34.379 | 113.803 | 10.583 | 1.00 | 56.48 | U |
| ATOM | 172 | NE2 | HIS | U | 320 | 35.644 | 114.177 | 10.655 | 1.00 | 56.71 | U |
| ATOM | 173 | C | HIS | U | 320 | 36.674 | 108.450 | 10.918 | 1.00 | 32.75 | U |
| ATOM | 174 | O | HIS | U | 320 | 37.456 | 108.498 | 11.863 | 1.00 | 36.77 | U |
| ATOM | 175 | N | ILE | U | 321 | 36.344 | 107.309 | 10.311 | 1.00 | 32.76 | U |
| ATOM | 176 | CA | ILE | U | 321 | 36.939 | 106.030 | 10.720 | 1.00 | 30.81 | U |
| ATOM | 177 | CB | ILE | U | 321 | 36.039 | 104.819 | 10.404 | 1.00 | 32.67 | U |
| ATOM | 178 | CG2 | ILE | U | 321 | 36.873 | 103.557 | 10.448 | 1.00 | 26.36 | U |
| ATOM | 179 | CG1 | ILE | U | 321 | 34.802 | 104.788 | 11.313 | 1.00 | 25.98 | U |
| ATOM | 180 | CD1 | ILE | U | 321 | 35.120 | 104.784 | 12.742 | 1.00 | 38.19 | U |
| ATOM | 181 | C | ILE | U | 321 | 38.172 | 105.883 | 9.852 | 1.00 | 30.90 | U |
| ATOM | 182 | O | ILE | U | 321 | 38.075 | 105.950 | 8.629 | 1.00 | 40.52 | U |
| ATOM | 183 | N | PRO | U | 322 | 39.342 | 105.684 | 10.461 | 1.00 | 28.11 | U |
| ATOM | 184 | CD | PRO | U | 322 | 39.609 | 105.683 | 11.907 | 1.00 | 29.42 | U |
| ATOM | 185 | CA | PRO | U | 322 | 40.588 | 105.540 | 9.710 | 1.00 | 28.24 | U |
| ATOM | 186 | CB | PRO | U | 322 | 41.553 | 105.017 | 10.759 | 1.00 | 27.46 | U |
| ATOM | 187 | CG | PRO | U | 322 | 41.128 | 105.777 | 11.954 | 1.00 | 32.35 | U |
| ATOM | 188 | C | PRO | U | 322 | 40.531 | 104.655 | 8.484 | 1.00 | 28.10 | U |
| ATOM | 189 | O | PRO | U | 322 | 40.343 | 103.452 | 8.604 | 1.00 | 40.32 | U |
| ATOM | 190 | N | HIS | U | 323 | 40.715 | 105.275 | 7.316 | 1.00 | 26.78 | U |
| ATOM | 191 | CA | HIS | U | 323 | 40.725 | 104.621 | 6.004 | 1.00 | 26.63 | U |
| ATOM | 192 | CB | HIS | U | 323 | 41.352 | 103.242 | 6.102 | 1.00 | 25.78 | U |
| ATOM | 193 | CG | HIS | U | 323 | 42.711 | 103.264 | 6.703 | 1.00 | 39.81 | U |
| ATOM | 194 | CD2 | HIS | U | 323 | 43.216 | 102.640 | 7.790 | 1.00 | 48.36 | U |
| ATOM | 195 | ND1 | HIS | U | 323 | 43.721 | 104.060 | 6.209 | 1.00 | 41.57 | U |
| ATOM | 196 | CE1 | HIS | U | 323 | 44.791 | 103.926 | 6.971 | 1.00 | 48.87 | U |
| ATOM | 197 | NE2 | HIS | U | 323 | 44.513 | 103.072 | 7.938 | 1.00 | 53.44 | U |
| ATOM | 198 | C | HIS | U | 323 | 39.404 | 104.494 | 5.273 | 1.00 | 30.76 | U |
| ATOM | 199 | O | HIS | U | 323 | 39.392 | 104.202 | 4.083 | 1.00 | 31.32 | U |
| ATOM | 200 | N | PHE | U | 324 | 38.295 | 104.709 | 5.970 | 1.00 | 35.37 | U |
| ATOM | 201 | CA | PHE | U | 324 | 36.990 | 104.584 | 5.340 | 1.00 | 36.68 | U |
| ATOM | 202 | CB | PHE | U | 324 | 35.886 | 105.074 | 6.275 | 1.00 | 30.95 | U |
| ATOM | 203 | CG | PHE | U | 324 | 34.511 | 104.881 | 5.707 | 1.00 | 38.02 | U |
| ATOM | 204 | CD1 | PHE | U | 324 | 33.991 | 103.587 | 5.532 | 1.00 | 32.62 | U |
| ATOM | 205 | CD2 | PHE | U | 324 | 33.763 | 105.979 | 5.261 | 1.00 | 34.48 | U |
| ATOM | 206 | CE1 | PHE | U | 324 | 32.750 | 103.397 | 4.911 | 1.00 | 29.22 | U |
| ATOM | 207 | CE2 | PHE | U | 324 | 32.511 | 105.800 | 4.636 | 1.00 | 31.30 | U |
| ATOM | 208 | CZ | PHE | U | 324 | 32.006 | 104.514 | 4.460 | 1.00 | 25.37 | U |
| ATOM | 209 | C | PHE | U | 324 | 36.886 | 105.351 | 4.015 | 1.00 | 41.75 | U |
| ATOM | 210 | O | PHE | U | 324 | 36.539 | 104.785 | 2.970 | 1.00 | 38.18 | U |
| ATOM | 211 | N | THR | U | 325 | 37.185 | 106.648 | 4.072 | 1.00 | 42.47 | U |
| ATOM | 212 | CA | THR | U | 325 | 37.104 | 107.509 | 2.903 | 1.00 | 37.33 | U |
| ATOM | 213 | CB | THR | U | 325 | 37.091 | 108.974 | 3.289 | 1.00 | 32.20 | U |
| ATOM | 214 | OG1 | THR | U | 325 | 38.139 | 109.224 | 4.227 | 1.00 | 37.87 | U |
| ATOM | 215 | CG2 | THR | U | 325 | 35.746 | 109.344 | 3.895 | 1.00 | 29.55 | U |
| ATOM | 216 | C | THR | U | 325 | 38.204 | 107.295 | 1.900 | 1.00 | 38.08 | U |
| ATOM | 217 | O | THR | U | 325 | 38.262 | 108.000 | 0.900 | 1.00 | 45.26 | U |
| ATOM | 218 | N | GLU | U | 326 | 39.086 | 106.339 | 2.156 | 1.00 | 35.21 | U |
| ATOM | 219 | CA | GLU | U | 326 | 40.130 | 106.050 | 1.197 | 1.00 | 35.59 | U |
| ATOM | 220 | CB | GLU | U | 326 | 41.426 | 105.642 | 1.898 | 1.00 | 43.50 | U |
| ATOM | 221 | CG | GLU | U | 326 | 41.942 | 106.651 | 2.935 | 1.00 | 52.68 | U |
| ATOM | 222 | CD | GLU | U | 326 | 43.381 | 106.359 | 3.366 | 1.00 | 56.78 | U |
| ATOM | 223 | OE1 | GLU | U | 326 | 43.728 | 105.172 | 3.600 | 1.00 | 52.62 | U |
| ATOM | 224 | OE2 | GLU | U | 326 | 44.166 | 107.324 | 3.477 | 1.00 | 55.12 | U |
| ATOM | 225 | C | GLU | U | 326 | 39.602 | 104.923 | 0.303 | 1.00 | 34.41 | U |
| ATOM | 226 | O | GLU | U | 326 | 40.253 | 104.499 | −0.638 | 1.00 | 39.17 | U |
| ATOM | 227 | N | LEU | U | 327 | 38.414 | 104.424 | 0.604 | 1.00 | 35.62 | U |
| ATOM | 228 | CA | LEU | U | 327 | 37.811 | 103.391 | −0.233 | 1.00 | 34.31 | U |
| ATOM | 229 | CB | LEU | U | 327 | 36.799 | 102.573 | 0.556 | 1.00 | 29.93 | U |
| ATOM | 230 | CG | LEU | U | 327 | 37.272 | 101.577 | 1.587 | 1.00 | 28.51 | U |
| ATOM | 231 | CD1 | LEU | U | 327 | 36.200 | 101.454 | 2.633 | 1.00 | 31.32 | U |
| ATOM | 232 | CD2 | LEU | U | 327 | 37.546 | 100.223 | 0.935 | 1.00 | 31.78 | U |
| ATOM | 233 | C | LEU | U | 327 | 37.077 | 104.099 | −1.380 | 1.00 | 34.86 | U |
| ATOM | 234 | O | LEU | U | 327 | 36.764 | 105.291 | −1.298 | 1.00 | 32.50 | U |
| ATOM | 235 | N | PRO | U | 328 | 36.783 | 103.368 | −2.461 | 1.00 | 34.61 | U |
| ATOM | 236 | CD | PRO | U | 328 | 37.056 | 101.942 | −2.704 | 1.00 | 36.41 | U |
| ATOM | 237 | CA | PRO | U | 328 | 36.081 | 103.965 | −3.601 | 1.00 | 36.93 | U |
| ATOM | 238 | CB | PRO | U | 328 | 35.954 | 102.792 | −4.566 | 1.00 | 42.49 | U |
| ATOM | 239 | CG | PRO | U | 328 | 37.112 | 101.898 | −4.186 | 1.00 | 35.91 | U |
| ATOM | 240 | C | PRO | U | 328 | 34.722 | 104.507 | −3.154 | 1.00 | 35.70 | U |
| ATOM | 241 | O | PRO | U | 328 | 33.939 | 103.798 | −2.539 | 1.00 | 40.26 | U |
| ATOM | 242 | N | VAL | U | 329 | 34.423 | 105.752 | −3.467 | 1.00 | 33.92 | U |
| ATOM | 243 | CA | VAL | U | 329 | 33.162 | 106.304 | −3.012 | 1.00 | 40.39 | U |
| ATOM | 244 | CB | VAL | U | 329 | 32.844 | 107.587 | −3.719 | 1.00 | 37.74 | U |
| ATOM | 245 | CG1 | VAL | U | 329 | 31.899 | 108.388 | −2.864 | 1.00 | 32.09 | U |
| ATOM | 246 | CG2 | VAL | U | 329 | 34.131 | 108.345 | −4.006 | 1.00 | 46.30 | U |
| ATOM | 247 | C | VAL | U | 329 | 31.940 | 105.397 | −3.140 | 1.00 | 45.16 | U |
| ATOM | 248 | O | VAL | U | 329 | 31.085 | 105.387 | −2.254 | 1.00 | 48.70 | U |
| ATOM | 249 | N | GLU | U | 330 | 31.853 | 104.648 | −4.235 | 1.00 | 47.59 | U |
| ATOM | 250 | CA | GLU | U | 330 | 30.727 | 103.748 | −4.479 | 1.00 | 49.23 | U |
| ATOM | 251 | CB | GLU | U | 330 | 30.852 | 103.100 | −5.862 | 1.00 | 57.88 | U |
| ATOM | 252 | CG | GLU | U | 330 | 31.492 | 104.010 | −6.919 | 1.00 | 75.31 | U |
| ATOM | 253 | CD | GLU | U | 330 | 33.028 | 104.105 | −6.791 | 1.00 | 85.37 | U |
| ATOM | 254 | OE1 | GLU | U | 330 | 33.717 | 103.102 | −7.112 | 1.00 | 88.41 | U |
| ATOM | 255 | OE2 | GLU | U | 330 | 33.545 | 105.175 | −6.370 | 1.00 | 81.90 | U |
| ATOM | 256 | C | GLU | U | 330 | 30.691 | 102.671 | −3.406 | 1.00 | 47.32 | U |
| ATOM | 257 | O | GLU | U | 330 | 29.619 | 102.265 | −2.959 | 1.00 | 47.52 | U |
| ATOM | 258 | N | ASP | U | 331 | 31.868 | 102.202 | −3.004 | 1.00 | 46.97 | U |
| ATOM | 259 | CA | ASP | U | 331 | 31.990 | 101.196 | −1.951 | 1.00 | 48.96 | U |
| ATOM | 260 | CB | ASP | U | 331 | 33.414 | 100.659 | −1.932 | 1.00 | 49.87 | U |
| ATOM | 261 | CG | ASP | U | 331 | 33.605 | 99.555 | −2.924 | 1.00 | 53.38 | U |
| ATOM | 262 | OD1 | ASP | U | 331 | 32.709 | 99.411 | −3.783 | 1.00 | 56.13 | U |
| ATOM | 263 | OD2 | ASP | U | 331 | 34.625 | 98.839 | −2.843 | 1.00 | 47.10 | U |
| ATOM | 264 | C | ASP | U | 331 | 31.610 | 101.797 | −0.588 | 1.00 | 49.54 | U |
| ATOM | 265 | O | ASP | U | 331 | 31.048 | 101.121 | 0.289 | 1.00 | 48.49 | U |
| ATOM | 266 | N | GLN | U | 332 | 31.930 | 103.074 | −0.421 | 1.00 | 44.30 | U |
| ATOM | 267 | CA | GLN | U | 332 | 31.575 | 103.789 | 0.783 | 1.00 | 43.21 | U |
| ATOM | 268 | CB | GLN | U | 332 | 32.120 | 105.208 | 0.724 | 1.00 | 43.36 | U |
| ATOM | 269 | CG | GLN | U | 332 | 33.592 | 105.290 | 0.982 | 1.00 | 47.06 | U |
| ATOM | 270 | CD | GLN | U | 332 | 34.061 | 106.702 | 1.038 | 1.00 | 50.14 | U |
| ATOM | 271 | OE1 | GLN | U | 332 | 33.377 | 107.574 | 1.575 | 1.00 | 50.22 | U |
| ATOM | 272 | NE2 | GLN | U | 332 | 35.235 | 106.948 | 0.493 | 1.00 | 53.82 | U |
| ATOM | 273 | C | GLN | U | 332 | 30.050 | 103.827 | 0.896 | 1.00 | 44.72 | U |
| ATOM | 274 | O | GLN | U | 332 | 39.500 | 103.632 | 1.972 | 1.00 | 49.65 | U |
| ATOM | 275 | N | VAL | U | 333 | 29.362 | 104.079 | −0.214 | 1.00 | 42.41 | U |
| ATOM | 276 | CA | VAL | U | 333 | 27.905 | 104.118 | −0.187 | 1.00 | 40.09 | U |
| ATOM | 277 | CB | VAL | U | 333 | 27.352 | 104.654 | −1.510 | 1.00 | 33.88 | U |
| ATOM | 278 | CG1 | VAL | U | 333 | 25.838 | 104.693 | −1.470 | 1.00 | 30.31 | U |
| ATOM | 279 | CG2 | VAL | U | 333 | 27.882 | 106.070 | −1.729 | 1.00 | 33.72 | U |
| ATOM | 280 | C | VAL | U | 333 | 27.364 | 102.723 | 0.107 | 1.00 | 40.39 | U |
| ATOM | 281 | O | VAL | U | 333 | 26.491 | 102.535 | 0.959 | 1.00 | 40.57 | U |
| ATOM | 282 | N | ILE | U | 334 | 27.907 | 101.742 | −0.588 | 1.00 | 36.75 | U |
| ATOM | 283 | CA | ILE | U | 334 | 27.525 | 100.361 | −0.367 | 1.00 | 39.58 | U |
| ATOM | 284 | CB | ILE | U | 334 | 28.405 | 99.427 | −1.207 | 1.00 | 42.58 | U |
| ATOM | 285 | CG2 | ILE | U | 334 | 28.248 | 97.979 | −0.742 | 1.00 | 45.90 | U |
| ATOM | 286 | CG1 | ILE | U | 334 | 28.075 | 99.618 | −2.685 | 1.00 | 39.08 | U |
| ATOM | 287 | CD1 | ILE | U | 334 | 28.839 | 98.687 | −3.563 | 1.00 | 41.96 | U |
| ATOM | 288 | C | ILE | U | 334 | 27.676 | 99.956 | 1.105 | 1.00 | 41.97 | U |
| ATOM | 289 | O | ILE | U | 334 | 26.748 | 99.416 | 1.721 | 1.00 | 41.77 | U |
| ATOM | 290 | N | LEU | U | 335 | 28.855 | 100.203 | 1.662 | 1.00 | 38.84 | U |
| ATOM | 291 | CA | LEU | U | 335 | 39.093 | 99.825 | 3.037 | 1.00 | 38.04 | U |
| ATOM | 292 | CB | LEU | U | 335 | 30.542 | 100.127 | 3.430 | 1.00 | 38.86 | U |
| ATOM | 293 | CG | LEU | U | 335 | 31.569 | 99.106 | 2.923 | 1.00 | 41.31 | U |
| ATOM | 294 | CD1 | LEU | U | 335 | 32.952 | 99.609 | 3.226 | 1.00 | 48.69 | U |
| ATOM | 295 | CD2 | LEU | U | 335 | 31.358 | 97.753 | 3.575 | 1.00 | 36.63 | U |
| ATOM | 296 | C | LEU | U | 335 | 28.125 | 100.474 | 4.014 | 1.00 | 34.75 | U |
| ATOM | 297 | O | LEU | U | 335 | 27.690 | 99.840 | 4.974 | 1.00 | 35.11 | U |
| ATOM | 298 | N | LEU | U | 336 | 27.780 | 101.728 | 3.765 | 1.00 | 30.31 | U |
| ATOM | 299 | CA | LEU | U | 336 | 26.864 | 102.451 | 4.635 | 1.00 | 32.58 | U |
| ATOM | 300 | CB | LEU | U | 336 | 27.018 | 103.970 | 4.452 | 1.00 | 34.63 | U |
| ATOM | 301 | CG | LEU | U | 336 | 28.050 | 104.689 | 5.334 | 1.00 | 35.17 | U |
| ATOM | 302 | CD1 | LEU | U | 336 | 28.378 | 106.062 | 4.769 | 1.00 | 29.95 | U |
| ATOM | 303 | CD2 | LEU | U | 336 | 27.496 | 104.800 | 6.744 | 1.00 | 31.44 | U |
| ATOM | 304 | C | LEU | U | 336 | 25.430 | 102.069 | 4.353 | 1.00 | 35.51 | U |
| ATOM | 305 | O | LEU | U | 336 | 24.641 | 101.884 | 5.281 | 1.00 | 35.28 | U |
| ATOM | 306 | N | LYS | U | 337 | 25.084 | 101.951 | 3.071 | 1.00 | 36.77 | U |
| ATOM | 307 | CA | LYS | U | 337 | 23.713 | 101.615 | 2.720 | 1.00 | 34.23 | U |
| ATOM | 308 | CB | LYS | U | 337 | 23.507 | 101.623 | 1.206 | 1.00 | 41.87 | U |
| ATOM | 309 | CG | LYS | U | 337 | 22.052 | 101.329 | 0.782 | 1.00 | 47.78 | U |
| ATOM | 310 | CD | LYS | U | 337 | 21.822 | 101.410 | −0.726 | 1.00 | 43.48 | U |
| ATOM | 311 | CE | LYS | U | 337 | 20.514 | 100.743 | −1.064 | 1.00 | 42.29 | U |
| ATOM | 312 | NZ | LYS | U | 337 | 20.043 | 101.077 | −2.425 | 1.00 | 54.28 | U |
| ATOM | 313 | C | LYS | U | 337 | 23.357 | 100.263 | 3.289 | 1.00 | 33.22 | U |
| ATOM | 314 | O | LYS | U | 337 | 22.211 | 100.016 | 3.656 | 1.00 | 37.11 | U |
| ATOM | 315 | N | SER | U | 338 | 24.354 | 99.393 | 3.370 | 1.00 | 25.79 | U |
| ATOM | 316 | CA | SER | U | 338 | 24.168 | 98.057 | 3.901 | 1.00 | 24.15 | U |
| ATOM | 317 | CB | SER | U | 338 | 25.161 | 97.124 | 3.203 | 1.00 | 21.99 | U |
| ATOM | 318 | OG | SER | U | 338 | 25.287 | 95.885 | 3.876 | 1.00 | 17.90 | U |
| ATOM | 319 | C | SER | U | 338 | 24.297 | 97.982 | 5.449 | 1.00 | 30.41 | U |
| ATOM | 320 | O | SER | U | 338 | 23.490 | 97.340 | 6.114 | 1.00 | 31.99 | U |
| ATOM | 321 | N | GLY | U | 339 | 25.301 | 98.637 | 6.030 | 1.00 | 32.94 | U |
| ATOM | 322 | CA | GLY | U | 339 | 25.457 | 98.604 | 7.476 | 1.00 | 28.86 | U |
| ATOM | 323 | C | GLY | U | 339 | 24.445 | 99.480 | 8.201 | 1.00 | 28.75 | U |
| ATOM | 324 | O | GLY | U | 339 | 24.231 | 99.328 | 9.402 | 1.00 | 26.26 | U |
| ATOM | 325 | N | TRP | U | 340 | 23.819 | 100.386 | 7.448 | 1.00 | 25.56 | U |
| ATOM | 326 | CA | TRP | U | 340 | 22.820 | 101.334 | 7.938 | 1.00 | 27.93 | U |
| ATOM | 327 | CB | TRP | U | 340 | 21.893 | 101.726 | 6.765 | 1.00 | 28.33 | U |
| ATOM | 328 | CG | TRP | U | 340 | 20.852 | 102.785 | 7.075 | 1.00 | 32.51 | U |
| ATOM | 329 | CD2 | TRP | U | 340 | 20.701 | 104.069 | 6.446 | 1.00 | 36.24 | U |
| ATOM | 330 | CE2 | TRP | U | 340 | 19.578 | 104.698 | 7.043 | 1.00 | 32.10 | U |
| ATOM | 331 | CE3 | TRP | U | 340 | 21.400 | 104.752 | 5.438 | 1.00 | 33.50 | U |
| ATOM | 332 | CD1 | TRP | U | 340 | 19.846 | 102.700 | 7.995 | 1.00 | 35.61 | U |
| ATOM | 333 | NE1 | TRP | U | 340 | 19.079 | 103.838 | 7.981 | 1.00 | 33.30 | U |
| ATOM | 334 | CZ2 | TRP | U | 340 | 19.146 | 105.978 | 6.673 | 1.00 | 22.67 | U |
| ATOM | 335 | CZ3 | TRP | U | 340 | 20.959 | 106.041 | 5.069 | 1.00 | 22.73 | U |
| ATOM | 336 | CH2 | TRP | U | 340 | 19.846 | 106.630 | 5.688 | 1.00 | 27.61 | U |
| ATOM | 337 | C | TRP | U | 340 | 22.007 | 100.896 | 9.179 | 1.00 | 30.07 | U |
| ATOM | 338 | O | TRP | U | 340 | 22.245 | 101.385 | 10.276 | 1.00 | 34.83 | U |
| ATOM | 339 | N | ASN | U | 341 | 21.067 | 99.973 | 9.040 | 1.00 | 30.01 | U |
| ATOM | 340 | CA | ASN | U | 341 | 20.276 | 99.597 | 10.204 | 1.00 | 29.52 | U |
| ATOM | 341 | CB | ASN | U | 341 | 19.227 | 98.570 | 9.804 | 1.00 | 32.18 | U |
| ATOM | 342 | CG | ASN | U | 341 | 18.089 | 99.182 | 9.016 | 1.00 | 31.81 | U |
| ATOM | 343 | OD1 | ASN | U | 341 | 17.206 | 98.477 | 8.518 | 1.00 | 32.06 | U |
| ATOM | 344 | ND2 | ASN | U | 341 | 18.099 | 100.497 | 8.900 | 1.00 | 28.83 | U |
| ATOM | 345 | C | ASN | U | 341 | 21.063 | 99.087 | 11.411 | 1.00 | 31.89 | U |
| ATOM | 346 | O | ASN | U | 341 | 20.911 | 99.592 | 12.526 | 1.00 | 34.91 | U |
| ATOM | 347 | N | GLU | U | 342 | 21.897 | 98.078 | 11.208 | 1.00 | 31.11 | U |
| ATOM | 348 | CA | GLU | U | 342 | 22.651 | 97.553 | 12.331 | 1.00 | 29.94 | U |
| ATOM | 349 | CB | GLU | U | 342 | 23.547 | 96.401 | 11.908 | 1.00 | 26.29 | U |
| ATOM | 350 | CG | GLU | U | 342 | 22.814 | 95.202 | 11.417 | 1.00 | 26.29 | U |
| ATOM | 351 | CD | GLU | U | 342 | 23.752 | 94.084 | 11.035 | 1.00 | 36.46 | U |
| ATOM | 352 | OE1 | GLU | U | 342 | 23.997 | 93.876 | 9.828 | 1.00 | 30.05 | U |
| ATOM | 353 | OE2 | GLU | U | 342 | 24.266 | 93.407 | 11.953 | 1.00 | 53.55 | U |
| ATOM | 354 | C | GLU | U | 342 | 23.505 | 98.632 | 12.965 | 1.00 | 33.66 | U |
| ATOM | 355 | O | GLU | U | 342 | 23.729 | 98.596 | 14.166 | 1.00 | 39.16 | U |
| ATOM | 356 | N | LEU | U | 343 | 23.984 | 99.592 | 12.178 | 1.00 | 29.29 | U |
| ATOM | 357 | CA | LEU | U | 343 | 24.817 | 100.634 | 12.747 | 1.00 | 30.23 | U |
| ATOM | 358 | CB | LEU | U | 343 | 25.555 | 101.417 | 11.653 | 1.00 | 27.80 | U |
| ATOM | 359 | CG | LEU | U | 343 | 26.745 | 100.750 | 10.930 | 1.00 | 32.16 | U |
| ATOM | 360 | CD1 | LEU | U | 343 | 27.165 | 101.600 | 9.723 | 1.00 | 23.65 | U |
| ATOM | 361 | CD2 | LEU | U | 343 | 27.904 | 100.562 | 11.880 | 1.00 | 16.27 | U |
| ATOM | 362 | C | LEU | U | 343 | 24.003 | 101.578 | 13.619 | 1.00 | 35.57 | U |
| ATOM | 363 | O | LEU | U | 343 | 24.399 | 101.870 | 14.758 | 1.00 | 33.58 | U |
| ATOM | 364 | N | LEU | U | 344 | 22.865 | 102.044 | 13.108 | 1.00 | 32.56 | U |
| ATOM | 365 | CA | LEU | U | 344 | 22.025 | 102.960 | 13.881 | 1.00 | 37.27 | U |
| ATOM | 366 | CB | LEU | U | 344 | 20.807 | 103.428 | 13.091 | 1.00 | 24.92 | U |
| ATOM | 367 | CG | LEU | U | 344 | 21.049 | 104.222 | 11.826 | 1.00 | 23.17 | U |
| ATOM | 368 | CD1 | LEU | U | 344 | 19.729 | 104.429 | 11.171 | 1.00 | 24.49 | U |
| ATOM | 369 | CD2 | LEU | U | 344 | 21.715 | 105.548 | 12.127 | 1.00 | 16.44 | U |
| ATOM | 370 | C | LEU | U | 344 | 21.517 | 102.291 | 15.137 | 1.00 | 41.23 | U |
| ATOM | 371 | O | LEU | U | 344 | 21.448 | 102.922 | 16.194 | 1.00 | 46.21 | U |
| ATOM | 372 | N | ILE | U | 345 | 21.148 | 101.018 | 15.004 | 1.00 | 39.57 | U |
| ATOM | 373 | CA | ILE | U | 345 | 20.607 | 100.245 | 16.112 | 1.00 | 37.50 | U |
| ATOM | 374 | CB | ILE | U | 345 | 20.150 | 98.857 | 15.627 | 1.00 | 36.70 | U |
| ATOM | 375 | CG2 | ILE | U | 345 | 19.928 | 97.911 | 16.816 | 1.00 | 33.77 | U |
| ATOM | 376 | CG1 | ILE | U | 345 | 18.882 | 99.009 | 14.792 | 1.00 | 31.92 | U |
| ATOM | 377 | CD1 | ILE | U | 345 | 18.361 | 97.689 | 14.247 | 1.00 | 36.61 | U |
| ATOM | 378 | C | ILE | U | 345 | 21.602 | 100.087 | 17.244 | 1.00 | 34.63 | U |
| ATOM | 379 | O | ILE | U | 345 | 21.305 | 100.393 | 18.385 | 1.00 | 33.90 | U |
| ATOM | 380 | N | ALA | U | 346 | 22.787 | 99.603 | 16.922 | 1.00 | 34.92 | U |
| ATOM | 381 | CA | ALA | U | 346 | 23.827 | 99.402 | 17.919 | 1.00 | 33.34 | U |
| ATOM | 382 | CB | ALA | U | 346 | 25.079 | 98.872 | 17.233 | 1.00 | 20.38 | U |
| ATOM | 383 | C | ALA | U | 346 | 24.125 | 100.720 | 18.640 | 1.00 | 35.49 | U |
| ATOM | 384 | O | ALA | U | 346 | 24.349 | 100.759 | 19.853 | 1.00 | 32.79 | U |
| ATOM | 385 | N | GLY | U | 347 | 24.120 | 101.800 | 17.864 | 1.00 | 39.18 | U |
| ATOM | 386 | CA | GLY | U | 347 | 24.386 | 103.121 | 18.399 | 1.00 | 37.65 | U |
| ATOM | 387 | C | GLY | U | 347 | 23.359 | 103.573 | 19.413 | 1.00 | 39.12 | U |
| ATOM | 388 | O | GLY | U | 347 | 23.711 | 103.796 | 20.563 | 1.00 | 37.43 | U |
| ATOM | 389 | N | PHE | U | 348 | 22.095 | 103.710 | 19.011 | 1.00 | 39.25 | U |
| ATOM | 390 | CA | PHE | U | 348 | 21.101 | 104.140 | 19.975 | 1.00 | 34.80 | U |
| ATOM | 391 | CB | PHE | U | 348 | 19.778 | 104.588 | 19.305 | 1.00 | 33.33 | U |
| ATOM | 392 | CG | PHE | U | 348 | 19.037 | 103.532 | 18.536 | 1.00 | 32.08 | U |
| ATOM | 393 | CD1 | PHE | U | 348 | 18.522 | 102.402 | 19.165 | 1.00 | 30.46 | U |
| ATOM | 394 | CD2 | PHE | U | 348 | 18.760 | 103.726 | 17.176 | 1.00 | 32.54 | U |
| ATOM | 395 | CE1 | PHE | U | 348 | 17.735 | 101.482 | 18.450 | 1.00 | 30.24 | U |
| ATOM | 396 | CE2 | PHE | U | 348 | 17.976 | 102.812 | 16.452 | 1.00 | 26.04 | U |
| ATOM | 397 | CZ | PHE | U | 348 | 17.465 | 101.694 | 17.085 | 1.00 | 27.51 | U |
| ATOM | 398 | C | PHE | U | 348 | 20.851 | 103.128 | 21.073 | 1.00 | 33.35 | U |
| ATOM | 399 | O | PHE | U | 348 | 20.269 | 103.477 | 22.096 | 1.00 | 32.57 | U |
| ATOM | 400 | N | SER | U | 349 | 21.324 | 101.894 | 20.877 | 1.00 | 33.74 | U |
| ATOM | 401 | CA | SER | U | 349 | 21.174 | 100.815 | 21.865 | 1.00 | 35.08 | U |
| ATOM | 402 | CB | SER | U | 349 | 21.520 | 99.457 | 21.254 | 1.00 | 35.19 | U |
| ATOM | 403 | OG | SER | U | 349 | 20.413 | 98.887 | 20.592 | 1.00 | 43.14 | U |
| ATOM | 404 | C | SER | U | 349 | 22.093 | 101.056 | 23.051 | 1.00 | 34.96 | U |
| ATOM | 405 | O | SER | U | 349 | 21.679 | 101.001 | 24.210 | 1.00 | 35.52 | U |
| ATOM | 406 | N | HIS | U | 350 | 23.354 | 101.314 | 22.738 | 1.00 | 33.89 | U |
| ATOM | 407 | CA | HIS | U | 350 | 24.354 | 101.575 | 23.750 | 1.00 | 38.26 | U |
| ATOM | 408 | CB | HIS | U | 350 | 25.738 | 101.609 | 23.115 | 1.00 | 37.36 | U |
| ATOM | 409 | CG | HIS | U | 350 | 26.837 | 101.892 | 24.088 | 1.00 | 35.12 | U |
| ATOM | 410 | CD2 | HIS | U | 350 | 27.588 | 101.058 | 24.851 | 1.00 | 32.12 | U |
| ATOM | 411 | ND1 | HIS | U | 350 | 27.293 | 103.166 | 24.351 | 1.00 | 27.40 | U |
| ATOM | 412 | CE1 | HIS | U | 350 | 28.284 | 103.103 | 25.224 | 1.00 | 31.15 | U |
| ATOM | 413 | NE2 | HIS | U | 350 | 28.480 | 101.834 | 25.541 | 1.00 | 24.80 | U |
| ATOM | 414 | C | HIS | U | 350 | 24.080 | 102.891 | 24.454 | 1.00 | 41.29 | U |
| ATOM | 415 | O | HIS | U | 350 | 24.221 | 102.971 | 25.665 | 1.00 | 43.64 | U |
| ATOM | 416 | N | ARG | U | 351 | 23.698 | 103.917 | 23.687 | 1.00 | 46.27 | U |
| ATOM | 417 | CA | ARG | U | 351 | 23.390 | 105.244 | 24.223 | 1.00 | 41.47 | U |
| ATOM | 418 | CB | ARG | U | 351 | 23.084 | 106.247 | 23.107 | 1.00 | 40.73 | U |
| ATOM | 419 | CG | ARG | U | 351 | 22.823 | 107.679 | 23.626 | 1.00 | 52.88 | U |
| ATOM | 420 | CD | ARG | U | 351 | 22.703 | 108.675 | 22.482 | 1.00 | 65.75 | U |
| ATOM | 421 | NE | ARG | U | 351 | 22.716 | 110.064 | 22.939 | 1.00 | 78.56 | U |
| ATOM | 422 | CZ | ARG | U | 351 | 22.786 | 111.133 | 22.134 | 1.00 | 85.00 | U |
| ATOM | 423 | NH1 | ARG | U | 351 | 22.852 | 110.999 | 20.807 | 1.00 | 82.69 | U |
| ATOM | 424 | NH2 | ARG | U | 351 | 22.800 | 112.354 | 22.661 | 1.00 | 89.42 | U |
| ATOM | 425 | C | ARG | U | 351 | 22.200 | 105.224 | 25.169 | 1.00 | 43.65 | U |
| ATOM | 426 | O | ARG | U | 351 | 22.019 | 106.139 | 25.971 | 1.00 | 46.07 | U |
| ATOM | 427 | N | SER | U | 352 | 21.380 | 104.193 | 25.095 | 1.00 | 37.12 | U |
| ATOM | 428 | CA | SER | U | 352 | 20.244 | 104.182 | 25.976 | 1.00 | 41.58 | U |
| ATOM | 429 | CB | SER | U | 352 | 18.961 | 103.999 | 25.155 | 1.00 | 41.72 | U |
| ATOM | 430 | OG | SER | U | 352 | 19.142 | 103.057 | 24.142 | 1.00 | 40.19 | U |
| ATOM | 431 | C | SER | U | 352 | 20.355 | 103.155 | 27.083 | 1.00 | 42.37 | U |
| ATOM | 432 | O | SER | U | 352 | 19.352 | 102.574 | 27.494 | 1.00 | 44.94 | U |
| ATOM | 433 | N | MET | U | 353 | 21.572 | 102.958 | 27.589 | 1.00 | 43.30 | U |
| ATOM | 434 | CA | MET | U | 353 | 21.803 | 101.974 | 28.653 | 1.00 | 41.91 | U |
| ATOM | 435 | CB | MET | U | 353 | 23.288 | 101.657 | 28.827 | 1.00 | 37.50 | U |
| ATOM | 436 | CG | MET | U | 353 | 23.872 | 100.675 | 27.854 | 1.00 | 38.15 | U |
| ATOM | 437 | SD | MET | U | 353 | 25.607 | 100.366 | 28.215 | 1.00 | 42.35 | U |
| ATOM | 438 | CE | MET | U | 353 | 26.326 | 101.885 | 27.776 | 1.00 | 36.09 | U |
| ATOM | 439 | C | MET | U | 353 | 21.287 | 102.486 | 29.965 | 1.00 | 40.18 | U |
| ATOM | 440 | O | MET | U | 353 | 20.763 | 101.729 | 30.784 | 1.00 | 38.44 | U |
| ATOM | 441 | N | SER | U | 354 | 21.453 | 103.785 | 30.157 | 1.00 | 41.70 | U |
| ATOM | 442 | CA | SER | U | 354 | 21.023 | 104.434 | 31.381 | 1.00 | 52.28 | U |
| ATOM | 443 | CB | SER | U | 354 | 21.788 | 105.741 | 31.561 | 1.00 | 50.59 | U |
| ATOM | 444 | OG | SER | U | 354 | 21.749 | 106.498 | 30.357 | 1.00 | 62.08 | U |
| ATOM | 445 | C | SER | U | 354 | 19.516 | 104.689 | 31.371 | 1.00 | 57.36 | U |
| ATOM | 446 | O | SER | U | 354 | 18.869 | 104.579 | 32.411 | 1.00 | 66.59 | U |
| ATOM | 447 | N | VAL | U | 355 | 18.960 | 105.021 | 30.207 | 1.00 | 53.96 | U |
| ATOM | 448 | CA | VAL | U | 355 | 17.528 | 105.263 | 30.098 | 1.00 | 49.81 | U |
| ATOM | 449 | CB | VAL | U | 355 | 17.129 | 105.517 | 28.631 | 1.00 | 48.65 | U |
| ATOM | 450 | CG1 | VAL | U | 355 | 15.632 | 105.396 | 28.471 | 1.00 | 38.73 | U |
| ATOM | 451 | CG2 | VAL | U | 355 | 17.606 | 106.901 | 28.190 | 1.00 | 42.68 | U |
| ATOM | 452 | C | VAL | U | 355 | 16.761 | 104.046 | 30.623 | 1.00 | 53.60 | U |
| ATOM | 453 | O | VAL | U | 355 | 17.146 | 102.901 | 30.365 | 1.00 | 54.54 | U |
| ATOM | 454 | N | LYS | U | 356 | 15.690 | 104.293 | 31.374 | 1.00 | 55.40 | U |
| ATOM | 455 | CA | LYS | U | 356 | 14.887 | 103.196 | 31.906 | 1.00 | 60.98 | U |
| ATOM | 456 | CB | LYS | U | 356 | 14.344 | 103.539 | 33.294 | 1.00 | 67.99 | U |
| ATOM | 457 | CG | LYS | U | 356 | 13.463 | 102.430 | 33.900 | 1.00 | 75.32 | U |
| ATOM | 458 | CD | LYS | U | 356 | 12.748 | 102.863 | 35.190 | 1.00 | 77.01 | U |
| ATOM | 459 | CE | LYS | U | 356 | 13.725 | 103.184 | 36.330 | 1.00 | 79.62 | U |
| ATOM | 460 | NZ | LYS | U | 356 | 13.051 | 103.384 | 37.659 | 1.00 | 75.59 | U |
| ATOM | 461 | C | LYS | U | 356 | 13.723 | 102.942 | 30.957 | 1.00 | 62.54 | U |
| ATOM | 462 | O | LYS | U | 356 | 13.055 | 103.885 | 30.519 | 1.00 | 60.53 | U |
| ATOM | 463 | N | ASP | U | 357 | 13.487 | 101.670 | 30.642 | 1.00 | 63.08 | U |
| ATOM | 464 | CA | ASP | U | 357 | 12.412 | 101.269 | 29.734 | 1.00 | 67.30 | U |
| ATOM | 465 | CB | ASP | U | 357 | 11.083 | 101.222 | 30.478 | 1.00 | 74.78 | U |
| ATOM | 466 | CG | ASP | U | 357 | 10.959 | 99.998 | 31.355 | 1.00 | 88.42 | U |
| ATOM | 467 | OD1 | ASP | U | 357 | 10.966 | 98.880 | 30.794 | 1.00 | 93.99 | U |
| ATOM | 468 | OD2 | ASP | U | 357 | 10.859 | 100.150 | 32.599 | 1.00 | 97.29 | U |
| ATOM | 469 | C | ASP | U | 357 | 12.260 | 102.132 | 28.483 | 1.00 | 67.53 | U |
| ATOM | 470 | O | ASP | U | 357 | 11.183 | 102.671 | 28.208 | 1.00 | 66.44 | U |
| ATOM | 471 | N | GLY | U | 358 | 13.340 | 102.247 | 27.715 | 1.00 | 66.29 | U |
| ATOM | 472 | CA | GLY | U | 358 | 13.281 | 103.031 | 26.504 | 1.00 | 57.58 | U |
| ATOM | 473 | C | GLY | U | 358 | 14.569 | 103.066 | 25.712 | 1.00 | 57.81 | U |
| ATOM | 474 | O | GLY | U | 358 | 15.602 | 102.523 | 26.118 | 1.00 | 59.38 | U |
| ATOM | 475 | N | ILE | U | 359 | 14.465 | 103.736 | 24.568 | 1.00 | 53.70 | U |
| ATOM | 476 | CA | ILE | U | 359 | 15.532 | 103.943 | 23.601 | 1.00 | 47.56 | U |
| ATOM | 477 | CB | ILE | U | 359 | 15.116 | 103.351 | 22.218 | 1.00 | 39.27 | U |
| ATOM | 478 | CG2 | ILE | U | 359 | 16.111 | 103.758 | 21.133 | 1.00 | 33.07 | U |
| ATOM | 479 | CG1 | ILE | U | 359 | 14.988 | 101.830 | 22.326 | 1.00 | 41.53 | U |
| ATOM | 480 | CD1 | ILE | U | 359 | 14.632 | 101.143 | 21.032 | 1.00 | 47.71 | U |
| ATOM | 481 | C | ILE | U | 359 | 15.725 | 105.453 | 23.435 | 1.00 | 50.08 | U |
| ATOM | 482 | O | ILE | U | 359 | 14.781 | 106.162 | 23.126 | 1.00 | 50.57 | U |
| ATOM | 483 | N | MET | U | 360 | 16.931 | 105.962 | 23.644 | 1.00 | 51.66 | U |
| ATOM | 484 | CA | MET | U | 360 | 17.153 | 107.392 | 23.447 | 1.00 | 54.19 | U |
| ATOM | 485 | CB | MET | U | 360 | 18.253 | 107.874 | 24.368 | 1.00 | 55.34 | U |
| ATOM | 486 | CG | MET | U | 360 | 18.559 | 109.320 | 24.191 | 1.00 | 63.76 | U |
| ATOM | 487 | SD | MET | U | 360 | 19.630 | 109.862 | 25.488 | 1.00 | 68.21 | U |
| ATOM | 488 | CE | MET | U | 360 | 18.423 | 110.302 | 26.681 | 1.00 | 63.93 | U |
| ATOM | 489 | C | MET | U | 360 | 17.550 | 107.636 | 21.986 | 1.00 | 57.24 | U |
| ATOM | 490 | O | MET | U | 360 | 18.051 | 106.731 | 21.331 | 1.00 | 62.64 | U |
| ATOM | 491 | N | LEU | U | 361 | 17.336 | 108.841 | 21.466 | 1.00 | 56.32 | U |
| ATOM | 492 | CA | LEU | U | 361 | 17.680 | 109.124 | 20.070 | 1.00 | 56.54 | U |
| ATOM | 493 | CB | LEU | U | 361 | 16.463 | 108.895 | 19.171 | 1.00 | 52.54 | U |
| ATOM | 494 | CG | LEU | U | 361 | 15.778 | 107.526 | 19.099 | 1.00 | 53.61 | U |
| ATOM | 495 | CD1 | LEU | U | 361 | 14.474 | 107.633 | 18.310 | 1.00 | 46.05 | U |
| ATOM | 496 | CD2 | LEU | U | 361 | 16.707 | 106.520 | 18.440 | 1.00 | 57.61 | U |
| ATOM | 497 | C | LEU | U | 361 | 18.157 | 110.556 | 19.870 | 1.00 | 62.43 | U |
| ATOM | 498 | O | LEU | U | 361 | 17.399 | 111.493 | 20.088 | 1.00 | 69.23 | U |
| ATOM | 499 | N | ALA | U | 362 | 19.397 | 110.732 | 19.430 | 1.00 | 72.75 | U |
| ATOM | 500 | CA | ALA | U | 362 | 19.945 | 112.077 | 19.207 | 1.00 | 81.97 | U |
| ATOM | 501 | CB | ALA | U | 362 | 19.448 | 112.637 | 17.875 | 1.00 | 82.62 | U |
| ATOM | 502 | C | ALA | U | 362 | 19.582 | 113.039 | 20.352 | 1.00 | 88.14 | U |
| ATOM | 503 | O | ALA | U | 362 | 18.788 | 113.989 | 20.170 | 1.00 | 86.81 | U |
| ATOM | 504 | N | THR | U | 363 | 20.186 | 112.762 | 21.516 | 1.00 | 90.21 | U |
| ATOM | 505 | CA | THR | U | 363 | 20.024 | 113.509 | 22.775 | 1.00 | 84.69 | U |
| ATOM | 506 | CB | THR | U | 363 | 20.732 | 114.906 | 22.742 | 1.00 | 84.81 | U |
| ATOM | 507 | OG1 | THR | U | 363 | 21.948 | 114.849 | 23.508 | 1.00 | 74.38 | U |
| ATOM | 508 | CG2 | THR | U | 363 | 19.823 | 115.996 | 23.318 | 1.00 | 78.85 | U |
| ATOM | 509 | C | THR | U | 363 | 18.579 | 113.700 | 23.162 | 1.00 | 80.56 | U |
| ATOM | 510 | O | THR | U | 363 | 17.762 | 114.165 | 22.365 | 1.00 | 76.72 | U |
| ATOM | 511 | N | GLY | U | 364 | 18.271 | 113.358 | 24.405 | 1.00 | 80.95 | U |
| ATOM | 512 | CA | GLY | U | 364 | 16.907 | 113.490 | 24.853 | 1.00 | 79.03 | U |
| ATOM | 513 | C | GLY | U | 364 | 16.090 | 112.686 | 23.863 | 1.00 | 78.51 | U |
| ATOM | 514 | O | GLY | U | 364 | 16.638 | 111.933 | 23.049 | 1.00 | 79.46 | U |
| ATOM | 515 | N | LEU | U | 365 | 14.780 | 112.876 | 23.903 | 1.00 | 72.04 | U |
| ATOM | 516 | CA | LEU | U | 365 | 13.866 | 112.142 | 23.049 | 1.00 | 64.93 | U |
| ATOM | 517 | CB | LEU | U | 365 | 14.132 | 112.386 | 21.557 | 1.00 | 54.02 | U |
| ATOM | 518 | CG | LEU | U | 365 | 12.824 | 112.119 | 20.785 | 1.00 | 50.92 | U |
| ATOM | 519 | CD1 | LEU | U | 365 | 11.779 | 113.069 | 21.304 | 1.00 | 38.89 | U |
| ATOM | 520 | CD2 | LEU | U | 365 | 12.974 | 112.315 | 19.302 | 1.00 | 51.79 | U |
| ATOM | 521 | C | LEU | U | 365 | 13.990 | 110.657 | 23.367 | 1.00 | 64.92 | U |
| ATOM | 522 | O | LEU | U | 365 | 14.497 | 109.863 | 22.574 | 1.00 | 69.33 | U |
| ATOM | 523 | N | VAL | U | 366 | 13.542 | 110.297 | 24.560 | 1.00 | 56.23 | U |
| ATOM | 524 | CA | VAL | U | 366 | 13.563 | 108.923 | 24.978 | 1.00 | 53.91 | U |
| ATOM | 525 | CB | VAL | U | 366 | 13.728 | 108.814 | 26.476 | 1.00 | 51.37 | U |
| ATOM | 526 | CG1 | VAL | U | 366 | 13.522 | 107.380 | 26.913 | 1.00 | 48.12 | U |
| ATOM | 527 | CG2 | VAL | U | 366 | 15.102 | 109.303 | 26.861 | 1.00 | 53.75 | U |
| ATOM | 528 | C | VAL | U | 366 | 12.246 | 108.291 | 24.580 | 1.00 | 57.47 | U |
| ATOM | 529 | O | VAL | U | 366 | 11.224 | 108.464 | 25.240 | 1.00 | 68.08 | U |
| ATOM | 530 | N | VAL | U | 367 | 12.273 | 107.578 | 23.470 | 1.00 | 55.00 | U |
| ATOM | 531 | CA | VAL | U | 367 | 11.100 | 106.891 | 22.965 | 1.00 | 49.29 | U |
| ATOM | 532 | CB | VAL | U | 367 | 11.436 | 106.249 | 21.619 | 1.00 | 35.95 | U |
| ATOM | 533 | CG1 | VAL | U | 367 | 10.261 | 105.484 | 21.083 | 1.00 | 39.11 | U |
| ATOM | 534 | CG2 | VAL | U | 367 | 11.881 | 107.314 | 20.660 | 1.00 | 36.47 | U |
| ATOM | 535 | C | VAL | U | 367 | 10.679 | 105.789 | 23.945 | 1.00 | 53.36 | U |
| ATOM | 536 | O | VAL | U | 367 | 11.525 | 105.036 | 24.435 | 1.00 | 56.64 | U |
| ATOM | 537 | N | HIS | U | 368 | 9.388 | 105.706 | 24.258 | 1.00 | 54.80 | U |
| ATOM | 538 | CA | HIS | U | 368 | 8.915 | 104.634 | 25.133 | 1.00 | 53.25 | U |
| ATOM | 539 | CB | HIS | U | 368 | 8.081 | 105.170 | 26.287 | 1.00 | 54.77 | U |
| ATOM | 540 | CG | HIS | U | 368 | 8.879 | 105.941 | 27.285 | 1.00 | 58.76 | U |
| ATOM | 541 | CD2 | HIS | U | 368 | 8.956 | 107.269 | 27.534 | 1.00 | 58.35 | U |
| ATOM | 542 | ND1 | HIS | U | 368 | 9.789 | 105.340 | 28.126 | 1.00 | 63.03 | U |
| ATOM | 543 | CE1 | HIS | U | 368 | 10.395 | 106.266 | 28.850 | 1.00 | 62.51 | U |
| ATOM | 544 | NE2 | HIS | U | 368 | 9.908 | 107.445 | 28.509 | 1.00 | 60.14 | U |
| ATOM | 545 | C | HIS | U | 368 | 8.093 | 103.700 | 24.280 | 1.00 | 50.90 | U |
| ATOM | 546 | O | HIS | U | 368 | 7.641 | 104.062 | 23.196 | 1.00 | 48.41 | U |
| ATOM | 547 | N | ARG | U | 369 | 7.898 | 102.489 | 24.766 | 1.00 | 52.36 | U |
| ATOM | 548 | CA | ARG | U | 369 | 7.153 | 101.515 | 23.996 | 1.00 | 52.38 | U |
| ATOM | 549 | CB | ARG | U | 369 | 6.907 | 100.271 | 24.830 | 1.00 | 48.03 | U |
| ATOM | 550 | CG | ARG | U | 369 | 6.370 | 99.130 | 24.021 | 1.00 | 46.98 | U |
| ATOM | 551 | CD | ARG | U | 369 | 5.934 | 98.029 | 24.947 | 1.00 | 54.79 | U |
| ATOM | 552 | NE | ARG | U | 369 | 4.898 | 97.229 | 24.317 | 1.00 | 55.68 | U |
| ATOM | 553 | CZ | ARG | U | 369 | 5.144 | 96.193 | 23.533 | 1.00 | 55.09 | U |
| ATOM | 554 | NH1 | ARG | U | 369 | 6.398 | 95.842 | 23.302 | 1.00 | 56.98 | U |
| ATOM | 555 | NH2 | ARG | U | 369 | 4.142 | 95.517 | 22.977 | 1.00 | 51.49 | U |
| ATOM | 556 | C | ARG | U | 369 | 5.833 | 102.060 | 23.486 | 1.00 | 54.06 | U |
| ATOM | 557 | O | ARG | U | 369 | 5.401 | 101.731 | 22.383 | 1.00 | 50.17 | U |
| ATOM | 558 | N | ASN | U | 370 | 5.210 | 102.922 | 24.282 | 1.00 | 57.80 | U |
| ATOM | 559 | CA | ASN | U | 370 | 3.914 | 103.493 | 23.923 | 1.00 | 61.00 | U |
| ATOM | 560 | CB | ASN | U | 370 | 3.341 | 104.296 | 25.092 | 1.00 | 63.08 | U |
| ATOM | 561 | CG | ASN | U | 370 | 3.963 | 105.667 | 25.214 | 1.00 | 68.85 | U |
| ATOM | 562 | OD1 | ASN | U | 370 | 5.139 | 105.805 | 25.561 | 1.00 | 71.99 | U |
| ATOM | 563 | ND2 | ASN | U | 370 | 3.175 | 106.697 | 24.924 | 1.00 | 71.17 | U |
| ATOM | 564 | C | ASN | U | 370 | 3.906 | 104.360 | 22.669 | 1.00 | 57.09 | U |
| ATOM | 565 | O | ASN | U | 370 | 2.895 | 104.962 | 22.343 | 1.00 | 58.09 | U |
| ATOM | 566 | N | CYS | U | 371 | 5.024 | 104.431 | 21.963 | 1.00 | 56.66 | U |
| ATOM | 567 | CA | CYS | U | 371 | 5.080 | 105.218 | 20.736 | 1.00 | 54.61 | U |
| ATOM | 568 | CB | CYS | U | 371 | 4.938 | 106.716 | 21.036 | 1.00 | 52.84 | U |
| ATOM | 569 | SG | CYS | U | 371 | 6.114 | 107.406 | 22.206 | 1.00 | 56.83 | U |
| ATOM | 570 | C | CYS | U | 371 | 6.378 | 104.914 | 20.003 | 1.00 | 53.71 | U |
| ATOM | 571 | O | CYS | U | 371 | 7.039 | 105.791 | 19.434 | 1.00 | 46.06 | U |
| ATOM | 572 | N | ALA | U | 372 | 6.742 | 103.638 | 20.048 | 1.00 | 50.90 | U |
| ATOM | 573 | CA | ALA | U | 372 | 7.916 | 103.168 | 19.358 | 1.00 | 49.08 | U |
| ATOM | 574 | CB | ALA | U | 372 | 8.202 | 101.735 | 19.748 | 1.00 | 46.77 | U |
| ATOM | 575 | C | ALA | U | 372 | 7.529 | 103.266 | 17.882 | 1.00 | 49.54 | U |
| ATOM | 576 | O | ALA | U | 372 | 8.364 | 103.574 | 17.032 | 1.00 | 44.53 | U |
| ATOM | 577 | N | HIS | U | 373 | 6.247 | 103.016 | 17.595 | 1.00 | 52.25 | U |
| ATOM | 578 | CA | HIS | U | 373 | 5.729 | 103.093 | 16.233 | 1.00 | 52.88 | U |
| ATOM | 579 | CB | HIS | U | 373 | 4.258 | 102.706 | 16.171 | 1.00 | 49.41 | U |
| ATOM | 580 | CG | HIS | U | 373 | 3.764 | 102.502 | 14.772 | 1.00 | 60.05 | U |
| ATOM | 581 | CD2 | HIS | U | 373 | 3.170 | 103.351 | 13.903 | 1.00 | 57.01 | U |
| ATOM | 582 | ND1 | HIS | U | 373 | 3.948 | 101.317 | 14.084 | 1.00 | 70.47 | U |
| ATOM | 583 | CE1 | HIS | U | 373 | 3.489 | 101.451 | 12.851 | 1.00 | 64.76 | U |
| ATOM | 584 | NE2 | HIS | U | 373 | 3.012 | 102.674 | 12.712 | 1.00 | 57.34 | U |
| ATOM | 585 | C | HIS | U | 373 | 5.898 | 104.508 | 15.674 | 1.00 | 55.40 | U |
| ATOM | 586 | O | HIS | U | 373 | 6.297 | 104.675 | 14.521 | 1.00 | 53.94 | U |
| ATOM | 587 | N | GLN | U | 374 | 5.575 | 105.528 | 16.472 | 1.00 | 58.34 | U |
| ATOM | 588 | CA | GLN | U | 374 | 5.765 | 106.904 | 16.016 | 1.00 | 60.92 | U |
| ATOM | 589 | CB | GLN | U | 374 | 5.473 | 107.935 | 17.121 | 1.00 | 72.96 | U |
| ATOM | 590 | CG | GLN | U | 374 | 4.001 | 108.340 | 17.317 | 1.00 | 90.65 | U |
| ATOM | 591 | CD | GLN | U | 374 | 3.824 | 109.665 | 18.108 | 1.00 | 96.81 | U |
| ATOM | 592 | OE1 | GLN | U | 374 | 4.387 | 109.853 | 19.198 | 1.00 | 95.94 | U |
| ATOM | 593 | NE2 | GLN | U | 374 | 3.022 | 110.576 | 17.553 | 1.00 | 99.87 | U |
| ATOM | 594 | C | GLN | U | 374 | 7.233 | 107.049 | 15.634 | 1.00 | 56.05 | U |
| ATOM | 595 | O | GLN | U | 374 | 7.556 | 107.320 | 14.480 | 1.00 | 54.14 | U |
| ATOM | 596 | N | ALA | U | 375 | 8.105 | 106.858 | 16.627 | 1.00 | 50.29 | U |
| ATOM | 597 | CA | ALA | U | 375 | 9.562 | 106.968 | 16.481 | 1.00 | 45.90 | U |
| ATOM | 598 | CB | ALA | U | 375 | 10.237 | 106.440 | 17.733 | 1.00 | 36.92 | U |
| ATOM | 599 | C | ALA | U | 375 | 10.164 | 106.285 | 15.244 | 1.00 | 46.33 | U |
| ATOM | 600 | O | ALA | U | 375 | 11.085 | 106.810 | 14.620 | 1.00 | 41.94 | U |
| ATOM | 601 | N | GLY | U | 376 | 9.664 | 105.106 | 14.902 | 1.00 | 45.56 | U |
| ATOM | 602 | CA | GLY | U | 376 | 10.186 | 104.425 | 13.740 | 1.00 | 42.75 | U |
| ATOM | 603 | C | GLY | U | 376 | 11.000 | 103.226 | 14.126 | 1.00 | 43.80 | U |
| ATOM | 604 | O | GLY | U | 376 | 11.761 | 102.687 | 13.311 | 1.00 | 48.74 | U |
| ATOM | 605 | N | VAL | U | 377 | 10.845 | 102.826 | 15.381 | 1.00 | 39.82 | U |
| ATOM | 606 | CA | VAL | U | 377 | 11.552 | 101.679 | 15.915 | 1.00 | 42.16 | U |
| ATOM | 607 | CB | VAL | U | 377 | 12.483 | 102.067 | 17.072 | 1.00 | 38.64 | U |
| ATOM | 608 | CG1 | VAL | U | 377 | 13.687 | 102.855 | 16.538 | 1.00 | 41.57 | U |
| ATOM | 609 | CG2 | VAL | U | 377 | 11.712 | 102.851 | 18.108 | 1.00 | 24.01 | U |
| ATOM | 610 | C | VAL | U | 377 | 10.509 | 100.714 | 16.431 | 1.00 | 47.28 | U |
| ATOM | 611 | O | VAL | U | 377 | 10.682 | 100.074 | 17.479 | 1.00 | 50.04 | U |
| ATOM | 612 | N | GLY | U | 378 | 9.421 | 100.611 | 15.679 | 1.00 | 47.61 | U |
| ATOM | 613 | CA | GLY | U | 378 | 8.338 | 99.744 | 16.087 | 1.00 | 42.89 | U |
| ATOM | 614 | C | GLY | U | 378 | 8.637 | 98.279 | 15.932 | 1.00 | 41.14 | U |
| ATOM | 615 | O | GLY | U | 378 | 8.259 | 97.478 | 16.761 | 1.00 | 48.83 | U |
| ATOM | 616 | N | ALA | U | 379 | 9.336 | 97.917 | 14.876 | 1.00 | 45.44 | U |
| ATOM | 617 | CA | ALA | U | 379 | 9.627 | 96.511 | 14.631 | 1.00 | 50.56 | U |
| ATOM | 618 | CB | ALA | U | 379 | 10.009 | 96.318 | 13.159 | 1.00 | 54.17 | U |
| ATOM | 619 | C | ALA | U | 379 | 10.712 | 95.919 | 15.507 | 1.00 | 52.59 | U |
| ATOM | 620 | O | ALA | U | 379 | 10.821 | 94.703 | 15.606 | 1.00 | 54.95 | U |
| ATOM | 621 | N | ILE | U | 380 | 11.497 | 96.771 | 16.158 | 1.00 | 52.15 | U |
| ATOM | 622 | CA | ILE | U | 380 | 12.619 | 96.302 | 16.957 | 1.00 | 43.86 | U |
| ATOM | 623 | CB | ILE | U | 380 | 13.917 | 96.838 | 16.346 | 1.00 | 42.13 | U |
| ATOM | 624 | CG2 | ILE | U | 380 | 14.102 | 96.283 | 14.945 | 1.00 | 38.36 | U |
| ATOM | 625 | CG1 | ILE | U | 380 | 13.844 | 98.366 | 16.296 | 1.00 | 29.73 | U |
| ATOM | 626 | CD1 | ILE | U | 380 | 15.114 | 99.023 | 15.880 | 1.00 | 32.82 | U |
| ATOM | 627 | C | ILE | U | 380 | 12.638 | 96.647 | 18.442 | 1.00 | 45.23 | U |
| ATOM | 628 | O | ILE | U | 380 | 13.364 | 96.019 | 19.206 | 1.00 | 45.34 | U |
| ATOM | 629 | N | PHE | U | 381 | 11.856 | 97.639 | 18.852 | 1.00 | 41.47 | U |
| ATOM | 630 | CA | PHE | U | 381 | 11.834 | 98.077 | 20.250 | 1.00 | 39.86 | U |
| ATOM | 631 | CB | PHE | U | 381 | 10.536 | 98.812 | 20.520 | 1.00 | 40.79 | U |
| ATOM | 632 | CG | PHE | U | 381 | 10.577 | 99.631 | 21.751 | 1.00 | 38.51 | U |
| ATOM | 633 | CD1 | PHE | U | 381 | 11.041 | 100.945 | 21.702 | 1.00 | 43.23 | U |
| ATOM | 634 | CD2 | PHE | U | 381 | 10.225 | 99.076 | 22.976 | 1.00 | 31.79 | U |
| ATOM | 635 | CE1 | PHE | U | 381 | 11.160 | 101.712 | 22.875 | 1.00 | 50.56 | U |
| ATOM | 636 | CE2 | PHE | U | 381 | 10.338 | 99.820 | 24.153 | 1.00 | 40.15 | U |
| ATOM | 637 | CZ | PHE | U | 381 | 10.809 | 101.145 | 24.107 | 1.00 | 46.64 | U |
| ATOM | 638 | C | PHE | U | 381 | 12.081 | 97.057 | 21.393 | 1.00 | 38.24 | U |
| ATOM | 639 | O | PHE | U | 381 | 13.041 | 97.195 | 22.153 | 1.00 | 35.88 | U |
| ATOM | 640 | N | ASP | U | 382 | 11.214 | 96.057 | 21.530 | 1.00 | 36.81 | U |
| ATOM | 641 | CA | ASP | U | 382 | 11.370 | 95.040 | 22.584 | 1.00 | 41.40 | U |
| ATOM | 642 | CB | ASP | U | 382 | 10.287 | 93.949 | 22.481 | 1.00 | 48.31 | U |
| ATOM | 643 | CG | ASP | U | 382 | 8.897 | 94.435 | 22.894 | 1.00 | 53.81 | U |
| ATOM | 644 | OD1 | ASP | U | 382 | 8.780 | 95.150 | 23.924 | 1.00 | 54.60 | U |
| ATOM | 645 | OD2 | ASP | U | 382 | 7.919 | 94.071 | 22.189 | 1.00 | 51.69 | U |
| ATOM | 646 | C | ASP | U | 382 | 12.731 | 94.345 | 22.594 | 1.00 | 39.65 | U |
| ATOM | 647 | O | ASP | U | 382 | 13.439 | 94.371 | 23.592 | 1.00 | 41.97 | U |
| ATOM | 648 | N | ARG | U | 383 | 13.077 | 93.697 | 21.487 | 1.00 | 43.16 | U |
| ATOM | 649 | CA | ARG | U | 383 | 14.355 | 93.000 | 21.361 | 1.00 | 39.23 | U |
| ATOM | 650 | CB | ARG | U | 383 | 14.447 | 92.333 | 19.984 | 1.00 | 33.41 | U |
| ATOM | 651 | CG | ARG | U | 383 | 14.855 | 90.844 | 19.953 | 1.00 | 43.37 | U |
| ATOM | 652 | CD | ARG | U | 383 | 15.016 | 90.366 | 18.484 | 1.00 | 50.80 | U |
| ATOM | 653 | NE | ARG | U | 383 | 15.557 | 89.018 | 18.332 | 1.00 | 57.23 | U |
| ATOM | 654 | CZ | AEG | U | 383 | 16.858 | 88.718 | 18.348 | 1.00 | 74.35 | U |
| ATOM | 655 | NH1 | ARG | U | 383 | 17.795 | 89.675 | 18.511 | 1.00 | 59.21 | U |
| ATOM | 656 | NH2 | ARG | U | 383 | 17.221 | 87.441 | 18.196 | 1.00 | 76.48 | U |
| ATOM | 657 | C | ARG | U | 383 | 15.542 | 93.970 | 21.575 | 1.00 | 38.25 | U |
| ATOM | 658 | O | ARG | U | 383 | 16.539 | 93.600 | 22.189 | 1.00 | 38.14 | U |
| ATOM | 659 | N | VAL | U | 384 | 15.454 | 95.204 | 21.083 | 1.00 | 31.99 | U |
| ATOM | 660 | CA | VAL | U | 384 | 16.556 | 96.127 | 21.303 | 1.00 | 30.96 | U |
| ATOM | 661 | CB | VAL | U | 384 | 16.273 | 97.531 | 20.729 | 1.00 | 29.82 | U |
| ATOM | 662 | CG1 | VAL | U | 384 | 17.224 | 98.561 | 21.338 | 1.00 | 21.88 | U |
| ATOM | 663 | CG2 | VAL | U | 384 | 16.485 | 97.518 | 19.241 | 1.00 | 25.51 | U |
| ATOM | 664 | C | VAL | U | 384 | 16.838 | 96.232 | 22.800 | 1.00 | 34.32 | U |
| ATOM | 665 | O | VAL | U | 384 | 17.976 | 96.051 | 23.233 | 1.00 | 41.88 | U |
| ATOM | 666 | N | LEU | U | 385 | 15.817 | 96.493 | 23.602 | 1.00 | 30.94 | U |
| ATOM | 667 | CA | LEU | U | 385 | 16.029 | 96.608 | 25.036 | 1.00 | 30.42 | U |
| ATOM | 668 | CB | LEU | U | 385 | 14.720 | 96.993 | 25.724 | 1.00 | 28.36 | U |
| ATOM | 669 | CG | LEU | U | 385 | 14.070 | 98.331 | 25.373 | 1.00 | 37.72 | U |
| ATOM | 670 | CD1 | LEU | U | 385 | 12.820 | 98.477 | 26.174 | 1.00 | 31.55 | U |
| ATOM | 671 | CD2 | LEU | U | 385 | 14.994 | 99.495 | 25.684 | 1.00 | 38.29 | U |
| ATOM | 672 | C | LEU | U | 385 | 16.595 | 95.351 | 25.713 | 1.00 | 31.91 | U |
| ATOM | 673 | O | LEU | U | 385 | 17.476 | 95.439 | 26.569 | 1.00 | 33.34 | U |
| ATOM | 674 | N | THR | U | 386 | 16.109 | 94.179 | 25.321 | 1.00 | 26.65 | U |
| ATOM | 675 | CA | THR | U | 386 | 16.535 | 92.940 | 25.971 | 1.00 | 31.55 | U |
| ATOM | 676 | CB | THR | U | 386 | 15.385 | 91.910 | 26.010 | 1.00 | 34.28 | U |
| ATOM | 677 | OG1 | THR | U | 386 | 15.014 | 91.541 | 24.677 | 1.00 | 44.67 | U |
| ATOM | 678 | CG2 | THR | U | 386 | 14.171 | 92.497 | 26.708 | 1.00 | 31.26 | U |
| ATOM | 679 | C | THR | U | 386 | 17.764 | 92.215 | 25.458 | 1.00 | 36.23 | U |
| ATOM | 680 | O | THR | U | 386 | 18.501 | 91.638 | 26.254 | 1.00 | 40.01 | U |
| ATOM | 681 | N | GLU | U | 387 | 17.966 | 92.219 | 24.138 | 1.00 | 40.86 | U |
| ATOM | 682 | CA | GLU | U | 387 | 19.108 | 91.561 | 23.510 | 1.00 | 36.51 | U |
| ATOM | 683 | CB | GLU | U | 387 | 18.713 | 91.013 | 22.137 | 1.00 | 38.33 | U |
| ATOM | 684 | CG | GLU | U | 387 | 17.485 | 90.160 | 22.161 | 1.00 | 42.62 | U |
| ATOM | 685 | CD | GLU | U | 387 | 17.482 | 89.162 | 23.304 | 1.00 | 47.11 | U |
| ATOM | 686 | OE1 | GLU | U | 387 | 18.256 | 88.166 | 23.254 | 1.00 | 44.10 | U |
| ATOM | 687 | OE2 | GLU | U | 387 | 16.696 | 89.387 | 24.256 | 1.00 | 42.20 | U |
| ATOM | 688 | C | GLU | U | 387 | 20.337 | 92.468 | 23.350 | 1.00 | 36.93 | U |
| ATOM | 689 | O | GLU | U | 387 | 21.450 | 91.966 | 23.160 | 1.00 | 37.80 | U |
| ATOM | 690 | N | LEU | U | 388 | 20.141 | 93.787 | 23.401 | 1.00 | 28.40 | U |
| ATOM | 691 | CA | LEU | U | 388 | 21.253 | 94.710 | 23.267 | 1.00 | 30.94 | U |
| ATOM | 692 | CB | LEU | U | 388 | 21.128 | 95.542 | 21.990 | 1.00 | 28.29 | U |
| ATOM | 693 | CG | LEU | U | 388 | 21.185 | 94.698 | 20.704 | 1.00 | 30.69 | U |
| ATOM | 694 | CD1 | LEU | U | 388 | 20.963 | 95.564 | 19.485 | 1.00 | 27.49 | U |
| ATOM | 695 | CD2 | LEU | U | 388 | 22.517 | 93.980 | 20.626 | 1.00 | 22.93 | U |
| ATOM | 696 | C | LEU | U | 388 | 21.365 | 95.623 | 24.467 | 1.00 | 35.06 | U |
| ATOM | 697 | O | LEU | U | 388 | 22.353 | 95.562 | 25.203 | 1.00 | 36.04 | U |
| ATOM | 698 | N | VAL | U | 389 | 20.358 | 96.450 | 24.701 | 1.00 | 32.72 | U |
| ATOM | 699 | CA | VAL | U | 389 | 20.456 | 97.352 | 25.834 | 1.00 | 31.84 | U |
| ATOM | 700 | CB | VAL | U | 389 | 19.187 | 98.198 | 26.050 | 1.00 | 33.37 | U |
| ATOM | 701 | CG1 | VAL | U | 389 | 19.404 | 99.110 | 27.259 | 1.00 | 27.98 | U |
| ATOM | 702 | CG2 | VAL | U | 389 | 18.888 | 99.043 | 24.821 | 1.00 | 24.09 | U |
| ATOM | 703 | C | VAL | U | 389 | 20.769 | 96.626 | 27.132 | 1.00 | 30.80 | U |
| ATOM | 704 | O | VAL | U | 389 | 21.739 | 96.963 | 27.801 | 1.00 | 38.49 | U |
| ATOM | 705 | N | ALA | U | 390 | 19.962 | 95.636 | 27.494 | 1.00 | 32.86 | U |
| ATOM | 706 | CA | ALA | U | 390 | 20.193 | 94.893 | 28.736 | 1.00 | 33.12 | U |
| ATOM | 707 | CB | ALA | U | 390 | 19.120 | 93.861 | 28.927 | 1.00 | 30.29 | U |
| ATOM | 708 | C | ALA | U | 390 | 21.561 | 94.218 | 28.756 | 1.00 | 33.32 | U |
| ATOM | 709 | O | ALA | U | 390 | 22.336 | 94.396 | 29.680 | 1.00 | 32.84 | U |
| ATOM | 710 | N | LYS | U | 391 | 21.867 | 93.440 | 27.733 | 1.00 | 37.03 | U |
| ATOM | 711 | CA | LYS | U | 391 | 23.150 | 92.763 | 27.681 | 1.00 | 38.70 | U |
| ATOM | 712 | CB | LYS | U | 391 | 23.208 | 91.883 | 26.430 | 1.00 | 40.17 | U |
| ATOM | 713 | CG | LYS | U | 391 | 22.157 | 90.771 | 26.412 | 1.00 | 35.70 | U |
| ATOM | 714 | CD | LYS | U | 391 | 22.468 | 89.712 | 27.453 | 1.00 | 40.05 | U |
| ATOM | 715 | CE | LYS | U | 391 | 21.234 | 88.915 | 27.878 | 1.00 | 45.83 | U |
| ATOM | 716 | NZ | LYS | U | 391 | 21.521 | 88.077 | 29.099 | 1.00 | 49.07 | U |
| ATOM | 717 | C | LYS | U | 391 | 24.328 | 93.747 | 27.710 | 1.00 | 40.62 | U |
| ATOM | 718 | O | LYS | U | 391 | 25.410 | 93.424 | 28.234 | 1.00 | 33.01 | U |
| ATOM | 719 | N | MET | U | 392 | 24.115 | 94.942 | 27.153 | 1.00 | 40.44 | U |
| ATOM | 720 | CA | MET | U | 392 | 25.163 | 95.974 | 27.106 | 1.00 | 40.66 | U |
| ATOM | 721 | CB | MET | U | 392 | 24.731 | 97.145 | 26.229 | 1.00 | 35.96 | U |
| ATOM | 722 | CG | MET | U | 392 | 25.072 | 96.961 | 24.791 | 1.00 | 33.56 | U |
| ATOM | 723 | SD | MET | U | 392 | 24.440 | 98.258 | 23.783 | 1.00 | 40.35 | U |
| ATOM | 724 | CE | MET | U | 392 | 24.913 | 97.647 | 22.178 | 1.00 | 27.49 | U |
| ATOM | 725 | C | MET | U | 392 | 25.473 | 96.497 | 28.484 | 1.00 | 38.99 | U |
| ATOM | 726 | O | MET | U | 392 | 26.630 | 96.648 | 28.881 | 1.00 | 39.17 | U |
| ATOM | 727 | N | ARG | U | 393 | 24.398 | 96.790 | 29.191 | 1.00 | 37.45 | U |
| ATOM | 728 | CA | ARG | U | 393 | 24.453 | 97.291 | 30.541 | 1.00 | 32.99 | U |
| ATOM | 729 | CB | ARG | U | 393 | 23.045 | 97.654 | 30.969 | 1.00 | 34.44 | U |
| ATOM | 730 | CG | ARG | U | 393 | 22.929 | 98.436 | 32.223 | 1.00 | 41.19 | U |
| ATOM | 731 | CD | ARG | U | 393 | 21.610 | 99.172 | 32.177 | 1.00 | 47.37 | U |
| ATOM | 732 | NE | ARG | U | 393 | 20.517 | 98.254 | 31.880 | 1.00 | 55.20 | U |
| ATOM | 733 | CZ | ARG | U | 393 | 19.379 | 98.621 | 31.302 | 1.00 | 60.08 | U |
| ATOM | 734 | NH1 | ARG | U | 393 | 19.185 | 99.893 | 30.958 | 1.00 | 50.39 | U |
| ATOM | 735 | NH2 | ARG | U | 393 | 18.442 | 97.709 | 31.056 | 1.00 | 66.60 | U |
| ATOM | 736 | C | ARG | U | 393 | 25.025 | 96.191 | 31.419 | 1.00 | 28.95 | U |
| ATOM | 737 | O | ARG | U | 393 | 25.947 | 96.435 | 32.187 | 1.00 | 27.65 | U |
| ATOM | 738 | N | GLU | U | 394 | 24.505 | 94.973 | 31.287 | 1.00 | 26.38 | U |
| ATOM | 739 | CA | GLU | U | 394 | 25.006 | 93.862 | 32.089 | 1.00 | 32.40 | U |
| ATOM | 740 | CB | GLU | U | 394 | 24.355 | 92.548 | 31.658 | 1.00 | 34.10 | U |
| ATOM | 741 | CG | GLU | U | 394 | 22.857 | 92.493 | 31.857 | 1.00 | 47.11 | U |
| ATOM | 742 | CD | GLU | U | 394 | 22.255 | 91.185 | 31.350 | 1.00 | 57.63 | U |
| ATOM | 743 | OE1 | GLU | U | 394 | 23.042 | 90.269 | 30.997 | 1.00 | 60.19 | U |
| ATOM | 744 | OE2 | GLU | U | 394 | 21.005 | 91.071 | 31.308 | 1.00 | 57.06 | U |
| ATOM | 745 | C | GLU | U | 394 | 26.538 | 93.707 | 32.056 | 1.00 | 33.86 | U |
| ATOM | 746 | O | GLU | U | 394 | 27.146 | 93.459 | 33.082 | 1.00 | 33.65 | U |
| ATOM | 747 | N | MET | U | 395 | 27.161 | 93.854 | 30.888 | 1.00 | 37.99 | U |
| ATOM | 748 | CA | MET | U | 395 | 28.613 | 93.706 | 30.779 | 1.00 | 32.98 | U |
| ATOM | 749 | CB | MET | U | 395 | 29.005 | 93.009 | 29.468 | 1.00 | 36.56 | U |
| ATOM | 750 | CG | MET | U | 395 | 28.678 | 93.778 | 28.183 | 1.00 | 33.30 | U |
| ATOM | 751 | SD | MET | U | 395 | 29.054 | 92.793 | 26.707 | 1.00 | 37.42 | U |
| ATOM | 752 | CE | MET | U | 395 | 30.636 | 92.184 | 27.124 | 1.00 | 33.07 | U |
| ATOM | 753 | C | MET | U | 395 | 29.320 | 95.041 | 30.854 | 1.00 | 35.39 | U |
| ATOM | 754 | O | MET | U | 395 | 30.538 | 95.094 | 30.788 | 1.00 | 35.28 | U |
| ATOM | 755 | N | LYS | U | 396 | 28.567 | 96.125 | 30.980 | 1.00 | 27.78 | U |
| ATOM | 756 | CA | LYS | U | 396 | 29.203 | 97.411 | 31.059 | 1.00 | 29.20 | U |
| ATOM | 757 | CB | LYS | U | 396 | 30.001 | 97.486 | 32.358 | 1.00 | 32.38 | U |
| ATOM | 758 | CG | LYS | U | 396 | 29.118 | 97.516 | 33.605 | 1.00 | 43.32 | U |
| ATOM | 759 | CD | LYS | U | 396 | 29.916 | 97.831 | 34.872 | 1.00 | 54.00 | U |
| ATOM | 760 | CE | LYS | U | 396 | 29.011 | 97.989 | 36.092 | 1.00 | 57.36 | U |
| ATOM | 761 | NZ | LYS | U | 396 | 29.788 | 98.387 | 37.300 | 1.00 | 62.62 | U |
| ATOM | 762 | C | LYS | U | 396 | 30.108 | 97.621 | 29.847 | 1.00 | 30.91 | U |
| ATOM | 763 | O | LYS | U | 396 | 31.296 | 97.917 | 29.978 | 1.00 | 36.92 | U |
| ATOM | 764 | N | MET | U | 397 | 29.535 | 97.448 | 28.661 | 1.00 | 31.95 | U |
| ATOM | 765 | CA | MET | U | 397 | 30.265 | 97.620 | 27.407 | 1.00 | 35.62 | U |
| ATOM | 766 | CB | MET | U | 397 | 29.401 | 97.107 | 26.240 | 1.00 | 35.63 | U |
| ATOM | 767 | CG | MET | U | 397 | 29.977 | 97.373 | 24.858 | 1.00 | 34.00 | U |
| ATOM | 768 | SD | MET | U | 397 | 28.756 | 97.263 | 23.531 | 1.00 | 30.97 | U |
| ATOM | 769 | CE | MET | U | 397 | 28.855 | 95.574 | 23.156 | 1.00 | 31.90 | U |
| ATOM | 770 | C | MET | U | 397 | 30.575 | 99.109 | 27.224 | 1.00 | 33.23 | U |
| ATOM | 771 | O | MET | U | 397 | 29.668 | 99.926 | 27.139 | 1.00 | 36.18 | U |
| ATOM | 772 | N | ASP | U | 398 | 31.848 | 99.468 | 27.152 | 1.00 | 32.65 | U |
| ATOM | 773 | CA | ASP | U | 398 | 32.200 | 100.879 | 26.997 | 1.00 | 34.42 | U |
| ATOM | 774 | CB | ASP | U | 398 | 33.582 | 101.169 | 27.614 | 1.00 | 35.85 | U |
| ATOM | 775 | CG | ASP | U | 398 | 34.728 | 100.423 | 26.925 | 1.00 | 35.68 | U |
| ATOM | 776 | OD1 | ASP | U | 398 | 34.792 | 100.441 | 25.677 | 1.00 | 38.98 | U |
| ATOM | 777 | OD2 | ASP | U | 398 | 35.580 | 99.837 | 27.633 | 1.00 | 29.28 | U |
| ATOM | 778 | C | ASP | U | 398 | 32.167 | 101.405 | 25.559 | 1.00 | 39.08 | U |
| ATOM | 779 | O | ASP | U | 398 | 31.967 | 100.636 | 24.598 | 1.00 | 35.26 | U |
| ATOM | 780 | N | LYS | U | 399 | 32.366 | 102.724 | 25.441 | 1.00 | 37.68 | U |
| ATOM | 781 | CA | LYS | U | 399 | 32.378 | 103.450 | 24.165 | 1.00 | 36.84 | U |
| ATOM | 782 | CB | LYS | U | 399 | 32.656 | 104.953 | 24.387 | 1.00 | 42.08 | U |
| ATOM | 783 | CG | LYS | U | 399 | 31.637 | 105.771 | 25.183 | 1.00 | 48.45 | U |
| ATOM | 784 | CD | LYS | U | 399 | 32.218 | 107.182 | 25.482 | 1.00 | 53.38 | U |
| ATOM | 785 | CE | LYS | U | 399 | 31.441 | 107.945 | 26.589 | 1.00 | 53.01 | U |
| ATOM | 786 | NZ | LYS | U | 399 | 32.125 | 109.197 | 27.093 | 1.00 | 46.97 | U |
| ATOM | 787 | C | LYS | U | 399 | 33.442 | 102.926 | 23.172 | 1.00 | 35.69 | U |
| ATOM | 788 | O | LYS | U | 399 | 33.209 | 102.897 | 21.962 | 1.00 | 34.85 | U |
| ATOM | 789 | N | THR | U | 400 | 34.610 | 102.536 | 23.673 | 1.00 | 26.07 | U |
| ATOM | 790 | CA | THR | U | 400 | 35.684 | 102.061 | 22.814 | 1.00 | 29.08 | U |
| ATOM | 791 | CB | THR | U | 400 | 36.964 | 101.761 | 23.627 | 1.00 | 32.30 | U |
| ATOM | 792 | OG1 | THR | U | 400 | 37.430 | 102.962 | 24.238 | 1.00 | 27.35 | U |
| ATOM | 793 | CG2 | THR | U | 400 | 38.052 | 101.229 | 22.752 | 1.00 | 22.92 | U |
| ATOM | 794 | C | THR | U | 400 | 35.229 | 100.794 | 22.133 | 1.00 | 35.87 | U |
| ATOM | 795 | O | THR | U | 400 | 35.336 | 100.640 | 20.905 | 1.00 | 38.99 | U |
| ATOM | 796 | N | GLU | U | 401 | 34.715 | 99.887 | 22.953 | 1.00 | 36.25 | U |
| ATOM | 797 | CA | GLU | U | 401 | 34.226 | 98.604 | 22.484 | 1.00 | 34.75 | U |
| ATOM | 798 | CB | GLU | U | 401 | 33.757 | 97.799 | 23.691 | 1.00 | 30.59 | U |
| ATOM | 799 | CG | GLU | U | 401 | 34.890 | 97.654 | 24.684 | 1.00 | 35.88 | U |
| ATOM | 800 | CD | GLU | U | 401 | 34.537 | 96.872 | 25.915 | 1.00 | 36.12 | U |
| ATOM | 801 | OE1 | GLU | U | 401 | 33.547 | 97.229 | 26.595 | 1.00 | 29.09 | U |
| ATOM | 802 | OE2 | GLU | U | 401 | 35.271 | 95.903 | 26.205 | 1.00 | 36.39 | U |
| ATOM | 803 | C | GLU | U | 401 | 33.110 | 98.811 | 21.463 | 1.00 | 33.50 | U |
| ATOM | 804 | O | GLU | U | 401 | 33.123 | 98.217 | 20.376 | 1.00 | 32.04 | U |
| ATOM | 805 | N | LEU | U | 402 | 32.162 | 99.682 | 21.806 | 1.00 | 27.64 | U |
| ATOM | 806 | CA | LEU | U | 402 | 31.056 | 99.962 | 20.924 | 1.00 | 21.36 | U |
| ATOM | 807 | CB | LEU | U | 402 | 30.185 | 101.088 | 21.444 | 1.00 | 24.42 | U |
| ATOM | 808 | CG | LEU | U | 402 | 28.932 | 101.101 | 20.566 | 1.00 | 24.82 | U |
| ATOM | 809 | CD1 | LEU | U | 402 | 28.024 | 99.963 | 21.003 | 1.00 | 35.56 | U |
| ATOM | 810 | CD2 | LEU | U | 402 | 28.204 | 102.372 | 20.704 | 1.00 | 21.56 | U |
| ATOM | 811 | C | LEU | U | 402 | 31.580 | 100.373 | 19.586 | 1.00 | 24.94 | U |
| ATOM | 812 | O | LEU | U | 402 | 31.253 | 99.746 | 18.586 | 1.00 | 32.83 | U |
| ATOM | 813 | N | GLY | U | 403 | 32.375 | 101.442 | 19.561 | 1.00 | 24.62 | U |
| ATOM | 814 | CA | GLY | U | 403 | 32.945 | 101.925 | 18.311 | 1.00 | 22.95 | U |
| ATOM | 815 | C | GLY | U | 403 | 33.596 | 100.807 | 17.518 | 1.00 | 27.61 | U |
| ATOM | 816 | O | GLY | U | 403 | 33.468 | 100.738 | 16.303 | 1.00 | 26.58 | U |
| ATOM | 817 | N | CYS | U | 404 | 34.300 | 99.924 | 18.219 | 1.00 | 31.04 | U |
| ATOM | 818 | CA | CYS | U | 404 | 34.964 | 98.784 | 17.600 | 1.00 | 29.61 | U |
| ATOM | 819 | CB | CYS | U | 404 | 35.710 | 97.977 | 18.651 | 1.00 | 33.74 | U |
| ATOM | 820 | SG | CYS | U | 404 | 37.344 | 98.576 | 19.006 | 1.00 | 30.16 | U |
| ATOM | 821 | C | CYS | U | 404 | 33.988 | 97.861 | 16.897 | 1.00 | 35.64 | U |
| ATOM | 822 | O | CYS | U | 404 | 34.179 | 97.501 | 15.726 | 1.00 | 37.95 | U |
| ATOM | 823 | N | LEU | U | 405 | 32.952 | 97.450 | 17.617 | 1.00 | 29.69 | U |
| ATOM | 824 | CA | LEU | U | 405 | 31.977 | 96.564 | 17.020 | 1.00 | 31.19 | U |
| ATOM | 825 | CB | LEU | U | 405 | 30.935 | 96.136 | 18.052 | 1.00 | 23.77 | U |
| ATOM | 826 | CG | LEU | U | 405 | 31.439 | 95.309 | 19.227 | 1.00 | 23.97 | U |
| ATOM | 827 | CD1 | LEU | U | 405 | 30.371 | 95.289 | 20.274 | 1.00 | 20.63 | U |
| ATOM | 828 | CD2 | LEU | U | 405 | 31.838 | 93.905 | 18.782 | 1.00 | 13.06 | U |
| ATOM | 829 | C | LEU | U | 405 | 31.305 | 97.259 | 15.844 | 1.00 | 27.59 | U |
| ATOM | 830 | O | LEU | U | 405 | 31.135 | 96.668 | 14.782 | 1.00 | 31.16 | U |
| ATOM | 831 | N | ARG | U | 406 | 30.929 | 98.517 | 16.029 | 1.00 | 23.58 | U |
| ATOM | 832 | CA | ARG | U | 406 | 30.272 | 99.263 | 14.968 | 1.00 | 27.91 | U |
| ATOM | 833 | CB | ARG | U | 406 | 30.021 | 100.691 | 15.418 | 1.00 | 20.43 | U |
| ATOM | 834 | CG | ARG | U | 406 | 28.578 | 101.027 | 15.672 | 1.00 | 24.55 | U |
| ATOM | 835 | CD | ARG | U | 406 | 28.504 | 102.506 | 15.940 | 1.00 | 34.29 | U |
| ATOM | 836 | NE | ARG | U | 406 | 27.260 | 103.138 | 15.517 | 1.00 | 34.43 | U |
| ATOM | 837 | CZ | ARG | U | 406 | 27.092 | 104.457 | 15.521 | 1.00 | 39.30 | U |
| ATOM | 838 | NH1 | ARG | U | 406 | 28.087 | 105.231 | 15.926 | 1.00 | 35.42 | U |
| ATOM | 839 | NH2 | ARG | U | 406 | 25.953 | 105.011 | 15.114 | 1.00 | 41.12 | U |
| ATOM | 840 | C | ARG | U | 406 | 31.106 | 99.266 | 13.693 | 1.00 | 31.32 | U |
| ATOM | 841 | O | ARG | U | 406 | 30.575 | 99.237 | 12.584 | 1.00 | 32.23 | U |
| ATOM | 842 | N | SER | U | 407 | 32.419 | 99.298 | 13.866 | 1.00 | 29.69 | U |
| ATOM | 843 | CA | SER | U | 407 | 33.349 | 99.305 | 12.755 | 1.00 | 26.40 | U |
| ATOM | 844 | CB | SER | U | 407 | 34.711 | 99.770 | 13.230 | 1.00 | 23.92 | U |
| ATOM | 845 | OG | SER | U | 407 | 34.570 | 101.107 | 13.640 | 1.00 | 18.62 | U |
| ATOM | 846 | C | SER | U | 407 | 33.461 | 97.959 | 12.101 | 1.00 | 28.35 | U |
| ATOM | 847 | O | SER | U | 407 | 33.672 | 97.870 | 10.893 | 1.00 | 28.39 | U |
| ATOM | 848 | N | ILE | U | 408 | 33.357 | 96.908 | 12.907 | 1.00 | 33.99 | U |
| ATOM | 849 | CA | ILE | U | 408 | 33.386 | 95.554 | 12.372 | 1.00 | 30.44 | U |
| ATOM | 850 | CB | ILE | U | 408 | 33.331 | 94.526 | 13.492 | 1.00 | 23.83 | U |
| ATOM | 851 | CG2 | ILE | U | 408 | 33.150 | 93.161 | 12.896 | 1.00 | 11.06 | U |
| ATOM | 852 | CG1 | ILE | U | 408 | 34.582 | 94.660 | 14.365 | 1.00 | 20.58 | U |
| ATOM | 853 | CD1 | ILE | U | 408 | 34.814 | 93.494 | 15.302 | 1.00 | 24.44 | U |
| ATOM | 854 | C | ILE | U | 408 | 32.132 | 95.431 | 11.489 | 1.00 | 32.15 | U |
| ATOM | 855 | O | ILE | U | 408 | 32.138 | 94.755 | 10.464 | 1.00 | 35.94 | U |
| ATOM | 856 | N | VAL | U | 409 | 31.065 | 96.119 | 11.894 | 1.00 | 31.92 | U |
| ATOM | 857 | CA | VAL | U | 409 | 29.816 | 96.134 | 11.146 | 1.00 | 32.54 | U |
| ATOM | 858 | CB | VAL | U | 409 | 28.635 | 96.731 | 11.992 | 1.00 | 34.95 | U |
| ATOM | 859 | CG1 | VAL | U | 409 | 27.335 | 96.777 | 11.170 | 1.00 | 26.70 | U |
| ATOM | 860 | CG2 | VAL | U | 409 | 28.431 | 95.893 | 13.251 | 1.00 | 24.09 | U |
| ATOM | 861 | C | VAL | U | 409 | 29.988 | 96.955 | 9.878 | 1.00 | 31.14 | U |
| ATOM | 862 | O | VAL | U | 409 | 29.588 | 96.523 | 8.816 | 1.00 | 38.10 | U |
| ATOM | 863 | N | LEU | U | 410 | 30.584 | 98.138 | 9.978 | 1.00 | 34.55 | U |
| ATOM | 864 | CA | LEU | U | 410 | 30.784 | 98.989 | 8.797 | 1.00 | 32.93 | U |
| ATOM | 865 | CB | LEU | U | 410 | 31.380 | 100.337 | 9.202 | 1.00 | 25.79 | U |
| ATOM | 866 | CG | LEU | U | 410 | 31.701 | 101.291 | 8.053 | 1.00 | 26.02 | U |
| ATOM | 867 | CD1 | LEU | U | 410 | 30.479 | 102.056 | 7.707 | 1.00 | 29.87 | U |
| ATOM | 868 | CD2 | LEU | U | 410 | 32.825 | 102.247 | 8.436 | 1.00 | 27.59 | U |
| ATOM | 869 | C | LEU | U | 410 | 31.705 | 98.340 | 7.751 | 1.00 | 36.39 | U |
| ATOM | 870 | O | LEU | U | 410 | 31.441 | 98.413 | 6.538 | 1.00 | 37.38 | U |
| ATOM | 871 | N | PHE | U | 411 | 32.782 | 97.711 | 8.226 | 1.00 | 36.01 | U |
| ATOM | 872 | CA | PHE | U | 411 | 33.752 | 97.074 | 7.345 | 1.00 | 36.64 | U |
| ATOM | 873 | CB | PHE | U | 411 | 35.177 | 97.147 | 7.920 | 1.00 | 40.37 | U |
| ATOM | 874 | CG | PHE | U | 411 | 35.781 | 98.533 | 7.883 | 1.00 | 42.32 | U |
| ATOM | 875 | CD1 | PHE | U | 411 | 35.823 | 99.257 | 6.696 | 1.00 | 40.55 | U |
| ATOM | 876 | CD2 | PHE | U | 411 | 36.264 | 99.127 | 9.036 | 1.00 | 39.00 | U |
| ATOM | 877 | CE1 | PHE | U | 411 | 36.328 | 100.548 | 6.662 | 1.00 | 37.25 | U |
| ATOM | 878 | CE2 | PHE | U | 411 | 36.768 | 100.414 | 9.001 | 1.00 | 42.65 | U |
| ATOM | 879 | CZ | PHE | U | 411 | 36.798 | 101.124 | 7.810 | 1.00 | 42.34 | U |
| ATOM | 880 | C | PHE | U | 411 | 33.394 | 95.640 | 7.106 | 1.00 | 38.55 | U |
| ATOM | 881 | O | PHE | U | 411 | 34.123 | 94.741 | 7.523 | 1.00 | 32.24 | U |
| ATOM | 882 | N | ASN | U | 412 | 32.270 | 95.456 | 6.412 | 1.00 | 40.99 | U |
| ATOM | 883 | CA | ASN | U | 412 | 31.729 | 94.155 | 6.048 | 1.00 | 39.36 | U |
| ATOM | 884 | CB | ASN | U | 412 | 30.235 | 94.148 | 6.360 | 1.00 | 38.79 | U |
| ATOM | 885 | CG | ASN | U | 412 | 29.476 | 93.047 | 5.644 | 1.00 | 40.78 | U |
| ATOM | 886 | OD1 | ASN | U | 412 | 29.958 | 91.915 | 5.492 | 1.00 | 24.43 | U |
| ATOM | 887 | ND2 | ASN | U | 412 | 28.255 | 93.374 | 5.217 | 1.00 | 36.88 | U |
| ATOM | 888 | C | ASN | U | 412 | 31.998 | 93.825 | 4.578 | 1.00 | 41.92 | U |
| ATOM | 889 | O | ASN | U | 412 | 31.423 | 94.426 | 3.661 | 1.00 | 40.06 | U |
| ATOM | 890 | N | PRO | U | 413 | 32.902 | 92.862 | 4.338 | 1.00 | 45.60 | U |
| ATOM | 891 | CD | PRO | U | 413 | 33.680 | 92.144 | 5.365 | 1.00 | 41.57 | U |
| ATOM | 892 | CA | PRO | U | 413 | 33.285 | 92.424 | 2.991 | 1.00 | 44.83 | U |
| ATOM | 893 | CB | PRO | U | 413 | 34.516 | 91.566 | 3.250 | 1.00 | 39.25 | U |
| ATOM | 894 | CG | PRO | U | 413 | 34.223 | 90.974 | 4.588 | 1.00 | 39.15 | U |
| ATOM | 895 | C | PRO | U | 413 | 32.201 | 91.676 | 2.234 | 1.00 | 45.73 | U |
| ATOM | 896 | O | PRO | U | 413 | 32.222 | 91.626 | 1.002 | 1.00 | 52.42 | U |
| ATOM | 897 | N | GLU | U | 414 | 31.251 | 91.091 | 2.956 | 1.00 | 43.05 | U |
| ATOM | 898 | CA | GLU | U | 414 | 30.183 | 90.374 | 2.291 | 1.00 | 39.64 | U |
| ATOM | 899 | CB | GLU | U | 414 | 29.562 | 89.320 | 3.197 | 1.00 | 42.37 | U |
| ATOM | 900 | CG | GLU | U | 414 | 30.499 | 88.603 | 4.149 | 1.00 | 60.36 | U |
| ATOM | 901 | CD | GLU | U | 414 | 31.506 | 87.697 | 3.463 | 1.00 | 71.15 | U |
| ATOM | 902 | OE1 | GLU | U | 414 | 32.022 | 86.778 | 4.147 | 1.00 | 71.72 | U |
| ATOM | 903 | OE2 | GLU | U | 414 | 31.786 | 87.910 | 2.256 | 1.00 | 74.25 | U |
| ATOM | 904 | C | GLU | U | 414 | 29.111 | 91.386 | 1.911 | 1.00 | 42.28 | U |
| ATOM | 905 | O | GLU | U | 414 | 28.000 | 91.010 | 1.542 | 1.00 | 41.06 | U |
| ATOM | 906 | N | ALA | U | 415 | 29.414 | 92.678 | 2.033 | 1.00 | 44.92 | U |
| ATOM | 907 | CA | ALA | U | 415 | 28.420 | 93.676 | 1.646 | 1.00 | 46.88 | U |
| ATOM | 908 | CB | ALA | U | 415 | 28.942 | 95.101 | 1.877 | 1.00 | 39.65 | U |
| ATOM | 909 | C | ALA | U | 415 | 28.148 | 93.420 | 0.158 | 1.00 | 47.70 | U |
| ATOM | 910 | O | ALA | U | 415 | 29.050 | 93.055 | −0.615 | 1.00 | 40.83 | U |
| ATOM | 911 | N | LYS | U | 416 | 26.890 | 93.587 | −0.216 | 1.00 | 50.22 | U |
| ATOM | 912 | CA | LYS | U | 416 | 26.416 | 93.351 | −1.577 | 1.00 | 54.17 | U |
| ATOM | 913 | CB | LYS | U | 416 | 24.889 | 93.491 | −1.608 | 1.00 | 61.27 | U |
| ATOM | 914 | CG | LYS | U | 416 | 24.209 | 93.154 | −0.266 | 1.00 | 67.54 | U |
| ATOM | 915 | CD | LYS | U | 416 | 24.543 | 94.157 | 0.855 | 1.00 | 57.37 | U |
| ATOM | 916 | CE | LYS | U | 416 | 24.303 | 93.533 | 2.211 | 1.00 | 50.72 | U |
| ATOM | 917 | NZ | LYS | U | 416 | 22.901 | 93.042 | 2.362 | 1.00 | 56.29 | U |
| ATOM | 918 | C | LYS | U | 416 | 27.014 | 94.280 | −2.623 | 1.00 | 50.92 | U |
| ATOM | 919 | O | LYS | U | 416 | 26.849 | 95.493 | −2.551 | 1.00 | 51.14 | U |
| ATOM | 920 | N | GLY | U | 417 | 27.705 | 93.712 | −3.599 | 1.00 | 48.01 | U |
| ATOM | 921 | CA | GLY | U | 417 | 28.281 | 94.530 | −4.654 | 1.00 | 49.51 | U |
| ATOM | 922 | C | GLY | U | 417 | 29.534 | 95.307 | −4.303 | 1.00 | 49.64 | U |
| ATOM | 923 | O | GLY | U | 417 | 30.071 | 96.041 | −5.129 | 1.00 | 44.68 | U |
| ATOM | 924 | N | LEU | U | 418 | 29.991 | 95.165 | −3.066 | 1.00 | 54.51 | U |
| ATOM | 925 | CA | LEU | U | 418 | 31.205 | 95.837 | −2.622 | 1.00 | 48.92 | U |
| ATOM | 926 | CB | LEU | U | 418 | 31.587 | 95.340 | −1.225 | 1.00 | 41.73 | U |
| ATOM | 927 | CG | LEU | U | 418 | 32.899 | 95.860 | −0.671 | 1.00 | 35.06 | U |
| ATOM | 928 | CD1 | LEU | U | 418 | 32.768 | 97.354 | −0.506 | 1.00 | 40.21 | U |
| ATOM | 929 | CD2 | LEU | U | 418 | 33.224 | 95.171 | 0.639 | 1.00 | 28.86 | U |
| ATOM | 930 | C | LEU | U | 418 | 32.291 | 95.459 | −3.633 | 1.00 | 45.94 | U |
| ATOM | 931 | O | LEU | U | 418 | 32.422 | 94.297 | −4.026 | 1.00 | 36.89 | U |
| ATOM | 932 | N | LYS | U | 419 | 33.069 | 96.433 | −4.060 | 1.00 | 45.84 | U |
| ATOM | 933 | CA | LYS | U | 419 | 34.106 | 96.134 | −5.024 | 1.00 | 49.15 | U |
| ATOM | 934 | CB | LYS | U | 419 | 34.329 | 97.351 | −5.928 | 1.00 | 52.16 | U |
| ATOM | 935 | CG | LYS | U | 419 | 35.194 | 97.128 | −7.152 | 1.00 | 56.76 | U |
| ATOM | 936 | CD | LYS | U | 419 | 35.818 | 98.459 | −7.609 | 1.00 | 70.83 | U |
| ATOM | 937 | CE | LYS | U | 419 | 34.778 | 99.608 | −7.755 | 1.00 | 77.52 | U |
| ATOM | 938 | NZ | LYS | U | 419 | 35.404 | 100.967 | −8.007 | 1.00 | 72.38 | U |
| ATOM | 939 | C | LYS | U | 419 | 35.395 | 95.751 | −4.307 | 1.00 | 48.58 | U |
| ATOM | 940 | O | LYS | U | 419 | 35.939 | 94.670 | −4.542 | 1.00 | 51.80 | U |
| ATOM | 941 | N | SER | U | 420 | 35.863 | 96.619 | −3.412 | 1.00 | 42.58 | U |
| ATOM | 942 | CA | SER | U | 420 | 37.110 | 96.376 | −2.695 | 1.00 | 44.19 | U |
| ATOM | 943 | CB | SER | U | 420 | 37.672 | 97.697 | −2.194 | 1.00 | 45.46 | U |
| ATOM | 944 | OG | SER | U | 420 | 36.928 | 98.781 | −2.726 | 1.00 | 46.57 | U |
| ATOM | 945 | C | SER | U | 420 | 36.933 | 95.435 | −1.525 | 1.00 | 47.56 | U |
| ATOM | 946 | O | SER | U | 420 | 37.248 | 95.784 | −0.391 | 1.00 | 52.59 | U |
| ATOM | 947 | N | THR | U | 421 | 36.443 | 94.233 | −1.792 | 1.00 | 44.38 | U |
| ATOM | 948 | CA | THR | U | 421 | 36.224 | 93.283 | −0.720 | 1.00 | 42.89 | U |
| ATOM | 949 | CB | THR | U | 421 | 35.577 | 91.984 | −1.239 | 1.00 | 38.16 | U |
| ATOM | 950 | OG1 | THR | U | 421 | 36.442 | 91.353 | −2.182 | 1.00 | 39.40 | U |
| ATOM | 951 | CG2 | THR | U | 421 | 34.240 | 92.292 | −1.907 | 1.00 | 45.42 | U |
| ATOM | 952 | C | THR | U | 421 | 37.495 | 92.945 | 0.044 | 1.00 | 43.04 | U |
| ATOM | 953 | O | THR | U | 421 | 37.505 | 92.954 | 1.272 | 1.00 | 46.52 | U |
| ATOM | 954 | N | GLN | U | 422 | 38.575 | 92.666 | −0.670 | 1.00 | 44.07 | U |
| ATOM | 955 | CA | GLN | U | 422 | 39.810 | 92.331 | 0.009 | 1.00 | 44.59 | U |
| ATOM | 956 | CB | GLN | U | 422 | 40.896 | 91.968 | −1.003 | 1.00 | 50.43 | U |
| ATOM | 957 | CG | GLN | U | 422 | 42.105 | 91.358 | −0.319 | 1.00 | 56.93 | U |
| ATOM | 958 | CD | GLN | U | 422 | 41.709 | 90.282 | 0.702 | 1.00 | 66.07 | U |
| ATOM | 959 | OE1 | GLN | U | 422 | 41.338 | 89.162 | 0.331 | 1.00 | 71.38 | U |
| ATOM | 960 | NE2 | GLN | U | 422 | 41.776 | 90.626 | 1.993 | 1.00 | 64.40 | U |
| ATOM | 961 | C | GLN | U | 422 | 40.289 | 93.466 | 0.919 | 1.00 | 43.80 | U |
| ATOM | 962 | O | GLN | U | 422 | 40.727 | 93.234 | 2.042 | 1.00 | 36.35 | U |
| ATOM | 963 | N | GLN | U | 423 | 40.206 | 94.700 | 0.440 | 1.00 | 45.96 | U |
| ATOM | 964 | CA | GLN | U | 423 | 40.638 | 95.823 | 1.256 | 1.00 | 43.51 | U |
| ATOM | 965 | CB | GLN | U | 423 | 40.496 | 97.120 | 0.447 | 1.00 | 44.53 | U |
| ATOM | 966 | CG | GLN | U | 423 | 40.954 | 98.390 | 1.169 | 1.00 | 49.41 | U |
| ATOM | 967 | CD | GLN | U | 423 | 42.060 | 98.155 | 2.198 | 1.00 | 51.38 | U |
| ATOM | 968 | OE1 | GLN | U | 423 | 43.087 | 97.541 | 1.909 | 1.00 | 55.81 | U |
| ATOM | 969 | NE2 | GLN | U | 423 | 41.848 | 98.655 | 3.408 | 1.00 | 50.67 | U |
| ATOM | 970 | C | GLN | U | 423 | 39.819 | 95.862 | 2.555 | 1.00 | 43.42 | U |
| ATOM | 971 | O | GLN | U | 423 | 40.362 | 95.696 | 3.658 | 1.00 | 39.70 | U |
| ATOM | 972 | N | VAL | U | 424 | 38.511 | 96.066 | 2.398 | 1.00 | 39.62 | U |
| ATOM | 973 | CA | VAL | U | 424 | 37.543 | 96.121 | 3.495 | 1.00 | 33.70 | U |
| ATOM | 974 | CB | VAL | U | 424 | 36.119 | 96.067 | 2.908 | 1.00 | 32.61 | U |
| ATOM | 975 | CG1 | VAL | U | 424 | 35.101 | 95.797 | 3.983 | 1.00 | 24.92 | U |
| ATOM | 976 | CG2 | VAL | U | 424 | 35.818 | 97.385 | 2.187 | 1.00 | 36.03 | U |
| ATOM | 977 | C | VAL | U | 424 | 37.740 | 94.988 | 4.526 | 1.00 | 37.29 | U |
| ATOM | 978 | O | VAL | U | 424 | 37.546 | 95.162 | 5.730 | 1.00 | 39.08 | U |
| ATOM | 979 | N | GLU | U | 425 | 38.130 | 93.819 | 4.053 | 1.00 | 32.96 | U |
| ATOM | 980 | CA | GLU | U | 425 | 38.366 | 92.713 | 4.943 | 1.00 | 27.56 | U |
| ATOM | 981 | CB | GLU | U | 425 | 38.551 | 91.455 | 4.117 | 1.00 | 24.43 | U |
| ATOM | 982 | CG | GLU | U | 425 | 39.041 | 90.274 | 4.883 | 1.00 | 31.65 | U |
| ATOM | 983 | CD | GLU | U | 425 | 38.097 | 89.845 | 5.974 | 1.00 | 38.65 | U |
| ATOM | 984 | OE1 | GLU | U | 425 | 36.902 | 90.192 | 5.878 | 1.00 | 46.22 | U |
| ATOM | 985 | OE2 | GLU | U | 425 | 38.551 | 89.154 | 6.916 | 1.00 | 35.34 | U |
| ATOM | 986 | C | GLU | U | 425 | 39.593 | 92.989 | 5.818 | 1.00 | 32.07 | U |
| ATOM | 987 | O | GLU | U | 425 | 39.536 | 92.800 | 7.030 | 1.00 | 25.52 | U |
| ATOM | 988 | N | ASN | U | 426 | 40.694 | 93.450 | 5.219 | 1.00 | 35.40 | U |
| ATOM | 989 | CA | ASN | U | 426 | 41.911 | 93.733 | 5.993 | 1.00 | 39.46 | U |
| ATOM | 990 | CB | ASN | U | 426 | 43.056 | 94.262 | 5.121 | 1.00 | 44.98 | U |
| ATOM | 991 | CG | ASN | U | 426 | 43.538 | 93.242 | 4.099 | 1.00 | 56.28 | U |
| ATOM | 992 | OD1 | ASN | U | 426 | 43.488 | 92.026 | 4.332 | 1.00 | 60.50 | U |
| ATOM | 993 | ND2 | ASN | U | 426 | 44.021 | 93.732 | 2.964 | 1.00 | 53.75 | U |
| ATOM | 994 | C | ASN | U | 426 | 41.613 | 94.755 | 7.058 | 1.00 | 39.97 | U |
| ATOM | 995 | O | ASN | U | 426 | 42.164 | 94.690 | 8.166 | 1.00 | 40.06 | U |
| ATOM | 996 | N | LEU | U | 427 | 40.747 | 95.705 | 6.719 | 1.00 | 36.37 | U |
| ATOM | 997 | CA | LEU | U | 427 | 40.353 | 96.735 | 7.669 | 1.00 | 34.77 | U |
| ATOM | 998 | CB | LEU | U | 427 | 39.507 | 97.801 | 6.973 | 1.00 | 33.42 | U |
| ATOM | 999 | CG | LEU | U | 427 | 40.177 | 98.753 | 5.989 | 1.00 | 34.01 | U |
| ATOM | 1000 | CD1 | LEU | U | 427 | 39.120 | 99.513 | 5.193 | 1.00 | 28.60 | U |
| ATOM | 1001 | CD2 | LEU | U | 427 | 41.060 | 99.710 | 6.754 | 1.00 | 26.72 | U |
| ATOM | 1002 | C | LEU | U | 427 | 39.563 | 96.117 | 8.832 | 1.00 | 37.46 | U |
| ATOM | 1003 | O | LEU | U | 427 | 39.790 | 96.444 | 9.996 | 1.00 | 36.46 | U |
| ATOM | 1004 | N | ARG | U | 428 | 38.631 | 95.226 | 8.518 | 1.00 | 36.70 | U |
| ATOM | 1005 | CA | ARG | U | 428 | 37.836 | 94.581 | 9.564 | 1.00 | 39.47 | U |
| ATOM | 1006 | CB | ARG | U | 428 | 36.839 | 93.580 | 8.953 | 1.00 | 40.08 | U |
| ATOM | 1007 | CG | ARG | U | 428 | 35.825 | 92.988 | 9.921 | 1.00 | 23.37 | U |
| ATOM | 1008 | CD | ARG | U | 428 | 35.405 | 91.634 | 9.431 | 1.00 | 40.22 | U |
| ATOM | 1009 | NE | ARG | U | 428 | 34.019 | 91.230 | 9.725 | 1.00 | 43.20 | U |
| ATOM | 1010 | CZ | ARG | U | 428 | 32.941 | 91.863 | 9.283 | 1.00 | 27.82 | U |
| ATOM | 1011 | NH1 | ARG | U | 428 | 33.071 | 92.943 | 8.544 | 1.00 | 40.71 | U |
| ATOM | 1012 | NH2 | ARG | U | 428 | 31.742 | 91.386 | 9.523 | 1.00 | 27.69 | U |
| ATOM | 1013 | C | ARG | U | 428 | 38.746 | 93.825 | 10.515 | 1.00 | 37.61 | U |
| ATOM | 1014 | O | ARG | U | 428 | 38.546 | 93.840 | 11.730 | 1.00 | 35.39 | U |
| ATOM | 1015 | N | GLU | U | 429 | 39.742 | 93.163 | 9.938 | 1.00 | 37.57 | U |
| ATOM | 1016 | CA | GLU | U | 429 | 40.678 | 92.366 | 10.699 | 1.00 | 39.44 | U |
| ATOM | 1017 | CB | GLU | U | 429 | 41.512 | 91.530 | 9.751 | 1.00 | 43.58 | U |
| ATOM | 1018 | CG | GLU | U | 429 | 40.680 | 90.717 | 8.782 | 1.00 | 51.00 | U |
| ATOM | 1019 | CD | GLU | U | 429 | 41.442 | 89.532 | 8.227 | 1.00 | 54.03 | U |
| ATOM | 1020 | OE1 | GLU | U | 429 | 42.136 | 88.874 | 9.025 | 1.00 | 59.29 | U |
| ATOM | 1021 | OE2 | GLU | U | 429 | 41.341 | 89.239 | 7.015 | 1.00 | 53.59 | U |
| ATOM | 1022 | C | GLU | U | 429 | 41.567 | 93.166 | 11.641 | 1.00 | 42.36 | U |
| ATOM | 1023 | O | GLU | U | 429 | 42.022 | 92.619 | 12.649 | 1.00 | 45.72 | U |
| ATOM | 1024 | N | LYS | U | 430 | 41.821 | 94.442 | 11.323 | 1.00 | 40.65 | U |
| ATOM | 1025 | CA | LYS | U | 430 | 42.610 | 95.317 | 12.204 | 1.00 | 36.40 | U |
| ATOM | 1026 | CB | LYS | U | 430 | 42.848 | 96.691 | 11.573 | 1.00 | 38.62 | U |
| ATOM | 1027 | CG | LYS | U | 430 | 43.540 | 96.660 | 10.241 | 1.00 | 47.84 | U |
| ATOM | 1028 | CD | LYS | U | 430 | 44.948 | 96.101 | 10.323 | 1.00 | 54.43 | U |
| ATOM | 1029 | CE | LYS | U | 430 | 45.672 | 96.253 | 8.984 | 1.00 | 55.55 | U |
| ATOM | 1030 | NZ | LYS | U | 430 | 47.129 | 95.979 | 9.116 | 1.00 | 58.55 | U |
| ATOM | 1031 | C | LYS | U | 430 | 41.773 | 95.511 | 13.469 | 1.00 | 29.86 | U |
| ATOM | 1032 | O | LYS | U | 430 | 42.252 | 95.277 | 14.582 | 1.00 | 32.21 | U |
| ATOM | 1033 | N | VAL | U | 431 | 40.525 | 95.943 | 13.281 | 1.00 | 20.84 | U |
| ATOM | 1034 | CA | VAL | U | 431 | 39.594 | 96.137 | 14.384 | 1.00 | 25.88 | U |
| ATOM | 1035 | CB | VAL | U | 431 | 38.143 | 96.411 | 13.873 | 1.00 | 23.91 | U |
| ATOM | 1036 | CG1 | VAL | U | 431 | 37.212 | 96.606 | 15.064 | 1.00 | 27.22 | U |
| ATOM | 1037 | CG2 | VAL | U | 432 | 38.091 | 97.652 | 12.988 | 1.00 | 13.93 | U |
| ATOM | 1038 | C | VAL | U | 431 | 39.561 | 94.901 | 15.319 | 1.00 | 32.07 | U |
| ATOM | 1039 | O | VAL | U | 431 | 39.465 | 95.028 | 16.545 | 1.00 | 34.56 | U |
| ATOM | 1040 | N | TYR | U | 432 | 39.634 | 93.702 | 14.750 | 1.00 | 29.60 | U |
| ATOM | 1041 | CA | TYR | U | 432 | 39.610 | 92.504 | 15.574 | 1.00 | 28.79 | U |
| ATOM | 1042 | CB | TYR | U | 432 | 39.681 | 91.250 | 14.707 | 1.00 | 26.26 | U |
| ATOM | 1043 | CG | TYR | U | 432 | 38.407 | 90.812 | 14.019 | 1.00 | 28.57 | U |
| ATOM | 1044 | CD1 | TYR | U | 432 | 38.467 | 89.932 | 12.942 | 1.00 | 31.46 | U |
| ATOM | 1045 | CE1 | TYR | U | 432 | 37.338 | 89.530 | 12.271 | 1.00 | 30.13 | U |
| ATOM | 1046 | CD2 | TYR | U | 432 | 37.154 | 91.279 | 14.413 | 1.00 | 35.44 | U |
| ATOM | 1047 | CE2 | TYR | U | 432 | 36.000 | 90.877 | 13.731 | 1.00 | 33.27 | U |
| ATOM | 1048 | CZ | TYR | U | 432 | 36.108 | 89.998 | 12.661 | 1.00 | 32.17 | U |
| ATOM | 1049 | OH | TYR | U | 432 | 34.996 | 89.551 | 11.979 | 1.00 | 35.83 | U |
| ATOM | 1050 | C | TYR | U | 432 | 40.812 | 92.512 | 16.505 | 1.00 | 30.84 | U |
| ATOM | 1051 | O | TYR | U | 432 | 40.716 | 92.191 | 17.692 | 1.00 | 34.48 | U |
| ATOM | 1052 | N | ALA | U | 433 | 41.955 | 92.869 | 15.934 | 1.00 | 31.75 | U |
| ATOM | 1053 | CA | ALA | U | 433 | 43.211 | 92.907 | 16.657 | 1.00 | 28.58 | U |
| ATOM | 1054 | CB | ALA | U | 433 | 44.318 | 93.194 | 15.725 | 1.00 | 18.16 | U |
| ATOM | 1055 | C | ALA | U | 433 | 43.172 | 93.961 | 17.709 | 1.00 | 29.75 | U |
| ATOM | 1056 | O | ALA | U | 433 | 43.452 | 93.689 | 18.880 | 1.00 | 31.28 | U |
| ATOM | 1057 | N | ILE | U | 434 | 42.838 | 95.172 | 17.282 | 1.00 | 26.79 | U |
| ATOM | 1058 | CA | ILE | U | 434 | 42.760 | 96.313 | 18.187 | 1.00 | 28.71 | U |
| ATOM | 1059 | CB | ILE | U | 434 | 42.248 | 97.533 | 17.435 | 1.00 | 28.12 | U |
| ATOM | 1060 | CG2 | ILE | U | 434 | 41.861 | 98.618 | 18.399 | 1.00 | 20.04 | U |
| ATOM | 1061 | CG1 | ILE | U | 434 | 43.308 | 97.986 | 16.431 | 1.00 | 32.44 | U |
| ATOM | 1062 | CD1 | ILE | U | 434 | 42.846 | 99.059 | 15.470 | 1.00 | 37.48 | U |
| ATOM | 1063 | C | ILE | U | 434 | 41.844 | 96.008 | 19.369 | 1.00 | 32.28 | U |
| ATOM | 1064 | O | ILE | U | 434 | 42.242 | 96.126 | 20.521 | 1.00 | 31.88 | U |
| ATOM | 1065 | N | LEU | U | 435 | 40.622 | 95.590 | 19.061 | 1.00 | 34.58 | U |
| ATOM | 1066 | CA | LEU | U | 435 | 39.626 | 95.255 | 20.064 | 1.00 | 30.55 | U |
| ATOM | 1067 | CB | LEU | U | 435 | 38.332 | 94.848 | 19.383 | 1.00 | 24.76 | U |
| ATOM | 1068 | CG | LEU | U | 435 | 37.245 | 94.435 | 20.358 | 1.00 | 22.75 | U |
| ATOM | 1069 | CD1 | LEU | U | 435 | 36.888 | 95.597 | 21.287 | 1.00 | 14.85 | U |
| ATOM | 1070 | CD2 | LEU | U | 435 | 36.055 | 93.992 | 19.572 | 1.00 | 20.23 | U |
| ATOM | 1071 | C | LEU | U | 435 | 40.061 | 94.137 | 21.000 | 1.00 | 33.58 | U |
| ATOM | 1072 | O | LEU | U | 435 | 39.782 | 94.189 | 22.193 | 1.00 | 36.34 | U |
| ATOM | 1073 | N | GLU | U | 436 | 40.728 | 93.115 | 20.470 | 1.00 | 35.92 | U |
| ATOM | 1074 | CA | GLU | U | 436 | 41.175 | 92.010 | 21.307 | 1.00 | 36.91 | U |
| ATOM | 1075 | CB | GLU | U | 436 | 41.803 | 90.914 | 20.448 | 1.00 | 39.15 | U |
| ATOM | 1076 | CG | GLU | U | 436 | 42.215 | 89.619 | 21.195 | 1.00 | 47.97 | U |
| ATOM | 1077 | CD | GLU | U | 436 | 43.422 | 89.797 | 22.130 | 1.00 | 57.74 | U |
| ATOM | 1078 | OE1 | GLU | U | 436 | 44.370 | 90.522 | 21.752 | 1.00 | 57.59 | U |
| ATOM | 1079 | OE2 | GLU | U | 436 | 43.431 | 89.197 | 23.237 | 1.00 | 59.28 | U |
| ATOM | 1080 | C | GLU | U | 436 | 42.192 | 92.546 | 22.308 | 1.00 | 39.00 | U |
| ATOM | 1081 | O | GLU | U | 436 | 42.077 | 92.330 | 23.516 | 1.00 | 43.08 | U |
| ATOM | 1082 | N | GLU | U | 437 | 43.179 | 93.272 | 21.806 | 1.00 | 33.63 | U |
| ATOM | 1083 | CA | GLU | U | 437 | 44.205 | 93.830 | 22.666 | 1.00 | 33.97 | U |
| ATOM | 1084 | CB | GLU | U | 437 | 45.288 | 94.465 | 21.788 | 1.00 | 35.76 | U |
| ATOM | 1085 | CG | GLU | U | 437 | 45.654 | 95.892 | 22.072 | 1.00 | 42.35 | U |
| ATOM | 1086 | CD | GLU | U | 437 | 46.806 | 95.998 | 23.013 | 1.00 | 46.27 | U |
| ATOM | 1087 | OE1 | GLU | U | 437 | 47.829 | 95.318 | 22.791 | 1.00 | 52.89 | U |
| ATOM | 1088 | OE2 | GLU | U | 437 | 46.690 | 96.773 | 23.977 | 1.00 | 54.39 | U |
| ATOM | 1089 | C | GLU | U | 437 | 43.620 | 94.828 | 23.666 | 1.00 | 37.00 | U |
| ATOM | 1090 | O | GLU | U | 437 | 44.014 | 94.850 | 24.831 | 1.00 | 35.19 | U |
| ATOM | 1091 | N | TYR | U | 438 | 42.655 | 95.633 | 23.229 | 1.00 | 38.30 | U |
| ATOM | 1092 | CA | TYR | U | 438 | 42.041 | 96.622 | 24.111 | 1.00 | 31.99 | U |
| ATOM | 1093 | CB | TYR | U | 438 | 41.003 | 97.431 | 23.367 | 1.00 | 21.67 | U |
| ATOM | 1094 | CG | TYR | U | 438 | 40.195 | 98.266 | 24.315 | 1.00 | 31.24 | U |
| ATOM | 1095 | CD1 | TYR | U | 438 | 40.634 | 99.513 | 24.705 | 1.00 | 27.07 | U |
| ATOM | 1096 | CE1 | TYR | U | 438 | 39.894 | 100.282 | 25.577 | 1.00 | 22.39 | U |
| ATOM | 1097 | CD2 | TYR | U | 438 | 38.983 | 97.801 | 24.837 | 1.00 | 35.07 | U |
| ATOM | 1098 | CE2 | TYR | U | 438 | 38.237 | 98.573 | 25.713 | 1.00 | 30.79 | U |
| ATOM | 1099 | CZ | TYR | U | 438 | 38.704 | 99.817 | 26.074 | 1.00 | 25.62 | U |
| ATOM | 1100 | OH | TYR | U | 438 | 37.974 | 100.624 | 26.909 | 1.00 | 33.17 | U |
| ATOM | 1101 | C | TYR | U | 438 | 41.372 | 96.015 | 25.330 | 1.00 | 31.47 | U |
| ATOM | 1102 | O | TYR | U | 438 | 41.455 | 96.573 | 26.424 | 1.00 | 25.85 | U |
| ATOM | 1103 | N | CYS | U | 439 | 40.673 | 94.897 | 25.123 | 1.00 | 31.38 | U |
| ATOM | 1104 | CA | CYS | U | 439 | 39.992 | 94.203 | 26.211 | 1.00 | 36.18 | U |
| ATOM | 1105 | CB | CYS | U | 439 | 39.130 | 93.070 | 25.688 | 1.00 | 31.50 | U |
| ATOM | 1106 | SG | CYS | U | 439 | 37.746 | 93.633 | 24.744 | 1.00 | 47.00 | U |
| ATOM | 1107 | C | CYS | U | 439 | 41.020 | 93.623 | 27.136 | 1.00 | 37.71 | U |
| ATOM | 1108 | O | CYS | U | 439 | 40.955 | 93.808 | 28.342 | 1.00 | 43.56 | U |
| ATOM | 1109 | N | ARG | U | 440 | 41.974 | 92.914 | 26.550 | 1.00 | 35.22 | U |
| ATOM | 1110 | CA | ARG | U | 440 | 43.046 | 92.292 | 27.302 | 1.00 | 33.73 | U |
| ATOM | 1111 | CB | ARG | U | 440 | 44.042 | 91.688 | 26.319 | 1.00 | 33.72 | U |
| ATOM | 1112 | CG | ARG | U | 440 | 44.799 | 90.454 | 26.800 | 1.00 | 33.46 | U |
| ATOM | 1113 | CD | ARG | U | 440 | 45.700 | 89.954 | 25.690 | 1.00 | 36.28 | U |
| ATOM | 1114 | NE | ARG | U | 440 | 46.605 | 91.014 | 25.248 | 1.00 | 49.83 | U |
| ATOM | 1115 | CZ | ARG | U | 440 | 46.961 | 91.203 | 23.982 | 1.00 | 58.58 | U |
| ATOM | 1116 | NH1 | ARG | U | 440 | 46.485 | 90.394 | 23.038 | 1.00 | 65.49 | U |
| ATOM | 1117 | NH2 | ARG | U | 440 | 47.780 | 92.201 | 23.657 | 1.00 | 54.79 | U |
| ATOM | 1118 | C | ARG | U | 440 | 43.737 | 93.314 | 28.209 | 1.00 | 37.07 | U |
| ATOM | 1119 | O | ARG | U | 440 | 43.996 | 93.045 | 29.383 | 1.00 | 36.27 | U |
| ATOM | 1120 | N | GLN | U | 441 | 44.032 | 94.491 | 27.664 | 1.00 | 38.15 | U |
| ATOM | 1121 | CA | GLN | U | 441 | 44.685 | 95.551 | 28.440 | 1.00 | 37.13 | U |
| ATOM | 1122 | CB | GLN | U | 441 | 45.169 | 96.684 | 27.541 | 1.00 | 41.00 | U |
| ATOM | 1123 | CG | GLN | U | 441 | 46.452 | 96.417 | 26.807 | 1.00 | 36.46 | U |
| ATOM | 1124 | CD | GLN | U | 441 | 47.543 | 96.001 | 27.729 | 1.00 | 38.92 | U |
| ATOM | 1125 | OE1 | GLN | U | 441 | 47.541 | 96.353 | 28.918 | 1.00 | 36.95 | U |
| ATOM | 1126 | NE2 | GLN | U | 441 | 48.505 | 95.252 | 27.196 | 1.00 | 37.41 | U |
| ATOM | 1127 | C | GLN | U | 441 | 43.790 | 96.183 | 29.476 | 1.00 | 35.04 | U |
| ATOM | 1128 | O | GLN | U | 441 | 44.202 | 96.334 | 30.614 | 1.00 | 36.54 | U |
| ATOM | 1129 | N | THR | U | 442 | 42.580 | 96.569 | 29.060 | 1.00 | 32.77 | U |
| ATOM | 1130 | CA | THR | U | 442 | 41.606 | 97.232 | 29.929 | 1.00 | 33.05 | U |
| ATOM | 1131 | CB | THR | U | 442 | 40.476 | 97.923 | 29.102 | 1.00 | 35.63 | U |
| ATOM | 1132 | OG1 | THR | U | 442 | 41.051 | 98.748 | 28.084 | 1.00 | 37.35 | U |
| ATOM | 1133 | CG2 | THR | U | 442 | 39.603 | 98.811 | 30.001 | 1.00 | 28.11 | U |
| ATOM | 1134 | C | THR | U | 442 | 40.947 | 96.338 | 30.972 | 1.00 | 32.93 | U |
| ATOM | 1135 | O | THR | U | 442 | 40.877 | 96.715 | 32.136 | 1.00 | 33.34 | U |
| ATOM | 1136 | N | TYR | U | 443 | 40.454 | 95.173 | 30.559 | 1.00 | 31.00 | U |
| ATOM | 1137 | CA | TYR | U | 443 | 39.788 | 94.234 | 31.475 | 1.00 | 32.69 | U |
| ATOM | 1138 | CB | TYR | U | 443 | 38.331 | 94.011 | 31.067 | 1.00 | 26.91 | U |
| ATOM | 1139 | CG | TYR | U | 443 | 37.568 | 95.283 | 30.832 | 1.00 | 31.76 | U |
| ATOM | 1140 | CD1 | TYR | U | 443 | 37.137 | 96.074 | 31.908 | 1.00 | 23.68 | U |
| ATOM | 1141 | CE1 | TYR | U | 443 | 36.466 | 97.271 | 31.689 | 1.00 | 27.05 | U |
| ATOM | 1142 | CD2 | TYR | U | 443 | 37.306 | 95.726 | 29.522 | 1.00 | 30.21 | U |
| ATOM | 1143 | CE2 | TYR | U | 443 | 36.640 | 96.925 | 29.291 | 1.00 | 28.42 | U |
| ATOM | 1144 | CZ | TYR | U | 443 | 36.221 | 97.692 | 30.379 | 1.00 | 34.16 | U |
| ATOM | 1145 | OH | TYR | U | 443 | 35.542 | 98.863 | 30.143 | 1.00 | 37.08 | U |
| ATOM | 1146 | C | TYR | U | 443 | 40.533 | 92.916 | 31.405 | 1.00 | 36.13 | U |
| ATOM | 1147 | O | TYR | U | 443 | 40.115 | 91.969 | 30.731 | 1.00 | 35.11 | U |
| ATOM | 1148 | N | PRO | U | 444 | 41.661 | 92.840 | 32.107 | 1.00 | 41.53 | U |
| ATOM | 1149 | CD | PRO | U | 444 | 42.276 | 93.918 | 32.892 | 1.00 | 37.80 | U |
| ATOM | 1150 | CA | PRO | U | 444 | 42.501 | 91.643 | 32.132 | 1.00 | 40.98 | U |
| ATOM | 1151 | CB | PRO | U | 444 | 43.748 | 92.120 | 32.855 | 1.00 | 29.62 | U |
| ATOM | 1152 | CG | PRO | U | 444 | 43.715 | 93.610 | 32.698 | 1.00 | 37.30 | U |
| ATOM | 1153 | C | PRO | U | 444 | 41.872 | 90.442 | 32.829 | 1.00 | 44.98 | U |
| ATOM | 1154 | O | PRO | U | 444 | 42.137 | 89.302 | 32.461 | 1.00 | 41.03 | U |
| ATOM | 1155 | N | ASP | U | 445 | 41.037 | 90.695 | 33.831 | 1.00 | 50.06 | U |
| ATOM | 1156 | CA | ASP | U | 445 | 40.432 | 89.600 | 34.570 | 1.00 | 52.26 | U |
| ATOM | 1157 | CB | ASP | U | 445 | 40.207 | 90.007 | 36.036 | 1.00 | 57.60 | U |
| ATOM | 1158 | CG | ASP | U | 445 | 41.510 | 90.407 | 36.744 | 1.00 | 69.88 | U |
| ATOM | 1159 | OD1 | ASP | U | 445 | 42.602 | 89.947 | 36.326 | 1.00 | 72.62 | U |
| ATOM | 1160 | OD2 | ASP | U | 445 | 41.441 | 91.177 | 37.731 | 1.00 | 79.62 | U |
| ATOM | 1161 | C | ASP | U | 445 | 39.145 | 89.094 | 33.942 | 1.00 | 51.13 | U |
| ATOM | 1162 | O | ASP | U | 445 | 38.335 | 88.447 | 34.598 | 1.00 | 55.43 | U |
| ATOM | 1163 | N | GLN | U | 446 | 38.956 | 89.377 | 32.663 | 1.00 | 43.93 | U |
| ATOM | 1164 | CA | GLN | U | 446 | 37.769 | 88.914 | 31.974 | 1.00 | 40.93 | U |
| ATOM | 1165 | CB | GLN | U | 446 | 36.891 | 90.091 | 31.610 | 1.00 | 36.72 | U |
| ATOM | 1166 | CG | GLN | U | 446 | 36.322 | 90.767 | 32.812 | 1.00 | 28.56 | U |
| ATOM | 1167 | CD | GLN | U | 446 | 35.470 | 91.937 | 32.460 | 1.00 | 32.74 | U |
| ATOM | 1168 | OE1 | GLN | U | 446 | 34.587 | 91.855 | 31.595 | 1.00 | 38.58 | U |
| ATOM | 1169 | NE2 | GLN | U | 446 | 35.709 | 93.044 | 33.135 | 1.00 | 29.24 | U |
| ATOM | 1170 | C | GLN | U | 446 | 38.199 | 88.199 | 30.724 | 1.00 | 44.06 | U |
| ATOM | 1171 | O | GLN | U | 446 | 38.212 | 88.798 | 29.648 | 1.00 | 49.92 | U |
| ATOM | 1172 | N | SER | U | 447 | 38.533 | 86.918 | 30.858 | 1.00 | 44.27 | U |
| ATOM | 1173 | CA | SER | U | 447 | 39.019 | 86.137 | 29.724 | 1.00 | 49.37 | U |
| ATOM | 1174 | CB | SER | U | 447 | 39.460 | 84.743 | 30.173 | 1.00 | 50.92 | U |
| ATOM | 1175 | OG | SER | U | 447 | 38.340 | 83.931 | 30.471 | 1.00 | 56.10 | U |
| ATOM | 1176 | C | SER | U | 447 | 38.043 | 85.983 | 28.571 | 1.00 | 49.09 | U |
| ATOM | 1177 | O | SER | U | 447 | 38.468 | 85.757 | 27.432 | 1.00 | 54.13 | U |
| ATOM | 1178 | N | GLY | U | 448 | 36.749 | 86.111 | 28.845 | 1.00 | 41.71 | U |
| ATOM | 1179 | CA | GLY | U | 448 | 35.791 | 85.935 | 27.774 | 1.00 | 40.85 | U |
| ATOM | 1180 | C | GLY | U | 448 | 35.196 | 87.190 | 27.186 | 1.00 | 38.14 | U |
| ATOM | 1181 | O | GLY | U | 448 | 34.415 | 87.123 | 26.239 | 1.00 | 40.33 | U |
| ATOM | 1182 | N | ARG | U | 449 | 35.562 | 88.340 | 27.730 | 1.00 | 34.32 | U |
| ATOM | 1183 | CA | ARG | U | 449 | 35.002 | 89.582 | 27.249 | 1.00 | 31.28 | U |
| ATOM | 1184 | CB | ARG | U | 449 | 35.658 | 90.760 | 27.959 | 1.00 | 31.25 | U |
| ATOM | 1185 | CG | ARG | U | 449 | 35.204 | 92.103 | 27.409 | 1.00 | 30.81 | U |
| ATOM | 1186 | CD | ARG | U | 449 | 35.491 | 93.203 | 28.385 | 1.00 | 28.49 | U |
| ATOM | 1187 | NE | ARG | U | 449 | 34.317 | 93.406 | 29.207 | 1.00 | 25.55 | U |
| ATOM | 1188 | CZ | ARG | U | 449 | 33.560 | 94.491 | 29.155 | 1.00 | 35.25 | U |
| ATOM | 1189 | NH1 | ARG | U | 449 | 33.861 | 95.481 | 28.326 | 1.00 | 29.71 | U |
| ATOM | 1190 | NH2 | ARG | U | 449 | 32.483 | 94.569 | 29.910 | 1.00 | 35.38 | U |
| ATOM | 1191 | C | ARG | U | 449 | 35.053 | 89.771 | 25.734 | 1.00 | 32.37 | U |
| ATOM | 1192 | O | ARG | U | 449 | 34.026 | 90.069 | 25.115 | 1.00 | 24.02 | U |
| ATOM | 1193 | N | PHE | U | 450 | 36.226 | 89.590 | 25.130 | 1.00 | 32.68 | U |
| ATOM | 1194 | CA | PHE | U | 450 | 36.344 | 89.770 | 23.685 | 1.00 | 33.86 | U |
| ATOM | 1195 | CB | PHE | U | 450 | 37.740 | 89.346 | 23.208 | 1.00 | 32.48 | U |
| ATOM | 1196 | CG | PHE | U | 450 | 37.964 | 89.516 | 21.724 | 1.00 | 27.55 | U |
| ATOM | 1197 | CD1 | PHE | U | 450 | 38.552 | 88.505 | 20.986 | 1.00 | 18.94 | U |
| ATOM | 1198 | CD2 | PHE | U | 450 | 37.604 | 90.694 | 21.079 | 1.00 | 32.79 | U |
| ATOM | 1199 | CE1 | PHE | U | 450 | 38.783 | 88.656 | 19.635 | 1.00 | 26.39 | U |
| ATOM | 1200 | CE2 | PHE | U | 450 | 37.829 | 90.862 | 19.720 | 1.00 | 31.98 | U |
| ATOM | 1201 | CZ | PHE | U | 450 | 38.420 | 89.844 | 18.996 | 1.00 | 28.81 | U |
| ATOM | 1202 | C | PHE | U | 450 | 35.255 | 88.994 | 22.926 | 1.00 | 35.97 | U |
| ATOM | 1203 | O | PHE | U | 450 | 34.476 | 89.576 | 22.141 | 1.00 | 29.59 | U |
| ATOM | 1204 | N | ALA | U | 451 | 35.193 | 87.685 | 23.147 | 1.00 | 34.16 | U |
| ATOM | 1205 | CA | ALA | U | 451 | 34.165 | 86.919 | 22.465 | 1.00 | 40.68 | U |
| ATOM | 1206 | CB | ALA | U | 451 | 34.369 | 85.403 | 22.679 | 1.00 | 37.32 | U |
| ATOM | 1207 | C | ALA | U | 451 | 32.763 | 87.358 | 22.937 | 1.00 | 37.16 | U |
| ATOM | 1208 | O | ALA | U | 451 | 31.811 | 87.373 | 22.149 | 1.00 | 30.92 | U |
| ATOM | 1209 | N | LYS | U | 452 | 32.632 | 87.742 | 24.206 | 1.00 | 33.43 | U |
| ATOM | 1210 | CA | LYS | U | 452 | 31.328 | 88.149 | 24.694 | 1.00 | 35.13 | U |
| ATOM | 1211 | CB | LYS | U | 452 | 31.388 | 88.491 | 26.194 | 1.00 | 33.79 | U |
| ATOM | 1212 | CG | LYS | U | 452 | 30.100 | 89.149 | 26.709 | 1.00 | 30.21 | U |
| ATOM | 1213 | CD | LYS | U | 452 | 29.938 | 89.171 | 28.205 | 1.00 | 25.99 | U |
| ATOM | 1214 | CE | LYS | U | 452 | 29.242 | 87.904 | 28.704 | 1.00 | 33.85 | U |
| ATOM | 1215 | NZ | LYS | U | 452 | 30.147 | 86.688 | 28.723 | 1.00 | 45.04 | U |
| ATOM | 1216 | C | LYS | U | 452 | 30.796 | 89.325 | 23.870 | 1.00 | 38.79 | U |
| ATOM | 1217 | O | LYS | U | 452 | 29.623 | 89.323 | 23.450 | 1.00 | 41.46 | U |
| ATOM | 1218 | N | LEU | U | 453 | 31.669 | 90.307 | 23.632 | 1.00 | 36.24 | U |
| ATOM | 1219 | CA | LEU | U | 453 | 31.346 | 91.504 | 22.853 | 1.00 | 35.60 | U |
| ATOM | 1220 | CB | LEU | U | 453 | 32.529 | 92.491 | 22.859 | 1.00 | 37.67 | U |
| ATOM | 1221 | CG | LEU | U | 453 | 32.925 | 93.408 | 24.017 | 1.00 | 37.01 | U |
| ATOM | 1222 | CD1 | LEU | U | 453 | 34.319 | 93.957 | 23.772 | 1.00 | 26.98 | U |
| ATOM | 1223 | CD2 | LEU | U | 453 | 31.929 | 94.536 | 24.126 | 1.00 | 28.37 | U |
| ATOM | 1224 | C | LEU | U | 453 | 31.089 | 91.120 | 21.399 | 1.00 | 36.84 | U |
| ATOM | 1225 | O | LEU | U | 453 | 30.185 | 91.625 | 20.754 | 1.00 | 39.36 | U |
| ATOM | 1226 | N | LEU | U | 454 | 31.923 | 90.236 | 20.875 | 1.00 | 30.51 | U |
| ATOM | 1227 | CA | LEU | U | 454 | 31.796 | 89.814 | 19.502 | 1.00 | 27.98 | U |
| ATOM | 1228 | CB | LEU | U | 454 | 32.978 | 88.931 | 19.162 | 1.00 | 19.45 | U |
| ATOM | 1229 | CG | LEU | U | 454 | 34.193 | 89.742 | 18.766 | 1.00 | 17.60 | U |
| ATOM | 1230 | CD1 | LEU | U | 454 | 35.314 | 88.828 | 18.323 | 1.00 | 10.12 | U |
| ATOM | 1231 | CD2 | LEU | U | 454 | 33.776 | 90.682 | 17.625 | 1.00 | 16.90 | U |
| ATOM | 1232 | C | LEU | U | 454 | 30.491 | 89.081 | 19.225 | 1.00 | 36.94 | U |
| ATOM | 1233 | O | LEU | U | 454 | 29.952 | 89.135 | 18.103 | 1.00 | 40.85 | U |
| ATOM | 1234 | N | LEU | U | 455 | 29.976 | 88.400 | 20.249 | 1.00 | 33.97 | U |
| ATOM | 1235 | CA | LEU | U | 455 | 28.748 | 87.645 | 20.107 | 1.00 | 29.01 | U |
| ATOM | 1236 | CB | LEU | U | 455 | 28.768 | 86.451 | 21.045 | 1.00 | 20.81 | U |
| ATOM | 1237 | CG | LEU | U | 455 | 29.785 | 85.367 | 20.661 | 1.00 | 31.44 | U |
| ATOM | 1238 | CD1 | LEU | U | 455 | 29.688 | 84.190 | 21.655 | 1.00 | 28.59 | U |
| ATOM | 1239 | CD2 | LEU | U | 455 | 29.546 | 84.885 | 19.212 | 1.00 | 24.17 | U |
| ATOM | 1240 | C | LEU | U | 455 | 27.434 | 88.418 | 20.241 | 1.00 | 34.24 | U |
| ATOM | 1241 | O | LEU | U | 455 | 26.362 | 87.828 | 20.338 | 1.00 | 40.87 | U |
| ATOM | 1242 | N | ARG | U | 456 | 27.494 | 89.739 | 20.246 | 1.00 | 34.86 | U |
| ATOM | 1243 | CA | ARG | U | 456 | 26.258 | 90.507 | 20.261 | 1.00 | 30.18 | U |
| ATOM | 1244 | CB | ARG | U | 456 | 26.347 | 91.717 | 21.177 | 1.00 | 19.46 | U |
| ATOM | 1245 | CG | ARG | U | 456 | 26.244 | 91.353 | 22.611 | 1.00 | 26.45 | U |
| ATOM | 1246 | CD | ARG | U | 456 | 24.835 | 91.561 | 23.174 | 1.00 | 36.76 | U |
| ATOM | 1247 | NE | ARG | U | 456 | 23.845 | 90.491 | 22.947 | 1.00 | 37.17 | U |
| ATOM | 1248 | CZ | ARG | U | 456 | 24.110 | 89.187 | 22.931 | 1.00 | 35.22 | U |
| ATOM | 1249 | NH1 | ARG | U | 456 | 25.366 | 88.747 | 23.109 | 1.00 | 31.08 | U |
| ATOM | 1250 | NH2 | ARG | U | 456 | 23.098 | 88.328 | 22.788 | 1.00 | 26.55 | U |
| ATOM | 1251 | C | ARG | U | 456 | 26.073 | 90.957 | 18.824 | 1.00 | 28.29 | U |
| ATOM | 1252 | O | ARG | U | 456 | 25.095 | 91.612 | 18.482 | 1.00 | 28.43 | U |
| ATOM | 1253 | N | LEU | U | 457 | 27.023 | 90.577 | 17.976 | 1.00 | 29.12 | U |
| ATOM | 1254 | CA | LEU | U | 457 | 26.969 | 90.955 | 16.574 | 1.00 | 35.79 | U |
| ATOM | 1255 | CB | LEU | U | 457 | 28.343 | 90.751 | 15.924 | 1.00 | 32.44 | U |
| ATOM | 1256 | CG | LEU | U | 457 | 29.240 | 91.945 | 16.283 | 1.00 | 29.42 | U |
| ATOM | 1257 | CD1 | LEU | U | 457 | 30.596 | 91.843 | 15.600 | 1.00 | 20.16 | U |
| ATOM | 1258 | CD2 | LEU | U | 457 | 28.507 | 93.239 | 15.902 | 1.00 | 7.52 | U |
| ATOM | 1259 | C | LEU | U | 457 | 25.864 | 90.210 | 15.846 | 1.00 | 35.57 | U |
| ATOM | 1260 | O | LEU | U | 457 | 25.176 | 90.781 | 14.994 | 1.00 | 37.45 | U |
| ATOM | 1261 | N | PRO | U | 458 | 25.697 | 88.917 | 16.154 | 1.00 | 39.69 | U |
| ATOM | 1262 | CD | PRO | U | 458 | 26.651 | 88.043 | 16.861 | 1.00 | 42.46 | U |
| ATOM | 1263 | CA | PRO | U | 458 | 24.639 | 88.129 | 15.520 | 1.00 | 40.64 | U |
| ATOM | 1264 | CB | PRO | U | 458 | 24.874 | 86.732 | 16.072 | 1.00 | 39.74 | U |
| ATOM | 1265 | CG | PRO | U | 458 | 26.341 | 86.690 | 16.281 | 1.00 | 39.34 | U |
| ATOM | 1266 | C | PRO | U | 458 | 23.328 | 88.732 | 16.043 | 1.00 | 39.56 | U |
| ATOM | 1267 | O | PRO | U | 458 | 22.474 | 89.151 | 15.285 | 1.00 | 37.25 | U |
| ATOM | 1268 | N | ALA | U | 459 | 23.194 | 88.803 | 17.360 | 1.00 | 42.78 | U |
| ATOM | 1269 | CA | ALA | U | 459 | 21.995 | 89.371 | 17.966 | 1.00 | 45.25 | U |
| ATOM | 1270 | CB | ALA | U | 459 | 22.237 | 89.654 | 19.453 | 1.00 | 50.98 | U |
| ATOM | 1271 | C | ALA | U | 459 | 21.536 | 90.644 | 17.272 | 1.00 | 42.47 | U |
| ATOM | 1272 | O | ALA | U | 459 | 20.336 | 90.835 | 17.045 | 1.00 | 45.28 | U |
| ATOM | 1273 | N | LEU | U | 460 | 22.504 | 91.505 | 16.964 | 1.00 | 36.60 | U |
| ATOM | 1274 | CA | LEU | U | 460 | 22.288 | 92.793 | 16.304 | 1.00 | 33.20 | U |
| ATOM | 1275 | CB | LEU | U | 460 | 23.596 | 93.582 | 16.274 | 1.00 | 27.00 | U |
| ATOM | 1276 | CG | LEU | U | 460 | 23.699 | 94.850 | 15.426 | 1.00 | 22.57 | U |
| ATOM | 1277 | CD1 | LEU | U | 460 | 22.852 | 95.941 | 15.999 | 1.00 | 16.07 | U |
| ATOM | 1278 | CD2 | LEU | U | 460 | 25.142 | 95.282 | 15.383 | 1.00 | 15.66 | U |
| ATOM | 1279 | C | LEU | U | 460 | 21.827 | 92.574 | 14.885 | 1.00 | 32.62 | U |
| ATOM | 1280 | O | LEU | U | 460 | 21.011 | 93.318 | 14.352 | 1.00 | 36.27 | U |
| ATOM | 1281 | N | ARG | U | 461 | 22.388 | 91.549 | 14.265 | 1.00 | 30.57 | U |
| ATOM | 1282 | CA | ARG | U | 461 | 22.033 | 91.214 | 12.907 | 1.00 | 28.42 | U |
| ATOM | 1283 | CB | ARG | U | 461 | 22.848 | 90.023 | 12.421 | 1.00 | 23.68 | U |
| ATOM | 1284 | CG | ARG | U | 461 | 22.351 | 89.510 | 11.121 | 1.00 | 21.90 | U |
| ATOM | 1285 | CD | ARG | U | 461 | 22.355 | 90.634 | 10.094 | 1.00 | 21.59 | U |
| ATOM | 1286 | NE | ARG | U | 461 | 23.722 | 90.984 | 9.723 | 1.00 | 25.86 | U |
| ATOM | 1287 | CZ | ARG | U | 461 | 24.559 | 90.155 | 9.108 | 1.00 | 24.69 | U |
| ATOM | 1288 | NH1 | ARG | U | 461 | 24.173 | 88.932 | 8.775 | 1.00 | 31.52 | U |
| ATOM | 1289 | NH2 | ARG | U | 461 | 25.796 | 90.525 | 8.867 | 1.00 | 13.72 | U |
| ATOM | 1290 | C | ARG | U | 461 | 20.560 | 90.872 | 12.882 | 1.00 | 28.82 | U |
| ATOM | 1291 | O | ARG | U | 461 | 19.788 | 91.477 | 12.151 | 1.00 | 34.06 | U |
| ATOM | 1292 | N | SER | U | 462 | 20.170 | 89.904 | 13.701 | 1.00 | 23.31 | U |
| ATOM | 1293 | CA | SER | U | 462 | 18.785 | 89.476 | 13.778 | 1.00 | 20.62 | U |
| ATOM | 1294 | CB | SER | U | 462 | 18.634 | 88.487 | 14.916 | 1.00 | 10.85 | U |
| ATOM | 1295 | OG | SER | U | 462 | 19.088 | 87.222 | 14.478 | 1.00 | 24.21 | U |
| ATOM | 1296 | C | SER | U | 462 | 17.789 | 90.626 | 13.932 | 1.00 | 25.13 | U |
| ATOM | 1297 | O | SER | U | 462 | 16.749 | 90.651 | 13.258 | 1.00 | 27.26 | U |
| ATOM | 1298 | N | ILE | U | 463 | 18.101 | 91.570 | 14.819 | 1.00 | 25.00 | U |
| ATOM | 1299 | CA | ILE | U | 463 | 17.235 | 92.719 | 15.035 | 1.00 | 22.59 | U |
| ATOM | 1300 | CB | ILE | U | 463 | 17.723 | 93.606 | 16.213 | 1.00 | 21.95 | U |
| ATOM | 1301 | CG2 | ILE | U | 463 | 16.767 | 94.803 | 16.417 | 1.00 | 9.54 | U |
| ATOM | 1302 | CG1 | ILE | U | 463 | 17.754 | 92.784 | 17.495 | 1.00 | 29.80 | U |
| ATOM | 1303 | CD1 | ILE | U | 463 | 18.007 | 93.613 | 18.746 | 1.00 | 36.94 | U |
| ATOM | 1304 | C | ILE | U | 463 | 17.242 | 93.543 | 13.757 | 1.00 | 23.31 | U |
| ATOM | 1305 | O | ILE | U | 463 | 16.273 | 94.209 | 13.414 | 1.00 | 23.92 | U |
| ATOM | 1306 | N | GLY | U | 464 | 18.352 | 93.503 | 13.044 | 1.00 | 24.89 | U |
| ATOM | 1307 | CA | GLY | U | 464 | 18.426 | 94.260 | 11.809 | 1.00 | 33.03 | U |
| ATOM | 1308 | C | GLY | U | 464 | 17.482 | 93.699 | 10.764 | 1.00 | 31.59 | U |
| ATOM | 1309 | O | GLY | U | 464 | 16.797 | 94.458 | 10.077 | 1.00 | 31.92 | U |
| ATOM | 1310 | N | LEU | U | 465 | 17.437 | 92.370 | 10.648 | 1.00 | 30.09 | U |
| ATOM | 1311 | CA | LEU | U | 465 | 16.574 | 91.721 | 9.665 | 1.00 | 29.21 | U |
| ATOM | 1312 | CB | LEU | U | 465 | 16.778 | 90.196 | 9.651 | 1.00 | 28.45 | U |
| ATOM | 1313 | CG | LEU | U | 465 | 18.025 | 89.446 | 9.155 | 1.00 | 32.34 | U |
| ATOM | 1314 | CD1 | LEU | U | 465 | 18.509 | 89.967 | 7.832 | 1.00 | 16.72 | U |
| ATOM | 1315 | CD2 | LEU | U | 465 | 19.099 | 89.569 | 10.168 | 1.00 | 36.94 | U |
| ATOM | 1316 | C | LEU | U | 465 | 15.096 | 92.018 | 9.899 | 1.00 | 31.95 | U |
| ATOM | 1317 | O | LEU | U | 465 | 14.296 | 91.852 | 8.999 | 1.00 | 33.33 | U |
| ATOM | 1318 | N | LYS | U | 466 | 14.722 | 92.449 | 11.100 | 1.00 | 33.32 | U |
| ATOM | 1319 | CA | LYS | U | 466 | 13.323 | 92.747 | 11.375 | 1.00 | 35.15 | U |
| ATOM | 1320 | CB | LYS | U | 466 | 13.063 | 92.648 | 12.877 | 1.00 | 33.26 | U |
| ATOM | 1321 | CG | LYS | U | 466 | 13.287 | 91.263 | 13.412 | 1.00 | 41.55 | U |
| ATOM | 1322 | CD | LYS | U | 466 | 12.814 | 91.084 | 14.855 | 1.00 | 53.25 | U |
| ATOM | 1323 | CE | LYS | U | 466 | 12.802 | 89.586 | 15.233 | 1.00 | 58.29 | U |
| ATOM | 1324 | NZ | LYS | U | 466 | 12.566 | 89.308 | 16.689 | 1.00 | 55.16 | U |
| ATOM | 1325 | C | LYS | U | 466 | 12.858 | 94.113 | 10.826 | 1.00 | 40.67 | U |
| ATOM | 1326 | O | LYS | U | 466 | 11.662 | 94.425 | 10.834 | 1.00 | 43.41 | U |
| ATOM | 1327 | N | CYS | U | 467 | 13.805 | 94.918 | 10.342 | 1.00 | 42.42 | U |
| ATOM | 1328 | CA | CYS | U | 467 | 13.501 | 96.232 | 9.780 | 1.00 | 45.65 | U |
| ATOM | 1329 | CB | CYS | U | 467 | 14.704 | 97.150 | 9.903 | 1.00 | 41.08 | U |
| ATOM | 1330 | SG | CYS | U | 467 | 15.338 | 97.319 | 11.533 | 1.00 | 44.12 | U |
| ATOM | 1331 | C | CYS | U | 467 | 13.198 | 96.093 | 8.301 | 1.00 | 51.69 | U |
| ATOM | 1332 | O | CYS | U | 467 | 14.115 | 95.838 | 7.515 | 1.00 | 54.82 | U |
| ATOM | 1333 | N | LEU | U | 468 | 11.942 | 96.277 | 7.905 | 1.00 | 52.02 | U |
| ATOM | 1334 | CA | LEU | U | 468 | 11.587 | 96.159 | 6.486 | 1.00 | 55.73 | U |
| ATOM | 1335 | CB | LEU | U | 468 | 10.107 | 95.800 | 6.334 | 1.00 | 56.80 | U |
| ATOM | 1336 | CG | LEU | U | 468 | 9.600 | 94.477 | 6.915 | 1.00 | 51.27 | U |
| ATOM | 1337 | CD1 | LEU | U | 468 | 8.101 | 94.283 | 6.550 | 1.00 | 43.06 | U |
| ATOM | 1338 | CD2 | LEU | U | 468 | 10.452 | 93.341 | 6.373 | 1.00 | 34.14 | U |
| ATOM | 1339 | C | LEU | U | 468 | 11.856 | 97.455 | 5.724 | 1.00 | 56.78 | U |
| ATOM | 1340 | O | LEU | U | 468 | 11.130 | 97.789 | 4.785 | 1.00 | 59.23 | U |
| ATOM | 1341 | N | GLU | U | 469 | 12.913 | 98.163 | 6.120 | 1.00 | 55.46 | U |
| ATOM | 1342 | CA | GLU | U | 469 | 13.264 | 99.455 | 5.528 | 1.00 | 51.29 | U |
| ATOM | 1343 | CB | GLU | U | 469 | 12.166 | 100.452 | 5.858 | 1.00 | 53.54 | U |
| ATOM | 1344 | CG | GLU | U | 469 | 11.871 | 100.484 | 7.366 | 1.00 | 50.33 | U |
| ATOM | 1345 | CD | GLU | U | 469 | 11.356 | 101.823 | 7.837 | 1.00 | 58.50 | U |
| ATOM | 1346 | OE1 | GLU | U | 469 | 10.734 | 102.528 | 7.004 | 1.00 | 53.57 | U |
| ATOM | 1347 | OE2 | GLU | U | 469 | 11.564 | 102.154 | 9.037 | 1.00 | 60.67 | U |
| ATOM | 1348 | C | GLU | U | 469 | 14.542 | 99.958 | 6.185 | 1.00 | 49.04 | U |
| ATOM | 1349 | O | GLU | U | 469 | 15.025 | 99.352 | 7.145 | 1.00 | 45.50 | U |
| ATOM | 1350 | N | HIS | U | 470 | 15.089 | 101.066 | 5.686 | 1.00 | 41.63 | U |
| ATOM | 1351 | CA | HIS | U | 470 | 16.276 | 101.617 | 6.317 | 1.00 | 35.57 | U |
| ATOM | 1352 | CB | HIS | U | 470 | 17.156 | 102.333 | 5.304 | 1.00 | 42.93 | U |
| ATOM | 1353 | CG | HIS | U | 470 | 17.949 | 101.411 | 4.433 | 1.00 | 50.13 | U |
| ATOM | 1354 | CD2 | HIS | U | 470 | 19.285 | 101.191 | 4.348 | 1.00 | 54.17 | U |
| ATOM | 1355 | ND1 | HIS | U | 470 | 17.366 | 100.565 | 3.520 | 1.00 | 54.29 | U |
| ATOM | 1356 | CE1 | HIS | U | 470 | 18.306 | 99.861 | 2.909 | 1.00 | 53.53 | U |
| ATOM | 1357 | NE2 | HIS | U | 470 | 19.478 | 100.225 | 3.395 | 1.00 | 46.80 | U |
| ATOM | 1358 | C | HIS | U | 470 | 15.778 | 102.598 | 7.368 | 1.00 | 35.43 | U |
| ATOM | 1359 | O | HIS | U | 470 | 15.225 | 103.642 | 7.034 | 1.00 | 33.29 | U |
| ATOM | 1360 | N | LEU | U | 471 | 15.953 | 102.257 | 8.640 | 1.00 | 30.44 | U |
| ATOM | 1361 | CA | LEU | U | 471 | 15.483 | 103.105 | 9.731 | 1.00 | 33.39 | U |
| ATOM | 1362 | CB | LEU | U | 471 | 16.213 | 102.720 | 11.025 | 1.00 | 35.77 | U |
| ATOM | 1363 | CG | LEU | U | 471 | 15.997 | 101.303 | 11.567 | 1.00 | 33.74 | U |
| ATOM | 1364 | CD1 | LEU | U | 471 | 16.933 | 101.053 | 12.723 | 1.00 | 32.19 | U |
| ATOM | 1365 | CD2 | LEU | U | 471 | 14.558 | 101.135 | 11.994 | 1.00 | 29.88 | U |
| ATOM | 1366 | C | LEU | U | 471 | 15.577 | 104.634 | 9.502 | 1.00 | 31.56 | U |
| ATOM | 1367 | O | LEU | U | 471 | 16.566 | 105.134 | 8.992 | 1.00 | 32.92 | U |
| ATOM | 1368 | N | PHE | U | 472 | 14.518 | 105.343 | 9.903 | 1.00 | 31.99 | U |
| ATOM | 1369 | CA | PHE | U | 472 | 14.348 | 106.803 | 9.819 | 1.00 | 29.89 | U |
| ATOM | 1370 | CB | PHE | U | 472 | 15.328 | 107.516 | 10.754 | 1.00 | 29.21 | U |
| ATOM | 1371 | CG | PHE | U | 472 | 15.469 | 106.869 | 12.084 | 1.00 | 27.18 | U |
| ATOM | 1372 | CD1 | PHE | U | 472 | 16.681 | 106.337 | 12.478 | 1.00 | 32.98 | U |
| ATOM | 1373 | CD2 | PHE | U | 472 | 14.390 | 106.751 | 12.932 | 1.00 | 33.76 | U |
| ATOM | 1374 | CE1 | PHE | U | 472 | 16.811 | 105.690 | 13.683 | 1.00 | 29.18 | U |
| ATOM | 1375 | CE2 | PHE | U | 472 | 14.516 | 106.091 | 14.161 | 1.00 | 30.06 | U |
| ATOM | 1376 | CZ | PHE | U | 472 | 15.725 | 105.568 | 14.527 | 1.00 | 36.30 | U |
| ATOM | 1377 | C | PHE | U | 472 | 14.441 | 107.442 | 8.430 | 1.00 | 33.97 | U |
| ATOM | 1378 | O | PHE | U | 472 | 14.275 | 108.661 | 8.290 | 1.00 | 31.69 | U |
| ATOM | 1379 | N | PHE | U | 473 | 14.676 | 106.634 | 7.401 | 1.00 | 32.14 | U |
| ATOM | 1380 | CA | PHE | U | 473 | 14.808 | 107.182 | 6.059 | 1.00 | 33.71 | U |
| ATOM | 1381 | CB | PHE | U | 473 | 15.157 | 106.097 | 5.049 | 1.00 | 17.58 | U |
| ATOM | 1382 | CG | PHE | U | 473 | 15.498 | 106.647 | 3.705 | 1.00 | 31.82 | U |
| ATOM | 1383 | CD1 | PHE | U | 473 | 16.670 | 107.393 | 3.522 | 1.00 | 30.74 | U |
| ATOM | 1384 | CD2 | PHE | U | 473 | 14.640 | 106.459 | 2.616 | 1.00 | 36.15 | U |
| ATOM | 1385 | CE1 | PHE | U | 473 | 16.983 | 107.940 | 2.275 | 1.00 | 34.57 | U |
| ATOM | 1386 | CE2 | PHE | U | 473 | 14.940 | 107.000 | 1.361 | 1.00 | 32.37 | U |
| ATOM | 1387 | CZ | PHE | U | 473 | 16.118 | 107.742 | 1.192 | 1.00 | 33.69 | U |
| ATOM | 1388 | C | PHE | U | 473 | 13.626 | 107.983 | 5.510 | 1.00 | 33.99 | U |
| ATOM | 1389 | O | PHE | U | 473 | 13.816 | 109.005 | 4.868 | 1.00 | 39.78 | U |
| ATOM | 1390 | N | PHE | U | 474 | 12.404 | 107.545 | 5.734 | 1.00 | 36.94 | U |
| ATOM | 1391 | CA | PHE | U | 474 | 11.314 | 108.313 | 5.172 | 1.00 | 43.73 | U |
| ATOM | 1392 | CB | PHE | U | 474 | 10.131 | 107.387 | 4.853 | 1.00 | 39.72 | U |
| ATOM | 1393 | CG | PHE | U | 474 | 10.459 | 106.315 | 3.836 | 1.00 | 34.24 | U |
| ATOM | 1394 | CD1 | PHE | U | 474 | 10.470 | 104.966 | 4.195 | 1.00 | 36.54 | U |
| ATOM | 1395 | CD2 | PHE | U | 474 | 10.758 | 106.653 | 2.523 | 1.00 | 28.23 | U |
| ATOM | 1396 | CE1 | PHE | U | 474 | 10.768 | 103.973 | 3.267 | 1.00 | 32.02 | U |
| ATOM | 1397 | CE2 | PHE | U | 474 | 11.057 | 105.672 | 1.585 | 1.00 | 29.66 | U |
| ATOM | 1398 | CZ | PHE | U | 474 | 11.062 | 104.326 | 1.961 | 1.00 | 36.99 | U |
| ATOM | 1399 | C | PHE | U | 474 | 10.907 | 109.506 | 6.044 | 1.00 | 45.36 | U |
| ATOM | 1400 | O | PHE | U | 474 | 9.974 | 110.240 | 5.712 | 1.00 | 47.56 | U |
| ATOM | 1401 | N | LYS | U | 475 | 11.616 | 109.699 | 7.153 | 1.00 | 47.58 | U |
| ATOM | 1402 | CA | LYS | U | 475 | 11.347 | 110.832 | 8.036 | 1.00 | 49.62 | U |
| ATOM | 1403 | CB | LYS | U | 475 | 11.546 | 110.475 | 9.512 | 1.00 | 38.43 | U |
| ATOM | 1404 | CG | LYS | U | 475 | 10.376 | 109.777 | 10.134 | 1.00 | 37.81 | U |
| ATOM | 1405 | CD | LYS | U | 475 | 10.394 | 109.905 | 11.648 | 1.00 | 34.22 | U |
| ATOM | 1406 | CE | LYS | U | 415 | 9.368 | 108.993 | 12.279 | 1.00 | 24.58 | U |
| ATOM | 1407 | NZ | LYS | U | 475 | 9.543 | 107.623 | 11.704 | 1.00 | 31.52 | U |
| ATOM | 1408 | C | LYS | U | 475 | 12.334 | 111.922 | 7.668 | 1.00 | 53.12 | U |
| ATOM | 1409 | O | LYS | U | 475 | 12.257 | 113.050 | 8.176 | 1.00 | 50.05 | U |
| ATOM | 1410 | N | LEU | U | 476 | 13.271 | 111.565 | 6.788 | 1.00 | 54.37 | U |
| ATOM | 1411 | CA | LEU | U | 476 | 14.298 | 112.498 | 6.349 | 1.00 | 55.80 | U |
| ATOM | 1412 | CB | LEU | U | 476 | 15.409 | 111.780 | 5.586 | 1.00 | 46.03 | U |
| ATOM | 1413 | CG | LEU | U | 476 | 16.380 | 110.968 | 6.448 | 1.00 | 51.17 | U |
| ATOM | 1414 | CD1 | LEU | U | 476 | 17.554 | 110.476 | 5.596 | 1.00 | 51.60 | U |
| ATOM | 1415 | CD2 | LEU | U | 476 | 16.879 | 111.828 | 7.603 | 1.00 | 48.81 | U |
| ATOM | 1416 | C | LEU | U | 476 | 13.680 | 113.563 | 5.482 | 1.00 | 61.13 | U |
| ATOM | 1417 | O | LEU | U | 476 | 12.765 | 113.289 | 4.704 | 1.00 | 61.06 | U |
| ATOM | 1418 | N | VAL | U | 477 | 14.188 | 114.781 | 5.628 | 1.00 | 69.05 | U |
| ATOM | 1419 | CA | VAL | U | 477 | 13.677 | 115.924 | 4.889 | 1.00 | 75.86 | U |
| ATOM | 1420 | CB | VAL | U | 477 | 14.451 | 117.218 | 5.297 | 1.00 | 75.38 | U |
| ATOM | 1421 | CG1 | VAL | U | 477 | 15.932 | 116.920 | 5.452 | 1.00 | 71.51 | U |
| ATOM | 1422 | CG2 | VAL | U | 477 | 14.219 | 118.317 | 4.278 | 1.00 | 74.25 | U |
| ATOM | 1423 | C | VAL | U | 477 | 13.692 | 115.722 | 3.376 | 1.00 | 78.42 | U |
| ATOM | 1424 | O | VAL | U | 477 | 12.658 | 115.853 | 2.723 | 1.00 | 80.89 | U |
| ATOM | 1425 | N | GLY | U | 478 | 14.847 | 115.384 | 2.819 | 1.00 | 77.79 | U |
| ATOM | 1426 | CA | GLY | U | 478 | 14.920 | 115.183 | 1.383 | 1.00 | 82.22 | U |
| ATOM | 1427 | C | GLY | U | 478 | 14.152 | 113.968 | 0.900 | 1.00 | 84.69 | U |
| ATOM | 1428 | O | GLY | U | 478 | 13.911 | 113.030 | 1.673 | 1.00 | 85.31 | U |
| ATOM | 1429 | N | ASN | U | 479 | 13.773 | 113.977 | −0.380 | 1.00 | 83.70 | U |
| ATOM | 1430 | CA | ASN | U | 479 | 13.031 | 112.863 | −0.975 | 1.00 | 83.67 | U |
| ATOM | 1431 | CB | ASN | U | 479 | 11.621 | 113.323 | −1.353 | 1.00 | 85.15 | U |
| ATOM | 1432 | CG | ASN | U | 479 | 10.543 | 112.379 | −0.846 | 1.00 | 90.54 | U |
| ATOM | 1433 | OD1 | ASN | U | 479 | 9.355 | 112.695 | −0.914 | 1.00 | 97.66 | U |
| ATOM | 1434 | ND2 | ASN | U | 479 | 10.951 | 111.210 | −0.340 | 1.00 | 87.84 | U |
| ATOM | 1435 | C | ASN | U | 479 | 13.753 | 112.285 | −2.202 | 1.00 | 79.08 | U |
| ATOM | 1436 | O | ASN | U | 479 | 13.276 | 112.379 | −3.335 | 1.00 | 78.53 | U |
| ATOM | 1437 | N | THR | U | 480 | 14.907 | 111.676 | −1.954 | 1.00 | 73.32 | U |
| ATOM | 1438 | CA | THR | U | 480 | 15.722 | 111.093 | −3.011 | 1.00 | 68.11 | U |
| ATOM | 1439 | CB | THR | U | 480 | 17.147 | 111.650 | −2.957 | 1.00 | 68.33 | U |
| ATOM | 1440 | OG1 | THR | U | 480 | 17.732 | 111.290 | −1.705 | 1.00 | 77.35 | U |
| ATOM | 1441 | CG2 | THR | U | 480 | 17.144 | 113.162 | −3.054 | 1.00 | 62.29 | U |
| ATOM | 1442 | C | THR | U | 480 | 15.789 | 109.582 | −2.816 | 1.00 | 63.60 | U |
| ATOM | 1443 | O | THR | U | 480 | 14.882 | 108.995 | −2.225 | 1.00 | 64.45 | U |
| ATOM | 1444 | N | SER | U | 481 | 16.856 | 108.960 | −3.316 | 1.00 | 54.01 | U |
| ATOM | 1445 | CA | SER | U | 481 | 17.030 | 107.521 | −3.172 | 1.00 | 58.62 | U |
| ATOM | 1446 | CB | SER | U | 481 | 17.533 | 106.898 | −4.467 | 1.00 | 62.02 | U |
| ATOM | 1447 | OG | SER | U | 481 | 18.908 | 107.173 | −4.665 | 1.00 | 59.14 | U |
| ATOM | 1448 | C | SER | U | 481 | 18.092 | 107.351 | −2.105 | 1.00 | 61.40 | U |
| ATOM | 1449 | O | SER | U | 481 | 18.921 | 108.234 | −1.949 | 1.00 | 60.96 | U |
| ATOM | 1450 | N | ILE | U | 482 | 18.081 | 106.230 | −1.382 | 1.00 | 62.19 | U |
| ATOM | 1451 | CA | ILE | U | 482 | 19.061 | 106.012 | −0.324 | 1.00 | 59.95 | U |
| ATOM | 1452 | CB | ILE | U | 482 | 19.132 | 104.525 | 0.152 | 1.00 | 63.75 | U |
| ATOM | 1453 | CG2 | ILE | U | 482 | 19.994 | 104.415 | 1.428 | 1.00 | 56.37 | U |
| ATOM | 1454 | CG1 | ILE | U | 482 | 17.737 | 103.992 | 0.472 | 1.00 | 66.43 | U |
| ATOM | 1455 | CD1 | ILE | U | 482 | 17.207 | 104.421 | 1.805 | 1.00 | 68.96 | U |
| ATOM | 1456 | C | ILE | U | 482 | 20.436 | 106.392 | −0.852 | 1.00 | 63.08 | U |
| ATOM | 1457 | O | ILE | U | 482 | 21.151 | 107.172 | −0.216 | 1.00 | 66.14 | U |
| ATOM | 1458 | N | ASP | U | 483 | 20.790 | 105.864 | −2.027 | 1.00 | 61.42 | U |
| ATOM | 1459 | CA | ASP | U | 483 | 22.109 | 106.112 | −2.628 | 1.00 | 55.14 | U |
| ATOM | 1460 | CB | ASP | U | 483 | 22.286 | 105.279 | −3.895 | 1.00 | 46.69 | U |
| ATOM | 1461 | CG | ASP | U | 483 | 21.833 | 103.868 | −3.701 | 1.00 | 54.41 | U |
| ATOM | 1462 | OD1 | ASP | U | 483 | 22.701 | 102.974 | −3.646 | 1.00 | 44.31 | U |
| ATOM | 1463 | OD2 | ASP | U | 483 | 20.594 | 103.669 | −3.575 | 1.00 | 59.52 | U |
| ATOM | 1464 | C | ASP | U | 483 | 22.387 | 107.564 | −2.934 | 1.00 | 51.45 | U |
| ATOM | 1465 | O | ASP | U | 483 | 23.481 | 108.051 | −2.687 | 1.00 | 53.59 | U |
| ATOM | 1466 | N | SER | U | 484 | 21.410 | 108.269 | −3.476 | 1.00 | 48.55 | U |
| ATOM | 1467 | CA | SER | U | 484 | 21.636 | 109.665 | −3.767 | 1.00 | 50.31 | U |
| ATOM | 1468 | CB | SER | U | 484 | 20.457 | 110.233 | −4.519 | 1.00 | 50.14 | U |
| ATOM | 1469 | OG | SER | U | 484 | 20.387 | 111.624 | −4.289 | 1.00 | 63.43 | U |
| ATOM | 1470 | C | SER | U | 484 | 21.857 | 110.463 | −2.476 | 1.00 | 52.59 | U |
| ATOM | 1471 | O | SER | U | 484 | 22.683 | 111.387 | −2.432 | 1.00 | 53.49 | U |
| ATOM | 1472 | N | PHE | U | 485 | 21.109 | 110.099 | −1.434 | 1.00 | 50.76 | U |
| ATOM | 1473 | CA | PHE | U | 485 | 21.199 | 110.746 | −0.124 | 1.00 | 48.74 | U |
| ATOM | 1474 | CB | PHE | U | 485 | 20.119 | 110.208 | 0.821 | 1.00 | 42.60 | U |
| ATOM | 1475 | CG | PHE | U | 485 | 20.400 | 110.483 | 2.274 | 1.00 | 50.41 | U |
| ATOM | 1476 | CD1 | PHE | U | 485 | 20.501 | 111.790 | 2.746 | 1.00 | 47.90 | U |
| ATOM | 1477 | CD2 | PHE | U | 485 | 20.623 | 109.437 | 3.163 | 1.00 | 52.06 | U |
| ATOM | 1478 | CE1 | PHE | U | 485 | 20.821 | 112.053 | 4.068 | 1.00 | 37.72 | U |
| ATOM | 1479 | CE2 | PHE | U | 485 | 20.947 | 109.694 | 4.499 | 1.00 | 49.14 | U |
| ATOM | 1480 | CZ | PHE | U | 485 | 21.047 | 111.007 | 4.947 | 1.00 | 48.32 | U |
| ATOM | 1481 | C | PHE | U | 485 | 22.569 | 110.497 | 0.498 | 1.00 | 51.52 | U |
| ATOM | 1482 | O | PHE | U | 485 | 23.220 | 111.417 | 0.995 | 1.00 | 51.80 | U |
| ATOM | 1483 | N | LEU | U | 486 | 22.986 | 109.235 | 0.486 | 1.00 | 48.33 | U |
| ATOM | 1484 | CA | LEU | U | 486 | 24.275 | 108.838 | 1.030 | 1.00 | 42.40 | U |
| ATOM | 1485 | CB | LEU | U | 486 | 24.452 | 107.328 | 0.894 | 1.00 | 35.79 | U |
| ATOM | 1486 | CG | LEU | U | 486 | 23.728 | 106.462 | 1.915 | 1.00 | 33.49 | U |
| ATOM | 1487 | CD1 | LEU | U | 486 | 23.949 | 105.002 | 1.579 | 1.00 | 26.67 | U |
| ATOM | 1488 | CD2 | LEU | U | 486 | 24.243 | 106.790 | 3.305 | 1.00 | 22.57 | U |
| ATOM | 1489 | C | LEU | U | 486 | 25.409 | 109.529 | 0.298 | 1.00 | 46.74 | U |
| ATOM | 1490 | O | LEU | U | 486 | 26.260 | 110.184 | 0.888 | 1.00 | 46.00 | U |
| ATOM | 1491 | N | LEU | U | 487 | 25.417 | 109.362 | −1.010 | 1.00 | 52.71 | U |
| ATOM | 1492 | CA | LEU | U | 487 | 26.447 | 109.946 | −1.844 | 1.00 | 51.57 | U |
| ATOM | 1493 | CB | LEU | U | 487 | 26.095 | 109.686 | −3.299 | 1.00 | 51.34 | U |
| ATOM | 1494 | CG | LEU | U | 487 | 26.974 | 110.286 | −4.374 | 1.00 | 50.14 | U |
| ATOM | 1495 | CD1 | LEU | U | 487 | 28.435 | 110.205 | −4.001 | 1.00 | 49.39 | U |
| ATOM | 1496 | CD2 | LEU | U | 487 | 26.676 | 109.529 | −5.652 | 1.00 | 53.61 | U |
| ATOM | 1497 | C | LEU | U | 487 | 26.641 | 111.438 | −1.589 | 1.00 | 48.88 | U |
| ATOM | 1498 | O | LEU | U | 487 | 27.750 | 111.892 | −1.338 | 1.00 | 48.29 | U |
| ATOM | 1499 | N | SER | U | 488 | 25.561 | 112.201 | −1.626 | 1.00 | 49.63 | U |
| ATOM | 1500 | CA | SER | U | 488 | 25.674 | 113.637 | −1.408 | 1.00 | 52.15 | U |
| ATOM | 1501 | CB | SER | U | 488 | 24.370 | 114.327 | −1.829 | 1.00 | 48.26 | U |
| ATOM | 1502 | OG | SER | U | 488 | 23.425 | 114.316 | −0.786 | 1.00 | 45.39 | U |
| ATOM | 1503 | C | SER | U | 488 | 26.022 | 113.996 | 0.042 | 1.00 | 52.35 | U |
| ATOM | 1504 | O | SER | U | 488 | 26.669 | 115.007 | 0.323 | 1.00 | 54.71 | U |
| ATOM | 1505 | N | MET | U | 489 | 25.585 | 113.165 | 0.968 | 1.00 | 52.82 | U |
| ATOM | 1506 | CA | MET | U | 489 | 25.857 | 113.414 | 2.364 | 1.00 | 51.60 | U |
| ATOM | 1507 | CB | MET | U | 489 | 24.988 | 112.506 | 3.214 | 1.00 | 46.06 | U |
| ATOM | 1508 | CG | MET | U | 489 | 24.962 | 112.877 | 4.656 | 1.00 | 53.48 | U |
| ATOM | 1509 | SD | MET | U | 489 | 23.612 | 113.978 | 5.008 | 1.00 | 57.03 | U |
| ATOM | 1510 | CE | MET | U | 489 | 23.857 | 115.229 | 3.663 | 1.00 | 59.12 | U |
| ATOM | 1511 | C | MET | U | 489 | 27.334 | 113.124 | 2.616 | 1.00 | 57.18 | U |
| ATOM | 1512 | O | MET | U | 489 | 27.960 | 113.744 | 3.480 | 1.00 | 59.72 | U |
| ATOM | 1513 | N | LEU | U | 490 | 27.888 | 112.186 | 1.847 | 1.00 | 56.23 | U |
| ATOM | 1514 | CA | LEU | U | 490 | 29.289 | 111.810 | 1.990 | 1.00 | 55.97 | U |
| ATOM | 1515 | CB | LEU | U | 490 | 29.612 | 110.548 | 1.181 | 1.00 | 42.91 | U |
| ATOM | 1516 | CG | LEU | U | 490 | 29.308 | 109.231 | 1.904 | 1.00 | 45.20 | U |
| ATOM | 1517 | CD1 | LEU | U | 490 | 29.785 | 108.064 | 1.089 | 1.00 | 44.97 | U |
| ATOM | 1518 | CD2 | LEU | U | 490 | 29.998 | 109.206 | 3.256 | 1.00 | 41.97 | U |
| ATOM | 1519 | C | LEU | U | 490 | 30.203 | 112.945 | 1.576 | 1.00 | 63.45 | U |
| ATOM | 1520 | O | LEU | U | 490 | 31.196 | 113.206 | 2.251 | 1.00 | 69.94 | U |
| ATOM | 1521 | N | GLU | U | 491 | 29.859 | 113.614 | 0.472 | 1.00 | 69.84 | U |
| ATOM | 1522 | CA | GLU | U | 491 | 30.619 | 114.751 | −0.064 | 1.00 | 68.81 | U |
| ATOM | 1523 | CB | GLU | U | 491 | 30.415 | 114.811 | −1.578 | 1.00 | 66.97 | U |
| ATOM | 1524 | CG | GLU | U | 491 | 30.716 | 113.482 | −2.293 | 1.00 | 72.32 | U |
| ATOM | 1525 | CD | GLU | U | 491 | 30.162 | 113.407 | −3.726 | 1.00 | 78.93 | U |
| ATOM | 1526 | OE1 | GLU | U | 491 | 30.549 | 112.473 | −4.474 | 1.00 | 73.54 | U |
| ATOM | 1527 | OE2 | GLU | U | 491 | 29.334 | 114.273 | −4.102 | 1.00 | 77.88 | U |
| ATOM | 1528 | C | GLU | U | 491 | 30.078 | 116.023 | 0.609 | 1.00 | 70.94 | U |
| ATOM | 1529 | O | GLU | U | 491 | 29.054 | 116.563 | 0.200 | 1.00 | 70.28 | U |
| ATOM | 1530 | N | SER | U | 492 | 30.754 | 116.493 | 1.653 | 1.00 | 74.07 | U |
| ATOM | 1531 | CA | SER | U | 492 | 30.277 | 117.668 | 2.376 | 1.00 | 79.39 | U |
| ATOM | 1532 | CB | SER | U | 492 | 29.068 | 117.266 | 3.216 | 1.00 | 72.71 | U |
| ATOM | 1533 | OG | SER | U | 492 | 28.556 | 118.371 | 3.927 | 1.00 | 78.28 | U |
| ATOM | 1534 | C | SER | U | 492 | 31.344 | 118.316 | 3.275 | 1.00 | 87.95 | U |
| ATOM | 1535 | O | SER | U | 492 | 32.444 | 117.726 | 3.408 | 1.00 | 93.73 | U |
| ATOM | 1536 | OXT | SER | U | 492 | 31.074 | 119.409 | 3.839 | 1.00 | 90.10 | U |
| ATOM | 1537 | CB | PRO | E | 179 | 49.063 | 79.237 | 25.302 | 1.00 | 63.97 | E |
| ATOM | 1538 | CG | PRO | E | 179 | 49.523 | 80.699 | 25.165 | 1.00 | 57.54 | E |
| ATOM | 1539 | C | PRO | E | 179 | 47.730 | 78.002 | 27.104 | 1.00 | 62.42 | E |
| ATOM | 1540 | O | PRO | E | 179 | 47.490 | 77.949 | 28.307 | 1.00 | 55.36 | E |
| ATOM | 1541 | N | PRO | E | 179 | 48.555 | 80.420 | 27.318 | 1.00 | 59.63 | E |
| ATOM | 1542 | CD | PRO | E | 179 | 49.650 | 81.155 | 26.643 | 1.00 | 64.55 | E |
| ATOM | 1543 | CA | PRO | E | 179 | 48.032 | 79.363 | 26.427 | 1.00 | 62.65 | E |
| ATOM | 1544 | N | ILE | E | 180 | 47.712 | 76.941 | 26.281 | 1.00 | 63.43 | E |
| ATOM | 1545 | CA | ILE | E | 180 | 47.477 | 75.504 | 26.600 | 1.00 | 60.71 | E |
| ATOM | 1546 | CB | ILE | E | 180 | 48.481 | 74.973 | 27.657 | 1.00 | 56.02 | E |
| ATOM | 1547 | CG2 | ILE | E | 180 | 49.882 | 75.406 | 27.284 | 1.00 | 49.91 | E |
| ATOM | 1548 | CG1 | ILE | E | 180 | 48.084 | 75.440 | 29.052 | 1.00 | 56.69 | E |
| ATOM | 1549 | CD1 | ILE | E | 180 | 48.587 | 74.527 | 30.150 | 1.00 | 64.15 | E |
| ATOM | 1550 | C | ILE | E | 180 | 46.080 | 74.938 | 26.962 | 1.00 | 59.69 | E |
| ATOM | 1551 | O | ILE | E | 180 | 45.474 | 75.331 | 27.961 | 1.00 | 61.02 | E |
| ATOM | 1552 | N | THR | E | 181 | 45.605 | 73.986 | 26.146 | 1.00 | 53.78 | E |
| ATOM | 1553 | CA | THR | E | 181 | 44.295 | 73.329 | 26.312 | 1.00 | 48.33 | E |
| ATOM | 1554 | CB | THR | E | 181 | 43.927 | 72.425 | 25.050 | 1.00 | 44.47 | E |
| ATOM | 1555 | OG1 | THR | E | 181 | 43.969 | 73.171 | 23.825 | 1.00 | 32.66 | E |
| ATOM | 1556 | CG2 | THR | E | 181 | 42.536 | 71.818 | 25.215 | 1.00 | 43.04 | E |
| ATOM | 1557 | C | THR | E | 181 | 44.297 | 72.377 | 27.526 | 1.00 | 49.61 | E |
| ATOM | 1558 | O | THR | E | 181 | 45.224 | 71.592 | 27.667 | 1.00 | 54.75 | E |
| ATOM | 1559 | N | PRO | E | 182 | 43.278 | 72.436 | 28.417 | 1.00 | 52.24 | E |
| ATOM | 1560 | CD | PRO | E | 182 | 42.438 | 73.617 | 28.652 | 1.00 | 59.60 | E |
| ATOM | 1561 | CA | PRO | E | 182 | 43.213 | 71.536 | 29.586 | 1.00 | 51.59 | E |
| ATOM | 1562 | CB | PRO | E | 182 | 42.509 | 72.364 | 30.668 | 1.00 | 45.60 | E |
| ATOM | 1563 | CG | PRO | E | 182 | 42.477 | 73.733 | 30.159 | 1.00 | 53.53 | E |
| ATOM | 1564 | C | PRO | E | 182 | 42.367 | 70.303 | 29.265 | 1.00 | 55.33 | E |
| ATOM | 1565 | O | PRO | E | 182 | 42.700 | 69.492 | 28.398 | 1.00 | 54.95 | E |
| ATOM | 1566 | N | GLU | E | 183 | 41.242 | 70.208 | 29.983 | 1.00 | 64.63 | E |
| ATOM | 1567 | CA | GLU | E | 183 | 40.258 | 69.114 | 29.890 | 1.00 | 59.48 | E |
| ATOM | 1568 | CB | GLU | E | 183 | 39.918 | 68.677 | 31.303 | 1.00 | 63.94 | E |
| ATOM | 1569 | CG | GLU | E | 183 | 41.252 | 68.361 | 32.006 | 1.00 | 67.86 | E |
| ATOM | 1570 | CD | GLU | E | 183 | 42.391 | 68.176 | 30.967 | 1.00 | 61.39 | E |
| ATOM | 1571 | OE1 | GLU | E | 183 | 42.141 | 67.503 | 29.928 | 1.00 | 56.70 | E |
| ATOM | 1572 | OE2 | GLU | E | 183 | 43.501 | 68.717 | 31.171 | 1.00 | 48.71 | E |
| ATOM | 1573 | C | GLU | E | 183 | 39.062 | 69.539 | 29.083 | 1.00 | 56.09 | E |
| ATOM | 1574 | O | GLU | E | 183 | 37.914 | 69.212 | 29.332 | 1.00 | 44.53 | E |
| ATOM | 1575 | N | GLN | E | 184 | 39.428 | 70.318 | 28.087 | 1.00 | 58.78 | E |
| ATOM | 1576 | CA | GLN | E | 184 | 38.567 | 70.860 | 27.086 | 1.00 | 54.12 | E |
| ATOM | 1577 | CB | GLN | E | 184 | 39.066 | 72.249 | 26.756 | 1.00 | 48.24 | E |
| ATOM | 1578 | CG | GLN | E | 184 | 39.271 | 73.052 | 28.012 | 1.00 | 45.50 | E |
| ATOM | 1579 | CD | GLN | E | 184 | 39.414 | 74.527 | 27.747 | 1.00 | 50.65 | E |
| ATOM | 1580 | OE1 | GLN | E | 184 | 40.503 | 74.995 | 27.406 | 1.00 | 40.87 | E |
| ATOM | 1581 | NE2 | GLN | E | 184 | 38.302 | 75.282 | 27.898 | 1.00 | 49.00 | E |
| ATOM | 1582 | C | GLN | E | 184 | 38.949 | 69.870 | 26.015 | 1.00 | 55.11 | E |
| ATOM | 1583 | O | GLN | E | 184 | 38.202 | 69.589 | 25.094 | 1.00 | 59.90 | E |
| ATOM | 1584 | N | GLU | E | 185 | 40.151 | 69.335 | 26.177 | 1.00 | 53.21 | E |
| ATOM | 1585 | CA | GLU | E | 185 | 40.668 | 68.340 | 25.279 | 1.00 | 49.10 | E |
| ATOM | 1586 | CB | GLU | E | 185 | 42.028 | 67.858 | 25.755 | 1.00 | 51.38 | E |
| ATOM | 1587 | CG | GLU | E | 185 | 42.739 | 67.063 | 24.692 | 1.00 | 61.09 | E |
| ATOM | 1588 | CD | GLU | E | 185 | 42.925 | 67.889 | 23.431 | 1.00 | 62.65 | E |
| ATOM | 1589 | OE1 | GLU | E | 185 | 43.446 | 69.018 | 23.577 | 1.00 | 58.60 | E |
| ATOM | 1590 | OE2 | GLU | E | 185 | 42.560 | 67.424 | 22.318 | 1.00 | 60.26 | E |
| ATOM | 1591 | C | GLU | E | 185 | 39.692 | 67.173 | 25.272 | 1.00 | 46.01 | E |
| ATOM | 1592 | O | GLU | E | 185 | 39.426 | 66.590 | 24.222 | 1.00 | 44.96 | E |
| ATOM | 1593 | N | GLU | E | 186 | 39.159 | 66.826 | 26.443 | 1.00 | 42.99 | E |
| ATOM | 1594 | CA | GLU | E | 186 | 38.200 | 65.732 | 26.507 | 1.00 | 43.92 | E |
| ATOM | 1595 | CB | GLU | E | 186 | 37.841 | 65.329 | 27.927 | 1.00 | 50.55 | E |
| ATOM | 1596 | CG | GLU | E | 186 | 36.916 | 64.111 | 27.932 | 1.00 | 54.03 | E |
| ATOM | 1597 | CD | GLU | E | 186 | 36.276 | 63.838 | 29.276 | 1.00 | 55.18 | E |
| ATOM | 1598 | OE1 | GLU | E | 186 | 36.992 | 63.857 | 30.301 | 1.00 | 55.83 | E |
| ATOM | 1599 | OE2 | GLU | E | 186 | 35.051 | 63.588 | 29.304 | 1.00 | 56.58 | E |
| ATOM | 1600 | C | GLU | E | 186 | 36.929 | 66.198 | 25.863 | 1.00 | 44.75 | E |
| ATOM | 1601 | O | GLU | E | 186 | 36.283 | 65.443 | 25.131 | 1.00 | 46.38 | E |
| ATOM | 1602 | N | LEU | E | 187 | 36.552 | 67.439 | 26.171 | 1.00 | 41.28 | E |
| ATOM | 1603 | CA | LEU | E | 187 | 35.344 | 68.019 | 25.597 | 1.00 | 34.84 | E |
| ATOM | 1604 | CB | LEU | E | 187 | 35.166 | 69.442 | 26.107 | 1.00 | 27.70 | E |
| ATOM | 1605 | CG | LEU | E | 187 | 33.949 | 70.135 | 25.489 | 1.00 | 28.29 | E |
| ATOM | 1606 | CD1 | LEU | E | 187 | 32.628 | 69.518 | 25.994 | 1.00 | 25.76 | E |
| ATOM | 1607 | CD2 | LEU | E | 187 | 34.049 | 71.604 | 25.807 | 1.00 | 32.95 | E |
| ATOM | 1608 | C | LEU | E | 187 | 35.363 | 68.024 | 24.051 | 1.00 | 34.96 | E |
| ATOM | 1609 | O | LEU | E | 187 | 34.356 | 67.724 | 23.384 | 1.00 | 35.08 | E |
| ATOM | 1610 | N | ILE | E | 188 | 36.516 | 68.376 | 23.495 | 1.00 | 29.71 | E |
| ATOM | 1611 | CA | ILE | E | 188 | 36.687 | 68.432 | 22.060 | 1.00 | 30.00 | E |
| ATOM | 1612 | CB | ILE | E | 188 | 38.018 | 69.131 | 21.687 | 1.00 | 24.30 | E |
| ATOM | 1613 | CG2 | ILE | E | 188 | 38.142 | 69.293 | 20.166 | 1.00 | 11.17 | E |
| ATOM | 1614 | CG1 | ILE | E | 188 | 38.046 | 70.520 | 22.323 | 1.00 | 22.82 | E |
| ATOM | 1615 | CD1 | ILE | E | 188 | 39.347 | 71.238 | 22.144 | 1.00 | 20.00 | E |
| ATOM | 1616 | C | ILE | E | 188 | 36.625 | 67.033 | 21.454 | 1.00 | 37.51 | E |
| ATOM | 1617 | O | ILE | E | 188 | 35.999 | 66.845 | 20.401 | 1.00 | 41.11 | E |
| ATOM | 1618 | N | HIS | E | 189 | 37.247 | 66.044 | 22.091 | 1.00 | 34.59 | E |
| ATOM | 1619 | CA | HIS | E | 189 | 37.173 | 64.709 | 21.509 | 1.00 | 42.58 | E |
| ATOM | 1620 | CB | HIS | E | 189 | 37.961 | 63.699 | 22.350 | 1.00 | 52.40 | E |
| ATOM | 1621 | CG | HIS | E | 189 | 39.441 | 63.862 | 22.236 | 1.00 | 66.69 | E |
| ATOM | 1622 | CD2 | HIS | E | 189 | 40.188 | 64.667 | 21.441 | 1.00 | 69.80 | E |
| ATOM | 1623 | ND1 | HIS | E | 189 | 40.331 | 63.155 | 23.015 | 1.00 | 71.12 | E |
| ATOM | 1624 | CE1 | HIS | E | 189 | 41.565 | 63.522 | 22.706 | 1.00 | 76.28 | E |
| ATOM | 1625 | NE2 | HIS | E | 189 | 41.506 | 64.437 | 21.755 | 1.00 | 69.65 | E |
| ATOM | 1626 | C | HIS | E | 189 | 35.705 | 64.284 | 21.384 | 1.00 | 41.69 | E |
| ATOM | 1627 | O | HIS | E | 189 | 35.271 | 63.746 | 20.354 | 1.00 | 43.43 | E |
| ATOM | 1628 | N | ARG | E | 190 | 34.942 | 64.559 | 22.432 | 1.00 | 34.44 | E |
| ATOM | 1629 | CA | ARG | E | 190 | 33.540 | 64.212 | 22.451 | 1.00 | 38.70 | E |
| ATOM | 1630 | CB | ARG | E | 190 | 32.924 | 64.607 | 23.792 | 1.00 | 38.89 | E |
| ATOM | 1631 | CG | ARG | E | 190 | 31.427 | 64.807 | 23.737 | 1.00 | 36.05 | E |
| ATOM | 1632 | CD | ARG | E | 190 | 30.892 | 65.121 | 25.095 | 1.00 | 40.10 | E |
| ATOM | 1633 | NE | ARG | E | 190 | 31.170 | 64.036 | 26.022 | 1.00 | 40.47 | E |
| ATOM | 1634 | CZ | ARG | E | 190 | 32.040 | 64.137 | 27.013 | 1.00 | 47.56 | E |
| ATOM | 1635 | NH1 | ARG | E | 190 | 32.705 | 65.278 | 27.199 | 1.00 | 46.35 | E |
| ATOM | 1636 | NH2 | ARG | E | 190 | 32.249 | 63.099 | 27.806 | 1.00 | 44.64 | E |
| ATOM | 1637 | C | ARG | E | 190 | 32.746 | 64.859 | 21.314 | 1.00 | 38.24 | E |
| ATOM | 1638 | O | ARG | E | 190 | 32.020 | 64.166 | 20.593 | 1.00 | 36.82 | E |
| ATOM | 1639 | N | LEU | E | 191 | 32.858 | 66.175 | 21.158 | 1.00 | 30.98 | E |
| ATOM | 1640 | CA | LEU | E | 191 | 32.103 | 66.814 | 20.105 | 1.00 | 34.04 | E |
| ATOM | 1641 | CB | LEU | E | 191 | 32.219 | 68.324 | 20.178 | 1.00 | 30.97 | E |
| ATOM | 1642 | CG | LEU | E | 191 | 31.843 | 69.007 | 21.477 | 1.00 | 32.34 | E |
| ATOM | 1643 | CD1 | LEU | E | 191 | 32.052 | 70.484 | 21.254 | 1.00 | 32.17 | E |
| ATOM | 1644 | CD2 | LEU | E | 191 | 30.417 | 68.719 | 21.879 | 1.00 | 22.30 | E |
| ATOM | 1645 | C | LEU | E | 191 | 32.583 | 66.353 | 18.752 | 1.00 | 37.58 | E |
| ATOM | 1646 | O | LEU | E | 191 | 31.777 | 66.091 | 17.866 | 1.00 | 41.61 | E |
| ATOM | 1647 | N | VAL | E | 192 | 33.894 | 66.247 | 18.577 | 1.00 | 35.14 | E |
| ATOM | 1648 | CA | VAL | E | 192 | 34.409 | 65.822 | 17.286 | 1.00 | 34.28 | E |
| ATOM | 1649 | CB | VAL | E | 192 | 35.950 | 65.935 | 17.233 | 1.00 | 30.40 | E |
| ATOM | 1650 | CG1 | VAL | E | 192 | 36.466 | 65.458 | 15.902 | 1.00 | 18.28 | E |
| ATOM | 1651 | CG2 | VAL | E | 192 | 36.349 | 67.376 | 17.397 | 1.00 | 27.26 | E |
| ATOM | 1652 | C | VAL | E | 192 | 33.945 | 64.406 | 16.967 | 1.00 | 34.14 | E |
| ATOM | 1653 | O | VAL | E | 192 | 33.745 | 64.065 | 15.798 | 1.00 | 32.27 | E |
| ATOM | 1654 | N | TYR | E | 193 | 33.743 | 63.606 | 18.015 | 1.00 | 34.68 | E |
| ATOM | 1655 | CA | TYR | E | 193 | 33.280 | 62.211 | 17.895 | 1.00 | 38.61 | E |
| ATOM | 1656 | CB | TYR | E | 193 | 33.429 | 61.502 | 19.253 | 1.00 | 41.45 | E |
| ATOM | 1657 | CG | TYR | E | 193 | 33.063 | 60.027 | 19.294 | 1.00 | 37.93 | E |
| ATOM | 1658 | CD1 | TYR | E | 193 | 34.011 | 59.040 | 19.042 | 1.00 | 39.12 | E |
| ATOM | 1659 | CE1 | TYR | E | 193 | 33.692 | 57.682 | 19.117 | 1.00 | 44.28 | E |
| ATOM | 1660 | CD2 | TYR | E | 193 | 31.778 | 59.625 | 19.623 | 1.00 | 44.95 | E |
| ATOM | 1661 | CE2 | TYR | E | 193 | 31.441 | 58.278 | 19.702 | 1.00 | 50.21 | E |
| ATOM | 1662 | CZ | TYR | E | 193 | 32.399 | 57.305 | 19.451 | 1.00 | 53.79 | E |
| ATOM | 1663 | OH | TYR | E | 193 | 32.045 | 55.967 | 19.540 | 1.00 | 61.14 | E |
| ATOM | 1664 | C | TYR | E | 193 | 31.816 | 62.175 | 17.454 | 1.00 | 37.90 | E |
| ATOM | 1665 | O | TYR | E | 193 | 31.477 | 61.575 | 16.444 | 1.00 | 40.94 | E |
| ATOM | 1666 | N | PHE | E | 194 | 30.958 | 62.829 | 18.230 | 1.00 | 40.65 | E |
| ATOM | 1667 | CA | PHE | E | 194 | 29.525 | 62.900 | 17.957 | 1.00 | 39.80 | E |
| ATOM | 1668 | CB | PHE | E | 194 | 28.819 | 63.590 | 19.115 | 1.00 | 38.32 | E |
| ATOM | 1669 | CG | PHE | E | 194 | 28.760 | 62.751 | 20.345 | 1.00 | 52.35 | E |
| ATOM | 1670 | CD1 | PHE | E | 194 | 29.002 | 63.306 | 21.596 | 1.00 | 53.88 | E |
| ATOM | 1671 | CD2 | PHE | E | 194 | 28.480 | 61.386 | 20.249 | 1.00 | 50.61 | E |
| ATOM | 1672 | CE1 | PHE | E | 194 | 28.970 | 62.515 | 22.733 | 1.00 | 59.51 | E |
| ATOM | 1673 | CE2 | PHE | E | 194 | 28.445 | 60.585 | 21.374 | 1.00 | 41.95 | E |
| ATOM | 1674 | CZ | PHE | E | 194 | 28.690 | 61.143 | 22.620 | 1.00 | 54.73 | E |
| ATOM | 1675 | C | PHE | E | 194 | 29.197 | 63.613 | 16.670 | 1.00 | 37.86 | E |
| ATOM | 1676 | O | PHE | E | 194 | 28.162 | 63.360 | 16.066 | 1.00 | 38.35 | E |
| ATOM | 1677 | N | GLN | E | 195 | 30.064 | 64.529 | 16.264 | 1.00 | 34.83 | E |
| ATOM | 1678 | CA | GLN | E | 195 | 29.854 | 65.244 | 15.017 | 1.00 | 38.45 | E |
| ATOM | 1679 | CB | GLN | E | 195 | 30.969 | 66.277 | 14.824 | 1.00 | 38.16 | E |
| ATOM | 1680 | CG | GLN | E | 195 | 31.237 | 66.701 | 13.391 | 1.00 | 32.13 | E |
| ATOM | 1681 | CD | GLN | E | 195 | 31.768 | 68.113 | 13.297 | 1.00 | 34.61 | E |
| ATOM | 1682 | OE1 | GLN | E | 195 | 31.005 | 69.095 | 13.290 | 1.00 | 33.03 | E |
| ATOM | 1683 | NE2 | GLN | E | 195 | 33.084 | 68.228 | 13.242 | 1.00 | 43.63 | E |
| ATOM | 1684 | C | GLN | E | 195 | 29.912 | 64.173 | 13.937 | 1.00 | 39.42 | E |
| ATOM | 1685 | O | GLN | E | 195 | 28.977 | 63.974 | 13.150 | 1.00 | 36.82 | E |
| ATOM | 1686 | N | ASN | E | 196 | 31.037 | 63.477 | 13.941 | 1.00 | 41.99 | E |
| ATOM | 1687 | CA | ASN | E | 196 | 31.318 | 62.403 | 13.023 | 1.00 | 41.31 | E |
| ATOM | 1688 | CB | ASN | E | 196 | 32.646 | 61.775 | 13.450 | 1.00 | 45.98 | E |
| ATOM | 1689 | CG | ASN | E | 196 | 32.971 | 60.506 | 12.706 | 1.00 | 55.28 | E |
| ATOM | 1690 | OD1 | ASN | E | 196 | 32.127 | 59.621 | 12.541 | 1.00 | 59.91 | E |
| ATOM | 1691 | ND2 | ASN | E | 196 | 34.217 | 60.393 | 12.273 | 1.00 | 62.85 | E |
| ATOM | 1692 | C | ASN | E | 196 | 30.161 | 61.399 | 13.095 | 1.00 | 40.61 | E |
| ATOM | 1693 | O | ASN | E | 196 | 29.547 | 61.069 | 12.082 | 1.00 | 43.29 | E |
| ATOM | 1694 | N | GLU | E | 197 | 29.843 | 60.934 | 14.296 | 1.00 | 33.92 | E |
| ATOM | 1695 | CA | GLU | E | 197 | 28.774 | 59.963 | 14.451 | 1.00 | 35.85 | E |
| ATOM | 1696 | CB | GLU | E | 197 | 28.578 | 59.602 | 15.907 | 1.00 | 35.07 | E |
| ATOM | 1697 | CG | GLU | E | 197 | 27.342 | 58.754 | 16.113 | 1.00 | 39.99 | E |
| ATOM | 1698 | CD | GLU | E | 197 | 27.250 | 58.206 | 17.519 | 1.00 | 50.67 | E |
| ATOM | 1699 | OE1 | GLU | E | 197 | 28.078 | 58.612 | 18.363 | 1.00 | 63.46 | E |
| ATOM | 1700 | OE2 | GLU | E | 197 | 26.348 | 57.382 | 17.787 | 1.00 | 46.48 | E |
| ATOM | 1701 | C | GLU | E | 197 | 27.421 | 60.346 | 13.870 | 1.00 | 39.95 | E |
| ATOM | 1702 | O | GLU | E | 197 | 26.810 | 59.548 | 13.167 | 1.00 | 45.46 | E |
| ATOM | 1703 | N | TYR | E | 198 | 26.940 | 61.551 | 14.156 | 1.00 | 40.87 | E |
| ATOM | 1704 | CA | TYR | E | 198 | 25.643 | 61.971 | 13.628 | 1.00 | 41.62 | E |
| ATOM | 1705 | CB | TYR | E | 198 | 24.890 | 62.790 | 14.673 | 1.00 | 44.79 | E |
| ATOM | 1706 | CG | TYR | E | 198 | 24.575 | 62.013 | 15.919 | 1.00 | 43.32 | E |
| ATOM | 1707 | CD1 | TYR | E | 198 | 25.219 | 62.305 | 17.123 | 1.00 | 46.37 | E |
| ATOM | 1708 | CE1 | TYR | E | 198 | 24.942 | 61.588 | 18.274 | 1.00 | 44.89 | E |
| ATOM | 1709 | CD2 | TYR | E | 198 | 23.645 | 60.984 | 15.895 | 1.00 | 33.72 | E |
| ATOM | 1710 | CE2 | TYR | E | 198 | 23.362 | 60.262 | 17.033 | 1.00 | 41.24 | E |
| ATOM | 1711 | CZ | TYR | E | 198 | 24.008 | 60.569 | 18.221 | 1.00 | 46.02 | E |
| ATOM | 1712 | OH | TYR | E | 198 | 23.702 | 59.875 | 19.363 | 1.00 | 43.60 | E |
| ATOM | 1713 | C | TYR | E | 198 | 25.665 | 62.739 | 12.309 | 1.00 | 40.33 | E |
| ATOM | 1714 | O | TYR | E | 198 | 24.735 | 63.478 | 12.001 | 1.00 | 37.35 | E |
| ATOM | 1715 | N | GLU | E | 199 | 26.705 | 62.544 | 11.510 | 1.00 | 42.59 | E |
| ATOM | 1716 | CA | GLU | E | 199 | 26.798 | 63.254 | 10.246 | 1.00 | 44.32 | E |
| ATOM | 1717 | CB | GLU | E | 199 | 28.234 | 63.271 | 9.753 | 1.00 | 45.64 | E |
| ATOM | 1718 | CG | GLU | E | 199 | 28.358 | 63.729 | 8.323 | 1.00 | 60.30 | E |
| ATOM | 1719 | CD | GLU | E | 199 | 29.612 | 64.535 | 8.097 | 1.00 | 80.08 | E |
| ATOM | 1720 | OE1 | GLU | E | 199 | 29.902 | 64.857 | 6.918 | 1.00 | 88.69 | E |
| ATOM | 1721 | OE2 | GLU | E | 199 | 30.299 | 64.855 | 9.104 | 1.00 | 85.89 | E |
| ATOM | 1722 | C | GLU | E | 199 | 25.920 | 62.766 | 9.116 | 1.00 | 42.47 | E |
| ATOM | 1723 | O | GLU | E | 199 | 25.411 | 63.571 | 8.355 | 1.00 | 42.38 | E |
| ATOM | 1724 | N | HIS | E | 200 | 25.752 | 61.452 | 8.999 | 1.00 | 48.21 | E |
| ATOM | 1725 | CA | HIS | E | 200 | 24.969 | 60.879 | 7.904 | 1.00 | 50.56 | E |
| ATOM | 1726 | CB | HIS | E | 200 | 25.862 | 59.945 | 7.067 | 1.00 | 51.56 | E |
| ATOM | 1727 | CG | HIS | E | 200 | 27.160 | 60.579 | 6.659 | 1.00 | 66.93 | E |
| ATOM | 1728 | CD2 | HIS | E | 200 | 27.416 | 61.710 | 5.949 | 1.00 | 70.41 | E |
| ATOM | 1729 | ND1 | HIS | E | 200 | 28.388 | 60.115 | 7.083 | 1.00 | 71.63 | E |
| ATOM | 1730 | CE1 | HIS | E | 200 | 29.343 | 60.929 | 6.662 | 1.00 | 68.48 | E |
| ATOM | 1731 | NE2 | HIS | E | 200 | 28.776 | 61.906 | 5.973 | 1.00 | 73.24 | E |
| ATOM | 1732 | C | HIS | E | 200 | 23.746 | 60.137 | 8.385 | 1.00 | 48.07 | E |
| ATOM | 1733 | O | HIS | E | 200 | 23.768 | 59.497 | 9.435 | 1.00 | 47.76 | E |
| ATOM | 1734 | N | PRO | E | 201 | 22.640 | 60.258 | 7.646 | 1.00 | 45.24 | E |
| ATOM | 1735 | CD | PRO | E | 201 | 22.407 | 61.202 | 6.545 | 1.00 | 41.22 | E |
| ATOM | 1736 | CA | PRO | E | 201 | 21.408 | 59.565 | 8.029 | 1.00 | 48.15 | E |
| ATOM | 1737 | CB | PRO | E | 201 | 20.338 | 60.262 | 7.188 | 1.00 | 43.73 | E |
| ATOM | 1738 | CG | PRO | E | 201 | 21.101 | 60.721 | 5.992 | 1.00 | 46.22 | E |
| ATOM | 1739 | C | PRO | E | 201 | 21.576 | 58.082 | 7.693 | 1.00 | 51.43 | E |
| ATOM | 1740 | O | PRO | E | 201 | 22.348 | 57.726 | 6.795 | 1.00 | 52.90 | E |
| ATOM | 1741 | N | SER | E | 202 | 20.868 | 57.219 | 8.416 | 1.00 | 54.88 | E |
| ATOM | 1742 | CA | SER | E | 202 | 20.977 | 55.772 | 8.202 | 1.00 | 54.89 | E |
| ATOM | 1743 | CB | SER | E | 202 | 20.054 | 55.012 | 9.142 | 1.00 | 51.86 | E |
| ATOM | 1744 | OG | SER | E | 202 | 18.719 | 55.146 | 8.697 | 1.00 | 53.06 | E |
| ATOM | 1745 | C | SER | E | 202 | 20.621 | 55.388 | 6.784 | 1.00 | 55.91 | E |
| ATOM | 1746 | O | SER | E | 202 | 19.596 | 55.808 | 6.255 | 1.00 | 60.73 | E |
| ATOM | 1747 | N | PRO | E | 203 | 21.446 | 54.551 | 6.154 | 1.00 | 56.97 | E |
| ATOM | 1748 | CD | PRO | E | 203 | 22.606 | 53.808 | 6.670 | 1.00 | 47.11 | E |
| ATOM | 1749 | CA | PRO | E | 203 | 21.130 | 54.163 | 4.776 | 1.00 | 56.59 | E |
| ATOM | 1750 | CB | PRO | E | 203 | 22.259 | 53.203 | 4.433 | 1.00 | 52.66 | E |
| ATOM | 1751 | CG | PRO | E | 203 | 22.602 | 52.614 | 5.795 | 1.00 | 45.14 | E |
| ATOM | 1752 | C | PRO | E | 203 | 19.725 | 53.565 | 4.573 | 1.00 | 54.90 | E |
| ATOM | 1753 | O | PRO | E | 203 | 19.269 | 53.448 | 3.437 | 1.00 | 54.58 | E |
| ATOM | 1754 | N | GLU | E | 204 | 19.039 | 53.195 | 5.653 | 1.00 | 52.27 | E |
| ATOM | 1755 | CA | GLU | E | 204 | 17.691 | 52.640 | 5.524 | 1.00 | 60.56 | E |
| ATOM | 1756 | CB | GLU | E | 204 | 17.271 | 51.958 | 6.805 | 1.00 | 65.25 | E |
| ATOM | 1757 | CG | GLU | E | 204 | 18.292 | 51.010 | 7.350 | 1.00 | 87.18 | E |
| ATOM | 1758 | CD | GLU | E | 204 | 18.017 | 50.695 | 8.809 | 1.00 | 102.16 | E |
| ATOM | 1759 | OE1 | GLU | E | 204 | 18.179 | 51.609 | 9.654 | 1.00 | 104.48 | E |
| ATOM | 1760 | OE2 | GLU | E | 204 | 17.621 | 49.541 | 9.111 | 1.00 | 109.20 | E |
| ATOM | 1761 | C | GLU | E | 204 | 16.703 | 53.763 | 5.249 | 1.00 | 63.74 | E |
| ATOM | 1762 | O | GLU | E | 204 | 15.758 | 53.607 | 4.465 | 1.00 | 59.93 | E |
| ATOM | 1763 | N | ASP | E | 205 | 16.928 | 54.891 | 5.927 | 1.00 | 67.57 | E |
| ATOM | 1764 | CA | ASP | E | 205 | 16.097 | 56.088 | 5.800 | 1.00 | 62.39 | E |
| ATOM | 1765 | CB | ASP | E | 205 | 16.433 | 57.085 | 6.901 | 1.00 | 60.01 | E |
| ATOM | 1766 | CG | ASP | E | 205 | 15.984 | 56.617 | 8.259 | 1.00 | 60.01 | E |
| ATOM | 1767 | OD1 | ASP | E | 205 | 15.093 | 55.744 | 8.312 | 1.00 | 54.54 | E |
| ATOM | 1768 | OD2 | ASP | E | 205 | 16.505 | 57.138 | 9.270 | 1.00 | 66.85 | E |
| ATOM | 1769 | C | ASP | E | 205 | 16.278 | 56.758 | 4.453 | 1.00 | 60.40 | E |
| ATOM | 1770 | O | ASP | E | 205 | 15.343 | 57.341 | 3.918 | 1.00 | 61.44 | E |
| ATOM | 1771 | N | ILE | E | 206 | 17.497 | 56.693 | 3.931 | 1.00 | 58.45 | E |
| ATOM | 1772 | CA | ILE | E | 206 | 17.825 | 57.250 | 2.625 | 1.00 | 57.04 | E |
| ATOM | 1773 | CB | ILE | E | 206 | 19.361 | 57.355 | 2.443 | 1.00 | 54.15 | E |
| ATOM | 1774 | CG2 | ILE | E | 206 | 19.709 | 57.780 | 1.021 | 1.00 | 41.45 | E |
| ATOM | 1775 | CG1 | ILE | E | 206 | 19.930 | 58.357 | 3.443 | 1.00 | 51.02 | E |
| ATOM | 1776 | CD1 | ILE | E | 206 | 21.440 | 58.304 | 3.548 | 1.00 | 65.11 | E |
| ATOM | 1777 | C | ILE | E | 206 | 17.222 | 56.299 | 1.586 | 1.00 | 60.40 | E |
| ATOM | 1778 | O | ILE | E | 206 | 16.886 | 56.710 | 0.472 | 1.00 | 60.60 | E |
| ATOM | 1779 | N | LYS | E | 207 | 17.091 | 55.024 | 1.966 | 1.00 | 65.28 | E |
| ATOM | 1780 | CA | LYS | E | 207 | 16.484 | 54.006 | 1.104 | 1.00 | 66.30 | E |
| ATOM | 1781 | CB | LYS | E | 207 | 16.533 | 52.609 | 1.753 | 1.00 | 74.28 | E |
| ATOM | 1782 | CG | LYS | E | 207 | 15.997 | 51.466 | 0.852 | 1.00 | 78.22 | E |
| ATOM | 1783 | CD | LYS | E | 207 | 15.241 | 50.353 | 1.618 | 1.00 | 79.75 | E |
| ATOM | 1784 | CE | LYS | E | 207 | 16.129 | 49.605 | 2.611 | 1.00 | 81.47 | E |
| ATOM | 1785 | NZ | LYS | E | 207 | 15.502 | 48.353 | 3.141 | 1.00 | 77.97 | E |
| ATOM | 1786 | C | LYS | E | 207 | 15.025 | 54.432 | 0.958 | 1.00 | 62.85 | E |
| ATOM | 1787 | O | LYS | E | 207 | 14.591 | 54.757 | −0.143 | 1.00 | 63.37 | E |
| ATOM | 1788 | N | ARG | E | 208 | 14.284 | 54.448 | 2.072 | 1.00 | 55.10 | E |
| ATOM | 1789 | CA | ARG | E | 208 | 12.883 | 54.860 | 2.053 | 1.00 | 53.30 | E |
| ATOM | 1790 | CB | ARG | E | 208 | 12.403 | 55.269 | 3.434 | 1.00 | 51.20 | E |
| ATOM | 1791 | CG | ARG | E | 208 | 12.086 | 54.150 | 4.345 | 1.00 | 55.85 | E |
| ATOM | 1792 | CD | ARG | E | 208 | 11.595 | 54.684 | 5.675 | 1.00 | 66.14 | E |
| ATOM | 1793 | NE | ARG | E | 208 | 11.687 | 53.662 | 6.717 | 1.00 | 77.33 | E |
| ATOM | 1794 | CZ | ARG | E | 208 | 12.812 | 53.323 | 7.347 | 1.00 | 79.98 | E |
| ATOM | 1795 | NH1 | ARG | E | 208 | 13.956 | 53.933 | 7.052 | 1.00 | 76.94 | E |
| ATOM | 1796 | NH2 | ARG | E | 208 | 12.797 | 52.360 | 8.264 | 1.00 | 82.91 | E |
| ATOM | 1797 | C | ARG | E | 208 | 12.629 | 56.037 | 1.136 | 1.00 | 55.84 | E |
| ATOM | 1798 | O | ARG | E | 208 | 11.614 | 56.079 | 0.449 | 1.00 | 60.42 | E |
| ATOM | 1799 | N | ILE | E | 209 | 13.538 | 57.007 | 1.136 | 1.00 | 58.02 | E |
| ATOM | 1800 | CA | ILE | E | 209 | 13.361 | 58.179 | 0.297 | 1.00 | 58.86 | E |
| ATOM | 1801 | CB | ILE | E | 209 | 14.403 | 59.276 | 0.606 | 1.00 | 53.27 | E |
| ATOM | 1802 | CG2 | ILE | E | 209 | 14.405 | 60.337 | −0.493 | 1.00 | 52.48 | E |
| ATOM | 1803 | CG1 | ILE | E | 209 | 14.059 | 59.938 | 1.936 | 1.00 | 41.03 | E |
| ATOM | 1804 | CD1 | ILE | E | 209 | 14.868 | 61.134 | 2.232 | 1.00 | 45.96 | E |
| ATOM | 1805 | C | ILE | E | 209 | 13.399 | 57.859 | −1.182 | 1.00 | 61.16 | E |
| ATOM | 1806 | O | ILE | E | 209 | 12.535 | 58.315 | −1.928 | 1.00 | 68.46 | E |
| ATOM | 1807 | N | VAL | E | 210 | 14.380 | 57.074 | −1.615 | 1.00 | 61.10 | E |
| ATOM | 1808 | CA | VAL | E | 210 | 14.464 | 56.743 | −3.035 | 1.00 | 66.44 | E |
| ATOM | 1809 | CB | VAL | E | 210 | 15.785 | 56.059 | −3.376 | 1.00 | 60.54 | E |
| ATOM | 1810 | CG1 | VAL | E | 210 | 16.015 | 56.173 | −4.872 | 1.00 | 49.11 | E |
| ATOM | 1811 | CG2 | VAL | E | 210 | 16.924 | 56.678 | −2.577 | 1.00 | 49.95 | E |
| ATOM | 1812 | C | VAL | E | 210 | 13.315 | 55.845 | −3.519 | 1.00 | 70.77 | E |
| ATOM | 1813 | O | VAL | E | 210 | 12.790 | 56.032 | −4.618 | 1.00 | 74.50 | E |
| ATOM | 1814 | N | ASN | E | 211 | 12.935 | 54.869 | −2.701 | 1.00 | 70.69 | E |
| ATOM | 1815 | CA | ASN | E | 211 | 11.846 | 53.965 | −3.043 | 1.00 | 70.57 | E |
| ATOM | 1816 | CB | ASN | E | 211 | 11.959 | 52.632 | −2.301 | 1.00 | 74.41 | E |
| ATOM | 1817 | CG | ASN | E | 211 | 13.300 | 51.954 | −2.503 | 1.00 | 78.30 | E |
| ATOM | 1818 | OD1 | ASN | E | 211 | 14.015 | 52.225 | −3.476 | 1.00 | 76.46 | E |
| ATOM | 1819 | ND2 | ASN | E | 211 | 13.646 | 51.050 | −1.583 | 1.00 | 80.75 | E |
| ATOM | 1820 | C | ASN | E | 211 | 10.582 | 54.627 | −2.587 | 1.00 | 71.71 | E |
| ATOM | 1821 | O | ASN | E | 211 | 9.880 | 54.091 | −1.734 | 1.00 | 73.49 | E |
| ATOM | 1822 | N | ALA | E | 212 | 10.308 | 55.804 | −3.128 | 1.00 | 73.73 | E |
| ATOM | 1823 | CA | ALA | E | 212 | 9.107 | 56.543 | −2.770 | 1.00 | 76.95 | E |
| ATOM | 1824 | CB | ALA | E | 212 | 9.397 | 57.541 | −1.640 | 1.00 | 72.06 | E |
| ATOM | 1825 | C | ALA | E | 212 | 8.639 | 57.274 | −4.011 | 1.00 | 79.72 | E |
| ATOM | 1826 | O | ALA | E | 212 | 7.513 | 57.782 | −4.053 | 1.00 | 80.85 | E |
| ATOM | 1827 | N | ALA | E | 213 | 9.507 | 57.313 | −5.024 | 1.00 | 79.03 | E |
| ATOM | 1828 | CA | ALA | E | 213 | 9.187 | 57.987 | −6.280 | 1.00 | 85.44 | E |
| ATOM | 1829 | CB | ALA | E | 213 | 10.189 | 57.592 | −7.361 | 1.00 | 74.35 | E |
| ATOM | 1830 | C | ALA | E | 213 | 7.766 | 57.643 | −6.728 | 1.00 | 92.69 | E |
| ATOM | 1831 | O | ALA | E | 213 | 7.411 | 56.471 | −6.832 | 1.00 | 97.04 | E |
| ATOM | 1832 | N | PRO | E | 214 | 6.922 | 58.662 | −6.969 | 1.00 | 96.04 | E |
| ATOM | 1833 | CD | PRO | E | 214 | 7.085 | 60.071 | −6.580 | 1.00 | 97.64 | E |
| ATOM | 1834 | CA | PRO | E | 214 | 5.547 | 58.414 | −7.406 | 1.00 | 97.66 | E |
| ATOM | 1835 | CB | PRO | E | 214 | 4.898 | 59.793 | −7.305 | 1.00 | 97.44 | E |
| ATOM | 1836 | CG | PRO | E | 214 | 5.672 | 60.454 | −6.238 | 1.00 | 98.77 | E |
| ATOM | 1837 | C | PRO | E | 214 | 5.509 | 57.886 | −8.839 | 1.00 | 100.95 | E |
| ATOM | 1838 | O | PRO | E | 214 | 6.551 | 57.609 | −9.448 | 1.00 | 99.62 | E |
| ATOM | 1839 | N | GLU | E | 215 | 4.297 | 57.759 | −9.369 | 1.00 | 103.11 | E |
| ATOM | 1840 | CA | GLU | E | 215 | 4.088 | 57.287 | −10.731 | 1.00 | 103.57 | E |
| ATOM | 1841 | CB | GLU | E | 215 | 2.587 | 57.206 | −11.013 | 1.00 | 105.61 | E |
| ATOM | 1842 | CG | GLU | E | 215 | 1.748 | 56.844 | −9.792 | 1.00 | 110.38 | E |
| ATOM | 1843 | CD | GLU | E | 215 | 0.278 | 57.221 | −9.949 | 1.00 | 115.86 | E |
| ATOM | 1844 | OE1 | GLU | E | 215 | −0.015 | 58.406 | −10.237 | 1.00 | 115.76 | E |
| ATOM | 1845 | OE2 | GLU | E | 215 | −0.587 | 56.334 | −9.774 | 1.00 | 119.71 | E |
| ATOM | 1846 | C | GLU | E | 215 | 4.749 | 58.302 | −11.671 | 1.00 | 103.96 | E |
| ATOM | 1847 | O | GLU | E | 215 | 4.225 | 59.406 | −11.872 | 1.00 | 102.63 | E |
| ATOM | 1848 | N | GLU | E | 216 | 5.901 | 57.932 | −12.231 | 1.00 | 103.93 | E |
| ATOM | 1849 | CA | GLU | E | 216 | 6.641 | 58.819 | −13.133 | 1.00 | 102.83 | E |
| ATOM | 1850 | CB | GLU | E | 216 | 5.908 | 58.949 | −14.465 | 1.00 | 103.19 | E |
| ATOM | 1851 | CG | GLU | E | 216 | 5.919 | 57.666 | −15.277 | 1.00 | 106.65 | E |
| ATOM | 1852 | CD | GLU | E | 216 | 5.094 | 57.763 | −16.544 | 1.00 | 107.70 | E |
| ATOM | 1853 | OE1 | GLU | E | 216 | 5.312 | 58.715 | −17.323 | 1.00 | 111.42 | E |
| ATOM | 1854 | OE2 | GLU | E | 216 | 4.234 | 56.884 | −16.767 | 1.00 | 107.18 | E |
| ATOM | 1855 | C | GLU | E | 216 | 6.810 | 60.193 | −12.495 | 1.00 | 101.72 | E |
| ATOM | 1856 | O | GLU | E | 216 | 6.120 | 61.153 | −12.857 | 1.00 | 98.48 | E |
| ATOM | 1857 | N | GLU | E | 217 | 7.741 | 60.267 | −11.545 | 1.00 | 101.00 | E |
| ATOM | 1858 | CA | GLU | E | 217 | 8.006 | 61.492 | −10.810 | 1.00 | 96.68 | E |
| ATOM | 1859 | CB | GLU | E | 217 | 9.136 | 61.271 | −9.803 | 1.00 | 95.92 | E |
| ATOM | 1860 | CG | GLU | E | 217 | 9.075 | 62.255 | −8.647 | 1.00 | 92.06 | E |
| ATOM | 1861 | CD | GLU | E | 217 | 9.841 | 61.786 | −7.433 | 1.00 | 87.31 | E |
| ATOM | 1862 | OE1 | GLU | E | 217 | 9.603 | 62.357 | −6.344 | 1.00 | 78.36 | E |
| ATOM | 1863 | OE2 | GLU | E | 217 | 10.672 | 60.856 | −7.577 | 1.00 | 82.01 | E |
| ATOM | 1864 | C | GLU | E | 217 | 8.327 | 62.675 | −11.712 | 1.00 | 95.99 | E |
| ATOM | 1865 | O | GLU | E | 217 | 9.045 | 62.546 | −12.709 | 1.00 | 91.78 | E |
| ATOM | 1866 | N | ASN | E | 218 | 7.780 | 63.828 | −11.328 | 1.00 | 94.78 | E |
| ATOM | 1867 | CA | ASN | E | 218 | 7.915 | 65.094 | −12.048 | 1.00 | 90.55 | E |
| ATOM | 1868 | CB | ASN | E | 218 | 7.362 | 66.227 | −11.172 | 1.00 | 84.77 | E |
| ATOM | 1869 | CG | ASN | E | 218 | 5.951 | 65.933 | −10.674 | 1.00 | 80.53 | E |
| ATOM | 1870 | OD1 | ASN | E | 218 | 5.067 | 65.588 | −11.460 | 1.00 | 84.63 | E |
| ATOM | 1871 | ND2 | ASN | E | 218 | 5.734 | 66.067 | −9.373 | 1.00 | 67.42 | E |
| ATOM | 1872 | C | ASN | E | 218 | 9.310 | 65.452 | −12.577 | 1.00 | 89.43 | E |
| ATOM | 1873 | O | ASN | E | 218 | 9.467 | 66.499 | −13.221 | 1.00 | 84.83 | E |
| ATOM | 1874 | N | VAL | E | 219 | 10.297 | 64.585 | −12.302 | 1.00 | 85.75 | E |
| ATOM | 1875 | CA | VAL | E | 219 | 11.692 | 64.734 | −12.751 | 1.00 | 83.21 | E |
| ATOM | 1876 | CB | VAL | E | 219 | 11.754 | 64.884 | −14.302 | 1.00 | 86.51 | E |
| ATOM | 1877 | CG1 | VAL | E | 219 | 11.816 | 66.377 | −14.704 | 1.00 | 86.68 | E |
| ATOM | 1878 | CG2 | VAL | E | 219 | 12.940 | 64.101 | −14.855 | 1.00 | 84.48 | E |
| ATOM | 1879 | C | VAL | E | 219 | 12.431 | 65.909 | −12.099 | 1.00 | 82.44 | E |
| ATOM | 1880 | O | VAL | E | 219 | 13.665 | 65.933 | −12.023 | 1.00 | 71.78 | E |
| ATOM | 1881 | N | ALA | E | 220 | 11.649 | 66.894 | −11.665 | 1.00 | 83.97 | E |
| ATOM | 1882 | CA | ALA | E | 220 | 12.147 | 68.082 | −10.994 | 1.00 | 80.39 | E |
| ATOM | 1883 | CB | ALA | E | 220 | 11.562 | 69.330 | −11.621 | 1.00 | 75.61 | E |
| ATOM | 1884 | C | ALA | E | 220 | 11.622 | 67.912 | −9.590 | 1.00 | 82.06 | E |
| ATOM | 1885 | O | ALA | E | 220 | 11.923 | 68.690 | −8.694 | 1.00 | 86.36 | E |
| ATOM | 1886 | N | GLU | E | 221 | 10.810 | 66.879 | −9.417 | 1.00 | 81.39 | E |
| ATOM | 1887 | CA | GLU | E | 221 | 10.246 | 66.580 | −8.125 | 1.00 | 86.99 | E |
| ATOM | 1888 | CB | GLU | E | 221 | 8.853 | 66.022 | −8.297 | 1.00 | 92.66 | E |
| ATOM | 1889 | CG | GLU | E | 221 | 7.796 | 67.052 | −8.041 | 1.00 | 102.40 | E |
| ATOM | 1890 | CD | GLU | E | 221 | 7.877 | 67.609 | −6.633 | 1.00 | 109.23 | E |
| ATOM | 1891 | OE1 | GLU | E | 221 | 8.134 | 66.810 | −5.697 | 1.00 | 109.14 | E |
| ATOM | 1892 | OE2 | GLU | E | 221 | 7.673 | 68.837 | −6.465 | 1.00 | 109.67 | E |
| ATOM | 1893 | C | GLU | E | 221 | 11.132 | 65.590 | −7.397 | 1.00 | 90.81 | E |
| ATOM | 1894 | O | GLU | E | 221 | 11.192 | 65.578 | −6.165 | 1.00 | 89.18 | E |
| ATOM | 1895 | N | GLU | E | 222 | 11.828 | 64.760 | −8.167 | 1.00 | 94.11 | E |
| ATOM | 1896 | CA | GLU | E | 222 | 12.733 | 63.781 | −7.583 | 1.00 | 94.68 | E |
| ATOM | 1897 | CB | GLU | E | 222 | 13.042 | 62.658 | −8.584 | 1.00 | 98.88 | E |
| ATOM | 1898 | CG | GLU | E | 222 | 13.844 | 61.481 | −8.021 | 1.00 | 101.14 | E |
| ATOM | 1899 | CD | GLU | E | 222 | 15.339 | 61.589 | −8.301 | 1.00 | 104.36 | E |
| ATOM | 1900 | OE1 | GLU | E | 222 | 15.727 | 61.583 | −9.494 | 1.00 | 102.91 | E |
| ATOM | 1901 | OE2 | GLU | E | 222 | 16.125 | 61.679 | −7.330 | 1.00 | 104.65 | E |
| ATOM | 1902 | C | GLU | E | 222 | 13.997 | 64.539 | −7.205 | 1.00 | 92.44 | E |
| ATOM | 1903 | O | GLU | E | 222 | 15.042 | 63.948 | −6.946 | 1.00 | 99.56 | E |
| ATOM | 1904 | N | ARG | E | 223 | 13.895 | 65.863 | −7.204 | 1.00 | 83.62 | E |
| ATOM | 1905 | CA | ARG | E | 223 | 15.001 | 66.724 | −6.816 | 1.00 | 73.36 | E |
| ATOM | 1906 | CB | ARG | E | 223 | 15.361 | 67.710 | −7.927 | 1.00 | 68.97 | E |
| ATOM | 1907 | CG | ARG | E | 223 | 16.254 | 67.139 | −9.003 | 1.00 | 74.59 | E |
| ATOM | 1908 | CD | ARG | E | 223 | 17.702 | 67.074 | −8.553 | 1.00 | 80.35 | E |
| ATOM | 1909 | NE | ARG | E | 223 | 18.530 | 66.283 | −9.463 | 1.00 | 87.11 | E |
| ATOM | 1910 | CZ | ARG | E | 223 | 18.519 | 64.951 | −9.523 | 1.00 | 89.83 | E |
| ATOM | 1911 | NH1 | ARG | E | 223 | 17.725 | 64.249 | −8.720 | 1.00 | 89.06 | E |
| ATOM | 1912 | NH2 | ARG | E | 223 | 19.296 | 64.317 | −10.390 | 1.00 | 89.00 | E |
| ATOM | 1913 | C | ARG | E | 223 | 14.479 | 67.474 | −5.616 | 1.00 | 66.94 | E |
| ATOM | 1914 | O | ARG | E | 223 | 15.141 | 67.575 | −4.595 | 1.00 | 72.27 | E |
| ATOM | 1915 | N | PHE | E | 224 | 13.264 | 67.984 | −5.739 | 1.00 | 56.45 | E |
| ATOM | 1916 | CA | PHE | E | 224 | 12.652 | 68.729 | −4.657 | 1.00 | 51.72 | E |
| ATOM | 1917 | CB | PHE | E | 224 | 11.396 | 69.426 | −5.162 | 1.00 | 45.84 | E |
| ATOM | 1918 | CG | PHE | E | 224 | 10.612 | 70.114 | −4.081 | 1.00 | 44.08 | E |
| ATOM | 1919 | CD1 | PHE | E | 224 | 10.903 | 71.427 | −3.716 | 1.00 | 43.29 | E |
| ATOM | 1920 | CD2 | PHE | E | 224 | 9.575 | 69.450 | −3.427 | 1.00 | 33.81 | E |
| ATOM | 1921 | CE1 | PHE | E | 224 | 10.164 | 72.063 | −2.716 | 1.00 | 40.50 | E |
| ATOM | 1922 | CE2 | PHE | E | 224 | 8.836 | 70.082 | −2.435 | 1.00 | 32.02 | E |
| ATOM | 1923 | CZ | PHE | E | 224 | 9.131 | 71.389 | −2.081 | 1.00 | 34.88 | E |
| ATOM | 1924 | C | PHE | E | 224 | 12.297 | 67.844 | −3.465 | 1.00 | 54.07 | E |
| ATOM | 1925 | O | PHE | E | 224 | 12.523 | 68.212 | −2.313 | 1.00 | 52.27 | E |
| ATOM | 1926 | N | ARG | E | 225 | 11.730 | 66.674 | −3.746 | 1.00 | 59.88 | E |
| ATOM | 1927 | CA | ARG | E | 225 | 11.338 | 65.746 | −2.689 | 1.00 | 55.01 | E |
| ATOM | 1928 | CB | ARG | E | 225 | 10.632 | 64.517 | −3.275 | 1.00 | 57.72 | E |
| ATOM | 1929 | CG | ARG | E | 225 | 9.791 | 63.743 | −2.259 | 1.00 | 58.92 | E |
| ATOM | 1930 | CD | ARG | E | 225 | 9.419 | 62.354 | −2.766 | 1.00 | 58.97 | E |
| ATOM | 1931 | NE | ARG | E | 225 | 10.605 | 61.514 | −2.893 | 1.00 | 65.72 | E |
| ATOM | 1932 | CZ | ARG | E | 225 | 10.790 | 60.660 | −3.887 | 1.00 | 60.47 | E |
| ATOM | 1933 | NH1 | ARG | E | 225 | 9.857 | 60.551 | −4.812 | 1.00 | 66.80 | E |
| ATOM | 1934 | NH2 | ARG | E | 225 | 11.905 | 59.946 | −3.978 | 1.00 | 58.05 | E |
| ATOM | 1935 | C | ARG | E | 225 | 12.575 | 65.307 | −1.923 | 1.00 | 49.76 | E |
| ATOM | 1936 | O | ARG | E | 225 | 12.523 | 65.132 | −0.721 | 1.00 | 44.61 | E |
| ATOM | 1937 | N | HIS | E | 226 | 13.688 | 65.122 | −2.623 | 1.00 | 49.52 | E |
| ATOM | 1938 | CA | HIS | E | 226 | 14.916 | 64.721 | −1.956 | 1.00 | 50.69 | E |
| ATOM | 1939 | CB | HIS | E | 226 | 16.000 | 64.401 | −2.988 | 1.00 | 56.47 | E |
| ATOM | 1940 | CG | HIS | E | 226 | 15.992 | 62.973 | −3.446 | 1.00 | 61.46 | E |
| ATOM | 1941 | CD2 | HIS | E | 226 | 16.988 | 62.181 | −3.912 | 1.00 | 62.54 | E |
| ATOM | 1942 | ND1 | HIS | E | 226 | 14.846 | 62.202 | −3.467 | 1.00 | 56.15 | E |
| ATOM | 1943 | CE1 | HIS | E | 226 | 15.136 | 60.997 | −3.925 | 1.00 | 56.70 | E |
| ATOM | 1944 | NE2 | HIS | E | 226 | 16.429 | 60.958 | −4.203 | 1.00 | 65.13 | E |
| ATOM | 1945 | C | HIS | E | 226 | 15.370 | 65.824 | −1.010 | 1.00 | 49.73 | E |
| ATOM | 1946 | O | HIS | E | 226 | 15.709 | 65.569 | 0.149 | 1.00 | 54.63 | E |
| ATOM | 1947 | N | ILE | E | 227 | 15.347 | 67.056 | −1.501 | 1.00 | 45.93 | E |
| ATOM | 1948 | CA | ILE | E | 227 | 15.729 | 68.212 | −0.710 | 1.00 | 39.16 | E |
| ATOM | 1949 | CB | ILE | E | 227 | 15.551 | 69.481 | −1.532 | 1.00 | 33.64 | E |
| ATOM | 1950 | CG2 | ILE | E | 227 | 15.619 | 70.697 | −0.654 | 1.00 | 38.70 | E |
| ATOM | 1951 | CG1 | ILE | E | 227 | 16.637 | 69.529 | −2.596 | 1.00 | 39.45 | E |
| ATOM | 1952 | CD1 | ILE | E | 227 | 16.600 | 70.746 | −3.474 | 1.00 | 42.76 | E |
| ATOM | 1953 | C | ILE | E | 227 | 14.922 | 68.328 | 0.584 | 1.00 | 38.85 | E |
| ATOM | 1954 | O | ILE | E | 227 | 15.479 | 68.506 | 1.662 | 1.00 | 41.38 | E |
| ATOM | 1955 | N | THR | E | 228 | 13.608 | 68.206 | 0.493 | 1.00 | 36.96 | E |
| ATOM | 1956 | CA | THR | E | 228 | 12.797 | 68.336 | 1.692 | 1.00 | 39.83 | E |
| ATOM | 1957 | CB | THR | E | 228 | 11.390 | 68.903 | 1.339 | 1.00 | 32.96 | E |
| ATOM | 1958 | OG1 | THR | E | 228 | 10.535 | 67.865 | 0.866 | 1.00 | 29.56 | E |
| ATOM | 1959 | CG2 | THR | E | 228 | 11.515 | 69.940 | 0.256 | 1.00 | 28.02 | E |
| ATOM | 1960 | C | THR | E | 228 | 12.668 | 67.072 | 2.561 | 1.00 | 41.84 | E |
| ATOM | 1961 | O | THR | E | 228 | 12.536 | 67.162 | 3.785 | 1.00 | 45.14 | E |
| ATOM | 1962 | N | GLU | E | 229 | 12.723 | 65.902 | 1.941 | 1.00 | 41.17 | E |
| ATOM | 1963 | CA | GLU | E | 229 | 12.604 | 64.656 | 2.680 | 1.00 | 42.21 | E |
| ATOM | 1964 | CB | GLU | E | 229 | 12.345 | 63.516 | 1.713 | 1.00 | 52.27 | E |
| ATOM | 1965 | CG | GLU | E | 229 | 11.033 | 63.628 | 0.958 | 1.00 | 61.58 | E |
| ATOM | 1966 | CD | GLU | E | 229 | 9.879 | 62.936 | 1.663 | 1.00 | 67.04 | E |
| ATOM | 1967 | OE1 | GLU | E | 229 | 9.615 | 63.279 | 2.847 | 1.00 | 64.96 | E |
| ATOM | 1968 | OE2 | GLU | E | 229 | 9.244 | 62.056 | 1.015 | 1.00 | 68.59 | E |
| ATOM | 1969 | C | GLU | E | 229 | 13.883 | 64.388 | 3.458 | 1.00 | 43.81 | E |
| ATOM | 1970 | O | GLU | E | 229 | 13.854 | 64.107 | 4.668 | 1.00 | 41.33 | E |
| ATOM | 1971 | N | ILE | E | 230 | 15.013 | 64.471 | 2.761 | 1.00 | 38.60 | E |
| ATOM | 1972 | CA | ILE | E | 230 | 16.295 | 64.242 | 3.412 | 1.00 | 38.64 | E |
| ATOM | 1973 | CB | ILE | E | 230 | 17.452 | 64.356 | 2.401 | 1.00 | 33.51 | E |
| ATOM | 1974 | CG2 | ILE | E | 230 | 18.771 | 64.116 | 3.091 | 1.00 | 34.50 | E |
| ATOM | 1975 | CG1 | ILE | E | 230 | 17.275 | 63.334 | 1.288 | 1.00 | 30.46 | E |
| ATOM | 1976 | CD1 | ILE | E | 230 | 18.326 | 63.439 | 0.187 | 1.00 | 34.35 | E |
| ATOM | 1977 | C | ILE | E | 230 | 16.529 | 65.254 | 4.552 | 1.00 | 41.49 | E |
| ATOM | 1978 | O | ILE | E | 230 | 17.167 | 64.928 | 5.549 | 1.00 | 42.12 | E |
| ATOM | 1979 | N | THR | E | 231 | 16.009 | 66.474 | 4.398 | 1.00 | 38.22 | E |
| ATOM | 1980 | CA | THR | E | 231 | 16.188 | 67.526 | 5.389 | 1.00 | 37.71 | E |
| ATOM | 1981 | CB | THR | E | 231 | 15.690 | 68.880 | 4.846 | 1.00 | 38.83 | E |
| ATOM | 1982 | OG1 | THR | E | 231 | 16.599 | 69.343 | 3.845 | 1.00 | 36.93 | E |
| ATOM | 1983 | CG2 | THR | E | 231 | 15.596 | 69.926 | 5.958 | 1.00 | 37.65 | E |
| ATOM | 1984 | C | THR | E | 231 | 15.488 | 67.202 | 6.699 | 1.00 | 40.58 | E |
| ATOM | 1985 | O | THR | E | 231 | 15.800 | 67.779 | 7.746 | 1.00 | 39.82 | E |
| ATOM | 1986 | N | ILE | E | 232 | 14.528 | 66.288 | 6.642 | 1.00 | 38.47 | E |
| ATOM | 1987 | CA | ILE | E | 232 | 13.842 | 65.884 | 7.856 | 1.00 | 37.93 | E |
| ATOM | 1988 | CB | ILE | E | 232 | 12.709 | 64.919 | 7.576 | 1.00 | 35.83 | E |
| ATOM | 1989 | CG2 | ILE | E | 232 | 12.192 | 64.373 | 8.864 | 1.00 | 37.58 | E |
| ATOM | 1990 | CG1 | ILE | E | 232 | 11.588 | 65.633 | 6.840 | 1.00 | 34.90 | E |
| ATOM | 1991 | CD1 | ILE | E | 232 | 11.049 | 64.810 | 5.706 | 1.00 | 39.78 | E |
| ATOM | 1992 | C | ILE | E | 232 | 14.898 | 65.145 | 8.651 | 1.00 | 37.38 | E |
| ATOM | 1993 | O | ILE | E | 232 | 15.044 | 65.347 | 9.852 | 1.00 | 39.17 | E |
| ATOM | 1994 | N | LEU | E | 233 | 15.628 | 64.285 | 7.949 | 1.00 | 34.45 | E |
| ATOM | 1995 | CA | LEU | E | 233 | 16.723 | 63.503 | 8.517 | 1.00 | 35.30 | E |
| ATOM | 1996 | CB | LEU | E | 233 | 17.354 | 62.611 | 7.430 | 1.00 | 33.51 | E |
| ATOM | 1997 | CG | LEU | E | 233 | 16.711 | 61.228 | 7.214 | 1.00 | 30.84 | E |
| ATOM | 1998 | CD1 | LEU | E | 233 | 15.355 | 61.186 | 7.974 | 1.00 | 4.40 | E |
| ATOM | 1999 | CD2 | LEU | E | 233 | 16.583 | 60.926 | 5.695 | 1.00 | 23.27 | E |
| ATOM | 2000 | C | LEU | E | 233 | 17.804 | 64.391 | 9.113 | 1.00 | 35.91 | E |
| ATOM | 2001 | O | LEU | E | 233 | 18.360 | 64.069 | 10.166 | 1.00 | 29.18 | E |
| ATOM | 2002 | N | THR | E | 234 | 18.094 | 65.500 | 8.423 | 1.00 | 36.21 | E |
| ATOM | 2003 | CA | THR | E | 234 | 19.119 | 66.450 | 8.848 | 1.00 | 36.54 | E |
| ATOM | 2004 | CB | THR | E | 234 | 19.327 | 67.593 | 7.814 | 1.00 | 39.35 | E |
| ATOM | 2005 | OG1 | THR | E | 234 | 19.557 | 67.043 | 6.508 | 1.00 | 41.29 | E |
| ATOM | 2006 | CG2 | THR | E | 234 | 20.528 | 68.470 | 8.227 | 1.00 | 29.41 | E |
| ATOM | 2007 | C | THR | E | 234 | 18.741 | 67.081 | 10.181 | 1.00 | 37.17 | E |
| ATOM | 2008 | O | THR | E | 234 | 19.550 | 67.149 | 11.094 | 1.00 | 39.38 | E |
| ATOM | 2009 | N | VAL | E | 235 | 17.507 | 67.548 | 10.299 | 1.00 | 35.56 | E |
| ATOM | 2010 | CA | VAL | E | 235 | 17.086 | 68.169 | 11.540 | 1.00 | 33.16 | E |
| ATOM | 2011 | CB | VAL | E | 235 | 15.693 | 68.793 | 11.409 | 1.00 | 32.20 | E |
| ATOM | 2012 | CG1 | VAL | E | 235 | 15.227 | 69.325 | 12.759 | 1.00 | 22.81 | E |
| ATOM | 2013 | CG2 | VAL | E | 235 | 15.751 | 69.908 | 10.388 | 1.00 | 25.24 | E |
| ATOM | 2014 | C | VAL | E | 235 | 17.097 | 67.164 | 12.674 | 1.00 | 32.90 | E |
| ATOM | 2015 | O | VAL | E | 235 | 17.409 | 67.509 | 13.817 | 1.00 | 30.52 | E |
| ATOM | 2016 | N | GLN | E | 236 | 16.777 | 65.919 | 12.355 | 1.00 | 29.64 | E |
| ATOM | 2017 | CA | GLN | E | 236 | 16.772 | 64.883 | 13.361 | 1.00 | 35.12 | E |
| ATOM | 2018 | CB | GLN | E | 236 | 16.188 | 63.620 | 12.778 | 1.00 | 40.23 | E |
| ATOM | 2019 | CG | GLN | E | 236 | 14.822 | 63.861 | 12.211 | 1.00 | 53.81 | E |
| ATOM | 2020 | CD | GLN | E | 236 | 14.041 | 62.588 | 12.009 | 1.00 | 53.35 | E |
| ATOM | 2021 | OE1 | GLN | E | 236 | 14.462 | 61.695 | 11.265 | 1.00 | 49.34 | E |
| ATOM | 2022 | NE2 | GLN | E | 236 | 12.889 | 62.497 | 12.672 | 1.00 | 53.19 | E |
| ATOM | 2023 | C | GLN | E | 236 | 18.182 | 64.633 | 13.880 | 1.00 | 38.28 | E |
| ATOM | 2024 | O | GLN | E | 236 | 18.407 | 64.595 | 15.098 | 1.00 | 46.79 | E |
| ATOM | 2025 | N | LEU | E | 237 | 19.122 | 64.465 | 12.951 | 1.00 | 33.28 | E |
| ATOM | 2026 | CA | LEU | E | 237 | 20.525 | 64.260 | 13.276 | 1.00 | 28.95 | E |
| ATOM | 2027 | CB | LEU | E | 237 | 21.331 | 64.180 | 11.991 | 1.00 | 20.94 | E |
| ATOM | 2028 | CG | LEU | E | 237 | 21.164 | 62.888 | 11.220 | 1.00 | 24.35 | E |
| ATOM | 2029 | CD1 | LEU | E | 237 | 21.703 | 63.031 | 9.811 | 1.00 | 31.88 | E |
| ATOM | 2030 | CD2 | LEU | E | 237 | 21.877 | 61.798 | 11.981 | 1.00 | 12.59 | E |
| ATOM | 2031 | C | LEU | E | 237 | 21.023 | 65.445 | 14.124 | 1.00 | 32.97 | E |
| ATOM | 2032 | O | LEU | E | 237 | 21.847 | 65.279 | 15.031 | 1.00 | 34.52 | E |
| ATOM | 2033 | N | ILE | E | 238 | 20.529 | 66.644 | 13.823 | 1.00 | 27.69 | E |
| ATOM | 2034 | CA | ILE | E | 238 | 20.914 | 67.816 | 14.582 | 1.00 | 26.59 | E |
| ATOM | 2035 | CB | ILE | E | 238 | 20.332 | 69.082 | 13.973 | 1.00 | 21.55 | E |
| ATOM | 2036 | CG2 | ILE | E | 238 | 20.483 | 70.227 | 14.948 | 1.00 | 19.01 | E |
| ATOM | 2037 | CG1 | ILE | E | 238 | 21.036 | 69.366 | 12.639 | 1.00 | 26.28 | E |
| ATOM | 2038 | CD1 | ILE | E | 238 | 20.473 | 70.523 | 11.831 | 1.00 | 19.16 | E |
| ATOM | 2039 | C | ILE | E | 238 | 20.408 | 67.669 | 16.010 | 1.00 | 32.55 | E |
| ATOM | 2040 | O | ILE | E | 238 | 21.132 | 67.980 | 16.964 | 1.00 | 30.08 | E |
| ATOM | 2041 | N | VAL | E | 239 | 19.173 | 67.190 | 16.168 | 1.00 | 30.61 | E |
| ATOM | 2042 | CA | VAL | E | 239 | 18.644 | 67.023 | 17.507 | 1.00 | 33.98 | E |
| ATOM | 2043 | CB | VAL | E | 239 | 17.162 | 66.625 | 17.518 | 1.00 | 29.85 | E |
| ATOM | 2044 | CG1 | VAL | E | 239 | 16.647 | 66.660 | 18.940 | 1.00 | 37.96 | E |
| ATOM | 2045 | CG2 | VAL | E | 239 | 16.355 | 67.575 | 16.706 | 1.00 | 35.97 | E |
| ATOM | 2046 | C | VAL | E | 239 | 19.428 | 65.930 | 18.219 | 1.00 | 37.91 | E |
| ATOM | 2047 | O | VAL | E | 239 | 19.685 | 66.032 | 19.423 | 1.00 | 39.77 | E |
| ATOM | 2048 | N | GLU | E | 240 | 19.806 | 64.894 | 17.470 | 1.00 | 30.46 | E |
| ATOM | 2049 | CA | GLU | E | 240 | 20.551 | 63.767 | 18.020 | 1.00 | 34.21 | E |
| ATOM | 2050 | CB | GLU | E | 240 | 20.834 | 62.716 | 16.938 | 1.00 | 49.56 | E |
| ATOM | 2051 | CG | GLU | E | 240 | 19.662 | 61.785 | 16.653 | 1.00 | 65.08 | E |
| ATOM | 2052 | CD | GLU | E | 240 | 18.953 | 61.378 | 17.939 | 1.00 | 79.27 | E |
| ATOM | 2053 | OE1 | GLU | E | 240 | 17.993 | 62.086 | 18.344 | 1.00 | 83.63 | E |
| ATOM | 2054 | OE2 | GLU | E | 240 | 19.376 | 60.369 | 18.561 | 1.00 | 84.61 | E |
| ATOM | 2055 | C | GLU | E | 240 | 21.851 | 64.203 | 18.648 | 1.00 | 33.16 | E |
| ATOM | 2056 | O | GLU | E | 240 | 22.186 | 63.805 | 19.760 | 1.00 | 37.83 | E |
| ATOM | 2057 | N | PHE | E | 241 | 22.580 | 65.027 | 17.919 | 1.00 | 35.09 | E |
| ATOM | 2058 | CA | PHE | E | 241 | 23.854 | 65.559 | 18.375 | 1.00 | 33.75 | E |
| ATOM | 2059 | CB | PHE | E | 241 | 24.552 | 66.222 | 17.174 | 1.00 | 34.01 | E |
| ATOM | 2060 | CG | PHE | E | 241 | 25.842 | 66.924 | 17.498 | 1.00 | 27.48 | E |
| ATOM | 2061 | CD1 | PHE | E | 241 | 27.056 | 66.306 | 17.266 | 1.00 | 27.18 | E |
| ATOM | 2062 | CD2 | PHE | E | 241 | 25.838 | 68.228 | 18.004 | 1.00 | 32.42 | E |
| ATOM | 2063 | CE1 | PHE | E | 241 | 28.250 | 66.972 | 17.529 | 1.00 | 33.13 | E |
| ATOM | 2064 | CE2 | PHE | E | 241 | 27.032 | 68.911 | 18.272 | 1.00 | 27.18 | E |
| ATOM | 2065 | CZ | PHE | E | 241 | 28.238 | 68.281 | 18.035 | 1.00 | 30.10 | E |
| ATOM | 2066 | C | PHE | E | 241 | 23.639 | 66.562 | 19.524 | 1.00 | 32.50 | E |
| ATOM | 2067 | O | PHE | E | 241 | 24.338 | 66.527 | 20.522 | 1.00 | 30.55 | E |
| ATOM | 2068 | N | SER | E | 242 | 22.660 | 67.448 | 19.397 | 1.00 | 34.08 | E |
| ATOM | 2069 | CA | SER | E | 242 | 22.420 | 68.435 | 20.447 | 1.00 | 38.69 | E |
| ATOM | 2070 | CB | SER | E | 242 | 21.212 | 69.303 | 20.100 | 1.00 | 40.24 | E |
| ATOM | 2071 | OG | SER | E | 242 | 21.477 | 70.073 | 18.944 | 1.00 | 37.08 | E |
| ATOM | 2072 | C | SER | E | 242 | 22.220 | 67.837 | 21.833 | 1.00 | 39.84 | E |
| ATOM | 2073 | O | SER | E | 242 | 22.591 | 68.434 | 22.835 | 1.00 | 40.74 | E |
| ATOM | 2074 | N | LYS | E | 243 | 21.654 | 66.645 | 21.896 | 1.00 | 42.89 | E |
| ATOM | 2075 | CA | LYS | E | 243 | 21.397 | 66.023 | 23.180 | 1.00 | 41.67 | E |
| ATOM | 2076 | CB | LYS | E | 243 | 20.298 | 64.981 | 23.011 | 1.00 | 38.11 | E |
| ATOM | 2077 | CG | LYS | E | 243 | 18.939 | 65.557 | 22.638 | 1.00 | 41.39 | E |
| ATOM | 2078 | CD | LYS | E | 243 | 17.925 | 64.436 | 22.447 | 1.00 | 43.69 | E |
| ATOM | 2079 | CE | LYS | E | 243 | 16.573 | 64.963 | 22.086 | 1.00 | 50.92 | E |
| ATOM | 2080 | NZ | LYS | E | 243 | 15.715 | 63.865 | 21.585 | 1.00 | 55.82 | E |
| ATOM | 2081 | C | LYS | E | 243 | 22.615 | 65.401 | 23.856 | 1.00 | 38.18 | E |
| ATOM | 2082 | O | LYS | E | 243 | 22.668 | 65.274 | 25.082 | 1.00 | 43.36 | E |
| ATOM | 2083 | N | ARG | E | 244 | 23.600 | 65.008 | 23.070 | 1.00 | 36.51 | E |
| ATOM | 2084 | CA | ARG | E | 244 | 24.787 | 64.386 | 23.651 | 1.00 | 41.55 | E |
| ATOM | 2085 | CB | ARG | E | 244 | 25.474 | 63.478 | 22.616 | 1.00 | 37.53 | E |
| ATOM | 2086 | CG | ARG | E | 244 | 24.497 | 62.607 | 21.818 | 1.00 | 46.34 | E |
| ATOM | 2087 | CD | ARG | E | 244 | 24.227 | 61.262 | 22.459 | 1.00 | 48.64 | E |
| ATOM | 2088 | NE | ARG | E | 244 | 25.425 | 60.432 | 22.424 | 1.00 | 54.52 | E |
| ATOM | 2089 | CZ | ARG | E | 244 | 25.468 | 59.163 | 22.803 | 1.00 | 58.12 | E |
| ATOM | 2090 | NH1 | ARG | E | 244 | 24.366 | 58.566 | 23.245 | 1.00 | 67.20 | E |
| ATOM | 2091 | NH2 | ARG | E | 244 | 26.619 | 58.498 | 22.765 | 1.00 | 59.41 | E |
| ATOM | 2092 | C | ARG | E | 244 | 25.759 | 65.464 | 24.128 | 1.00 | 41.59 | E |
| ATOM | 2093 | O | ARG | E | 244 | 26.862 | 65.143 | 24.594 | 1.00 | 39.08 | E |
| ATOM | 2094 | N | LEU | E | 245 | 25.337 | 66.730 | 24.010 | 1.00 | 33.30 | E |
| ATOM | 2095 | CA | LEU | E | 245 | 26.164 | 67.870 | 24.412 | 1.00 | 33.61 | E |
| ATOM | 2096 | CB | LEU | E | 245 | 25.776 | 69.153 | 23.656 | 1.00 | 35.79 | E |
| ATOM | 2097 | CG | LEU | E | 245 | 25.948 | 69.308 | 22.141 | 1.00 | 37.15 | E |
| ATOM | 2098 | CD1 | LEU | E | 245 | 25.622 | 70.743 | 21.750 | 1.00 | 30.82 | E |
| ATOM | 2099 | CD2 | LEU | E | 245 | 27.353 | 68.971 | 21.727 | 1.00 | 26.22 | E |
| ATOM | 2100 | C | LEU | E | 245 | 26.071 | 68.168 | 25.897 | 1.00 | 35.48 | E |
| ATOM | 2101 | O | LEU | E | 245 | 24.978 | 68.418 | 26.433 | 1.00 | 38.59 | E |
| ATOM | 2102 | N | PRO | E | 246 | 27.227 | 68.149 | 26.588 | 1.00 | 34.31 | E |
| ATOM | 2103 | CD | PRO | E | 246 | 28.510 | 67.738 | 25.993 | 1.00 | 29.84 | E |
| ATOM | 2104 | CA | PRO | E | 246 | 27.380 | 68.418 | 28.025 | 1.00 | 31.73 | E |
| ATOM | 2105 | CB | PRO | E | 246 | 28.883 | 68.555 | 28.175 | 1.00 | 26.92 | E |
| ATOM | 2106 | CG | PRO | E | 246 | 29.395 | 67.539 | 27.200 | 1.00 | 23.72 | E |
| ATOM | 2107 | C | PRO | E | 246 | 26.652 | 69.705 | 28.425 | 1.00 | 34.47 | E |
| ATOM | 2108 | O | PRO | E | 246 | 26.919 | 70.764 | 27.891 | 1.00 | 26.51 | E |
| ATOM | 2109 | N | GLY | E | 247 | 25.719 | 69.614 | 29.355 | 1.00 | 42.98 | E |
| ATOM | 2110 | CA | GLY | E | 247 | 25.011 | 70.808 | 29.760 | 1.00 | 44.36 | E |
| ATOM | 2111 | C | GLY | E | 247 | 23.590 | 70.854 | 29.251 | 1.00 | 46.41 | E |
| ATOM | 2112 | O | GLY | E | 247 | 22.739 | 71.505 | 29.848 | 1.00 | 52.00 | E |
| ATOM | 2113 | N | PHE | E | 248 | 23.317 | 70.165 | 28.152 | 1.00 | 45.04 | E |
| ATOM | 2114 | CA | PHE | E | 248 | 21.964 | 70.173 | 27.598 | 1.00 | 43.31 | E |
| ATOM | 2115 | CB | PHE | E | 248 | 21.927 | 69.389 | 26.287 | 1.00 | 32.36 | E |
| ATOM | 2116 | CG | PHE | E | 248 | 20.738 | 69.697 | 25.438 | 1.00 | 33.24 | E |
| ATOM | 2117 | CD1 | PHE | E | 248 | 20.616 | 70.934 | 24.816 | 1.00 | 29.68 | E |
| ATOM | 2118 | CD2 | PHE | E | 248 | 19.732 | 68.758 | 25.259 | 1.00 | 40.57 | E |
| ATOM | 2119 | CE1 | PHE | E | 248 | 19.516 | 71.233 | 24.030 | 1.00 | 32.10 | E |
| ATOM | 2120 | CE2 | PHE | E | 248 | 18.621 | 69.051 | 24.471 | 1.00 | 40.63 | E |
| ATOM | 2121 | CZ | PHE | E | 248 | 18.516 | 70.291 | 23.857 | 1.00 | 34.65 | E |
| ATOM | 2122 | C | PHE | E | 248 | 20.949 | 69.583 | 28.586 | 1.00 | 41.89 | E |
| ATOM | 2123 | O | PHE | E | 248 | 19.786 | 69.973 | 28.623 | 1.00 | 38.90 | E |
| ATOM | 2124 | N | ASP | E | 249 | 21.409 | 68.637 | 29.390 | 1.00 | 42.84 | E |
| ATOM | 2125 | CA | ASP | E | 249 | 20.565 | 67.990 | 30.373 | 1.00 | 46.68 | E |
| ATOM | 2126 | CB | ASP | E | 249 | 21.216 | 66.686 | 30.794 | 1.00 | 49.98 | E |
| ATOM | 2127 | CG | ASP | E | 249 | 22.575 | 66.903 | 31.437 | 1.00 | 62.57 | E |
| ATOM | 2128 | OD1 | ASP | E | 249 | 23.126 | 65.933 | 32.000 | 1.00 | 70.44 | E |
| ATOM | 2129 | OD2 | ASP | E | 249 | 23.099 | 68.045 | 31.382 | 1.00 | 67.68 | E |
| ATOM | 2130 | C | ASP | E | 249 | 20.363 | 68.889 | 31.603 | 1.00 | 47.20 | E |
| ATOM | 2131 | O | ASP | E | 249 | 19.433 | 68.716 | 32.373 | 1.00 | 49.50 | E |
| ATOM | 2132 | N | LYS | E | 250 | 21.237 | 69.854 | 31.802 | 1.00 | 45.53 | E |
| ATOM | 2133 | CA | LYS | E | 250 | 21.080 | 70.730 | 32.938 | 1.00 | 45.70 | E |
| ATOM | 2134 | CB | LYS | E | 250 | 22.406 | 71.450 | 33.188 | 1.00 | 49.35 | E |
| ATOM | 2135 | CG | LYS | E | 250 | 23.622 | 70.517 | 33.347 | 1.00 | 56.63 | E |
| ATOM | 2136 | CD | LYS | E | 250 | 23.506 | 69.582 | 34.555 | 1.00 | 63.25 | E |
| ATOM | 2137 | CE | LYS | E | 250 | 24.826 | 68.887 | 34.883 | 1.00 | 67.60 | E |
| ATOM | 2138 | NZ | LYS | E | 250 | 25.438 | 68.134 | 33.723 | 1.00 | 81.91 | E |
| ATOM | 2139 | C | LYS | E | 250 | 19.935 | 71.744 | 32.697 | 1.00 | 48.33 | E |
| ATOM | 2140 | O | LYS | E | 250 | 19.618 | 72.560 | 33.567 | 1.00 | 55.90 | E |
| ATOM | 2141 | N | LEU | E | 251 | 19.309 | 71.678 | 31.522 | 1.00 | 44.08 | E |
| ATOM | 2142 | CA | LEU | E | 251 | 18.220 | 72.591 | 31.138 | 1.00 | 36.71 | E |
| ATOM | 2143 | CB | LEU | E | 251 | 18.399 | 73.054 | 29.685 | 1.00 | 31.98 | E |
| ATOM | 2144 | CG | LEU | E | 251 | 19.714 | 73.688 | 29.267 | 1.00 | 30.27 | E |
| ATOM | 2145 | CD1 | LEU | E | 251 | 19.603 | 74.195 | 27.846 | 1.00 | 26.21 | E |
| ATOM | 2146 | CD2 | LEU | E | 251 | 20.033 | 74.827 | 30.209 | 1.00 | 31.20 | E |
| ATOM | 2147 | C | LEU | E | 251 | 16.838 | 71.964 | 31.252 | 1.00 | 34.43 | E |
| ATOM | 2148 | O | LEU | E | 251 | 16.680 | 70.748 | 31.166 | 1.00 | 31.57 | E |
| ATOM | 2149 | N | ILE | E | 252 | 15.827 | 72.805 | 31.403 | 1.00 | 33.03 | E |
| ATOM | 2150 | CA | ILE | E | 252 | 14.459 | 72.310 | 31.509 | 1.00 | 37.31 | E |
| ATOM | 2151 | CB | ILE | E | 252 | 13.592 | 73.372 | 32.252 | 1.00 | 37.73 | E |
| ATOM | 2152 | CG2 | ILE | E | 252 | 14.493 | 74.225 | 33.126 | 1.00 | 36.05 | E |
| ATOM | 2153 | CG1 | ILE | E | 252 | 12.925 | 74.333 | 31.275 | 1.00 | 39.95 | E |
| ATOM | 2154 | CD1 | ILE | E | 252 | 12.218 | 75.477 | 31.937 | 1.00 | 27.77 | E |
| ATOM | 2155 | C | ILE | E | 252 | 13.889 | 71.941 | 30.108 | 1.00 | 40.70 | E |
| ATOM | 2156 | O | ILE | E | 252 | 14.277 | 72.542 | 29.101 | 1.00 | 36.30 | E |
| ATOM | 2157 | N | ARG | E | 253 | 12.990 | 70.952 | 30.040 | 1.00 | 41.30 | E |
| ATOM | 2158 | CA | ARG | E | 253 | 12.430 | 70.534 | 28.751 | 1.00 | 47.33 | E |
| ATOM | 2159 | CB | ARG | E | 253 | 11.251 | 69.575 | 28.927 | 1.00 | 51.86 | E |
| ATOM | 2160 | CG | ARG | E | 253 | 11.633 | 68.166 | 29.394 | 1.00 | 69.36 | E |
| ATOM | 2161 | CD | ARG | E | 253 | 10.418 | 67.208 | 29.302 | 1.00 | 81.36 | E |
| ATOM | 2162 | NE | ARG | E | 253 | 10.298 | 66.272 | 30.436 | 1.00 | 89.24 | E |
| ATOM | 2163 | CZ | ARG | E | 253 | 10.117 | 66.618 | 31.720 | 1.00 | 87.21 | E |
| ATOM | 2164 | NH1 | ARG | E | 253 | 10.031 | 67.898 | 32.079 | 1.00 | 84.79 | E |
| ATOM | 2165 | NH2 | ARG | E | 253 | 10.022 | 65.676 | 32.658 | 1.00 | 81.99 | E |
| ATOM | 2166 | C | ARG | E | 253 | 11.985 | 71.713 | 27.901 | 1.00 | 48.18 | E |
| ATOM | 2167 | O | ARG | E | 253 | 12.176 | 71.731 | 26.684 | 1.00 | 49.82 | E |
| ATOM | 2168 | N | GLU | E | 254 | 11.386 | 72.698 | 28.553 | 1.00 | 52.53 | E |
| ATOM | 2169 | CA | GLU | E | 254 | 10.903 | 73.902 | 27.889 | 1.00 | 50.59 | E |
| ATOM | 2170 | CB | GLU | E | 254 | 10.367 | 74.897 | 28.927 | 1.00 | 57.10 | E |
| ATOM | 2171 | CG | GLU | E | 254 | 9.074 | 74.487 | 29.649 | 1.00 | 66.58 | E |
| ATOM | 2172 | CD | GLU | E | 254 | 9.212 | 73.292 | 30.608 | 1.00 | 74.77 | E |
| ATOM | 2173 | OE1 | GLU | E | 254 | 10.146 | 73.265 | 31.451 | 1.00 | 71.89 | E |
| ATOM | 2174 | OE2 | GLU | E | 254 | 8.355 | 72.381 | 30.529 | 1.00 | 80.85 | E |
| ATOM | 2175 | C | GLU | E | 254 | 12.037 | 74.551 | 27.106 | 1.00 | 49.05 | E |
| ATOM | 2176 | O | GLU | E | 254 | 11.901 | 74.816 | 25.905 | 1.00 | 43.89 | E |
| ATOM | 2177 | N | ASP | E | 255 | 13.151 | 74.796 | 27.801 | 1.00 | 46.39 | E |
| ATOM | 2178 | CA | ASP | E | 255 | 14.327 | 75.423 | 27.207 | 1.00 | 47.30 | E |
| ATOM | 2179 | CB | ASP | E | 255 | 15.284 | 75.923 | 28.300 | 1.00 | 44.84 | E |
| ATOM | 2180 | CG | ASP | E | 255 | 14.756 | 77.171 | 29.041 | 1.00 | 50.25 | E |
| ATOM | 2181 | OD1 | ASP | E | 255 | 13.728 | 77.751 | 28.611 | 1.00 | 47.08 | E |
| ATOM | 2182 | OD2 | ASP | E | 255 | 15.383 | 77.575 | 30.053 | 1.00 | 38.00 | E |
| ATOM | 2183 | C | ASP | E | 255 | 15.064 | 74.495 | 26.245 | 1.00 | 46.83 | E |
| ATOM | 2184 | O | ASP | E | 255 | 15.597 | 74.944 | 25.239 | 1.00 | 45.90 | E |
| ATOM | 2185 | N | GLN | E | 256 | 15.099 | 73.202 | 26.545 | 1.00 | 47.36 | E |
| ATOM | 2186 | CA | GLN | E | 256 | 15.764 | 72.265 | 25.653 | 1.00 | 44.16 | E |
| ATOM | 2187 | CB | GLN | E | 256 | 15.623 | 70.840 | 26.155 | 1.00 | 39.83 | E |
| ATOM | 2188 | CG | GLN | E | 256 | 16.328 | 70.566 | 27.454 | 1.00 | 50.75 | E |
| ATOM | 2189 | CD | GLN | E | 256 | 16.201 | 69.116 | 27.891 | 1.00 | 51.86 | E |
| ATOM | 2190 | OE1 | GLN | E | 256 | 17.093 | 68.580 | 28.551 | 1.00 | 51.56 | E |
| ATOM | 2191 | NE2 | GLN | E | 256 | 15.092 | 68.477 | 27.535 | 1.00 | 51.80 | E |
| ATOM | 2192 | C | GLN | E | 256 | 15.082 | 72.371 | 24.306 | 1.00 | 47.75 | E |
| ATOM | 2193 | O | GLN | E | 256 | 15.729 | 72.557 | 23.282 | 1.00 | 52.70 | E |
| ATOM | 2194 | N | ILE | E | 257 | 13.760 | 72.266 | 24.315 | 1.00 | 45.22 | E |
| ATOM | 2195 | CA | ILE | E | 257 | 12.976 | 72.329 | 23.088 | 1.00 | 39.99 | E |
| ATOM | 2196 | CB | ILE | E | 257 | 11.483 | 72.029 | 23.401 | 1.00 | 34.69 | E |
| ATOM | 2197 | CG2 | ILE | E | 257 | 10.621 | 72.336 | 22.202 | 1.00 | 39.38 | E |
| ATOM | 2198 | CG1 | ILE | E | 257 | 11.326 | 70.557 | 23.790 | 1.00 | 33.50 | E |
| ATOM | 2199 | CD1 | ILE | E | 257 | 9.907 | 70.125 | 24.072 | 1.00 | 38.84 | E |
| ATOM | 2200 | C | ILE | E | 257 | 13.120 | 73.659 | 22.327 | 1.00 | 38.00 | E |
| ATOM | 2201 | O | ILE | E | 257 | 13.139 | 73.697 | 21.102 | 1.00 | 39.30 | E |
| ATOM | 2202 | N | ALA | E | 258 | 13.243 | 74.752 | 23.056 | 1.00 | 34.43 | E |
| ATOM | 2203 | CA | ALA | E | 258 | 13.383 | 76.048 | 22.425 | 1.00 | 31.32 | E |
| ATOM | 2204 | CB | ALA | E | 258 | 13.292 | 77.129 | 23.474 | 1.00 | 26.11 | E |
| ATOM | 2205 | C | ALA | E | 258 | 14.710 | 76.149 | 21.683 | 1.00 | 36.70 | E |
| ATOM | 2206 | O | ALA | E | 258 | 14.735 | 76.537 | 20.511 | 1.00 | 34.47 | E |
| ATOM | 2207 | N | LEU | E | 259 | 15.800 | 75.807 | 22.386 | 1.00 | 36.87 | E |
| ATOM | 2208 | CA | LEU | E | 259 | 17.165 | 75.842 | 21.856 | 1.00 | 35.79 | E |
| ATOM | 2209 | CB | LEU | E | 259 | 18.164 | 75.243 | 22.848 | 1.00 | 33.20 | E |
| ATOM | 2210 | CG | LEU | E | 259 | 18.509 | 76.009 | 24.116 | 1.00 | 34.52 | E |
| ATOM | 2211 | CD1 | LEU | E | 259 | 19.679 | 75.312 | 24.793 | 1.00 | 31.47 | E |
| ATOM | 2212 | CD2 | LEU | E | 259 | 18.849 | 77.447 | 23.784 | 1.00 | 30.22 | E |
| ATOM | 2213 | C | LEU | E | 259 | 17.288 | 75.065 | 20.572 | 1.00 | 36.97 | E |
| ATOM | 2214 | O | LEU | E | 259 | 17.930 | 75.522 | 19.617 | 1.00 | 33.83 | E |
| ATOM | 2215 | N | LEU | E | 260 | 16.684 | 73.876 | 20.593 | 1.00 | 34.00 | E |
| ATOM | 2216 | CA | LEU | E | 260 | 16.663 | 72.933 | 19.480 | 1.00 | 30.57 | E |
| ATOM | 2217 | CB | LEU | E | 260 | 15.896 | 71.677 | 19.886 | 1.00 | 31.05 | E |
| ATOM | 2218 | CG | LEU | E | 260 | 16.639 | 70.378 | 20.192 | 1.00 | 36.94 | E |
| ATOM | 2219 | CD1 | LEU | E | 260 | 17.999 | 70.651 | 20.816 | 1.00 | 42.80 | E |
| ATOM | 2220 | CD2 | LEU | E | 260 | 15.778 | 69.534 | 21.121 | 1.00 | 41.52 | E |
| ATOM | 2221 | C | LEU | E | 260 | 16.015 | 73.545 | 18.262 | 1.00 | 31.96 | E |
| ATOM | 2222 | O | LEU | E | 260 | 16.593 | 73.552 | 17.182 | 1.00 | 41.76 | E |
| ATOM | 2223 | N | LYS | E | 261 | 14.804 | 74.056 | 18.424 | 1.00 | 25.94 | E |
| ATOM | 2224 | CA | LYS | E | 261 | 14.112 | 74.662 | 17.309 | 1.00 | 28.1.9 | E |
| ATOM | 2225 | CB | LYS | E | 261 | 12.730 | 75.126 | 17.756 | 1.00 | 26.86 | E |
| ATOM | 2226 | CG | LYS | E | 261 | 11.690 | 74.070 | 17.942 | 1.00 | 24.39 | E |
| ATOM | 2227 | CD | LYS | E | 261 | 10.661 | 74.588 | 18.933 | 1.00 | 30.08 | E |
| ATOM | 2228 | CE | LYS | E | 261 | 9.261 | 74.465 | 18.427 | 1.00 | 29.30 | E |
| ATOM | 2229 | NZ | LYS | E | 261 | 9.188 | 75.240 | 17.165 | 1.00 | 41.92 | E |
| ATOM | 2230 | C | LYS | E | 261 | 14.902 | 75.864 | 16.759 | 1.00 | 32.55 | E |
| ATOM | 2231 | O | LYS | E | 261 | 15.088 | 76.003 | 15.543 | 1.00 | 38.75 | E |
| ATOM | 2232 | N | ALA | E | 262 | 15.368 | 76.728 | 17.657 | 1.00 | 31.41 | E |
| ATOM | 2233 | CA | ALA | E | 262 | 16.102 | 77.922 | 17.264 | 1.00 | 34.42 | E |
| ATOM | 2234 | CB | ALA | E | 262 | 16.422 | 78.744 | 18.487 | 1.00 | 35.30 | E |
| ATOM | 2235 | C | ALA | E | 262 | 17.376 | 77.655 | 16.474 | 1.00 | 38.56 | E |
| ATOM | 2236 | O | ALA | E | 262 | 17.635 | 78.311 | 15.473 | 1.00 | 44.36 | E |
| ATOM | 2237 | N | CYS | E | 263 | 18.175 | 76.693 | 16.917 | 1.00 | 37.93 | E |
| ATOM | 2238 | CA | CYS | E | 263 | 19.418 | 76.392 | 16.236 | 1.00 | 34.78 | E |
| ATOM | 2239 | CB | CYS | E | 263 | 20.393 | 75.681 | 17.195 | 1.00 | 40.98 | E |
| ATOM | 2240 | SG | CYS | E | 263 | 20.184 | 73.848 | 17.433 | 1.00 | 41.61 | E |
| ATOM | 2241 | C | CYS | E | 263 | 19.275 | 75.556 | 14.978 | 1.00 | 34.16 | E |
| ATOM | 2242 | O | CYS | E | 263 | 20.121 | 75.649 | 14.112 | 1.00 | 41.65 | E |
| ATOM | 2243 | N | SER | E | 264 | 18.218 | 74.751 | 14.857 | 1.00 | 33.30 | E |
| ATOM | 2244 | CA | SER | E | 264 | 18.059 | 73.861 | 13.694 | 1.00 | 31.64 | E |
| ATOM | 2245 | CB | SER | E | 264 | 16.630 | 73.301 | 13.619 | 1.00 | 25.54 | E |
| ATOM | 2246 | OG | SER | E | 264 | 15.703 | 74.290 | 13.231 | 1.00 | 44.41 | E |
| ATOM | 2247 | C | SER | E | 264 | 18.486 | 74.436 | 12.331 | 1.00 | 29.78 | E |
| ATOM | 2248 | O | SER | E | 264 | 19.457 | 73.954 | 11.772 | 1.00 | 27.24 | E |
| ATOM | 2249 | N | SER | E | 265 | 17.791 | 75.453 | 11.810 | 1.00 | 32.04 | E |
| ATOM | 2250 | CA | SER | E | 265 | 18.136 | 76.071 | 10.521 | 1.00 | 31.46 | E |
| ATOM | 2251 | CB | SER | E | 265 | 17.128 | 77.154 | 10.157 | 1.00 | 28.33 | E |
| ATOM | 2252 | OG | SER | E | 265 | 16.739 | 77.850 | 11.326 | 1.00 | 45.47 | E |
| ATOM | 2253 | C | SER | E | 265 | 19.529 | 76.686 | 10.458 | 1.00 | 32.67 | E |
| ATOM | 2254 | O | SER | E | 265 | 20.104 | 76.768 | 9.381 | 1.00 | 40.29 | E |
| ATOM | 2255 | N | GLU | E | 266 | 20.070 | 77.149 | 11.584 | 1.00 | 28.39 | E |
| ATOM | 2256 | CA | GLU | E | 266 | 21.410 | 77.725 | 11.566 | 1.00 | 25.88 | E |
| ATOM | 2257 | CB | GLU | E | 266 | 21.654 | 78.598 | 12.796 | 1.00 | 22.84 | E |
| ATOM | 2258 | CG | GLU | E | 266 | 20.888 | 79.877 | 12.778 | 1.00 | 25.52 | E |
| ATOM | 2259 | CD | GLU | E | 266 | 21.005 | 80.647 | 14.070 | 1.00 | 38.74 | E |
| ATOM | 2260 | OE1 | GLU | E | 266 | 21.291 | 80.020 | 15.109 | 1.00 | 41.08 | E |
| ATOM | 2261 | OE2 | GLU | E | 266 | 20.787 | 81.876 | 14.062 | 1.00 | 49.52 | E |
| ATOM | 2262 | C | GLU | E | 266 | 22.445 | 76.610 | 11.495 | 1.00 | 28.30 | E |
| ATOM | 2263 | O | GLU | E | 266 | 23.337 | 76.650 | 10.659 | 1.00 | 35.01 | E |
| ATOM | 2264 | N | VAL | E | 267 | 22.321 | 75.617 | 12.372 | 1.00 | 29.09 | E |
| ATOM | 2265 | CA | VAL | E | 267 | 23.225 | 74.468 | 12.393 | 1.00 | 31.93 | E |
| ATOM | 2266 | CB | VAL | E | 267 | 22.868 | 73.542 | 13.554 | 1.00 | 27.95 | E |
| ATOM | 2267 | CG1 | VAL | E | 267 | 23.643 | 72.276 | 13.464 | 1.00 | 32.35 | E |
| ATOM | 2268 | CG2 | VAL | E | 267 | 23.193 | 74.211 | 14.829 | 1.00 | 32.98 | E |
| ATOM | 2269 | C | VAL | E | 267 | 23.174 | 73.682 | 11.064 | 1.00 | 33.52 | E |
| ATOM | 2270 | O | VAL | E | 267 | 24.168 | 73.120 | 10.614 | 1.00 | 37.32 | E |
| ATOM | 2271 | N | MET | E | 268 | 22.007 | 73.659 | 10.443 | 1.00 | 30.10 | E |
| ATOM | 2272 | CA | MET | E | 268 | 21.810 | 72.991 | 9.175 | 1.00 | 31.44 | E |
| ATOM | 2273 | CB | MET | E | 268 | 20.400 | 73.307 | 8.677 | 1.00 | 33.01 | E |
| ATOM | 2274 | CG | MET | E | 268 | 19.993 | 72.670 | 7.385 | 1.00 | 40.68 | E |
| ATOM | 2275 | SD | MET | E | 268 | 18.434 | 73.392 | 6.866 | 1.00 | 50.26 | E |
| ATOM | 2276 | CE | MET | E | 268 | 17.453 | 72.084 | 7.257 | 1.00 | 59.46 | E |
| ATOM | 2277 | C | MET | E | 268 | 22.860 | 73.455 | 8.158 | 1.00 | 36.00 | E |
| ATOM | 2278 | O | MET | E | 268 | 23.431 | 72.634 | 7.435 | 1.00 | 36.45 | E |
| ATOM | 2279 | N | MET | E | 269 | 23.118 | 74.766 | 8.116 | 1.00 | 38.01 | E |
| ATOM | 2280 | CA | MET | E | 269 | 24.095 | 75.365 | 7.192 | 1.00 | 33.54 | E |
| ATOM | 2281 | CB | MET | E | 269 | 24.065 | 76.891 | 7.318 | 1.00 | 29.18 | E |
| ATOM | 2282 | CG | MET | E | 269 | 22.688 | 77.525 | 7.232 | 1.00 | 29.83 | E |
| ATOM | 2283 | SD | MET | E | 269 | 21.893 | 77.406 | 5.629 | 1.00 | 36.33 | E |
| ATOM | 2284 | CE | MET | E | 269 | 20.192 | 77.383 | 6.059 | 1.00 | 35.76 | E |
| ATOM | 2285 | C | MET | E | 269 | 25.540 | 74.848 | 7.385 | 1.00 | 32.59 | E |
| ATOM | 2286 | O | MET | E | 269 | 26.301 | 74.760 | 6.421 | 1.00 | 32.25 | E |
| ATOM | 2287 | N | PHE | E | 270 | 25.923 | 74.528 | 8.623 | 1.00 | 24.35 | E |
| ATOM | 2288 | CA | PHE | E | 270 | 27.253 | 73.974 | 8.888 | 1.00 | 28.18 | E |
| ATOM | 2289 | CB | PHE | E | 270 | 27.519 | 73.884 | 10.394 | 1.00 | 30.27 | E |
| ATOM | 2290 | CG | PHE | E | 270 | 27.954 | 75.173 | 11.033 | 1.00 | 37.91 | E |
| ATOM | 2291 | CD1 | PHE | E | 270 | 28.000 | 75.282 | 12.424 | 1.00 | 34.23 | E |
| ATOM | 2292 | CD2 | PHE | E | 270 | 28.322 | 76.275 | 10.264 | 1.00 | 41.91 | E |
| ATOM | 2293 | CE1 | PHE | E | 270 | 28.403 | 76.470 | 13.041 | 1.00 | 38.32 | E |
| ATOM | 2294 | CE2 | PHE | E | 270 | 28.727 | 77.473 | 10.871 | 1.00 | 41.49 | E |
| ATOM | 2295 | CZ | PHE | E | 270 | 28.767 | 77.569 | 12.260 | 1.00 | 41.60 | E |
| ATOM | 2296 | C | PHE | E | 270 | 27.300 | 72.546 | 8.300 | 1.00 | 31.49 | E |
| ATOM | 2297 | O | PHE | E | 270 | 28.315 | 72.099 | 7.746 | 1.00 | 25.21 | E |
| ATOM | 2298 | N | ARG | E | 271 | 26.192 | 71.824 | 8.439 | 1.00 | 29.59 | E |
| ATOM | 2299 | CA | ARG | E | 271 | 26.113 | 70.474 | 7.921 | 1.00 | 33.01 | E |
| ATOM | 2300 | CB | ARG | E | 271 | 24.797 | 69.844 | 8.329 | 1.00 | 29.47 | E |
| ATOM | 2301 | CG | ARG | E | 271 | 24.907 | 69.091 | 9.623 | 1.00 | 30.97 | E |
| ATOM | 2302 | CD | ARG | E | 271 | 23.553 | 66.707 | 10.130 | 1.00 | 28.88 | E |
| ATOM | 2303 | NE | ARG | E | 271 | 23.637 | 67.946 | 11.371 | 1.00 | 29.81 | E |
| ATOM | 2304 | CZ | ARG | E | 271 | 23.959 | 66.656 | 11.446 | 1.00 | 35.74 | E |
| ATOM | 2305 | NH1 | ARG | E | 271 | 24.241 | 65.954 | 10.353 | 1.00 | 32.07 | E |
| ATOM | 2306 | NH2 | ARG | E | 271 | 23.959 | 66.055 | 12.625 | 1.00 | 38.84 | E |
| ATOM | 2307 | C | ARG | E | 271 | 26.258 | 70.472 | 6.413 | 1.00 | 36.29 | E |
| ATOM | 2308 | O | ARG | E | 271 | 27.030 | 69.692 | 5.860 | 1.00 | 35.05 | E |
| ATOM | 2309 | N | MET | E | 272 | 25.503 | 71.351 | 5.762 | 1.00 | 35.74 | E |
| ATOM | 2310 | CA | MET | E | 272 | 25.549 | 71.495 | 4.321 | 1.00 | 27.95 | E |
| ATOM | 2311 | CB | MET | E | 272 | 24.566 | 72.554 | 3.856 | 1.00 | 28.37 | E |
| ATOM | 2312 | CG | MET | E | 272 | 24.877 | 73.091 | 2.477 | 1.00 | 28.17 | E |
| ATOM | 2313 | SD | MET | E | 272 | 24.337 | 74.810 | 2.240 | 1.00 | 42.72 | E |
| ATOM | 2314 | CE | MET | E | 272 | 22.938 | 74.576 | 1.238 | 1.00 | 32.88 | E |
| ATOM | 2315 | C | MET | E | 272 | 26.940 | 71.936 | 3.928 | 1.00 | 26.79 | E |
| ATOM | 2316 | O | MET | E | 272 | 27.552 | 71.365 | 3.045 | 1.00 | 23.84 | E |
| ATOM | 2317 | N | ALA | E | 273 | 27.452 | 72.965 | 4.578 | 1.00 | 23.50 | E |
| ATOM | 2318 | CA | ALA | E | 273 | 28.781 | 73.425 | 4.212 | 1.00 | 29.19 | E |
| ATOM | 2319 | CB | ALA | E | 273 | 29.236 | 74.533 | 5.153 | 1.00 | 27.08 | E |
| ATOM | 2320 | C | ALA | E | 273 | 29.807 | 72.287 | 4.185 | 1.00 | 30.27 | E |
| ATOM | 2321 | O | ALA | E | 273 | 30.670 | 72.264 | 3.321 | 1.00 | 28.90 | E |
| ATOM | 2322 | N | ARG | E | 274 | 29.694 | 71.326 | 5.102 | 1.00 | 35.93 | E |
| ATOM | 2323 | CA | ARG | E | 274 | 30.662 | 70.230 | 5.177 | 1.00 | 33.33 | E |
| ATOM | 2324 | CB | ARG | E | 274 | 30.580 | 69.531 | 6.528 | 1.00 | 33.92 | E |
| ATOM | 2325 | CG | ARG | E | 274 | 30.624 | 70.457 | 7.705 | 1.00 | 36.71 | E |
| ATOM | 2326 | CD | ARG | E | 274 | 30.645 | 69.665 | 8.982 | 1.00 | 39.66 | E |
| ATOM | 2327 | NE | ARG | E | 274 | 32.000 | 69.288 | 9.347 | 1.00 | 33.19 | E |
| ATOM | 2328 | CZ | ARG | E | 274 | 32.359 | 68.076 | 9.750 | 1.00 | 34.09 | E |
| ATOM | 2329 | NH1 | ARG | E | 274 | 31.473 | 67.080 | 9.841 | 1.00 | 22.87 | E |
| ATOM | 2330 | NH2 | ARG | E | 274 | 33.616 | 67.867 | 10.090 | 1.00 | 30.93 | E |
| ATOM | 2331 | C | ARG | E | 274 | 30.529 | 69.187 | 4.098 | 1.00 | 36.69 | E |
| ATOM | 2332 | O | ARG | E | 274 | 31.309 | 68.255 | 4.045 | 1.00 | 44.32 | E |
| ATOM | 2333 | N | ARG | E | 275 | 29.531 | 69.320 | 3.248 | 1.00 | 39.84 | E |
| ATOM | 2334 | CA | ARG | E | 275 | 29.343 | 68.368 | 2.169 | 1.00 | 42.70 | E |
| ATOM | 2335 | CB | ARG | E | 275 | 27.948 | 67.776 | 2.250 | 1.00 | 36.63 | E |
| ATOM | 2336 | CG | ARG | E | 275 | 27.762 | 67.124 | 3.545 | 1.00 | 42.72 | E |
| ATOM | 2337 | CD | ARG | E | 275 | 28.563 | 65.885 | 3.542 | 1.00 | 49.94 | E |
| ATOM | 2338 | NE | ARG | E | 275 | 27.721 | 64.813 | 3.043 | 1.00 | 65.23 | E |
| ATOM | 2339 | CZ | ARG | E | 275 | 28.175 | 63.643 | 2.627 | 1.00 | 67.98 | E |
| ATOM | 2340 | NH1 | ARG | E | 275 | 29.487 | 63.402 | 2.644 | 1.00 | 65.23 | E |
| ATOM | 2341 | NH2 | ARG | E | 275 | 27.312 | 62.719 | 2.220 | 1.00 | 66.96 | E |
| ATOM | 2342 | C | ARG | E | 275 | 29.523 | 69.149 | 0.880 | 1.00 | 47.12 | E |
| ATOM | 2343 | O | ARG | E | 275 | 29.007 | 68.775 | −0.184 | 1.00 | 46.68 | E |
| ATOM | 2344 | N | TYR | E | 276 | 30.266 | 70.245 | 0.993 | 1.00 | 44.28 | E |
| ATOM | 2345 | CA | TYR | E | 276 | 30.499 | 71.090 | −0.153 | 1.00 | 48.82 | E |
| ATOM | 2346 | CB | TYR | E | 276 | 30.672 | 72.550 | 0.262 | 1.00 | 47.70 | E |
| ATOM | 2347 | CG | TYR | E | 276 | 31.082 | 73.464 | −0.881 | 1.00 | 49.48 | E |
| ATOM | 2348 | CD1 | TYR | E | 276 | 30.233 | 73.696 | −1.966 | 1.00 | 51.80 | E |
| ATOM | 2349 | CE1 | TYR | E | 276 | 30.626 | 74.537 | −3.011 | 1.00 | 53.53 | E |
| ATOM | 2350 | CD2 | TYR | E | 276 | 32.330 | 74.094 | −0.874 | 1.00 | 51.84 | E |
| ATOM | 2351 | CE2 | TYR | E | 276 | 32.732 | 74.930 | −1.908 | 1.00 | 46.44 | E |
| ATOM | 2352 | CZ | TYR | E | 276 | 31.882 | 75.150 | −2.967 | 1.00 | 52.03 | E |
| ATOM | 2353 | OH | TYR | E | 276 | 32.292 | 76.002 | −3.964 | 1.00 | 54.71 | E |
| ATOM | 2354 | C | TYR | E | 276 | 31.715 | 70.648 | −0.904 | 1.00 | 50.90 | E |
| ATOM | 2355 | O | TYR | E | 276 | 32.775 | 70.435 | −0.309 | 1.00 | 53.09 | E |
| ATOM | 2356 | N | ASP | E | 277 | 31.547 | 70.510 | −2.214 | 1.00 | 52.50 | E |
| ATOM | 2357 | CA | ASP | E | 277 | 32.631 | 70.114 | −3.086 | 1.00 | 60.62 | E |
| ATOM | 2358 | CB | ASP | E | 277 | 32.161 | 69.120 | −4.146 | 1.00 | 64.18 | E |
| ATOM | 2359 | CG | ASP | E | 277 | 33.326 | 68.424 | −4.830 | 1.00 | 66.10 | E |
| ATOM | 2360 | OD1 | ASP | E | 277 | 34.220 | 69.108 | −5.385 | 1.00 | 60.79 | E |
| ATOM | 2361 | OD2 | ASP | E | 277 | 33.346 | 67.182 | −4.799 | 1.00 | 70.36 | E |
| ATOM | 2362 | C | ASP | E | 277 | 33.175 | 71.341 | −3.782 | 1.00 | 60.24 | E |
| ATOM | 2363 | O | ASP | E | 277 | 32.565 | 71.860 | −4.714 | 1.00 | 60.67 | E |
| ATOM | 2364 | N | ALA | E | 278 | 34.330 | 71.802 | −3.336 | 1.00 | 59.71 | E |
| ATOM | 2365 | CA | ALA | E | 278 | 34.922 | 72.979 | −3.936 | 1.00 | 65.16 | E |
| ATOM | 2366 | CB | ALA | E | 278 | 36.284 | 73.242 | −3.315 | 1.00 | 63.99 | E |
| ATOM | 2367 | C | ALA | E | 278 | 35.041 | 72.850 | −5.458 | 1.00 | 68.11 | E |
| ATOM | 2368 | O | ALA | E | 278 | 34.638 | 73.744 | −6.198 | 1.00 | 70.52 | E |
| ATOM | 2369 | N | GLU | E | 279 | 35.572 | 71.729 | −5.927 | 1.00 | 71.62 | E |
| ATOM | 2370 | CA | GLU | E | 279 | 35.759 | 71.526 | −7.362 | 1.00 | 74.80 | E |
| ATOM | 2371 | CB | GLU | E | 279 | 36.386 | 70.151 | −7.592 | 1.00 | 83.39 | E |
| ATOM | 2372 | CG | GLU | E | 279 | 36.945 | 69.934 | −8.991 | 1.00 | 98.26 | E |
| ATOM | 2373 | CD | GLU | E | 279 | 37.749 | 68.638 | −9.106 | 1.00 | 104.83 | E |
| ATOM | 2374 | OE1 | GLU | E | 279 | 37.181 | 67.553 | −8.829 | 1.00 | 107.81 | E |
| ATOM | 2375 | OE2 | GLU | E | 279 | 38.948 | 68.709 | −9.473 | 1.00 | 103.41 | E |
| ATOM | 2376 | C | GLU | E | 279 | 34.487 | 71.679 | −8.213 | 1.00 | 68.90 | E |
| ATOM | 2377 | O | GLU | E | 279 | 34.387 | 72.584 | −9.040 | 1.00 | 68.62 | E |
| ATOM | 2378 | N | THR | E | 280 | 33.525 | 70.790 | −7.995 | 1.00 | 62.18 | E |
| ATOM | 2379 | CA | THR | E | 280 | 32.257 | 70.776 | −8.719 | 1.00 | 55.38 | E |
| ATOM | 2380 | CB | THR | E | 280 | 31.582 | 69.399 | −8.584 | 1.00 | 53.11 | E |
| ATOM | 2381 | OG1 | THR | E | 280 | 30.881 | 69.347 | −7.337 | 1.00 | 59.74 | E |
| ATOM | 2382 | CG2 | THR | E | 280 | 32.607 | 68.286 | −8.571 | 1.00 | 50.32 | E |
| ATOM | 2383 | C | THR | E | 280 | 31.234 | 71.812 | −8.220 | 1.00 | 57.07 | E |
| ATOM | 2384 | O | THR | E | 280 | 30.085 | 71.817 | −8.686 | 1.00 | 55.58 | E |
| ATOM | 2385 | N | ASP | E | 281 | 31.643 | 72.671 | −7.283 | 1.00 | 55.91 | E |
| ATOM | 2386 | CA | ASP | E | 281 | 30.758 | 73.678 | −6.680 | 1.00 | 52.78 | E |
| ATOM | 2387 | CB | ASP | E | 281 | 30.663 | 74.932 | −7.568 | 1.00 | 51.99 | E |
| ATOM | 2388 | CG | ASP | E | 281 | 29.900 | 76.093 | −6.892 | 1.00 | 57.39 | E |
| ATOM | 2389 | OD1 | ASP | E | 281 | 30.274 | 76.520 | −5.776 | 1.00 | 54.28 | E |
| ATOM | 2390 | OD2 | ASP | E | 281 | 28.924 | 76.595 | −7.490 | 1.00 | 60.91 | E |
| ATOM | 2391 | C | ASP | E | 281 | 29.365 | 73.081 | −6.423 | 1.00 | 52.37 | E |
| ATOM | 2392 | O | ASP | E | 281 | 28.347 | 73.586 | −6.913 | 1.00 | 48.32 | E |
| ATOM | 2393 | N | SER | E | 282 | 29.342 | 71.998 | −5.643 | 1.00 | 53.44 | E |
| ATOM | 2394 | CA | SER | E | 282 | 28.105 | 71.294 | −5.303 | 1.00 | 52.09 | E |
| ATOM | 2395 | CB | SER | E | 282 | 27.960 | 70.070 | −6.200 | 1.00 | 49.84 | E |
| ATOM | 2396 | OG | SER | E | 282 | 28.868 | 69.068 | −5.794 | 1.00 | 52.87 | E |
| ATOM | 2397 | C | SER | E | 282 | 28.031 | 70.833 | −3.832 | 1.00 | 50.47 | E |
| ATOM | 2398 | O | SER | E | 282 | 28.975 | 70.985 | −3.062 | 1.00 | 50.07 | E |
| ATOM | 2399 | N | ILE | E | 283 | 26.889 | 70.276 | −3.452 | 1.00 | 46.03 | E |
| ATOM | 2400 | CA | ILE | E | 283 | 26.705 | 69.760 | −2.109 | 1.00 | 45.77 | E |
| ATOM | 2401 | CB | ILE | E | 283 | 25.578 | 70.470 | −1.370 | 1.00 | 44.28 | E |
| ATOM | 2402 | CG2 | ILE | E | 283 | 25.395 | 69.843 | 0.008 | 1.00 | 38.20 | E |
| ATOM | 2403 | CG1 | ILE | E | 283 | 25.905 | 71.954 | −1.258 | 1.00 | 44.46 | E |
| ATOM | 2404 | CD1 | ILE | E | 283 | 24.702 | 72.840 | −1.423 | 1.00 | 38.28 | E |
| ATOM | 2405 | C | ILE | E | 283 | 26.321 | 68.300 | −2.291 | 1.00 | 51.61 | E |
| ATOM | 2406 | O | ILE | E | 283 | 25.237 | 67.987 | −2.785 | 1.00 | 56.45 | E |
| ATOM | 2407 | N | LEU | E | 284 | 27.218 | 67.408 | −1.891 | 1.00 | 48.95 | E |
| ATOM | 2408 | CA | LEU | E | 284 | 26.998 | 65.984 | −2.029 | 1.00 | 42.78 | E |
| ATOM | 2409 | CB | LEU | E | 284 | 28.317 | 65.239 | −1.838 | 1.00 | 46.07 | E |
| ATOM | 2410 | CG | LEU | E | 284 | 28.257 | 63.712 | −1.744 | 1.00 | 48.34 | E |
| ATOM | 2411 | CD1 | LEU | E | 284 | 27.765 | 63.121 | −3.055 | 1.00 | 43.11 | E |
| ATOM | 2412 | CD2 | LEU | E | 284 | 29.633 | 63.191 | −1.397 | 1.00 | 44.44 | E |
| ATOM | 2413 | C | LEU | E | 284 | 25.997 | 65.469 | −1.032 | 1.00 | 43.09 | E |
| ATOM | 2414 | O | LEU | E | 284 | 26.358 | 65.177 | 0.103 | 1.00 | 48.13 | E |
| ATOM | 2415 | N | PHE | E | 285 | 24.744 | 65.351 | −1.452 | 1.00 | 39.45 | E |
| ATOM | 2416 | CA | PHE | E | 285 | 23.699 | 64.829 | −0.575 | 1.00 | 39.78 | E |
| ATOM | 2417 | CB | PHE | E | 285 | 22.365 | 64.722 | −1.329 | 1.00 | 37.53 | E |
| ATOM | 2418 | CG | PHE | E | 285 | 21.562 | 66.008 | −1.379 | 1.00 | 36.81 | E |
| ATOM | 2419 | CD1 | PHE | E | 285 | 22.191 | 67.260 | −1.363 | 1.00 | 33.71 | E |
| ATOM | 2420 | CD2 | PHE | E | 285 | 20.163 | 65.960 | −1.500 | 1.00 | 34.34 | E |
| ATOM | 2421 | CE1 | PHE | E | 285 | 21.438 | 68.442 | −1.472 | 1.00 | 28.76 | E |
| ATOM | 2422 | CE2 | PHE | E | 285 | 19.395 | 67.139 | −1.613 | 1.00 | 29.05 | E |
| ATOM | 2423 | CZ | PHE | E | 285 | 20.038 | 68.380 | −1.599 | 1.00 | 25.56 | E |
| ATOM | 2424 | C | PHE | E | 285 | 24.078 | 63.439 | −0.036 | 1.00 | 46.86 | E |
| ATOM | 2425 | O | PHE | E | 285 | 25.044 | 62.813 | −0.488 | 1.00 | 47.48 | E |
| ATOM | 2426 | N | ALA | E | 286 | 23.296 | 62.956 | 0.926 | 1.00 | 53.92 | E |
| ATOM | 2427 | CA | ALA | E | 286 | 23.515 | 61.648 | 1.547 | 1.00 | 58.44 | E |
| ATOM | 2428 | CB | ALA | E | 286 | 22.754 | 61.559 | 2.885 | 1.00 | 61.45 | E |
| ATOM | 2429 | C | ALA | E | 286 | 23.084 | 60.511 | 0.620 | 1.00 | 56.92 | E |
| ATOM | 2430 | O | ALA | E | 286 | 23.152 | 59.340 | 0.994 | 1.00 | 60.75 | E |
| ATOM | 2431 | N | THR | E | 287 | 22.618 | 60.870 | −0.575 | 1.00 | 56.10 | E |
| ATOM | 2432 | CA | THR | E | 287 | 22.213 | 59.893 | −1.584 | 1.00 | 54.00 | E |
| ATOM | 2433 | CB | THR | E | 287 | 20.861 | 60.215 | −2.240 | 1.00 | 48.15 | E |
| ATOM | 2434 | OG1 | THR | E | 287 | 20.899 | 61.526 | −2.832 | 1.00 | 44.13 | E |
| ATOM | 2435 | CG2 | THR | E | 287 | 19.749 | 60.094 | −1.234 | 1.00 | 40.34 | E |
| ATOM | 2436 | C | THR | E | 287 | 23.260 | 59.940 | −2.683 | 1.00 | 58.66 | E |
| ATOM | 2437 | O | THR | E | 287 | 22.983 | 59.660 | −3.841 | 1.00 | 62.98 | E |
| ATOM | 2438 | N | ASN | E | 288 | 24.469 | 60.322 | −2.315 | 1.00 | 59.74 | E |
| ATOM | 2439 | CA | ASN | E | 288 | 25.547 | 60.392 | −3.276 | 1.00 | 61.45 | E |
| ATOM | 2440 | CB | ASN | E | 288 | 26.004 | 58.990 | −3.609 | 1.00 | 55.68 | E |
| ATOM | 2441 | CG | ASN | E | 288 | 27.445 | 58.799 | −3.297 | 1.00 | 58.87 | E |
| ATOM | 2442 | OD1 | ASN | E | 288 | 28.305 | 59.406 | −3.936 | 1.00 | 57.06 | E |
| ATOM | 2443 | ND2 | ASN | E | 288 | 27.734 | 57.986 | −2.287 | 1.00 | 58.96 | E |
| ATOM | 2444 | C | ASN | E | 288 | 25.253 | 61.169 | −4.559 | 1.00 | 64.23 | E |
| ATOM | 2445 | O | ASN | E | 288 | 25.977 | 61.057 | −5.548 | 1.00 | 62.43 | E |
| ATOM | 2446 | N | GLN | E | 289 | 24.187 | 61.958 | −4.533 | 1.00 | 65.98 | E |
| ATOM | 2447 | CA | GLN | E | 289 | 23.807 | 62.783 | −5.669 | 1.00 | 65.31 | E |
| ATOM | 2448 | CB | GLN | E | 289 | 22.279 | 62.911 | −5.729 | 1.00 | 69.73 | E |
| ATOM | 2449 | CG | GLN | E | 289 | 21.509 | 61.909 | −6.583 | 1.00 | 67.32 | E |
| ATOM | 2450 | CD | GLN | E | 289 | 19.988 | 62.139 | −6.498 | 1.00 | 75.69 | E |
| ATOM | 2451 | OE1 | GLN | E | 289 | 19.267 | 61.956 | −7.486 | 1.00 | 73.62 | E |
| ATOM | 2452 | NE2 | GLN | E | 289 | 19.500 | 62.536 | −5.307 | 1.00 | 69.96 | E |
| ATOM | 2453 | C | GLN | E | 289 | 24.418 | 64.188 | −5.482 | 1.00 | 62.08 | E |
| ATOM | 2454 | O | GLN | E | 289 | 24.241 | 64.817 | −4.435 | 1.00 | 64.89 | E |
| ATOM | 2455 | N | PRO | E | 290 | 25.190 | 64.673 | −6.463 | 1.00 | 55.91 | E |
| ATOM | 2456 | CD | PRO | E | 290 | 26.100 | 63.884 | −7.308 | 1.00 | 54.16 | E |
| ATOM | 2457 | CA | PRO | E | 290 | 25.738 | 66.021 | −6.256 | 1.00 | 54.20 | E |
| ATOM | 2458 | CB | PRO | E | 290 | 26.983 | 66.024 | −7.133 | 1.00 | 46.46 | E |
| ATOM | 2459 | CG | PRO | E | 290 | 27.414 | 64.593 | −7.085 | 1.00 | 52.27 | E |
| ATOM | 2460 | C | PRO | E | 290 | 24.724 | 67.087 | −6.684 | 1.00 | 51.49 | E |
| ATOM | 2461 | O | PRO | E | 290 | 24.237 | 67.068 | −7.814 | 1.00 | 55.18 | E |
| ATOM | 2462 | N | TYR | E | 291 | 24.386 | 68.003 | −5.782 | 1.00 | 47.44 | E |
| ATOM | 2463 | CA | TYR | E | 291 | 23.434 | 69.053 | −6.116 | 1.00 | 46.43 | E |
| ATOM | 2464 | CB | TYR | E | 291 | 22.458 | 69.290 | −4.954 | 1.00 | 42.30 | E |
| ATOM | 2465 | CG | TYR | E | 291 | 21.343 | 68.263 | −4.929 | 1.00 | 43.04 | E |
| ATOM | 2466 | CD1 | TYR | E | 291 | 21.631 | 66.892 | −4.802 | 1.00 | 37.33 | E |
| ATOM | 2467 | CE1 | TYR | E | 291 | 20.609 | 65.926 | −4.833 | 1.00 | 33.09 | E |
| ATOM | 2468 | CD2 | TYR | E | 291 | 20.000 | 68.644 | −5.083 | 1.00 | 37.03 | E |
| ATOM | 2469 | CE2 | TYR | E | 291 | 18.971 | 67.681 | −5.110 | 1.00 | 33.82 | E |
| ATOM | 2470 | CZ | TYR | E | 291 | 19.287 | 66.327 | −4.986 | 1.00 | 39.67 | E |
| ATOM | 2471 | OH | TYR | E | 291 | 18.299 | 65.364 | −5.005 | 1.00 | 48.43 | E |
| ATOM | 2472 | C | TYR | E | 291 | 24.173 | 70.329 | −6.468 | 1.00 | 48.61 | E |
| ATOM | 2473 | O | TYR | E | 291 | 25.176 | 70.660 | −5.837 | 1.00 | 51.77 | E |
| ATOM | 2474 | N | THR | E | 292 | 23.686 | 71.031 | −7.489 | 1.00 | 48.55 | E |
| ATOM | 2475 | CA | THR | E | 292 | 24.291 | 72.282 | −7.952 | 1.00 | 49.68 | E |
| ATOM | 2476 | CB | THR | E | 292 | 24.862 | 72.103 | −9.323 | 1.00 | 53.56 | E |
| ATOM | 2477 | OG1 | THR | E | 292 | 23.830 | 71.572 | −10.167 | 1.00 | 57.88 | E |
| ATOM | 2478 | CG2 | THR | E | 292 | 26.063 | 71.156 | −9.293 | 1.00 | 45.92 | E |
| ATOM | 2479 | C | THR | E | 292 | 23.249 | 73.395 | −8.054 | 1.00 | 52.34 | E |
| ATOM | 2480 | O | THR | E | 292 | 22.073 | 73.203 | −7.705 | 1.00 | 53.46 | E |
| ATOM | 2481 | N | ARG | E | 293 | 23.670 | 74.561 | −8.534 | 1.00 | 50.43 | E |
| ATOM | 2482 | CA | ARG | E | 293 | 22.726 | 75.667 | −8.673 | 1.00 | 56.45 | E |
| ATOM | 2483 | CB | ARG | E | 293 | 23.365 | 76.840 | −9.426 | 1.00 | 60.07 | E |
| ATOM | 2484 | CG | ARG | E | 293 | 22.452 | 78.055 | −9.641 | 1.00 | 65.01 | E |
| ATOM | 2485 | CD | ARG | E | 293 | 23.089 | 79.128 | −10.557 | 1.00 | 70.96 | E |
| ATOM | 2486 | NE | ARG | E | 293 | 24.363 | 79.662 | −10.053 | 1.00 | 79.55 | E |
| ATOM | 2487 | CZ | ARG | E | 293 | 25.568 | 79.135 | −10.291 | 1.00 | 83.43 | E |
| ATOM | 2488 | NH1 | ARG | E | 293 | 25.687 | 78.048 | −11.038 | 1.00 | 92.10 | E |
| ATOM | 2489 | NH2 | ARG | E | 293 | 26.663 | 79.689 | −9.779 | 1.00 | 83.65 | E |
| ATOM | 2490 | C | ARG | E | 293 | 21.536 | 75.142 | −9.460 | 1.00 | 59.86 | E |
| ATOM | 2491 | O | ARG | E | 293 | 20.395 | 75.540 | −9.225 | 1.00 | 58.30 | E |
| ATOM | 2492 | N | GLU | E | 294 | 21.808 | 74.231 | −10.391 | 1.00 | 65.91 | E |
| ATOM | 2493 | CA | GLU | E | 294 | 20.741 | 73.671 | −11.205 | 1.00 | 67.66 | E |
| ATOM | 2494 | CB | GLU | E | 294 | 21.299 | 72.777 | −12.307 | 1.00 | 73.63 | E |
| ATOM | 2495 | CG | GLU | E | 294 | 20.255 | 72.425 | −13.369 | 1.00 | 85.83 | E |
| ATOM | 2496 | CD | GLU | E | 294 | 20.698 | 71.291 | −14.290 | 1.00 | 93.99 | E |
| ATOM | 2497 | OE1 | GLU | E | 294 | 21.813 | 71.383 | −14.859 | 1.00 | 90.76 | E |
| ATOM | 2498 | OE2 | GLU | E | 294 | 19.922 | 70.313 | −14.451 | 1.00 | 96.20 | E |
| ATOM | 2499 | C | GLU | E | 294 | 19.819 | 72.865 | −10.317 | 1.00 | 66.48 | E |
| ATOM | 2500 | O | GLU | E | 294 | 18.653 | 73.228 | −10.138 | 1.00 | 65.85 | E |
| ATOM | 2501 | N | SER | E | 295 | 20.357 | 71.783 | −9.752 | 1.00 | 63.31 | E |
| ATOM | 2502 | CA | SER | E | 295 | 19.602 | 70.891 | −8.870 | 1.00 | 56.42 | E |
| ATOM | 2503 | CB | SER | E | 295 | 20.549 | 70.198 | −7.886 | 1.00 | 53.80 | E |
| ATOM | 2504 | OG | SER | E | 295 | 21.735 | 69.754 | −8.527 | 1.00 | 56.54 | E |
| ATOM | 2505 | C | SER | E | 295 | 18.544 | 71.661 | −8.093 | 1.00 | 52.70 | E |
| ATOM | 2506 | O | SER | E | 295 | 17.344 | 71.397 | −8.227 | 1.00 | 44.29 | E |
| ATOM | 2507 | N | TYR | E | 296 | 19.002 | 72.630 | −7.298 | 1.00 | 54.21 | E |
| ATOM | 2508 | CA | TYR | E | 296 | 18.099 | 73.442 | −6.476 | 1.00 | 57.94 | E |
| ATOM | 2509 | CB | TYR | E | 296 | 18.857 | 74.356 | −5.504 | 1.00 | 53.51 | E |
| ATOM | 2510 | CG | TYR | E | 296 | 19.457 | 73.665 | −4.308 | 1.00 | 51.18 | E |
| ATOM | 2511 | CD1 | TYR | E | 296 | 20.667 | 72.962 | −4.415 | 1.00 | 49.06 | E |
| ATOM | 2512 | CE1 | TYR | E | 296 | 21.254 | 72.369 | −3.310 | 1.00 | 45.23 | E |
| ATOM | 2513 | CD2 | TYR | E | 296 | 18.844 | 73.745 | −3.063 | 1.00 | 47.02 | E |
| ATOM | 2514 | CE2 | TYR | E | 296 | 19.425 | 73.156 | −1.948 | 1.00 | 49.14 | E |
| ATOM | 2515 | CZ | TYR | E | 296 | 20.631 | 72.474 | −2.079 | 1.00 | 47.12 | E |
| ATOM | 2516 | OH | TYR | E | 296 | 21.234 | 71.934 | −0.968 | 1.00 | 47.53 | E |
| ATOM | 2517 | C | TYR | E | 296 | 17.170 | 74.315 | −7.282 | 1.00 | 59.73 | E |
| ATOM | 2518 | O | TYR | E | 296 | 16.025 | 74.522 | −6.876 | 1.00 | 60.23 | E |
| ATOM | 2519 | N | THR | E | 297 | 17.660 | 74.854 | −8.401 | 1.00 | 59.81 | E |
| ATOM | 2520 | CA | THR | E | 297 | 16.821 | 75.712 | −9.223 | 1.00 | 57.01 | E |
| ATOM | 2521 | CB | THR | E | 297 | 17.584 | 76.369 | −10.373 | 1.00 | 57.33 | E |
| ATOM | 2522 | OG1 | THR | E | 297 | 18.672 | 77.147 | −9.855 | 1.00 | 60.03 | E |
| ATOM | 2523 | CG2 | THR | E | 297 | 16.653 | 77.298 | −11.131 | 1.00 | 52.97 | E |
| ATOM | 2524 | C | THR | E | 297 | 15.707 | 74.870 | −9.798 | 1.00 | 55.04 | E |
| ATOM | 2525 | O | THR | E | 297 | 14.567 | 75.316 | −9.848 | 1.00 | 52.58 | E |
| ATOM | 2526 | N | VAL | E | 298 | 16.043 | 73.643 | −10.199 | 1.00 | 51.86 | E |
| ATOM | 2527 | CA | VAL | E | 298 | 15.073 | 72.703 | −10.763 | 1.00 | 51.10 | E |
| ATOM | 2528 | CB | VAL | E | 298 | 15.741 | 71.362 | −11.201 | 1.00 | 53.10 | E |
| ATOM | 2529 | CG1 | VAL | E | 298 | 14.678 | 70.404 | −11.677 | 1.00 | 43.54 | E |
| ATOM | 2530 | CG2 | VAL | E | 298 | 16.770 | 71.589 | −12.315 | 1.00 | 52.63 | E |
| ATOM | 2531 | C | VAL | E | 298 | 13.975 | 72.362 | −9.759 | 1.00 | 52.09 | E |
| ATOM | 2532 | O | VAL | E | 298 | 12.823 | 72.199 | −10.124 | 1.00 | 57.65 | E |
| ATOM | 2533 | N | ALA | E | 299 | 14.334 | 72.229 | −8.493 | 1.00 | 55.66 | E |
| ATOM | 2534 | CA | ALA | E | 299 | 13.346 | 71.906 | −7.473 | 1.00 | 57.89 | E |
| ATOM | 2535 | CB | ALA | E | 299 | 14.035 | 71.374 | −6.239 | 1.00 | 65.04 | E |
| ATOM | 2536 | C | ALA | E | 299 | 12.532 | 73.127 | −7.108 | 1.00 | 57.74 | E |
| ATOM | 2537 | O | ALA | E | 299 | 11.611 | 73.049 | −6.300 | 1.00 | 60.91 | E |
| ATOM | 2538 | N | GLY | E | 300 | 12.893 | 74.258 | −7.698 | 1.00 | 53.99 | E |
| ATOM | 2539 | CA | GLY | E | 300 | 12.196 | 75.496 | −7.413 | 1.00 | 56.58 | E |
| ATOM | 2540 | C | GLY | E | 300 | 12.820 | 76.312 | −6.293 | 1.00 | 54.28 | E |
| ATOM | 2541 | O | GLY | E | 300 | 12.166 | 77.180 | −5.715 | 1.00 | 53.41 | E |
| ATOM | 2542 | N | MET | E | 301 | 14.087 | 76.048 | −5.991 | 1.00 | 53.48 | E |
| ATOM | 2543 | CA | MET | E | 301 | 14.782 | 76.762 | −4.926 | 1.00 | 57.30 | E |
| ATOM | 2544 | CB | MET | E | 301 | 15.327 | 75.763 | −3.901 | 1.00 | 59.17 | E |
| ATOM | 2545 | CG | MET | E | 301 | 14.397 | 74.603 | −3.580 | 1.00 | 61.86 | E |
| ATOM | 2546 | SD | MET | E | 301 | 13.307 | 74.869 | −2.181 | 1.00 | 57.45 | E |
| ATOM | 2547 | CE | MET | E | 301 | 14.421 | 74.386 | −0.828 | 1.00 | 68.06 | E |
| ATOM | 2548 | C | MET | E | 301 | 15.947 | 77.587 | −5.502 | 1.00 | 61.19 | E |
| ATOM | 2549 | O | MET | E | 301 | 16.963 | 77.816 | −4.813 | 1.00 | 61.18 | E |
| ATOM | 2550 | N | GLY | E | 302 | 15.791 | 78.024 | −6.757 | 1.00 | 56.02 | E |
| ATOM | 2551 | CA | GLY | E | 302 | 16.819 | 78.805 | −7.427 | 1.00 | 48.70 | E |
| ATOM | 2552 | C | GLY | E | 302 | 17.229 | 80.094 | −6.728 | 1.00 | 48.32 | E |
| ATOM | 2553 | O | GLY | E | 302 | 18.397 | 80.503 | −6.815 | 1.00 | 48.00 | E |
| ATOM | 2554 | N | ASP | E | 303 | 16.273 | 80.734 | −6.048 | 1.00 | 44.95 | E |
| ATOM | 2555 | CA | ASP | E | 303 | 16.491 | 81.981 | −5.314 | 1.00 | 47.27 | E |
| ATOM | 2556 | CB | ASP | E | 303 | 15.258 | 82.335 | −4.511 | 1.00 | 55.66 | E |
| ATOM | 2557 | CG | ASP | E | 303 | 14.077 | 82.688 | −5.369 | 1.00 | 72.49 | E |
| ATOM | 2558 | OD1 | ASP | E | 303 | 12.947 | 82.695 | −4.811 | 1.00 | 84.45 | E |
| ATOM | 2559 | OD2 | ASP | E | 303 | 14.269 | 82.970 | −6.577 | 1.00 | 74.53 | E |
| ATOM | 2560 | C | ASP | E | 303 | 17.634 | 81.883 | −4.320 | 1.00 | 53.13 | E |
| ATOM | 2561 | O | ASP | E | 303 | 18.690 | 82.501 | −4.479 | 1.00 | 58.14 | E |
| ATOM | 2562 | N | THR | E | 304 | 17.396 | 81.104 | −3.271 | 1.00 | 55.42 | E |
| ATOM | 2563 | CA | THR | E | 304 | 18.365 | 80.915 | −2.208 | 1.00 | 55.06 | E |
| ATOM | 2564 | CB | THR | E | 304 | 17.707 | 80.242 | −0.980 | 1.00 | 56.60 | E |
| ATOM | 2565 | OG1 | THR | E | 304 | 17.117 | 78.989 | −1.360 | 1.00 | 62.37 | E |
| ATOM | 2566 | CG2 | THR | E | 304 | 16.648 | 81.150 | −0.407 | 1.00 | 54.69 | E |
| ATOM | 2567 | C | THR | E | 304 | 19.620 | 80.132 | −2.574 | 1.00 | 52.08 | E |
| ATOM | 2568 | O | THR | E | 304 | 20.683 | 80.376 | −2.000 | 1.00 | 48.46 | E |
| ATOM | 2569 | N | VAL | E | 305 | 19.516 | 79.213 | −3.529 | 1.00 | 51.67 | E |
| ATOM | 2570 | CA | VAL | E | 305 | 20.672 | 78.397 | −3.898 | 1.00 | 50.53 | E |
| ATOM | 2571 | CB | VAL | E | 305 | 20.544 | 77.751 | −5.275 | 1.00 | 54.93 | E |
| ATOM | 2572 | CG1 | VAL | E | 305 | 20.739 | 78.814 | −6.381 | 1.00 | 51.08 | E |
| ATOM | 2573 | CG2 | VAL | E | 305 | 21.594 | 76.643 | −5.401 | 1.00 | 47.75 | E |
| ATOM | 2574 | C | VAL | E | 305 | 22.000 | 79.113 | −3.918 | 1.00 | 46.99 | E |
| ATOM | 2575 | O | VAL | E | 305 | 22.971 | 78.599 | −3.379 | 1.00 | 41.23 | E |
| ATOM | 2576 | N | GLU | E | 306 | 22.065 | 80.292 | −4.530 | 1.00 | 46.23 | E |
| ATOM | 2577 | CA | GLU | E | 306 | 23.350 | 80.971 | −4.595 | 1.00 | 46.84 | E |
| ATOM | 2578 | CB | GLU | E | 306 | 23.287 | 82.185 | −5.513 | 1.00 | 51.46 | E |
| ATOM | 2579 | CG | GLU | E | 306 | 24.683 | 82.744 | −5.884 | 1.00 | 61.37 | E |
| ATOM | 2580 | CD | GLU | E | 306 | 25.688 | 81.672 | −6.364 | 1.00 | 65.97 | E |
| ATOM | 2581 | OE1 | GLU | E | 306 | 25.336 | 80.876 | −7.266 | 1.00 | 60.45 | E |
| ATOM | 2582 | OE2 | GLU | E | 306 | 26.838 | 81.639 | −5.851 | 1.00 | 61.39 | E |
| ATOM | 2583 | C | GLU | E | 306 | 23.901 | 81.363 | −3.238 | 1.00 | 44.73 | E |
| ATOM | 2584 | O | GLU | E | 306 | 25.074 | 81.124 | −2.965 | 1.00 | 40.70 | E |
| ATOM | 2585 | N | ASP | E | 307 | 23.059 | 81.953 | −2.391 | 1.00 | 44.04 | E |
| ATOM | 2586 | CA | ASP | E | 307 | 23.477 | 82.340 | −1.045 | 1.00 | 41.14 | E |
| ATOM | 2587 | CB | ASP | E | 307 | 22.305 | 82.889 | −0.246 | 1.00 | 52.01 | E |
| ATOM | 2588 | CG | ASP | E | 307 | 21.757 | 84.161 | −0.827 | 1.00 | 60.75 | E |
| ATOM | 2589 | OD1 | ASP | E | 307 | 20.814 | 84.069 | −1.652 | 1.00 | 62.82 | E |
| ATOM | 2590 | OD2 | ASP | E | 307 | 22.283 | 85.244 | −0.464 | 1.00 | 64.89 | E |
| ATOM | 2591 | C | ASP | E | 307 | 24.043 | 81.145 | −0.297 | 1.00 | 36.51 | E |
| ATOM | 2592 | O | ASP | E | 307 | 25.036 | 81.263 | 0.414 | 1.00 | 33.26 | E |
| ATOM | 2593 | N | LEU | E | 308 | 23.386 | 80.001 | −0.445 | 1.00 | 32.45 | E |
| ATOM | 2594 | CA | LEU | E | 308 | 23.827 | 78.770 | 0.185 | 1.00 | 34.12 | E |
| ATOM | 2595 | CB | LEU | E | 308 | 22.864 | 77.639 | −0.142 | 1.00 | 27.15 | E |
| ATOM | 2596 | CG | LEU | E | 308 | 21.432 | 77.848 | 0.321 | 1.00 | 28.39 | E |
| ATOM | 2597 | CD1 | LEU | E | 308 | 20.546 | 76.727 | −0.217 | 1.00 | 28.97 | E |
| ATOM | 2598 | CD2 | LEU | E | 308 | 21.406 | 77.907 | 1.828 | 1.00 | 19.33 | E |
| ATOM | 2599 | C | LEU | E | 308 | 25.211 | 78.400 | −0.328 | 1.00 | 39.79 | E |
| ATOM | 2600 | O | LEU | E | 308 | 26.143 | 78.191 | 0.452 | 1.00 | 45.71 | E |
| ATOM | 2601 | N | LEU | E | 309 | 25.346 | 78.311 | −1.644 | 1.00 | 38.81 | E |
| ATOM | 2602 | CA | LEU | E | 309 | 26.628 | 77.955 | −2.226 | 1.00 | 42.34 | E |
| ATOM | 2603 | CB | LEU | E | 309 | 26.543 | 77.950 | −3.763 | 1.00 | 38.54 | E |
| ATOM | 2604 | CG | LEU | E | 309 | 26.325 | 76.618 | −4.504 | 1.00 | 33.64 | E |
| ATOM | 2605 | CD1 | LEU | E | 309 | 26.794 | 75.427 | −3.659 | 1.00 | 32.06 | E |
| ATOM | 2606 | CD2 | LEU | E | 309 | 24.858 | 76.473 | −4.851 | 1.00 | 27.68 | E |
| ATOM | 2607 | C | LEU | E | 309 | 27.749 | 78.896 | −1.767 | 1.00 | 43.90 | E |
| ATOM | 2608 | O | LEU | E | 309 | 28.866 | 78.456 | −1.467 | 1.00 | 42.98 | E |
| ATOM | 2609 | N | ARG | E | 310 | 27.440 | 80.187 | −1.711 | 1.00 | 42.19 | E |
| ATOM | 2610 | CA | ARG | E | 310 | 28.412 | 81.183 | −1.307 | 1.00 | 40.66 | E |
| ATOM | 2611 | CB | ARG | E | 310 | 27.777 | 82.570 | −1.380 | 1.00 | 40.07 | E |
| ATOM | 2612 | CG | ARG | E | 310 | 28.591 | 83.705 | −0.806 | 1.00 | 44.89 | E |
| ATOM | 2613 | CD | ARG | E | 310 | 27.831 | 85.033 | −0.902 | 1.00 | 58.85 | E |
| ATOM | 2614 | NE | ARG | E | 310 | 27.653 | 85.436 | −2.295 | 1.00 | 69.19 | E |
| ATOM | 2615 | CZ | ARG | E | 310 | 26.649 | 85.051 | −3.080 | 1.00 | 75.43 | E |
| ATOM | 2616 | NH1 | ARG | E | 310 | 25.700 | 84.251 | −2.610 | 1.00 | 81.52 | E |
| ATOM | 2617 | NH2 | ARG | E | 310 | 26.609 | 85.445 | −4.350 | 1.00 | 74.42 | E |
| ATOM | 2618 | C | ARG | E | 310 | 28.919 | 80.888 | 0.095 | 1.00 | 42.89 | E |
| ATOM | 2619 | O | ARG | E | 310 | 30.127 | 80.940 | 0.354 | 1.00 | 42.22 | E |
| ATOM | 2620 | N | PHE | E | 311 | 28.002 | 80.558 | 1.000 | 1.00 | 43.01 | E |
| ATOM | 2621 | CA | PHE | E | 311 | 28.385 | 80.257 | 2.373 | 1.00 | 40.21 | E |
| ATOM | 2622 | CB | PHE | E | 311 | 27.172 | 79.873 | 3.207 | 1.00 | 36.06 | E |
| ATOM | 2623 | CG | PHE | E | 311 | 27.501 | 79.624 | 4.641 | 1.00 | 36.68 | E |
| ATOM | 2624 | CD1 | PHE | E | 311 | 27.874 | 80.672 | 5.469 | 1.00 | 36.95 | E |
| ATOM | 2625 | CD2 | PHE | E | 311 | 27.498 | 78.337 | 5.152 | 1.00 | 35.64 | E |
| ATOM | 2626 | CE1 | PHE | E | 311 | 28.232 | 80.435 | 6.780 | 1.00 | 41.04 | E |
| ATOM | 2627 | CE2 | PHE | E | 311 | 27.854 | 78.100 | 6.457 | 1.00 | 37.74 | E |
| ATOM | 2628 | CZ | PHE | E | 311 | 28.226 | 79.152 | 7.273 | 1.00 | 38.51 | E |
| ATOM | 2629 | C | PHE | E | 311 | 29.369 | 79.103 | 2.392 | 1.00 | 46.01 | E |
| ATOM | 2630 | O | PHE | E | 311 | 30.416 | 79.169 | 3.051 | 1.00 | 49.74 | E |
| ATOM | 2631 | N | CYS | E | 312 | 29.004 | 78.040 | 1.674 | 1.00 | 42.54 | E |
| ATOM | 2632 | CA | CYS | E | 312 | 29.825 | 76.845 | 1.564 | 1.00 | 37.36 | E |
| ATOM | 2633 | CB | CYS | E | 312 | 29.220 | 75.874 | 0.556 | 1.00 | 38.37 | E |
| ATOM | 2634 | SG | CYS | E | 312 | 27.662 | 75.132 | 1.048 | 1.00 | 39.43 | E |
| ATOM | 2635 | C | CYS | E | 312 | 31.213 | 77.229 | 1.106 | 1.00 | 40.16 | E |
| ATOM | 2636 | O | CYS | E | 312 | 32.201 | 76.732 | 1.636 | 1.00 | 46.13 | E |
| ATOM | 2637 | N | ARG | E | 313 | 31.285 | 78.112 | 0.112 | 1.00 | 44.10 | E |
| ATOM | 2638 | CA | ARG | E | 313 | 32.567 | 78.561 | −0.423 | 1.00 | 42.60 | E |
| ATOM | 2639 | CB | ARG | E | 313 | 32.372 | 79.439 | −1.652 | 1.00 | 42.81 | E |
| ATOM | 2640 | CG | ARG | E | 313 | 32.170 | 78.695 | −2.958 | 1.00 | 47.52 | E |
| ATOM | 2641 | CD | ARG | E | 313 | 32.289 | 79.646 | −4.137 | 1.00 | 42.88 | E |
| ATOM | 2642 | NE | ARG | E | 313 | 31.119 | 80.498 | −4.330 | 1.00 | 40.06 | E |
| ATOM | 2643 | CZ | ARG | E | 313 | 29.954 | 80.041 | −4.775 | 1.00 | 50.01 | E |
| ATOM | 2644 | NH1 | ARG | E | 313 | 29.823 | 78.742 | −5.055 | 1.00 | 45.76 | E |
| ATOM | 2645 | NH2 | ARG | E | 313 | 28.938 | 80.879 | −4.977 | 1.00 | 43.24 | E |
| ATOM | 2646 | C | ARG | E | 313 | 33.361 | 79.340 | 0.594 | 1.00 | 44.06 | E |
| ATOM | 2647 | O | ARG | E | 313 | 34.555 | 79.105 | 0.743 | 1.00 | 40.75 | E |
| ATOM | 2648 | N | HIS | E | 314 | 32.707 | 80.279 | 1.281 | 1.00 | 45.97 | E |
| ATOM | 2649 | CA | HIS | E | 314 | 33.386 | 81.087 | 2.285 | 1.00 | 47.88 | E |
| ATOM | 2650 | CB | HIS | E | 314 | 32.449 | 82.171 | 2.838 | 1.00 | 58.09 | E |
| ATOM | 2651 | CG | HIS | E | 314 | 33.004 | 82.914 | 4.021 | 1.00 | 69.52 | E |
| ATOM | 2652 | CD2 | HIS | E | 314 | 32.496 | 83.130 | 5.258 | 1.00 | 72.88 | E |
| ATOM | 2653 | ND1 | HIS | E | 314 | 34.240 | 83.533 | 4.006 | 1.00 | 78.21 | E |
| ATOM | 2654 | CE1 | HIS | E | 314 | 34.467 | 84.093 | 5.180 | 1.00 | 75.35 | E |
| ATOM | 2655 | NE2 | HIS | E | 314 | 33.425 | 83.864 | 5.960 | 1.00 | 76.13 | E |
| ATOM | 2656 | C | HIS | E | 314 | 33.917 | 80.210 | 3.415 | 1.00 | 44.87 | E |
| ATOM | 2657 | O | HIS | E | 314 | 35.002 | 80.461 | 3.949 | 1.00 | 42.96 | E |
| ATOM | 2658 | N | MET | E | 315 | 33.171 | 79.169 | 3.769 | 1.00 | 41.07 | E |
| ATOM | 2659 | CA | MET | E | 315 | 33.608 | 78.274 | 4.843 | 1.00 | 46.97 | E |
| ATOM | 2660 | CB | MET | E | 315 | 32.445 | 77.374 | 5.283 | 1.00 | 45.05 | E |
| ATOM | 2661 | CG | MET | E | 315 | 31.303 | 78.114 | 5.987 | 1.00 | 44.79 | E |
| ATOM | 2662 | SD | MET | E | 315 | 31.793 | 78.853 | 7.570 | 1.00 | 49.50 | E |
| ATOM | 2663 | CE | MET | E | 315 | 32.000 | 77.438 | 8.591 | 1.00 | 41.10 | E |
| ATOM | 2664 | C | MET | E | 315 | 34.821 | 77.428 | 4.417 | 1.00 | 48.06 | E |
| ATOM | 2665 | O | MET | E | 315 | 35.790 | 77.237 | 5.164 | 1.00 | 49.75 | E |
| ATOM | 2666 | N | CYS | E | 316 | 34.755 | 76.923 | 3.199 | 1.00 | 50.73 | E |
| ATOM | 2667 | CA | CYS | E | 316 | 35.832 | 76.124 | 2.636 | 1.00 | 47.71 | E |
| ATOM | 2668 | CB | CYS | E | 316 | 35.422 | 75.668 | 1.244 | 1.00 | 49.10 | E |
| ATOM | 2669 | SG | CYS | E | 316 | 36.479 | 74.437 | 0.585 | 1.00 | 57.98 | E |
| ATOM | 2670 | C | CYS | E | 316 | 37.110 | 76.962 | 2.556 | 1.00 | 44.65 | E |
| ATOM | 2671 | O | CYS | E | 316 | 38.211 | 76.458 | 2.708 | 1.00 | 39.70 | E |
| ATOM | 2672 | N | ALA | E | 317 | 36.935 | 78.259 | 2.315 | 1.00 | 47.36 | E |
| ATOM | 2673 | CA | ALA | E | 317 | 38.031 | 79.214 | 2.201 | 1.00 | 39.33 | E |
| ATOM | 2674 | CB | ALA | E | 317 | 37.510 | 80.492 | 1.694 | 1.00 | 31.61 | E |
| ATOM | 2675 | C | ALA | E | 317 | 38.727 | 79.445 | 3.520 | 1.00 | 39.45 | E |
| ATOM | 2676 | O | ALA | E | 317 | 39.917 | 79.736 | 3.542 | 1.00 | 40.04 | E |
| ATOM | 2677 | N | MET | E | 318 | 37.973 | 79.326 | 4.613 | 1.00 | 40.99 | E |
| ATOM | 2678 | CA | MET | E | 318 | 38.506 | 79.499 | 5.964 | 1.00 | 38.70 | E |
| ATOM | 2679 | CB | MET | E | 318 | 37.397 | 79.880 | 6.924 | 1.00 | 35.12 | E |
| ATOM | 2680 | CG | MET | E | 318 | 36.922 | 81.282 | 6.742 | 1.00 | 42.22 | E |
| ATOM | 2681 | SD | MET | E | 318 | 36.025 | 81.798 | 8.198 | 1.00 | 49.96 | E |
| ATOM | 2682 | CE | MET | E | 318 | 34.367 | 81.205 | 7.716 | 1.00 | 55.22 | E |
| ATOM | 2683 | C | MET | E | 318 | 39.195 | 78.252 | 6.495 | 1.00 | 38.83 | E |
| ATOM | 2684 | O | MET | E | 318 | 39.875 | 78.295 | 7.525 | 1.00 | 38.43 | E |
| ATOM | 2685 | N | LYS | E | 319 | 39.001 | 77.139 | 5.798 | 1.00 | 39.64 | E |
| ATOM | 2686 | CA | LYS | E | 319 | 39.611 | 75.891 | 6.201 | 1.00 | 39.52 | E |
| ATOM | 2687 | CB | LYS | E | 319 | 41.122 | 76.011 | 6.052 | 1.00 | 36.73 | E |
| ATOM | 2688 | CG | LYS | E | 319 | 41.584 | 76.488 | 4.685 | 1.00 | 38.39 | E |
| ATOM | 2689 | CD | LYS | E | 319 | 43.119 | 76.570 | 4.618 | 1.00 | 44.38 | E |
| ATOM | 2690 | CE | LYS | E | 319 | 43.651 | 77.125 | 3.265 | 1.00 | 53.58 | E |
| ATOM | 2691 | NZ | LYS | E | 319 | 43.620 | 76.171 | 2.090 | 1.00 | 49.79 | E |
| ATOM | 2692 | C | LYS | E | 319 | 39.231 | 75.592 | 7.657 | 1.00 | 42.19 | E |
| ATOM | 2693 | O | LYS | E | 319 | 40.092 | 75.412 | 8.519 | 1.00 | 41.47 | E |
| ATOM | 2694 | N | VAL | E | 320 | 37.929 | 75.558 | 7.921 | 1.00 | 42.30 | E |
| ATOM | 2695 | CA | VAL | E | 320 | 37.404 | 75.293 | 9.257 | 1.00 | 39.90 | E |
| ATOM | 2696 | CB | VAL | E | 320 | 35.907 | 75.750 | 9.331 | 1.00 | 39.08 | E |
| ATOM | 2697 | CG1 | VAL | E | 320 | 35.325 | 75.516 | 10.715 | 1.00 | 28.09 | E |
| ATOM | 2698 | CG2 | VAL | E | 320 | 35.805 | 77.219 | 8.948 | 1.00 | 37.65 | E |
| ATOM | 2699 | C | VAL | E | 320 | 37.517 | 73.792 | 9.486 | 1.00 | 38.14 | E |
| ATOM | 2700 | O | VAL | E | 320 | 36.966 | 73.018 | 8.720 | 1.00 | 40.61 | E |
| ATOM | 2701 | N | ASP | E | 321 | 38.228 | 73.357 | 10.520 | 1.00 | 39.20 | E |
| ATOM | 2702 | CA | ASP | E | 321 | 38.349 | 71.916 | 10.739 | 1.00 | 38.88 | E |
| ATOM | 2703 | CB | ASP | E | 321 | 39.713 | 71.574 | 11.312 | 1.00 | 33.31 | E |
| ATOM | 2704 | CG | ASP | E | 321 | 39.871 | 72.017 | 12.743 | 1.00 | 41.47 | E |
| ATOM | 2705 | OD1 | ASP | E | 321 | 40.985 | 72.438 | 13.105 | 1.00 | 41.07 | E |
| ATOM | 2706 | OD2 | ASP | E | 321 | 38.897 | 71.929 | 13.515 | 1.00 | 48.12 | E |
| ATOM | 2707 | C | ASP | E | 321 | 37.262 | 71.351 | 11.641 | 1.00 | 43.03 | E |
| ATOM | 2708 | O | ASP | E | 321 | 36.427 | 72.083 | 12.166 | 1.00 | 50.90 | E |
| ATOM | 2709 | N | ASN | E | 322 | 37.288 | 70.043 | 11.841 | 1.00 | 40.47 | E |
| ATOM | 2710 | CA | ASN | E | 322 | 36.284 | 69.382 | 12.656 | 1.00 | 39.16 | E |
| ATOM | 2711 | CB | ASN | E | 322 | 36.584 | 67.899 | 12.740 | 1.00 | 42.19 | E |
| ATOM | 2712 | CG | ASN | E | 322 | 36.518 | 67.246 | 11.399 | 1.00 | 37.40 | E |
| ATOM | 2713 | OD1 | ASN | E | 322 | 36.073 | 67.861 | 10.434 | 1.00 | 39.07 | E |
| ATOM | 2714 | ND2 | ASN | E | 322 | 36.958 | 66.007 | 11.318 | 1.00 | 34.55 | E |
| ATOM | 2715 | C | ASN | E | 322 | 36.044 | 69.913 | 14.038 | 1.00 | 42.55 | E |
| ATOM | 2716 | O | ASN | E | 322 | 34.891 | 70.145 | 14.410 | 1.00 | 45.98 | E |
| ATOM | 2717 | N | ALA | E | 323 | 37.116 | 70.090 | 14.804 | 1.00 | 41.77 | E |
| ATOM | 2718 | CA | ALA | E | 323 | 36.995 | 70.611 | 16.165 | 1.00 | 39.19 | E |
| ATOM | 2719 | CB | ALA | E | 323 | 38.353 | 70.739 | 16.788 | 1.00 | 37.72 | E |
| ATOM | 2720 | C | ALA | E | 323 | 36.312 | 71.975 | 16.150 | 1.00 | 41.04 | E |
| ATOM | 2721 | O | ALA | E | 323 | 35.353 | 72.233 | 16.904 | 1.00 | 34.72 | E |
| ATOM | 2722 | N | GLU | E | 324 | 36.811 | 72.847 | 15.278 | 1.00 | 36.39 | E |
| ATOM | 2723 | CA | GLU | E | 324 | 36.262 | 74.186 | 15.158 | 1.00 | 37.10 | E |
| ATOM | 2724 | CB | GLU | E | 324 | 37.103 | 74.998 | 14.173 | 1.00 | 42.36 | E |
| ATOM | 2725 | CG | GLU | E | 324 | 38.579 | 74.983 | 14.512 | 1.00 | 43.03 | E |
| ATOM | 2726 | CD | GLU | E | 324 | 39.385 | 75.848 | 13.603 | 1.00 | 44.29 | E |
| ATOM | 2727 | OE1 | GLU | E | 324 | 39.213 | 75.716 | 12.376 | 1.00 | 45.28 | E |
| ATOM | 2728 | OE2 | GLU | E | 324 | 40.195 | 76.651 | 14.116 | 1.00 | 49.48 | E |
| ATOM | 2729 | C | GLU | E | 324 | 34.812 | 74.089 | 14.703 | 1.00 | 37.19 | E |
| ATOM | 2730 | O | GLU | E | 324 | 33.932 | 74.790 | 15.219 | 1.00 | 36.60 | E |
| ATOM | 2731 | N | TYR | E | 325 | 34.555 | 73.212 | 13.741 | 1.00 | 35.43 | E |
| ATOM | 2732 | CA | TYR | E | 325 | 33.195 | 73.050 | 13.280 | 1.00 | 33.83 | E |
| ATOM | 2733 | CB | TYR | E | 325 | 33.112 | 72.009 | 12.177 | 1.00 | 28.46 | E |
| ATOM | 2734 | CG | TYR | E | 325 | 32.815 | 72.613 | 10.823 | 1.00 | 32.50 | E |
| ATOM | 2735 | CD1 | TYR | E | 325 | 31.586 | 73.223 | 10.553 | 1.00 | 30.26 | E |
| ATOM | 2736 | CE1 | TYR | E | 325 | 31.326 | 73.781 | 9.291 | 1.00 | 33.88 | E |
| ATOM | 2737 | CD2 | TYR | E | 325 | 33.766 | 72.577 | 9.811 | 1.00 | 29.44 | E |
| ATOM | 2738 | CE2 | TYR | E | 325 | 33.517 | 73.127 | 8.570 | 1.00 | 29.18 | E |
| ATOM | 2739 | CZ | TYR | E | 325 | 32.309 | 73.728 | 8.301 | 1.00 | 31.70 | E |
| ATOM | 2740 | OH | TYR | E | 325 | 32.119 | 74.268 | 7.039 | 1.00 | 33.30 | E |
| ATOM | 2741 | C | TYR | E | 325 | 32.337 | 72.631 | 14.454 | 1.00 | 35.84 | E |
| ATOM | 2742 | O | TYR | E | 325 | 31.389 | 73.324 | 14.802 | 1.00 | 38.35 | E |
| ATOM | 2743 | N | ALA | E | 326 | 32.682 | 71.520 | 15.093 | 1.00 | 31.20 | E |
| ATOM | 2744 | CA | ALA | E | 326 | 31.881 | 71.052 | 16.207 | 1.00 | 30.02 | E |
| ATOM | 2745 | CB | ALA | E | 326 | 32.488 | 69.798 | 16.786 | 1.00 | 23.95 | E |
| ATOM | 2746 | C | ALA | E | 326 | 31.696 | 72.116 | 17.291 | 1.00 | 33.08 | E |
| ATOM | 2747 | O | ALA | E | 326 | 30.563 | 72.490 | 17.625 | 1.00 | 32.67 | E |
| ATOM | 2748 | N | LEU | E | 327 | 32.795 | 72.626 | 17.834 | 1.00 | 27.98 | E |
| ATOM | 2749 | CA | LEU | E | 327 | 32.686 | 73.626 | 18.894 | 1.00 | 30.89 | E |
| ATOM | 2750 | CB | LEU | E | 327 | 34.055 | 74.198 | 19.234 | 1.00 | 26.93 | E |
| ATOM | 2751 | CG | LEU | E | 327 | 34.937 | 73.266 | 20.036 | 1.00 | 24.04 | E |
| ATOM | 2752 | CD1 | LEU | E | 327 | 36.389 | 73.677 | 19.914 | 1.00 | 15.58 | E |
| ATOM | 2753 | CD2 | LEU | E | 327 | 34.428 | 73.266 | 21.464 | 1.00 | 20.01 | E |
| ATOM | 2754 | C | LEU | E | 327 | 31.776 | 74.757 | 18.481 | 1.00 | 35.13 | E |
| ATOM | 2755 | O | LEU | E | 327 | 30.962 | 75.242 | 19.267 | 1.00 | 39.12 | E |
| ATOM | 2756 | N | LEU | E | 328 | 31.923 | 75.170 | 17.231 | 1.00 | 33.20 | E |
| ATOM | 2757 | CA | LEU | E | 328 | 31.139 | 76.265 | 16.709 | 1.00 | 35.88 | E |
| ATOM | 2758 | CB | LEU | E | 328 | 31.634 | 76.617 | 15.298 | 1.00 | 36.15 | E |
| ATOM | 2759 | CG | LEU | E | 328 | 31.793 | 78.091 | 14.915 | 1.00 | 28.33 | E |
| ATOM | 2760 | CD1 | LEU | E | 328 | 32.335 | 78.935 | 16.063 | 1.00 | 34.96 | E |
| ATOM | 2761 | CD2 | LEU | E | 328 | 32.724 | 78.144 | 13.779 | 1.00 | 21.29 | E |
| ATOM | 2762 | C | LEU | E | 328 | 29.654 | 75.918 | 16.718 | 1.00 | 35.44 | E |
| ATOM | 2763 | O | LEU | E | 328 | 28.844 | 76.687 | 17.234 | 1.00 | 32.20 | E |
| ATOM | 2764 | N | THR | E | 329 | 29.290 | 74.758 | 16.179 | 1.00 | 33.93 | E |
| ATOM | 2765 | CA | THR | E | 329 | 27.882 | 74.389 | 16.167 | 1.00 | 32.32 | E |
| ATOM | 2766 | CB | THR | E | 329 | 27.595 | 73.010 | 15.454 | 1.00 | 27.74 | E |
| ATOM | 2767 | OG1 | THR | E | 329 | 27.341 | 72.023 | 16.449 | 1.00 | 29.80 | E |
| ATOM | 2768 | CG2 | THR | E | 329 | 28.756 | 72.546 | 14.579 | 1.00 | 17.77 | E |
| ATOM | 2769 | C | THR | E | 329 | 27.350 | 74.328 | 17.609 | 1.00 | 33.95 | E |
| ATOM | 2770 | O | THR | E | 329 | 26.179 | 74.646 | 17.848 | 1.00 | 33.69 | E |
| ATOM | 2771 | N | ALA | E | 330 | 28.199 | 73.937 | 18.567 | 1.00 | 31.78 | E |
| ATOM | 2772 | CA | ALA | E | 330 | 27.769 | 73.861 | 19.969 | 1.00 | 33.28 | E |
| ATOM | 2773 | CB | ALA | E | 330 | 28.817 | 73.142 | 20.814 | 1.00 | 31.03 | E |
| ATOM | 2774 | C | ALA | E | 330 | 27.487 | 75.258 | 20.547 | 1.00 | 35.12 | E |
| ATOM | 2775 | O | ALA | E | 330 | 26.546 | 75.438 | 21.330 | 1.00 | 28.86 | E |
| ATOM | 2776 | N | ILE | E | 331 | 28.306 | 76.240 | 20.161 | 1.00 | 32.84 | E |
| ATOM | 2777 | CA | ILE | E | 331 | 28.134 | 77.623 | 20.611 | 1.00 | 31.63 | E |
| ATOM | 2778 | CB | ILE | E | 331 | 29.307 | 78.508 | 20.087 | 1.00 | 28.80 | E |
| ATOM | 2779 | CG2 | ILE | E | 331 | 29.148 | 79.977 | 20.529 | 1.00 | 14.86 | E |
| ATOM | 2780 | CG1 | ILE | E | 331 | 30.622 | 77.921 | 20.594 | 1.00 | 22.26 | E |
| ATOM | 2781 | CD1 | ILE | E | 331 | 31.815 | 78.798 | 20.411 | 1.00 | 22.39 | E |
| ATOM | 2782 | C | ILE | E | 331 | 26.778 | 78.152 | 20.096 | 1.00 | 35.90 | E |
| ATOM | 2783 | O | ILE | E | 331 | 26.099 | 78.948 | 20.763 | 1.00 | 38.07 | E |
| ATOM | 2784 | N | VAL | E | 332 | 26.383 | 77.684 | 18.911 | 1.00 | 36.30 | E |
| ATOM | 2785 | CA | VAL | E | 332 | 25.123 | 78.079 | 18.280 | 1.00 | 32.82 | E |
| ATOM | 2786 | CB | VAL | E | 332 | 25.033 | 77.598 | 16.807 | 1.00 | 33.28 | E |
| ATOM | 2787 | CG1 | VAL | E | 332 | 23.624 | 77.856 | 16.253 | 1.00 | 20.84 | E |
| ATOM | 2788 | CG2 | VAL | E | 332 | 26.093 | 78.298 | 15.956 | 1.00 | 27.39 | E |
| ATOM | 2789 | C | VAL | E | 332 | 23.951 | 77.474 | 19.005 | 1.00 | 30.96 | E |
| ATOM | 2790 | O | VAL | E | 332 | 22.933 | 78.125 | 19.224 | 1.00 | 38.03 | E |
| ATOM | 2791 | N | ILE | E | 333 | 24.095 | 76.208 | 19.358 | 1.00 | 32.15 | E |
| ATOM | 2792 | CA | ILE | E | 333 | 23.033 | 75.496 | 20.042 | 1.00 | 33.48 | E |
| ATOM | 2793 | CB | ILE | E | 333 | 23.396 | 73.996 | 20.172 | 1.00 | 24.45 | E |
| ATOM | 2794 | CG2 | ILE | E | 333 | 22.414 | 73.288 | 21.054 | 1.00 | 19.03 | E |
| ATOM | 2795 | CG1 | ILE | E | 333 | 23.403 | 73.358 | 18.789 | 1.00 | 20.06 | E |
| ATOM | 2796 | CD1 | ILE | E | 333 | 23.824 | 71.910 | 18.787 | 1.00 | 19.42 | E |
| ATOM | 2797 | C | ILE | E | 333 | 22.716 | 76.125 | 21.404 | 1.00 | 33.41 | E |
| ATOM | 2798 | O | ILE | E | 333 | 21.562 | 76.100 | 21.847 | 1.00 | 35.50 | E |
| ATOM | 2799 | N | PHE | E | 334 | 23.732 | 76.715 | 22.033 | 1.00 | 28.65 | E |
| ATOM | 2800 | CA | PHE | E | 334 | 23.593 | 77.355 | 23.343 | 1.00 | 33.67 | E |
| ATOM | 2801 | CB | PHE | E | 334 | 24.717 | 76.917 | 24.278 | 1.00 | 32.69 | E |
| ATOM | 2802 | CG | PHE | E | 334 | 24.639 | 75.490 | 24.672 | 1.00 | 36.29 | E |
| ATOM | 2803 | CD1 | PHE | E | 334 | 23.507 | 75.003 | 25.321 | 1.00 | 32.87 | E |
| ATOM | 2804 | CD2 | PHE | E | 334 | 25.686 | 74.620 | 24.382 | 1.00 | 36.20 | E |
| ATOM | 2805 | CE1 | PHE | E | 334 | 23.409 | 73.675 | 25.675 | 1.00 | 31.24 | E |
| ATOM | 2806 | CE2 | PHE | E | 334 | 25.598 | 73.285 | 24.732 | 1.00 | 38.46 | E |
| ATOM | 2807 | CZ | PHE | E | 334 | 24.450 | 72.811 | 25.383 | 1.00 | 38.10 | E |
| ATOM | 2808 | C | PHE | E | 334 | 23.571 | 78.884 | 23.290 | 1.00 | 39.11 | E |
| ATOM | 2809 | O | PHE | E | 334 | 24.192 | 79.576 | 24.126 | 1.00 | 39.08 | E |
| ATOM | 2810 | N | SER | E | 335 | 22.847 | 79.410 | 22.315 | 1.00 | 33.86 | E |
| ATOM | 2811 | CA | SER | E | 335 | 22.742 | 80.837 | 22.167 | 1.00 | 35.91 | E |
| ATOM | 2812 | CB | SER | E | 335 | 22.707 | 81.171 | 20.689 | 1.00 | 32.42 | E |
| ATOM | 2813 | OG | SER | E | 335 | 23.897 | 80.693 | 20.096 | 1.00 | 29.88 | E |
| ATOM | 2814 | C | SER | E | 335 | 21.507 | 81.354 | 22.887 | 1.00 | 39.38 | E |
| ATOM | 2815 | O | SER | E | 335 | 20.399 | 81.149 | 22.430 | 1.00 | 45.11 | E |
| ATOM | 2816 | N | GLU | E | 336 | 21.707 | 82.006 | 24.030 | 1.00 | 43.04 | E |
| ATOM | 2817 | CA | GLU | E | 336 | 20.597 | 82.545 | 24.801 | 1.00 | 44.80 | E |
| ATOM | 2818 | CB | GLU | E | 336 | 21.094 | 83.297 | 26.021 | 1.00 | 49.84 | E |
| ATOM | 2819 | CG | GLU | E | 336 | 19.978 | 84.039 | 26.739 | 1.00 | 64.36 | E |
| ATOM | 2820 | CD | GLU | E | 336 | 20.432 | 84.685 | 28.046 | 1.00 | 75.48 | E |
| ATOM | 2821 | OE1 | GLU | E | 336 | 19.627 | 85.429 | 28.668 | 1.00 | 70.40 | E |
| ATOM | 2822 | OE2 | GLU | E | 336 | 21.594 | 84.443 | 28.451 | 1.00 | 76.31 | E |
| ATOM | 2823 | C | GLU | E | 336 | 19.723 | 83.481 | 23.991 | 1.00 | 47.53 | E |
| ATOM | 2824 | O | GLU | E | 336 | 20.094 | 84.642 | 23.735 | 1.00 | 49.22 | E |
| ATOM | 2825 | N | ARG | E | 337 | 18.559 | 82.953 | 23.608 | 1.00 | 47.43 | E |
| ATOM | 2826 | CA | ARG | E | 337 | 17.541 | 83.665 | 22.845 | 1.00 | 41.04 | E |
| ATOM | 2827 | CB | ARG | E | 337 | 16.787 | 82.688 | 21.951 | 1.00 | 38.57 | E |
| ATOM | 2828 | CG | ARG | E | 337 | 17.636 | 81.673 | 21.234 | 1.00 | 41.45 | E |
| ATOM | 2829 | CD | ARG | E | 337 | 17.898 | 82.058 | 19.792 | 1.00 | 38.02 | E |
| ATOM | 2830 | NE | ARG | E | 337 | 18.855 | 81.138 | 19.188 | 1.00 | 45.29 | E |
| ATOM | 2831 | CZ | ARG | E | 337 | 19.432 | 81.322 | 18.005 | 1.00 | 46.67 | E |
| ATOM | 2832 | NH1 | ARG | E | 337 | 19.142 | 82.405 | 17.284 | 1.00 | 28.51 | E |
| ATOM | 2833 | NH2 | ARG | E | 337 | 20.308 | 80.422 | 17.555 | 1.00 | 41.41 | E |
| ATOM | 2834 | C | ARG | E | 337 | 16.564 | 84.212 | 23.894 | 1.00 | 43.46 | E |
| ATOM | 2835 | O | ARG | E | 337 | 16.553 | 83.743 | 25.039 | 1.00 | 41.16 | E |
| ATOM | 2836 | N | PRO | E | 338 | 15.711 | 85.191 | 23.516 | 1.00 | 47.00 | E |
| ATOM | 2837 | CD | PRO | E | 338 | 15.414 | 85.666 | 22.153 | 1.00 | 43.34 | E |
| ATOM | 2838 | CA | PRO | E | 338 | 14.749 | 85.753 | 24.479 | 1.00 | 42.51 | E |
| ATOM | 2839 | CB | PRO | E | 338 | 14.112 | 86.905 | 23.714 | 1.00 | 37.53 | E |
| ATOM | 2840 | CG | PRO | E | 338 | 14.912 | 87.033 | 22.417 | 1.00 | 38.31 | E |
| ATOM | 2841 | C | PRO | E | 338 | 13.743 | 84.665 | 24.730 | 1.00 | 41.36 | E |
| ATOM | 2842 | O | PRO | E | 338 | 13.797 | 83.642 | 24.058 | 1.00 | 50.86 | E |
| ATOM | 2843 | N | SER | E | 339 | 12.830 | 84.846 | 25.674 | 1.00 | 43.71 | E |
| ATOM | 2844 | CA | SER | E | 339 | 11.810 | 83.807 | 25.910 | 1.00 | 51.70 | E |
| ATOM | 2845 | CB | SER | E | 339 | 11.104 | 83.440 | 24.608 | 1.00 | 53.32 | E |
| ATOM | 2846 | OG | SER | E | 339 | 11.824 | 82.413 | 23.939 | 1.00 | 58.35 | E |
| ATOM | 2847 | C | SER | E | 339 | 12.292 | 82.493 | 26.545 | 1.00 | 52.52 | E |
| ATOM | 2848 | O | SER | E | 339 | 11.477 | 81.726 | 27.052 | 1.00 | 55.04 | E |
| ATOM | 2849 | N | LEU | E | 340 | 13.585 | 82.189 | 26.461 | 1.00 | 50.66 | E |
| ATOM | 2850 | CA | LEU | E | 340 | 14.091 | 80.998 | 27.121 | 1.00 | 46.28 | E |
| ATOM | 2851 | CB | LEU | E | 340 | 15.599 | 80.918 | 26.997 | 1.00 | 40.25 | E |
| ATOM | 2852 | CG | LEU | E | 340 | 16.288 | 80.182 | 25.872 | 1.00 | 38.30 | E |
| ATOM | 2853 | CD1 | LEU | E | 340 | 17.763 | 80.548 | 25.914 | 1.00 | 44.30 | E |
| ATOM | 2854 | CD2 | LEU | E | 340 | 16.109 | 78.696 | 26.029 | 1.00 | 37.84 | E |
| ATOM | 2855 | C | LEU | E | 340 | 13.768 | 81.273 | 28.591 | 1.00 | 50.48 | E |
| ATOM | 2856 | O | LEU | E | 340 | 13.817 | 82.424 | 29.027 | 1.00 | 51.67 | E |
| ATOM | 2857 | N | SER | E | 341 | 13.447 | 80.240 | 29.361 | 1.00 | 50.33 | E |
| ATOM | 2858 | CA | SER | E | 341 | 13.149 | 80.444 | 30.768 | 1.00 | 46.30 | E |
| ATOM | 2859 | CB | SER | E | 341 | 12.533 | 79.209 | 31.375 | 1.00 | 44.20 | E |
| ATOM | 2860 | OG | SER | E | 341 | 12.684 | 79.286 | 32.778 | 1.00 | 52.17 | E |
| ATOM | 2861 | C | SER | E | 341 | 14.395 | 80.782 | 31.570 | 1.00 | 49.57 | E |
| ATOM | 2862 | O | SER | E | 341 | 14.440 | 81.792 | 32.264 | 1.00 | 51.49 | E |
| ATOM | 2863 | N | GLU | E | 342 | 15.410 | 79.930 | 31.492 | 1.00 | 49.30 | E |
| ATOM | 2864 | CA | GLU | E | 342 | 16.631 | 80.193 | 32.230 | 1.00 | 46.56 | E |
| ATOM | 2865 | CB | GLU | E | 342 | 17.078 | 78.970 | 32.996 | 1.00 | 54.05 | E |
| ATOM | 2866 | CG | GLU | E | 342 | 16.055 | 78.360 | 33.887 | 1.00 | 60.75 | E |
| ATOM | 2867 | CD | GLU | E | 342 | 16.557 | 77.045 | 34.412 | 1.00 | 67.98 | E |
| ATOM | 2868 | OE1 | GLU | E | 342 | 16.856 | 76.146 | 33.578 | 1.00 | 72.09 | E |
| ATOM | 2869 | OE2 | GLU | E | 342 | 16.666 | 76.924 | 35.650 | 1.00 | 69.64 | E |
| ATOM | 2870 | C | GLU | E | 342 | 17.759 | 80.602 | 31.316 | 1.00 | 42.61 | E |
| ATOM | 2871 | O | GLU | E | 342 | 18.784 | 79.929 | 31.245 | 1.00 | 37.29 | E |
| ATOM | 2872 | N | GLY | E | 343 | 17.579 | 81.717 | 30.627 | 1.00 | 40.60 | E |
| ATOM | 2873 | CA | GLY | E | 343 | 18.623 | 82.191 | 29.743 | 1.00 | 43.46 | E |
| ATOM | 2874 | C | GLY | E | 343 | 20.031 | 82.226 | 30.335 | 1.00 | 39.18 | E |
| ATOM | 2875 | O | GLY | E | 343 | 20.987 | 81.757 | 29.690 | 1.00 | 37.87 | E |
| ATOM | 2876 | N | TRP | E | 344 | 20.162 | 82.782 | 31.541 | 1.00 | 35.69 | E |
| ATOM | 2877 | CA | TRP | E | 344 | 21.455 | 82.894 | 32.217 | 1.00 | 41.77 | E |
| ATOM | 2878 | CB | TRP | E | 344 | 21.261 | 83.417 | 33.644 | 1.00 | 46.03 | E |
| ATOM | 2879 | CG | TRP | E | 344 | 20.536 | 82.476 | 34.537 | 1.00 | 51.15 | E |
| ATOM | 2880 | CD2 | TRP | E | 344 | 21.122 | 81.419 | 35.301 | 1.00 | 58.71 | E |
| ATOM | 2881 | CE2 | TRP | E | 344 | 20.058 | 80.672 | 35.862 | 1.00 | 56.54 | E |
| ATOM | 2882 | CE3 | TRP | E | 344 | 22.448 | 81.027 | 35.565 | 1.00 | 57.83 | E |
| ATOM | 2883 | CD1 | TRP | E | 344 | 19.182 | 82.341 | 34.669 | 1.00 | 50.37 | E |
| ATOM | 2884 | NE1 | TRP | E | 344 | 18.887 | 81.258 | 35.459 | 1.00 | 52.67 | E |
| ATOM | 2885 | CZ2 | TRP | E | 344 | 20.275 | 79.540 | 36.657 | 1.00 | 56.40 | E |
| ATOM | 2886 | CZ3 | TRP | E | 344 | 22.665 | 79.897 | 36.356 | 1.00 | 60.15 | E |
| ATOM | 2887 | CH2 | TRP | E | 344 | 21.581 | 79.169 | 36.896 | 1.00 | 58.16 | E |
| ATOM | 2888 | C | TRP | E | 344 | 22.235 | 81.576 | 32.265 | 1.00 | 44.23 | E |
| ATOM | 2889 | O | TRP | E | 344 | 23.474 | 81.563 | 32.209 | 1.00 | 44.69 | E |
| ATOM | 2890 | N | LYS | E | 345 | 21.504 | 80.469 | 32.360 | 1.00 | 44.02 | E |
| ATOM | 2891 | CA | LYS | E | 345 | 22.124 | 79.160 | 32.429 | 1.00 | 47.06 | E |
| ATOM | 2892 | CB | LYS | E | 345 | 21.127 | 78.153 | 33.009 | 1.00 | 46.11 | E |
| ATOM | 2893 | CG | LYS | E | 345 | 21.768 | 76.843 | 33.407 | 1.00 | 48.67 | E |
| ATOM | 2894 | CD | LYS | E | 345 | 20.744 | 75.779 | 33.786 | 1.00 | 58.19 | E |
| ATOM | 2895 | CE | LYS | E | 345 | 20.045 | 76.082 | 35.102 | 1.00 | 65.45 | E |
| ATOM | 2896 | NZ | LYS | E | 345 | 19.499 | 74.824 | 35.683 | 1.00 | 61.68 | E |
| ATOM | 2897 | C | LYS | E | 345 | 22.646 | 78.699 | 31.057 | 1.00 | 51.21 | E |
| ATOM | 2898 | O | LYS | E | 345 | 23.750 | 78.140 | 30.976 | 1.00 | 51.22 | E |
| ATOM | 2899 | N | VAL | E | 346 | 21.867 | 78.926 | 29.989 | 1.00 | 47.18 | E |
| ATOM | 2900 | CA | VAL | E | 346 | 22.306 | 78.552 | 28.641 | 1.00 | 42.50 | E |
| ATOM | 2901 | CB | VAL | E | 346 | 21.234 | 78.869 | 27.546 | 1.00 | 37.11 | E |
| ATOM | 2902 | CG1 | VAL | E | 346 | 21.778 | 78.546 | 26.199 | 1.00 | 34.24 | E |
| ATOM | 2903 | CG2 | VAL | E | 346 | 19.997 | 78.030 | 27.726 | 1.00 | 35.13 | E |
| ATOM | 2904 | C | VAL | E | 346 | 23.557 | 79.390 | 28.359 | 1.00 | 41.56 | E |
| ATOM | 2905 | O | VAL | E | 346 | 24.545 | 78.913 | 27.789 | 1.00 | 43.97 | E |
| ATOM | 2906 | N | GLU | E | 347 | 23.513 | 80.639 | 28.798 | 1.00 | 38.53 | E |
| ATOM | 2907 | CA | GLU | E | 347 | 24.616 | 81.568 | 28.607 | 1.00 | 41.57 | E |
| ATOM | 2908 | CB | GLU | E | 347 | 24.278 | 82.881 | 29.280 | 1.00 | 39.84 | E |
| ATOM | 2909 | CG | GLU | E | 347 | 25.069 | 84.033 | 28.792 | 1.00 | 49.68 | E |
| ATOM | 2910 | CD | GLU | E | 347 | 24.494 | 85.334 | 29.295 | 1.00 | 68.89 | E |
| ATOM | 2911 | OE1 | GLU | E | 347 | 25.028 | 85.866 | 30.297 | 1.00 | 81.64 | E |
| ATOM | 2912 | OE2 | GLU | E | 347 | 23.499 | 85.819 | 28.697 | 1.00 | 61.93 | E |
| ATOM | 2913 | C | GLU | E | 347 | 25.919 | 81.047 | 29.176 | 1.00 | 43.24 | E |
| ATOM | 2914 | O | GLU | E | 347 | 26.955 | 81.047 | 28.512 | 1.00 | 41.17 | E |
| ATOM | 2915 | N | LYS | E | 348 | 25.844 | 80.610 | 30.427 | 1.00 | 46.94 | E |
| ATOM | 2916 | CA | LYS | E | 348 | 26.983 | 80.085 | 31.171 | 1.00 | 47.97 | E |
| ATOM | 2917 | CB | LYS | E | 348 | 26.526 | 79.869 | 32.613 | 1.00 | 52.33 | E |
| ATOM | 2918 | CG | LYS | E | 348 | 27.545 | 79.271 | 33.568 | 1.00 | 61.82 | E |
| ATOM | 2919 | CD | LYS | E | 348 | 26.881 | 79.124 | 34.941 | 1.00 | 71.46 | E |
| ATOM | 2920 | CE | LYS | E | 348 | 27.773 | 78.466 | 35.978 | 1.00 | 75.28 | E |
| ATOM | 2921 | NZ | LYS | E | 348 | 27.124 | 78.532 | 37.323 | 1.00 | 75.30 | E |
| ATOM | 2922 | C | LYS | E | 348 | 27.573 | 78.795 | 30.570 | 1.00 | 46.87 | E |
| ATOM | 2923 | O | LYS | E | 348 | 28.794 | 78.596 | 30.575 | 1.00 | 42.83 | E |
| ATOM | 2924 | N | ILE | E | 349 | 26.692 | 77.928 | 30.065 | 1.00 | 43.26 | E |
| ATOM | 2925 | CA | ILE | E | 349 | 27.082 | 76.663 | 29.445 | 1.00 | 40.07 | E |
| ATOM | 2926 | CB | ILE | E | 349 | 25.845 | 75.789 | 29.149 | 1.00 | 38.98 | E |
| ATOM | 2927 | CG2 | ILE | E | 349 | 26.245 | 74.532 | 28.385 | 1.00 | 29.15 | E |
| ATOM | 2928 | CG1 | ILE | E | 349 | 25.150 | 75.440 | 30.462 | 1.00 | 35.25 | E |
| ATOM | 2929 | CD1 | ILE | E | 349 | 23.920 | 74.598 | 30.307 | 1.00 | 32.85 | E |
| ATOM | 2930 | C | ILE | E | 349 | 27.807 | 76.950 | 28.138 | 1.00 | 43.83 | E |
| ATOM | 2931 | O | ILE | E | 349 | 28.832 | 76.337 | 27.818 | 1.00 | 42.49 | E |
| ATOM | 2932 | N | GLN | E | 350 | 27.262 | 77.888 | 27.375 | 1.00 | 38.97 | E |
| ATOM | 2933 | CA | GLN | E | 350 | 27.886 | 78.253 | 26.126 | 1.00 | 39.89 | E |
| ATOM | 2934 | CB | GLN | E | 350 | 27.056 | 79.306 | 25.410 | 1.00 | 37.99 | E |
| ATOM | 2935 | CG | GLN | E | 350 | 27.766 | 79.885 | 24.209 | 1.00 | 34.17 | E |
| ATOM | 2936 | CD | GLN | E | 350 | 27.106 | 81.140 | 23.749 | 1.00 | 35.92 | E |
| ATOM | 2937 | OE1 | GLN | E | 350 | 26.843 | 82.019 | 24.552 | 1.00 | 39.58 | E |
| ATOM | 2938 | NE2 | GLN | E | 350 | 26.827 | 81.238 | 22.461 | 1.00 | 31.17 | E |
| ATOM | 2939 | C | GLN | E | 350 | 29.311 | 78.773 | 26.351 | 1.00 | 40.10 | E |
| ATOM | 2940 | O | GLN | E | 350 | 30.212 | 78.483 | 25.569 | 1.00 | 37.93 | E |
| ATOM | 2941 | N | GLU | E | 351 | 29.517 | 79.541 | 27.416 | 1.00 | 39.98 | E |
| ATOM | 2942 | CA | GLU | E | 351 | 30.843 | 80.079 | 27.702 | 1.00 | 42.25 | E |
| ATOM | 2943 | CB | GLU | E | 351 | 30.825 | 80.814 | 29.044 | 1.00 | 45.08 | E |
| ATOM | 2944 | CG | GLU | E | 351 | 29.972 | 82.086 | 29.014 | 1.00 | 66.01 | E |
| ATOM | 2945 | CD | GLU | E | 351 | 29.961 | 82.864 | 30.340 | 1.00 | 74.89 | E |
| ATOM | 2946 | OE1 | GLU | E | 351 | 29.392 | 83.996 | 30.385 | 1.00 | 66.89 | E |
| ATOM | 2947 | OE2 | GLU | E | 351 | 30.522 | 82.334 | 31.334 | 1.00 | 77.72 | E |
| ATOM | 2948 | C | GLU | E | 351 | 31.916 | 78.980 | 27.690 | 1.00 | 42.79 | E |
| ATOM | 2949 | O | GLU | E | 351 | 33.033 | 79.180 | 27.227 | 1.00 | 43.40 | E |
| ATOM | 2950 | N | ILE | E | 352 | 31.573 | 77.803 | 28.181 | 1.00 | 40.39 | E |
| ATOM | 2951 | CA | ILE | E | 352 | 32.524 | 76.703 | 28.196 | 1.00 | 36.14 | E |
| ATOM | 2952 | CB | ILE | E | 352 | 31.901 | 75.485 | 28.860 | 1.00 | 38.19 | E |
| ATOM | 2953 | CG2 | ILE | E | 352 | 32.758 | 74.270 | 28.640 | 1.00 | 23.85 | E |
| ATOM | 2954 | CG1 | ILE | E | 352 | 31.684 | 75.781 | 30.336 | 1.00 | 39.13 | E |
| ATOM | 2955 | CD1 | ILE | E | 352 | 30.936 | 74.680 | 31.072 | 1.00 | 46.43 | E |
| ATOM | 2956 | C | ILE | E | 352 | 33.012 | 76.312 | 26.792 | 1.00 | 37.13 | E |
| ATOM | 2957 | O | ILE | E | 352 | 34.195 | 76.032 | 26.599 | 1.00 | 35.23 | E |
| ATOM | 2958 | N | TYR | E | 353 | 32.110 | 76.280 | 25.815 | 1.00 | 36.67 | E |
| ATOM | 2959 | CA | TYR | E | 353 | 32.509 | 75.918 | 24.461 | 1.00 | 31.52 | E |
| ATOM | 2960 | CB | TYR | E | 353 | 31.299 | 75.528 | 23.630 | 1.00 | 23.57 | E |
| ATOM | 2961 | CG | TYR | E | 353 | 30.615 | 74.299 | 24.164 | 1.00 | 33.24 | E |
| ATOM | 2962 | CD1 | TYR | E | 353 | 29.497 | 74.406 | 24.997 | 1.00 | 33.74 | E |
| ATOM | 2963 | CE1 | TYR | E | 353 | 28.878 | 73.285 | 25.519 | 1.00 | 33.63 | E |
| ATOM | 2964 | CD2 | TYR | E | 353 | 31.102 | 73.026 | 23.866 | 1.00 | 30.53 | E |
| ATOM | 2965 | CE2 | TYR | E | 353 | 30.497 | 71.891 | 24.381 | 1.00 | 33.64 | E |
| ATOM | 2966 | CZ | TYR | E | 353 | 29.383 | 72.024 | 25.209 | 1.00 | 38.19 | E |
| ATOM | 2967 | OH | TYR | E | 353 | 28.772 | 70.897 | 25.719 | 1.00 | 29.68 | E |
| ATOM | 2968 | C | TYR | E | 353 | 33.250 | 77.046 | 23.783 | 1.00 | 30.62 | E |
| ATOM | 2969 | O | TYR | E | 353 | 34.147 | 76.815 | 22.974 | 1.00 | 32.71 | E |
| ATOM | 2970 | N | ILE | E | 354 | 32.871 | 78.274 | 24.121 | 1.00 | 30.90 | E |
| ATOM | 2971 | CA | ILE | E | 354 | 33.496 | 79.463 | 23.553 | 1.00 | 27.81 | E |
| ATOM | 2972 | CB | ILE | E | 354 | 32.832 | 80.742 | 24.097 | 1.00 | 19.96 | E |
| ATOM | 2973 | CG2 | ILE | E | 354 | 33.647 | 81.959 | 23.696 | 1.00 | 15.58 | E |
| ATOM | 2974 | CG1 | ILE | E | 354 | 31.384 | 80.804 | 23.626 | 1.00 | 22.76 | E |
| ATOM | 2975 | CD1 | ILE | E | 354 | 30.692 | 82.059 | 23.946 | 1.00 | 14.85 | E |
| ATOM | 2976 | C | ILE | E | 354 | 34.946 | 79.452 | 23.996 | 1.00 | 32.14 | E |
| ATOM | 2977 | O | ILE | E | 354 | 35.873 | 79.611 | 23.198 | 1.00 | 33.40 | E |
| ATOM | 2978 | N | GLU | E | 355 | 35.111 | 79.237 | 25.297 | 1.00 | 33.87 | E |
| ATOM | 2979 | CA | GLU | E | 355 | 36.399 | 79.203 | 25.943 | 1.00 | 29.98 | E |
| ATOM | 2980 | CB | GLU | E | 355 | 36.159 | 79.130 | 27.456 | 1.00 | 24.91 | E |
| ATOM | 2981 | CG | GLU | E | 355 | 37.368 | 79.460 | 28.311 | 1.00 | 49.56 | E |
| ATOM | 2982 | CD | GLU | E | 355 | 38.095 | 80.737 | 27.876 | 1.00 | 62.97 | E |
| ATOM | 2983 | OE1 | GLU | E | 355 | 37.526 | 81.854 | 28.028 | 1.00 | 62.84 | E |
| ATOM | 2984 | OE2 | GLU | E | 355 | 39.244 | 80.607 | 27.375 | 1.00 | 68.69 | E |
| ATOM | 2985 | C | GLU | E | 355 | 37.266 | 78.044 | 25.401 | 1.00 | 33.04 | E |
| ATOM | 2986 | O | GLU | E | 355 | 38.481 | 78.203 | 25.204 | 1.00 | 32.75 | E |
| ATOM | 2987 | N | ALA | E | 356 | 36.657 | 76.888 | 25.137 | 1.00 | 31.87 | E |
| ATOM | 2988 | CA | ALA | E | 356 | 37.411 | 75.765 | 24.590 | 1.00 | 33.45 | E |
| ATOM | 2989 | CB | ALA | E | 356 | 36.604 | 74.484 | 24.673 | 1.00 | 31.24 | E |
| ATOM | 2990 | C | ALA | E | 356 | 37.802 | 76.057 | 23.134 | 1.00 | 39.40 | E |
| ATOM | 2991 | O | ALA | E | 356 | 38.849 | 75.602 | 22.681 | 1.00 | 41.00 | E |
| ATOM | 2992 | N | LEU | E | 357 | 36.976 | 76.803 | 22.394 | 1.00 | 36.52 | E |
| ATOM | 2993 | CA | LEU | E | 357 | 37.327 | 77.142 | 21.015 | 1.00 | 32.92 | E |
| ATOM | 2994 | CB | LEU | E | 357 | 36.178 | 77.825 | 20.269 | 1.00 | 32.06 | E |
| ATOM | 2995 | CG | LEU | E | 357 | 36.474 | 78.270 | 18.827 | 1.00 | 25.48 | E |
| ATOM | 2996 | CD1 | LEU | E | 357 | 37.017 | 77.132 | 17.985 | 1.00 | 18.80 | E |
| ATOM | 2997 | CD2 | LEU | E | 357 | 35.199 | 78.780 | 18.219 | 1.00 | 22.88 | E |
| ATOM | 2998 | C | LEU | E | 357 | 38.513 | 78.083 | 21.046 | 1.00 | 34.90 | E |
| ATOM | 2999 | O | LEU | E | 357 | 39.450 | 77.907 | 20.262 | 1.00 | 36.03 | E |
| ATOM | 3000 | N | LYS | E | 358 | 38.475 | 79.082 | 21.940 | 1.00 | 34.91 | E |
| ATOM | 3001 | CA | LYS | E | 358 | 39.597 | 80.024 | 22.062 | 1.00 | 35.86 | E |
| ATOM | 3002 | CB | LYS | E | 358 | 39.393 | 81.073 | 23.177 | 1.00 | 30.78 | E |
| ATOM | 3003 | CG | LYS | E | 358 | 40.080 | 82.422 | 22.858 | 1.00 | 35.85 | E |
| ATOM | 3004 | CD | LYS | E | 358 | 40.114 | 83.450 | 24.006 | 1.00 | 40.68 | E |
| ATOM | 3005 | CE | LYS | E | 358 | 40.961 | 82.961 | 25.212 | 1.00 | 45.86 | E |
| ATOM | 3006 | NZ | LYS | E | 358 | 40.895 | 83.850 | 26.445 | 1.00 | 40.64 | E |
| ATOM | 3007 | C | LYS | E | 358 | 40.848 | 79.208 | 22.364 | 1.00 | 37.00 | E |
| ATOM | 3008 | O | LYS | E | 358 | 41.838 | 79.324 | 21.652 | 1.00 | 35.33 | E |
| ATOM | 3009 | N | ALA | E | 359 | 40.788 | 78.367 | 23.401 | 1.00 | 40.86 | E |
| ATOM | 3010 | CA | ALA | E | 359 | 41.917 | 77.513 | 23.788 | 1.00 | 39.35 | E |
| ATOM | 3011 | CB | ALA | E | 359 | 41.493 | 76.551 | 24.855 | 1.00 | 40.44 | E |
| ATOM | 3012 | C | ALA | E | 359 | 42.472 | 76.732 | 22.609 | 1.00 | 40.83 | E |
| ATOM | 3013 | O | ALA | E | 359 | 43.680 | 76.688 | 22.394 | 1.00 | 38.57 | E |
| ATOM | 3014 | N | TYR | E | 360 | 41.576 | 76.116 | 21.846 | 1.00 | 38.26 | E |
| ATOM | 3015 | CA | TYR | E | 360 | 41.956 | 75.317 | 20.694 | 1.00 | 32.22 | E |
| ATOM | 3016 | CB | TYR | E | 360 | 40.725 | 74.614 | 20.125 | 1.00 | 27.64 | E |
| ATOM | 3017 | CG | TYR | E | 360 | 40.977 | 73.808 | 18.858 | 1.00 | 25.35 | E |
| ATOM | 3018 | CD1 | TYR | E | 360 | 41.381 | 72.483 | 18.923 | 1.00 | 18.24 | E |
| ATOM | 3019 | CE1 | TYR | E | 360 | 41.611 | 71.751 | 17.773 | 1.00 | 20.01 | E |
| ATOM | 3020 | CD2 | TYR | E | 360 | 40.807 | 74.383 | 17.588 | 1.00 | 25.83 | E |
| ATOM | 3021 | CE2 | TYR | E | 360 | 41.037 | 73.659 | 16.433 | 1.00 | 23.29 | E |
| ATOM | 3022 | CZ | TYR | E | 360 | 41.436 | 72.337 | 16.530 | 1.00 | 28.18 | E |
| ATOM | 3023 | OH | TYR | E | 360 | 41.627 | 71.583 | 15.386 | 1.00 | 25.99 | E |
| ATOM | 3024 | C | TYR | E | 360 | 42.609 | 76.146 | 19.605 | 1.00 | 36.45 | E |
| ATOM | 3025 | O | TYR | E | 360 | 43.680 | 75.786 | 19.119 | 1.00 | 39.74 | E |
| ATOM | 3026 | N | VAL | E | 361 | 41.960 | 77.245 | 19.216 | 1.00 | 36.88 | E |
| ATOM | 3027 | CA | VAL | E | 361 | 42.467 | 78.113 | 18.146 | 1.00 | 40.55 | E |
| ATOM | 3028 | CB | VAL | E | 361 | 41.450 | 79.225 | 17.809 | 1.00 | 38.45 | E |
| ATOM | 3029 | CG1 | VAL | E | 361 | 41.931 | 80.026 | 16.619 | 1.00 | 30.69 | E |
| ATOM | 3030 | CG2 | VAL | E | 361 | 40.089 | 78.603 | 17.523 | 1.00 | 36.50 | E |
| ATOM | 3031 | C | VAL | E | 361 | 43.812 | 78.744 | 18.484 | 1.00 | 42.98 | E |
| ATOM | 3032 | O | VAL | E | 361 | 44.695 | 78.825 | 17.639 | 1.00 | 43.97 | E |
| ATOM | 3033 | N | GLU | E | 362 | 43.964 | 79.180 | 19.729 | 1.00 | 49.63 | E |
| ATOM | 3034 | CA | GLU | E | 362 | 45.214 | 79.775 | 20.182 | 1.00 | 50.89 | E |
| ATOM | 3035 | CB | GLU | E | 362 | 45.082 | 80.354 | 21.587 | 1.00 | 42.81 | E |
| ATOM | 3036 | CG | GLU | E | 362 | 44.453 | 81.704 | 21.618 | 1.00 | 48.93 | E |
| ATOM | 3037 | CD | GLU | E | 362 | 44.243 | 82.207 | 23.024 | 1.00 | 63.80 | E |
| ATOM | 3038 | OE1 | GLU | E | 362 | 43.499 | 81.561 | 23.799 | 1.00 | 70.08 | E |
| ATOM | 3039 | OE2 | GLU | E | 362 | 44.818 | 83.262 | 23.358 | 1.00 | 74.63 | E |
| ATOM | 3040 | C | GLU | E | 362 | 46.320 | 78.744 | 20.195 | 1.00 | 52.51 | E |
| ATOM | 3041 | O | GLU | E | 362 | 47.405 | 79.001 | 19.690 | 1.00 | 53.90 | E |
| ATOM | 3042 | N | ASN | E | 363 | 46.045 | 77.574 | 20.764 | 1.00 | 57.67 | E |
| ATOM | 3043 | CA | ASN | E | 363 | 47.052 | 76.525 | 20.841 | 1.00 | 57.57 | E |
| ATOM | 3044 | CB | ASN | E | 363 | 46.683 | 75.528 | 21.939 | 1.00 | 55.13 | E |
| ATOM | 3045 | CG | ASN | E | 363 | 47.074 | 76.027 | 23.314 | 1.00 | 60.47 | E |
| ATOM | 3046 | OD1 | ASN | E | 363 | 48.251 | 76.028 | 23.665 | 1.00 | 68.73 | E |
| ATOM | 3047 | ND2 | ASN | E | 363 | 46.095 | 76.475 | 24.091 | 1.00 | 59.89 | E |
| ATOM | 3048 | C | ASN | E | 363 | 47.414 | 75.817 | 19.539 | 1.00 | 57.23 | E |
| ATOM | 3049 | O | ASN | E | 363 | 48.215 | 74.898 | 19.549 | 1.00 | 60.35 | E |
| ATOM | 3050 | N | ARG | E | 364 | 46.827 | 76.229 | 18.419 | 1.00 | 62.49 | E |
| ATOM | 3051 | CA | ARG | E | 364 | 47.226 | 75.671 | 17.129 | 1.00 | 71.24 | E |
| ATOM | 3052 | CB | ARG | E | 364 | 46.214 | 75.966 | 16.032 | 1.00 | 68.25 | E |
| ATOM | 3053 | CG | ARG | E | 364 | 44.911 | 75.255 | 16.192 | 1.00 | 80.52 | E |
| ATOM | 3054 | CD | ARG | E | 364 | 45.079 | 73.745 | 16.101 | 1.00 | 88.65 | E |
| ATOM | 3055 | NE | ARG | E | 364 | 45.647 | 73.149 | 17.309 | 1.00 | 91.73 | E |
| ATOM | 3056 | CZ | ARG | E | 364 | 45.827 | 71.840 | 17.478 | 1.00 | 93.79 | E |
| ATOM | 3057 | NH1 | ARG | E | 364 | 45.481 | 70.993 | 16.512 | 1.00 | 92.83 | E |
| ATOM | 3058 | NH2 | ARG | E | 364 | 46.345 | 71.372 | 18.609 | 1.00 | 93.18 | E |
| ATOM | 3059 | C | ARG | E | 364 | 48.442 | 76.541 | 16.879 | 1.00 | 81.51 | E |
| ATOM | 3060 | O | ARG | E | 364 | 48.920 | 76.647 | 15.744 | 1.00 | 84.85 | E |
| ATOM | 3061 | N | ARG | E | 365 | 48.893 | 77.170 | 17.982 | 1.00 | 92.03 | E |
| ATOM | 3062 | CA | ARG | E | 365 | 50.030 | 78.117 | 18.098 | 1.00 | 96.21 | E |
| ATOM | 3063 | CB | ARG | E | 365 | 51.237 | 77.445 | 18.803 | 1.00 | 95.61 | E |
| ATOM | 3064 | CG | ARG | E | 365 | 52.264 | 78.397 | 19.455 | 1.00 | 91.12 | E |
| ATOM | 3065 | CD | ARG | E | 365 | 51.758 | 79.135 | 20.710 | 1.00 | 94.23 | E |
| ATOM | 3066 | NE | ARG | E | 365 | 51.599 | 78.281 | 21.899 | 1.00 | 104.40 | E |
| ATOM | 3067 | CZ | ARG | E | 365 | 51.437 | 78.725 | 23.156 | 1.00 | 105.97 | E |
| ATOM | 3068 | NH1 | ARG | E | 365 | 51.412 | 80.027 | 23.432 | 1.00 | 100.30 | E |
| ATOM | 3069 | NH2 | ARG | E | 365 | 51.290 | 77.855 | 24.154 | 1.00 | 106.95 | E |
| ATOM | 3070 | C | ARG | E | 365 | 50.407 | 78.678 | 16.729 | 1.00 | 99.02 | E |
| ATOM | 3071 | O | ARG | E | 365 | 51.579 | 78.784 | 16.360 | 1.00 | 103.34 | E |
| ATOM | 3072 | N | LYS | E | 366 | 49.361 | 79.040 | 15.992 | 1.00 | 99.93 | E |
| ATOM | 3073 | CA | LYS | E | 366 | 49.463 | 79.595 | 14.655 | 1.00 | 95.80 | E |
| ATOM | 3074 | CB | LYS | E | 366 | 48.296 | 79.085 | 13.813 | 1.00 | 92.63 | E |
| ATOM | 3075 | CG | LYS | E | 366 | 46.926 | 79.229 | 14.505 | 1.00 | 81.57 | E |
| ATOM | 3076 | CD | LYS | E | 366 | 45.835 | 78.705 | 13.592 | 1.00 | 83.83 | E |
| ATOM | 3077 | CE | LYS | E | 366 | 44.531 | 78.450 | 14.313 | 1.00 | 87.71 | E |
| ATOM | 3078 | NZ | LYS | E | 366 | 43.528 | 77.810 | 13.402 | 1.00 | 87.24 | E |
| ATOM | 3079 | C | LYS | E | 366 | 49.362 | 81.104 | 14.776 | 1.00 | 95.00 | E |
| ATOM | 3080 | O | LYS | E | 366 | 48.751 | 81.616 | 15.721 | 1.00 | 97.66 | E |
| ATOM | 3081 | N | PRO | E | 367 | 49.991 | 81.842 | 13.852 | 1.00 | 90.48 | E |
| ATOM | 3082 | CD | PRO | E | 367 | 51.023 | 81.481 | 12.867 | 1.00 | 85.71 | E |
| ATOM | 3083 | CA | PRO | E | 367 | 49.866 | 83.295 | 13.972 | 1.00 | 86.93 | E |
| ATOM | 3084 | CB | PRO | E | 367 | 50.800 | 83.803 | 12.883 | 1.00 | 81.22 | E |
| ATOM | 3085 | CG | PRO | E | 367 | 51.834 | 82.743 | 12.807 | 1.00 | 80.89 | E |
| ATOM | 3086 | C | PRO | E | 367 | 48.387 | 83.621 | 13.673 | 1.00 | 90.72 | E |
| ATOM | 3087 | O | PRO | E | 367 | 47.611 | 82.732 | 13.291 | 1.00 | 93.37 | E |
| ATOM | 3088 | N | TYR | E | 368 | 47.985 | 84.874 | 13.843 | 1.00 | 88.30 | E |
| ATOM | 3089 | CA | TYR | E | 368 | 46.604 | 85.272 | 13.558 | 1.00 | 87.82 | E |
| ATOM | 3090 | CB | TYR | E | 368 | 46.191 | 84.850 | 12.141 | 1.00 | 97.22 | E |
| ATOM | 3091 | CG | TYR | E | 368 | 47.285 | 84.791 | 11.088 | 1.00 | 100.81 | E |
| ATOM | 3092 | CD1 | TYR | E | 368 | 47.913 | 83.584 | 10.786 | 1.00 | 101.27 | E |
| ATOM | 3093 | CE1 | TYR | E | 368 | 48.840 | 83.488 | 9.773 | 1.00 | 103.17 | E |
| ATOM | 3094 | CD2 | TYR | E | 368 | 47.629 | 85.916 | 10.338 | 1.00 | 100.48 | E |
| ATOM | 3095 | CE2 | TYR | E | 368 | 48.562 | 85.830 | 9.310 | 1.00 | 103.55 | E |
| ATOM | 3096 | CZ | TYR | E | 368 | 49.160 | 84.605 | 9.032 | 1.00 | 105.22 | E |
| ATOM | 3097 | OH | TYR | E | 368 | 50.056 | 84.471 | 7.989 | 1.00 | 112.94 | E |
| ATOM | 3098 | C | TYR | E | 368 | 45.520 | 84.749 | 14.514 | 1.00 | 80.96 | E |
| ATOM | 3099 | O | TYR | E | 368 | 44.380 | 85.211 | 14.452 | 1.00 | 81.08 | E |
| ATOM | 3100 | N | ALA | E | 369 | 45.865 | 83.787 | 15.368 | 1.00 | 70.44 | E |
| ATOM | 3101 | CA | ALA | E | 369 | 44.932 | 83.191 | 16.328 | 1.00 | 62.48 | E |
| ATOM | 3102 | CB | ALA | E | 369 | 45.687 | 82.777 | 17.564 | 1.00 | 55.83 | E |
| ATOM | 3103 | C | ALA | E | 369 | 43.729 | 84.062 | 16.725 | 1.00 | 62.51 | E |
| ATOM | 3104 | O | ALA | E | 369 | 42.578 | 83.624 | 16.642 | 1.00 | 59.98 | E |
| ATOM | 3105 | N | THR | E | 370 | 43.973 | 85.293 | 17.160 | 1.00 | 58.77 | E |
| ATOM | 3106 | CA | THR | E | 370 | 42.855 | 86.142 | 17.549 | 1.00 | 56.88 | E |
| ATOM | 3107 | CB | THR | E | 370 | 43.338 | 87.422 | 18.308 | 1.00 | 52.55 | E |
| ATOM | 3108 | OG1 | THR | E | 370 | 43.149 | 88.595 | 17.492 | 1.00 | 40.99 | E |
| ATOM | 3109 | CG2 | THR | E | 370 | 44.797 | 87.248 | 18.723 | 1.00 | 43.43 | E |
| ATOM | 3110 | C | THR | E | 370 | 42.013 | 86.512 | 16.324 | 1.00 | 57.87 | E |
| ATOM | 3111 | O | THR | E | 370 | 40.783 | 86.590 | 16.400 | 1.00 | 58.49 | E |
| ATOM | 3112 | N | THR | E | 371 | 42.667 | 86.721 | 15.189 | 1.00 | 55.14 | E |
| ATOM | 3113 | CA | THR | E | 371 | 41.931 | 87.050 | 13.982 | 1.00 | 53.34 | E |
| ATOM | 3114 | CB | THR | E | 371 | 42.876 | 87.608 | 12.876 | 1.00 | 51.68 | E |
| ATOM | 3115 | OG1 | THR | E | 371 | 42.886 | 89.053 | 12.954 | 1.00 | 39.83 | E |
| ATOM | 3116 | CG2 | THR | E | 371 | 42.436 | 87.112 | 11.466 | 1.00 | 30.79 | E |
| ATOM | 3117 | C | THR | E | 371 | 41.148 | 85.835 | 13.477 | 1.00 | 54.94 | E |
| ATOM | 3118 | O | THR | E | 371 | 40.052 | 85.976 | 12.934 | 1.00 | 59.29 | E |
| ATOM | 3119 | N | ILE | E | 372 | 41.693 | 84.637 | 13.660 | 1.00 | 52.00 | E |
| ATOM | 3120 | CA | ILE | E | 372 | 40.982 | 83.439 | 13.221 | 1.00 | 46.89 | E |
| ATOM | 3121 | CB | ILE | E | 372 | 41.881 | 82.188 | 13.252 | 1.00 | 46.50 | E |
| ATOM | 3122 | CG2 | ILE | E | 372 | 41.110 | 80.989 | 12.767 | 1.00 | 46.12 | E |
| ATOM | 3123 | CG1 | ILE | E | 372 | 43.085 | 82.388 | 12.341 | 1.00 | 49.39 | E |
| ATOM | 3124 | CD1 | ILE | E | 372 | 44.280 | 81.540 | 12.713 | 1.00 | 66.77 | E |
| ATOM | 3125 | C | ILE | E | 372 | 39.797 | 83.245 | 14.156 | 1.00 | 43.94 | E |
| ATOM | 3126 | O | ILE | E | 372 | 38.676 | 83.080 | 13.705 | 1.00 | 50.56 | E |
| ATOM | 3127 | N | PHE | E | 373 | 40.038 | 83.281 | 15.458 | 1.00 | 34.23 | E |
| ATOM | 3128 | CA | PHE | E | 373 | 38.961 | 83.126 | 16.430 | 1.00 | 33.38 | E |
| ATOM | 3129 | CB | PHE | E | 373 | 39.517 | 83.371 | 17.828 | 1.00 | 29.69 | E |
| ATOM | 3130 | CG | PHE | E | 373 | 38.508 | 83.204 | 18.923 | 1.00 | 25.05 | E |
| ATOM | 3131 | CD1 | PHE | E | 373 | 38.117 | 81.945 | 19.339 | 1.00 | 32.12 | E |
| ATOM | 3132 | CD2 | PHE | E | 373 | 37.979 | 84.318 | 19.573 | 1.00 | 33.08 | E |
| ATOM | 3133 | CE1 | PHE | E | 373 | 37.213 | 81.799 | 20.389 | 1.00 | 37.40 | E |
| ATOM | 3134 | CE2 | PHE | E | 373 | 37.080 | 84.185 | 20.618 | 1.00 | 33.02 | E |
| ATOM | 3135 | CZ | PHE | E | 373 | 36.698 | 82.928 | 21.032 | 1.00 | 36.20 | E |
| ATOM | 3136 | C | PHE | E | 373 | 37.781 | 84.082 | 16.170 | 1.00 | 34.09 | E |
| ATOM | 3137 | O | PHE | E | 373 | 36.609 | 83.686 | 16.230 | 1.00 | 29.43 | E |
| ATOM | 3138 | N | ALA | E | 374 | 38.109 | 85.345 | 15.896 | 1.00 | 38.16 | E |
| ATOM | 3139 | CA | ALA | E | 374 | 37.110 | 86.380 | 15.628 | 1.00 | 39.91 | E |
| ATOM | 3140 | CB | ALA | E | 374 | 37.767 | 87.770 | 15.583 | 1.00 | 35.72 | E |
| ATOM | 3141 | C | ALA | E | 374 | 36.378 | 86.099 | 14.322 | 1.00 | 38.29 | E |
| ATOM | 3142 | O | ALA | E | 374 | 35.184 | 86.340 | 14.224 | 1.00 | 38.07 | E |
| ATOM | 3143 | N | LYS | E | 375 | 37.097 | 85.599 | 13.323 | 1.00 | 36.27 | E |
| ATOM | 3144 | CA | LYS | E | 375 | 36.495 | 85.266 | 12.042 | 1.00 | 34.18 | E |
| ATOM | 3145 | CB | LYS | E | 375 | 37.568 | 84.935 | 11.021 | 1.00 | 36.07 | E |
| ATOM | 3146 | CG | LYS | E | 375 | 38.357 | 86.088 | 10.476 | 1.00 | 45.35 | E |
| ATOM | 3147 | CD | LYS | E | 375 | 39.190 | 85.560 | 9.313 | 1.00 | 57.63 | E |
| ATOM | 3148 | CE | LYS | E | 375 | 39.943 | 86.638 | 8.568 | 1.00 | 64.98 | E |
| ATOM | 3149 | NZ | LYS | E | 375 | 40.527 | 86.117 | 7.286 | 1.00 | 71.22 | E |
| ATOM | 3150 | C | LYS | E | 375 | 35.557 | 84.054 | 12.180 | 1.00 | 33.41 | E |
| ATOM | 3151 | O | LYS | E | 375 | 34.530 | 83.985 | 11.511 | 1.00 | 32.39 | E |
| ATOM | 3152 | N | LEU | E | 376 | 35.921 | 83.092 | 13.031 | 1.00 | 32.18 | E |
| ATOM | 3153 | CA | LEU | E | 376 | 35.093 | 81.906 | 13.251 | 1.00 | 29.18 | E |
| ATOM | 3154 | CB | LEU | E | 376 | 35.787 | 80.883 | 14.164 | 1.00 | 26.70 | E |
| ATOM | 3155 | CG | LEU | E | 376 | 36.957 | 80.073 | 13.575 | 1.00 | 30.47 | E |
| ATOM | 3156 | CD1 | LEU | E | 376 | 37.561 | 79.226 | 14.664 | 1.00 | 35.46 | E |
| ATOM | 3157 | CD2 | LEU | E | 376 | 36.506 | 79.181 | 12.428 | 1.00 | 15.68 | E |
| ATOM | 3158 | C | LEU | E | 376 | 33.791 | 82.341 | 13.888 | 1.00 | 29.09 | E |
| ATOM | 3159 | O | LEU | E | 376 | 32.717 | 82.018 | 13.387 | 1.00 | 27.41 | E |
| ATOM | 3160 | N | LEU | E | 377 | 33.894 | 83.096 | 14.979 | 1.00 | 27.74 | E |
| ATOM | 3161 | CA | LEU | E | 377 | 32.716 | 83.581 | 15.682 | 1.00 | 29.99 | E |
| ATOM | 3162 | CB | LEU | E | 377 | 33.122 | 84.445 | 16.874 | 1.00 | 28.77 | E |
| ATOM | 3163 | CG | LEU | E | 377 | 33.681 | 83.778 | 18.120 | 1.00 | 27.79 | E |
| ATOM | 3164 | CD1 | LEU | E | 377 | 33.787 | 84.801 | 19.236 | 1.00 | 20.75 | E |
| ATOM | 3165 | CD2 | LEU | E | 377 | 32.759 | 82.647 | 18.523 | 1.00 | 23.07 | E |
| ATOM | 3166 | C | LEU | E | 377 | 31.763 | 84.382 | 14.799 | 1.00 | 34.20 | E |
| ATOM | 3167 | O | LEU | E | 377 | 30.549 | 84.280 | 14.940 | 1.00 | 41.58 | E |
| ATOM | 3168 | N | SER | E | 378 | 32.313 | 85.185 | 13.895 | 1.00 | 33.96 | E |
| ATOM | 3169 | CA | SER | E | 378 | 31.508 | 86.015 | 13.009 | 1.00 | 32.03 | E |
| ATOM | 3170 | CB | SER | E | 378 | 32.396 | 86.822 | 12.077 | 1.00 | 33.38 | E |
| ATOM | 3171 | OG | SER | E | 378 | 33.048 | 85.973 | 11.157 | 1.00 | 34.99 | E |
| ATOM | 3172 | C | SER | E | 378 | 30.591 | 85.178 | 12.159 | 1.00 | 36.66 | E |
| ATOM | 3173 | O | SER | E | 378 | 29.522 | 85.626 | 11.748 | 1.00 | 38.35 | E |
| ATOM | 3174 | N | VAL | E | 379 | 31.015 | 83.960 | 11.868 | 1.00 | 37.35 | E |
| ATOM | 3175 | CA | VAL | E | 379 | 30.197 | 83.093 | 11.057 | 1.00 | 36.78 | E |
| ATOM | 3176 | CB | VAL | E | 379 | 30.862 | 81.728 | 10.945 | 1.00 | 35.51 | E |
| ATOM | 3177 | CG1 | VAL | E | 379 | 29.847 | 80.651 | 10.612 | 1.00 | 37.59 | E |
| ATOM | 3178 | CG2 | VAL | E | 379 | 31.895 | 81.805 | 9.869 | 1.00 | 27.07 | E |
| ATOM | 3179 | C | VAL | E | 379 | 28.793 | 83.010 | 11.640 | 1.00 | 37.14 | E |
| ATOM | 3180 | O | VAL | E | 379 | 27.822 | 82.854 | 10.902 | 1.00 | 40.33 | E |
| ATOM | 3181 | N | LEU | E | 380 | 28.698 | 83.172 | 12.960 | 1.00 | 32.84 | E |
| ATOM | 3182 | CA | LEU | E | 380 | 27.429 | 83.126 | 13.685 | 1.00 | 28.02 | E |
| ATOM | 3183 | CB | LEU | E | 380 | 27.692 | 83.153 | 15.185 | 1.00 | 21.14 | E |
| ATOM | 3184 | CG | LEU | E | 380 | 28.411 | 81.949 | 15.815 | 1.00 | 29.29 | E |
| ATOM | 3185 | CD1 | LEU | E | 380 | 28.117 | 81.968 | 17.298 | 1.00 | 34.75 | E |
| ATOM | 3186 | CD2 | LEU | E | 380 | 27.948 | 80.612 | 15.249 | 1.00 | 22.54 | E |
| ATOM | 3187 | C | LEU | E | 380 | 26.438 | 84.226 | 13.312 | 1.00 | 29.99 | E |
| ATOM | 3188 | O | LEU | E | 380 | 25.225 | 84.042 | 13.434 | 1.00 | 38.27 | E |
| ATOM | 3189 | N | THR | E | 381 | 26.958 | 85.370 | 12.881 | 1.00 | 25.84 | E |
| ATOM | 3190 | CA | THR | E | 381 | 26.132 | 86.484 | 12.436 | 1.00 | 26.88 | E |
| ATOM | 3191 | CB | THR | E | 381 | 26.969 | 87.754 | 12.264 | 1.00 | 15.29 | E |
| ATOM | 3192 | OG1 | THR | E | 381 | 27.705 | 87.998 | 13.456 | 1.00 | 15.98 | E |
| ATOM | 3193 | CG2 | THR | E | 383 | 26.084 | 88.947 | 11.974 | 1.00 | 16.48 | E |
| ATOM | 3194 | C | THR | E | 381 | 25.595 | 86.098 | 11.052 | 1.00 | 32.06 | E |
| ATOM | 3195 | O | THR | E | 381 | 24.464 | 86.407 | 10.677 | 1.00 | 33.96 | E |
| ATOM | 3196 | N | GLU | E | 382 | 26.436 | 85.429 | 10.279 | 1.00 | 34.87 | E |
| ATOM | 3197 | CA | GLU | E | 382 | 26.042 | 85.012 | 8.961 | 1.00 | 30.44 | E |
| ATOM | 3198 | CB | GLU | E | 382 | 27.227 | 84.374 | 8.251 | 1.00 | 35.97 | E |
| ATOM | 3199 | CG | GLU | E | 382 | 26.967 | 84.073 | 6.769 | 1.00 | 57.41 | E |
| ATOM | 3200 | CD | GLU | E | 382 | 26.250 | 85.217 | 6.015 | 1.00 | 63.98 | E |
| ATOM | 3201 | OE1 | GLU | E | 382 | 26.453 | 86.415 | 6.402 | 1.00 | 59.25 | E |
| ATOM | 3202 | OE2 | GLU | E | 382 | 25.509 | 84.901 | 5.026 | 1.00 | 47.35 | E |
| ATOM | 3203 | C | GLU | E | 382 | 24.881 | 84.040 | 9.096 | 1.00 | 28.38 | E |
| ATOM | 3204 | O | GLU | E | 382 | 23.946 | 84.078 | 8.309 | 1.00 | 28.70 | E |
| ATOM | 3205 | N | LEU | E | 383 | 24.919 | 83.191 | 10.118 | 1.00 | 28.31 | E |
| ATOM | 3206 | CA | LEU | E | 383 | 23.845 | 82.228 | 10.339 | 1.00 | 22.49 | E |
| ATOM | 3207 | CB | LEU | E | 383 | 24.204 | 81.302 | 11.495 | 1.00 | 16.97 | E |
| ATOM | 3208 | CG | LEU | E | 383 | 25.357 | 80.359 | 11.141 | 1.00 | 26.83 | E |
| ATOM | 3209 | CD1 | LEU | E | 383 | 25.655 | 79.434 | 12.289 | 1.00 | 20.81 | E |
| ATOM | 3210 | CD2 | LEU | E | 383 | 25.011 | 79.564 | 9.884 | 1.00 | 21.75 | E |
| ATOM | 3211 | C | LEU | E | 383 | 22.480 | 82.887 | 10.566 | 1.00 | 23.24 | E |
| ATOM | 3212 | O | LEU | E | 383 | 21.463 | 82.372 | 10.133 | 1.00 | 27.48 | E |
| ATOM | 3213 | N | ARG | E | 384 | 22.441 | 84.029 | 11.236 | 1.00 | 22.78 | E |
| ATOM | 3214 | CA | ARG | E | 384 | 21.167 | 84.718 | 11.430 | 1.00 | 24.73 | E |
| ATOM | 3215 | CB | ARG | E | 384 | 21.303 | 85.993 | 12.288 | 1.00 | 22.96 | E |
| ATOM | 3216 | CG | ARG | E | 384 | 21.825 | 85.774 | 13.671 | 1.00 | 27.90 | E |
| ATOM | 3217 | CD | ARG | E | 384 | 20.953 | 84.840 | 14.429 | 1.00 | 18.45 | E |
| ATOM | 3218 | NE | ARG | E | 384 | 21.607 | 84.408 | 15.654 | 1.00 | 35.88 | E |
| ATOM | 3219 | CZ | ARG | E | 384 | 21.479 | 85.008 | 16.835 | 1.00 | 34.93 | E |
| ATOM | 3220 | NH1 | ARG | E | 384 | 20.708 | 86.084 | 16.979 | 1.00 | 14.64 | E |
| ATOM | 3221 | NH2 | ARG | E | 384 | 22.138 | 84.515 | 17.876 | 1.00 | 32.02 | E |
| ATOM | 3222 | C | ARG | E | 384 | 20.558 | 85.124 | 10.081 | 1.00 | 26.38 | E |
| ATOM | 3223 | O | ARG | E | 384 | 19.338 | 85.168 | 9.950 | 1.00 | 33.40 | E |
| ATOM | 3224 | N | THR | E | 385 | 21.353 | 85.449 | 9.070 | 1.00 | 19.82 | E |
| ATOM | 3225 | CA | THR | E | 385 | 20.658 | 85.822 | 7.871 | 1.00 | 27.51 | E |
| ATOM | 3226 | CB | THR | E | 385 | 21.451 | 86.826 | 6.978 | 1.00 | 34.59 | E |
| ATOM | 3227 | OG1 | THR | E | 385 | 22.673 | 86.246 | 6.547 | 1.00 | 42.75 | E |
| ATOM | 3228 | CG2 | THR | E | 385 | 21.737 | 88.116 | 7.737 | 1.00 | 36.75 | E |
| ATOM | 3229 | C | THR | E | 385 | 20.286 | 84.574 | 7.103 | 1.00 | 30.02 | E |
| ATOM | 3230 | O | THR | E | 385 | 19.223 | 84.522 | 6.494 | 1.00 | 36.26 | E |
| ATOM | 3231 | N | LEU | E | 386 | 21.133 | 83.553 | 7.160 | 1.00 | 27.82 | E |
| ATOM | 3232 | CA | LEU | E | 386 | 20.866 | 82.311 | 6.445 | 1.00 | 26.77 | E |
| ATOM | 3233 | CB | LEU | E | 386 | 22.080 | 81.381 | 6.533 | 1.00 | 17.31 | E |
| ATOM | 3234 | CG | LEU | E | 386 | 23.232 | 81.678 | 5.585 | 1.00 | 11.79 | E |
| ATOM | 3235 | CD1 | LEU | E | 386 | 24.377 | 80.771 | 5.881 | 1.00 | 29.55 | E |
| ATOM | 3236 | CD2 | LEU | E | 386 | 22.799 | 81.476 | 4.158 | 1.00 | 19.90 | E |
| ATOM | 3237 | C | LEU | E | 386 | 19.634 | 81.600 | 7.004 | 1.00 | 33.17 | E |
| ATOM | 3238 | O | LEU | E | 386 | 18.835 | 81.000 | 6.261 | 1.00 | 32.97 | E |
| ATOM | 3239 | N | GLY | E | 387 | 19.491 | 81.674 | 8.323 | 1.00 | 29.30 | E |
| ATOM | 3240 | CA | GLY | E | 387 | 18.387 | 81.017 | 8.979 | 1.00 | 31.66 | E |
| ATOM | 3241 | C | GLY | E | 387 | 17.113 | 81.760 | 8.690 | 1.00 | 35.31 | E |
| ATOM | 3242 | O | GLY | E | 387 | 16.025 | 81.170 | 8.692 | 1.00 | 38.04 | E |
| ATOM | 3243 | N | ASN | E | 388 | 17.254 | 83.063 | 8.465 | 1.00 | 35.06 | E |
| ATOM | 3244 | CA | ASN | E | 388 | 16.124 | 83.938 | 8.171 | 1.00 | 35.25 | E |
| ATOM | 3245 | CB | ASN | E | 388 | 16.562 | 85.393 | 8.258 | 1.00 | 34.73 | E |
| ATOM | 3246 | CG | ASN | E | 388 | 15.441 | 86.353 | 7.972 | 1.00 | 35.48 | E |
| ATOM | 3247 | OD1 | ASN | E | 388 | 14.368 | 86.264 | 8.554 | 1.00 | 38.42 | E |
| ATOM | 3248 | ND2 | ASN | E | 388 | 15.690 | 87.294 | 7.080 | 1.00 | 40.60 | E |
| ATOM | 3249 | C | ASN | E | 388 | 15.684 | 83.613 | 6.758 | 1.00 | 37.77 | E |
| ATOM | 3250 | O | ASN | E | 388 | 14.496 | 83.507 | 6.461 | 1.00 | 33.42 | E |
| ATOM | 3251 | N | MET | E | 389 | 16.678 | 83.452 | 5.893 | 1.00 | 38.07 | E |
| ATOM | 3252 | CA | MET | E | 389 | 16.459 | 83.097 | 4.507 | 1.00 | 33.49 | E |
| ATOM | 3253 | CB | MET | E | 389 | 17.788 | 82.928 | 3.804 | 1.00 | 34.37 | E |
| ATOM | 3254 | CG | MET | E | 389 | 17.674 | 82.389 | 2.416 | 1.00 | 35.98 | E |
| ATOM | 3255 | SD | MET | E | 389 | 19.307 | 82.315 | 1.703 | 1.00 | 61.87 | E |
| ATOM | 3256 | CE | MET | E | 389 | 19.736 | 80.608 | 2.072 | 1.00 | 48.34 | E |
| ATOM | 3257 | C | MET | E | 389 | 15.715 | 81.779 | 4.456 | 1.00 | 33.16 | E |
| ATOM | 3258 | O | MET | E | 389 | 14.760 | 81.614 | 3.699 | 1.00 | 37.81 | E |
| ATOM | 3259 | N | ASN | E | 390 | 16.146 | 80.823 | 5.259 | 1.00 | 27.59 | E |
| ATOM | 3260 | CA | ASN | E | 390 | 15.458 | 79.562 | 5.215 | 1.00 | 28.20 | E |
| ATOM | 3261 | CB | ASN | E | 390 | 16.097 | 78.568 | 6.166 | 1.00 | 30.32 | E |
| ATOM | 3262 | CG | ASN | E | 390 | 15.584 | 77.175 | 5.949 | 1.00 | 35.90 | E |
| ATOM | 3263 | OD1 | ASN | E | 390 | 14.735 | 76.692 | 6.706 | 1.00 | 31.78 | E |
| ATOM | 3264 | ND2 | ASN | E | 390 | 16.082 | 76.518 | 4.891 | 1.00 | 38.32 | E |
| ATOM | 3265 | C | ASN | E | 390 | 13.978 | 79.731 | 5.519 | 1.00 | 28.45 | E |
| ATOM | 3266 | O | ASN | E | 390 | 13.134 | 79.201 | 4.797 | 1.00 | 28.72 | E |
| ATOM | 3267 | N | SER | E | 391 | 13.668 | 80.483 | 6.572 | 1.00 | 30.90 | E |
| ATOM | 3268 | CA | SER | E | 391 | 12.282 | 80.730 | 6.974 | 1.00 | 31.85 | E |
| ATOM | 3269 | CB | SER | E | 391 | 12.220 | 81.620 | 8.217 | 1.00 | 32.14 | E |
| ATOM | 3270 | OG | SER | E | 391 | 12.727 | 80.950 | 9.360 | 1.00 | 45.82 | E |
| ATOM | 3271 | C | SER | E | 391 | 11.514 | 81.393 | 5.857 | 1.00 | 31.51 | E |
| ATOM | 3272 | O | SER | E | 391 | 10.342 | 81.097 | 5.642 | 1.00 | 31.42 | E |
| ATOM | 3273 | N | GLU | E | 392 | 12.196 | 82.294 | 5.158 | 1.00 | 35.32 | E |
| ATOM | 3274 | CA | GLU | E | 392 | 11.634 | 83.027 | 4.042 | 1.00 | 37.79 | E |
| ATOM | 3275 | CB | GLU | E | 392 | 12.646 | 84.068 | 3.560 | 1.00 | 45.73 | E |
| ATOM | 3276 | CG | GLU | E | 392 | 12.050 | 85.146 | 2.665 | 1.00 | 71.70 | E |
| ATOM | 3277 | CD | GLU | E | 392 | 10.594 | 85.493 | 3.038 | 1.00 | 81.65 | E |
| ATOM | 3278 | OE1 | GLU | E | 392 | 10.318 | 85.713 | 4.248 | 1.00 | 82.26 | E |
| ATOM | 3279 | OE2 | GLU | E | 392 | 9.733 | 85.551 | 2.117 | 1.00 | 83.41 | E |
| ATOM | 3280 | C | GLU | E | 392 | 11.292 | 82.029 | 2.942 | 1.00 | 37.32 | E |
| ATOM | 3281 | O | GLU | E | 392 | 10.222 | 82.099 | 2.357 | 1.00 | 40.36 | E |
| ATOM | 3282 | N | THR | E | 393 | 12.198 | 81.090 | 2.676 | 1.00 | 34.79 | E |
| ATOM | 3283 | CA | THR | E | 393 | 11.963 | 80.045 | 1.683 | 1.00 | 31.23 | E |
| ATOM | 3284 | CB | THR | E | 393 | 13.169 | 79.075 | 1.591 | 1.00 | 31.08 | E |
| ATOM | 3285 | OG1 | THR | E | 393 | 14.331 | 79.765 | 1.116 | 1.00 | 27.30 | E |
| ATOM | 3286 | CG2 | THR | E | 393 | 12.856 | 77.922 | 0.647 | 1.00 | 27.02 | E |
| ATOM | 3287 | C | THR | E | 393 | 10.711 | 79.220 | 2.074 | 1.00 | 32.60 | E |
| ATOM | 3288 | O | THR | E | 393 | 9.917 | 78.822 | 1.235 | 1.00 | 36.48 | E |
| ATOM | 3289 | N | CYS | E | 394 | 10.527 | 78.956 | 3.355 | 1.00 | 34.75 | E |
| ATOM | 3290 | CA | CYS | E | 394 | 9.360 | 78.182 | 3.769 | 1.00 | 37.83 | E |
| ATOM | 3291 | CB | CYS | E | 394 | 9.454 | 77.808 | 5.240 | 1.00 | 30.63 | E |
| ATOM | 3292 | SG | CYS | E | 394 | 10.513 | 76.386 | 5.489 | 1.00 | 41.29 | E |
| ATOM | 3293 | C | CYS | E | 394 | 8.066 | 78.908 | 3.519 | 1.00 | 38.54 | E |
| ATOM | 3294 | O | CYS | E | 394 | 7.069 | 78.292 | 3.163 | 1.00 | 35.21 | E |
| ATOM | 3295 | N | PHE | E | 395 | 8.087 | 80.223 | 3.711 | 1.00 | 40.13 | E |
| ATOM | 3296 | CA | PHE | E | 395 | 6.903 | 81.044 | 3.505 | 1.00 | 37.82 | E |
| ATOM | 3297 | CB | PHE | E | 395 | 7.205 | 82.518 | 3.772 | 1.00 | 29.50 | E |
| ATOM | 3298 | CG | PHE | E | 395 | 6.025 | 83.420 | 3.567 | 1.00 | 29.76 | E |
| ATOM | 3299 | CD1 | PHE | E | 395 | 4.968 | 83.421 | 4.483 | 1.00 | 31.00 | E |
| ATOM | 3300 | CD2 | PHE | E | 395 | 5.938 | 84.232 | 2.433 | 1.00 | 28.66 | E |
| ATOM | 3301 | CE1 | PHE | E | 395 | 3.834 | 84.215 | 4.272 | 1.00 | 30.82 | E |
| ATOM | 3302 | CE2 | PHE | E | 395 | 4.804 | 85.032 | 2.209 | 1.00 | 26.06 | E |
| ATOM | 3303 | CZ | PHE | E | 395 | 3.754 | 85.022 | 3.130 | 1.00 | 27.11 | E |
| ATOM | 3304 | C | PHE | E | 395 | 6.459 | 80.914 | 2.069 | 1.00 | 39.43 | E |
| ATOM | 3305 | O | PHE | E | 395 | 5.274 | 80.746 | 1.785 | 1.00 | 39.90 | E |
| ATOM | 3306 | N | SER | E | 396 | 7.437 | 80.997 | 1.174 | 1.00 | 34.64 | E |
| ATOM | 3307 | CA | SER | E | 396 | 7.205 | 80.937 | −0.249 | 1.00 | 36.33 | E |
| ATOM | 3308 | CB | SER | E | 396 | 8.474 | 81.306 | −0.970 | 1.00 | 32.84 | E |
| ATOM | 3309 | OG | SER | E | 396 | 8.784 | 82.633 | −0.581 | 1.00 | 51.02 | E |
| ATOM | 3310 | C | SER | E | 396 | 6.683 | 79.626 | −0.743 | 1.00 | 39.02 | E |
| ATOM | 3311 | O | SER | E | 396 | 5.778 | 79.600 | −1.572 | 1.00 | 42.24 | E |
| ATOM | 3312 | N | LEU | E | 397 | 7.246 | 78.531 | −0.247 | 1.00 | 43.94 | E |
| ATOM | 3313 | CA | LEU | E | 397 | 6.775 | 77.214 | −0.643 | 1.00 | 42.57 | E |
| ATOM | 3314 | CB | LEU | E | 397 | 7.629 | 76.123 | 0.000 | 1.00 | 37.99 | E |
| ATOM | 3315 | CG | LEU | E | 397 | 9.079 | 76.179 | −0.432 | 1.00 | 31.14 | E |
| ATOM | 3316 | CD1 | LEU | E | 397 | 9.839 | 75.035 | 0.159 | 1.00 | 35.00 | E |
| ATOM | 3317 | CD2 | LEU | E | 397 | 9.131 | 76.124 | −1.929 | 1.00 | 40.62 | E |
| ATOM | 3318 | C | LEU | E | 397 | 5.330 | 77.103 | −0.173 | 1.00 | 41.49 | E |
| ATOM | 3319 | O | LEU | E | 397 | 4.520 | 76.450 | −0.799 | 1.00 | 49.39 | E |
| ATOM | 3320 | N | LYS | E | 398 | 5.013 | 77.767 | 0.928 | 1.00 | 37.15 | E |
| ATOM | 3321 | CA | LYS | E | 398 | 3.675 | 77.742 | 1.473 | 1.00 | 39.00 | E |
| ATOM | 3322 | CB | LYS | E | 398 | 3.724 | 78.275 | 2.904 | 1.00 | 43.96 | E |
| ATOM | 3323 | CG | LYS | E | 398 | 2.869 | 77.504 | 3.889 | 1.00 | 52.32 | E |
| ATOM | 3324 | CD | LYS | E | 398 | 3.114 | 75.996 | 3.792 | 1.00 | 59.84 | E |
| ATOM | 3325 | CE | LYS | E | 398 | 1.853 | 75.225 | 4.218 | 1.00 | 65.06 | E |
| ATOM | 3326 | NZ | LYS | E | 398 | 1.812 | 73.819 | 3.706 | 1.00 | 68.83 | E |
| ATOM | 3327 | C | LYS | E | 398 | 2.709 | 78.571 | 0.613 | 1.00 | 44.04 | E |
| ATOM | 3328 | O | LYS | E | 398 | 1.513 | 78.308 | 0.579 | 1.00 | 47.81 | E |
| ATOM | 3329 | N | LEU | E | 399 | 3.244 | 79.569 | −0.084 | 1.00 | 50.06 | E |
| ATOM | 3330 | CA | LEU | E | 399 | 2.476 | 80.464 | −0.947 | 1.00 | 42.86 | E |
| ATOM | 3331 | CB | LEU | E | 399 | 3.303 | 81.714 | −1.247 | 1.00 | 43.06 | E |
| ATOM | 3332 | CG | LEU | E | 399 | 2.649 | 82.840 | −2.039 | 1.00 | 42.04 | E |
| ATOM | 3333 | CD1 | LEU | E | 399 | 1.534 | 83.421 | −1.201 | 1.00 | 37.94 | E |
| ATOM | 3334 | CD2 | LEU | E | 399 | 3.653 | 83.902 | −2.378 | 1.00 | 24.82 | E |
| ATOM | 3335 | C | LEU | E | 399 | 2.181 | 79.749 | −2.246 | 1.00 | 43.34 | E |
| ATOM | 3336 | O | LEU | E | 399 | 1.043 | 79.720 | −2.717 | 1.00 | 48.77 | E |
| ATOM | 3337 | N | LYS | E | 400 | 3.240 | 79.194 | −2.826 | 1.00 | 45.96 | E |
| ATOM | 3338 | CA | LYS | E | 400 | 3.174 | 78.444 | −4.078 | 1.00 | 49.43 | E |
| ATOM | 3339 | CB | LYS | E | 400 | 4.580 | 78.113 | −4.567 | 1.00 | 47.72 | E |
| ATOM | 3340 | CG | LYS | E | 400 | 5.514 | 79.302 | −4.753 | 1.00 | 47.05 | E |
| ATOM | 3341 | CD | LYS | E | 400 | 6.930 | 78.752 | −4.789 | 1.00 | 55.17 | E |
| ATOM | 3342 | CE | LYS | E | 400 | 8.009 | 79.787 | −5.015 | 1.00 | 51.25 | E |
| ATOM | 3343 | NZ | LYS | E | 400 | 9.325 | 79.087 | −4.846 | 1.00 | 53.55 | E |
| ATOM | 3344 | C | LYS | E | 400 | 2.451 | 77.140 | −3.797 | 1.00 | 49.29 | E |
| ATOM | 3345 | O | LYS | E | 400 | 2.167 | 76.359 | −4.698 | 1.00 | 48.52 | E |
| ATOM | 3346 | N | ASN | E | 401 | 2.159 | 76.926 | −2.523 | 1.00 | 49.33 | E |
| ATOM | 3347 | CA | ASN | E | 401 | 1.494 | 75.722 | −2.051 | 1.00 | 51.53 | E |
| ATOM | 3348 | CB | ASN | E | 401 | 0.045 | 75.690 | −2.502 | 1.00 | 53.59 | E |
| ATOM | 3349 | CG | ASN | E | 401 | −0.762 | 74.657 | −1.740 | 1.00 | 60.06 | E |
| ATOM | 3350 | OD1 | ASN | E | 401 | −1.768 | 74.151 | −2.237 | 1.00 | 56.19 | E |
| ATOM | 3351 | ND2 | ASN | E | 401 | −0.326 | 74.342 | −0.512 | 1.00 | 62.85 | E |
| ATOM | 3352 | C | ASN | E | 401 | 2.187 | 74.426 | −2.497 | 1.00 | 50.38 | E |
| ATOM | 3353 | O | ASN | E | 401 | 1.641 | 73.654 | −3.279 | 1.00 | 47.98 | E |
| ATOM | 3354 | N | ARG | E | 402 | 3.395 | 74.201 | −1.989 | 1.00 | 49.15 | E |
| ATOM | 3355 | CA | ARG | E | 402 | 4.169 | 73.017 | −2.301 | 1.00 | 50.62 | E |
| ATOM | 3356 | CB | ARG | E | 402 | 5.515 | 73.416 | −2.866 | 1.00 | 56.48 | E |
| ATOM | 3357 | CG | ARG | E | 402 | 5.397 | 74.361 | −4.038 | 1.00 | 64.09 | E |
| ATOM | 3358 | CD | ARG | E | 402 | 6.740 | 74.500 | −4.694 | 1.00 | 71.64 | E |
| ATOM | 3359 | NE | ARG | E | 402 | 7.126 | 73.261 | −5.359 | 1.00 | 72.37 | E |
| ATOM | 3360 | CZ | ARG | E | 402 | 8.352 | 73.003 | −5.806 | 1.00 | 73.62 | E |
| ATOM | 3361 | NH1 | ARG | E | 402 | 9.320 | 73.904 | −5.649 | 1.00 | 65.04 | E |
| ATOM | 3362 | NH2 | ARG | E | 402 | 8.601 | 71.849 | −6.424 | 1.00 | 73.00 | E |
| ATOM | 3363 | C | ARG | E | 402 | 4.334 | 72.356 | −0.965 | 1.00 | 53.42 | E |
| ATOM | 3364 | O | ARG | E | 402 | 4.430 | 73.057 | 0.040 | 1.00 | 57.21 | E |
| ATOM | 3365 | N | LYS | E | 403 | 4.362 | 71.020 | −0.951 | 1.00 | 54.79 | E |
| ATOM | 3366 | CA | LYS | E | 403 | 4.461 | 70.238 | 0.293 | 1.00 | 52.75 | E |
| ATOM | 3367 | CB | LYS | E | 403 | 4.269 | 68.743 | 0.006 | 1.00 | 53.56 | E |
| ATOM | 3368 | CG | LYS | E | 403 | 2.871 | 68.363 | −0.456 | 1.00 | 68.79 | E |
| ATOM | 3369 | CD | LYS | E | 403 | 2.804 | 66.872 | −0.808 | 1.00 | 77.87 | E |
| ATOM | 3370 | CE | LYS | E | 403 | 1.437 | 66.457 | −1.360 | 1.00 | 79.58 | E |
| ATOM | 3371 | NZ | LYS | E | 403 | 1.277 | 64.966 | −1.360 | 1.00 | 81.94 | E |
| ATOM | 3372 | C | LYS | E | 403 | 5.718 | 70.399 | 1.142 | 1.00 | 49.96 | E |
| ATOM | 3373 | O | LYS | E | 403 | 6.812 | 69.931 | 0.764 | 1.00 | 46.03 | E |
| ATOM | 3374 | N | VAL | E | 404 | 5.556 | 71.058 | 2.291 | 1.00 | 38.89 | E |
| ATOM | 3375 | CA | VAL | E | 404 | 6.672 | 71.223 | 3.205 | 1.00 | 40.29 | E |
| ATOM | 3376 | CB | VAL | E | 404 | 6.693 | 72.581 | 3.922 | 1.00 | 41.80 | E |
| ATOM | 3377 | CG1 | VAL | E | 404 | 8.022 | 72.711 | 4.686 | 1.00 | 24.81 | E |
| ATOM | 3378 | CG2 | VAL | E | 404 | 6.448 | 73.735 | 2.944 | 1.00 | 39.16 | E |
| ATOM | 3379 | C | VAL | E | 404 | 6.427 | 70.189 | 4.293 | 1.00 | 48.27 | E |
| ATOM | 3380 | O | VAL | E | 404 | 5.442 | 70.290 | 5.020 | 1.00 | 55.74 | E |
| ATOM | 3381 | N | PRO | E | 405 | 7.325 | 69.197 | 4.440 | 1.00 | 45.50 | E |
| ATOM | 3382 | CD | PRO | E | 405 | 8.679 | 69.161 | 3.858 | 1.00 | 42.67 | E |
| ATOM | 3383 | CA | PRO | E | 405 | 7.173 | 68.154 | 5.463 | 1.00 | 40.57 | E |
| ATOM | 3384 | CB | PRO | E | 405 | 8.572 | 67.580 | 5.571 | 1.00 | 41.08 | E |
| ATOM | 3385 | CG | PRO | E | 405 | 9.123 | 67.780 | 4.203 | 1.00 | 40.01 | E |
| ATOM | 3386 | C | PRO | E | 405 | 6.698 | 68.714 | 6.803 | 1.00 | 45.93 | E |
| ATOM | 3387 | O | PRO | E | 405 | 7.339 | 69.593 | 7.386 | 1.00 | 46.37 | E |
| ATOM | 3388 | N | SER | E | 406 | 5.586 | 68.195 | 7.304 | 1.00 | 52.64 | E |
| ATOM | 3389 | CA | SER | E | 406 | 5.045 | 68.680 | 8.568 | 1.00 | 57.41 | E |
| ATOM | 3390 | CB | SER | E | 406 | 3.901 | 67.787 | 9.032 | 1.00 | 56.58 | E |
| ATOM | 3391 | OG | SER | E | 406 | 3.121 | 68.480 | 9.988 | 1.00 | 67.22 | E |
| ATOM | 3392 | C | SER | E | 406 | 6.085 | 68.814 | 9.693 | 1.00 | 59.81 | E |
| ATOM | 3393 | O | SER | E | 406 | 6.068 | 69.798 | 10.438 | 1.00 | 63.18 | E |
| ATOM | 3394 | N | PHE | E | 407 | 6.983 | 67.837 | 9.819 | 1.00 | 57.14 | E |
| ATOM | 3395 | CA | PHE | E | 407 | 8.013 | 67.877 | 10.853 | 1.00 | 51.13 | E |
| ATOM | 3396 | CB | PHE | E | 407 | 9.027 | 66.751 | 10.625 | 1.00 | 48.43 | E |
| ATOM | 3397 | CG | PHE | E | 407 | 10.186 | 66.743 | 11.610 | 1.00 | 50.11 | E |
| ATOM | 3398 | CD1 | PHE | E | 407 | 9.961 | 66.780 | 12.990 | 1.00 | 47.64 | E |
| ATOM | 3399 | CD2 | PHE | E | 407 | 11.501 | 66.629 | 11.156 | 1.00 | 49.90 | E |
| ATOM | 3400 | CE1 | PHE | E | 407 | 11.019 | 66.693 | 13.891 | 1.00 | 37.99 | E |
| ATOM | 3401 | CE2 | PHE | E | 407 | 12.559 | 66.544 | 12.052 | 1.00 | 52.16 | E |
| ATOM | 3402 | CZ | PHE | E | 407 | 12.314 | 66.574 | 13.425 | 1.00 | 44.66 | E |
| ATOM | 3403 | C | PHE | E | 407 | 8.722 | 69.225 | 10.820 | 1.00 | 49.58 | E |
| ATOM | 3404 | O | PHE | E | 407 | 8.924 | 69.855 | 11.868 | 1.00 | 48.50 | E |
| ATOM | 3405 | N | LEU | E | 408 | 9.087 | 69.650 | 9.606 | 1.00 | 46.20 | E |
| ATOM | 3406 | CA | LEU | E | 408 | 9.788 | 70.907 | 9.376 | 1.00 | 46.76 | E |
| ATOM | 3407 | CB | LEU | E | 408 | 10.269 | 70.977 | 7.928 | 1.00 | 40.00 | E |
| ATOM | 3408 | CG | LEU | E | 408 | 11.276 | 69.905 | 7.527 | 1.00 | 38.59 | E |
| ATOM | 3409 | CD1 | LEU | E | 408 | 11.706 | 70.112 | 6.089 | 1.00 | 40.41 | E |
| ATOM | 3410 | CD2 | LEU | E | 408 | 12.468 | 69.961 | 8.439 | 1.00 | 37.21 | E |
| ATOM | 3411 | C | LEU | E | 408 | 8.920 | 72.125 | 9.701 | 1.00 | 52.01 | E |
| ATOM | 3412 | O | LEU | E | 408 | 9.408 | 73.132 | 10.233 | 1.00 | 55.68 | E |
| ATOM | 3413 | N | GLU | E | 409 | 7.634 | 72.043 | 9.382 | 1.00 | 49.74 | E |
| ATOM | 3414 | CA | GLU | E | 409 | 6.746 | 73.145 | 9.684 | 1.00 | 50.76 | E |
| ATOM | 3415 | CB | GLU | E | 409 | 5.387 | 72.879 | 9.039 | 1.00 | 52.54 | E |
| ATOM | 3416 | CG | GLU | E | 409 | 5.512 | 72.621 | 7.526 | 1.00 | 65.10 | E |
| ATOM | 3417 | CD | GLU | E | 409 | 4.191 | 72.709 | 6.761 | 1.00 | 69.80 | E |
| ATOM | 3418 | OE1 | GLU | E | 409 | 3.226 | 71.997 | 7.120 | 1.00 | 74.10 | E |
| ATOM | 3419 | OE2 | GLU | E | 409 | 4.122 | 73.489 | 5.788 | 1.00 | 71.29 | E |
| ATOM | 3420 | C | GLU | E | 409 | 6.663 | 73.266 | 11.214 | 1.00 | 51.62 | E |
| ATOM | 3421 | O | GLU | E | 409 | 6.819 | 74.348 | 11.775 | 1.00 | 47.23 | E |
| ATOM | 3422 | N | GLU | E | 410 | 6.465 | 72.130 | 11.876 | 1.00 | 53.40 | E |
| ATOM | 3423 | CA | GLU | E | 410 | 6.371 | 72.044 | 13.335 | 1.00 | 52.41 | E |
| ATOM | 3424 | CB | GLU | E | 410 | 6.220 | 70.583 | 13.768 | 1.00 | 56.30 | E |
| ATOM | 3425 | CG | GLU | E | 410 | 5.008 | 69.847 | 13.207 | 1.00 | 63.66 | E |
| ATOM | 3426 | CD | GLU | E | 410 | 5.119 | 68.337 | 13.360 | 1.00 | 65.12 | E |
| ATOM | 3427 | OE1 | GLU | E | 410 | 4.133 | 67.627 | 13.076 | 1.00 | 57.01 | E |
| ATOM | 3428 | OE2 | GLU | E | 410 | 6.202 | 67.856 | 13.755 | 1.00 | 75.09 | E |
| ATOM | 3429 | C | GLU | E | 410 | 7.591 | 72.599 | 14.060 | 1.00 | 50.63 | E |
| ATOM | 3430 | O | GLU | E | 410 | 7.465 | 73.224 | 15.109 | 1.00 | 51.62 | E |
| ATOM | 3431 | N | ILE | E | 411 | 8.774 | 72.351 | 13.508 | 1.00 | 44.17 | E |
| ATOM | 3432 | CA | ILE | E | 411 | 10.016 | 72.788 | 14.147 | 1.00 | 42.52 | E |
| ATOM | 3433 | CB | ILE | E | 411 | 11.178 | 71.838 | 13.803 | 1.00 | 29.79 | E |
| ATOM | 3434 | CG2 | ILE | E | 411 | 12.472 | 72.388 | 14.324 | 1.00 | 19.62 | E |
| ATOM | 3435 | CG1 | ILE | E | 411 | 10.885 | 70.464 | 14.389 | 1.00 | 26.63 | E |
| ATOM | 3436 | CD1 | ILE | E | 411 | 12.014 | 69.562 | 14.352 | 1.00 | 32.83 | E |
| ATOM | 3437 | C | ILE | E | 411 | 10.455 | 74.198 | 13.838 | 1.00 | 47.45 | E |
| ATOM | 3438 | O | ILE | E | 411 | 10.963 | 74.908 | 14.710 | 1.00 | 45.98 | E |
| ATOM | 3439 | N | TRP | E | 412 | 10.266 | 74.596 | 12.587 | 1.00 | 50.84 | E |
| ATOM | 3440 | CA | TRP | E | 412 | 10.638 | 75.928 | 12.158 | 1.00 | 47.10 | E |
| ATOM | 3441 | CB | TRP | E | 412 | 11.106 | 75.897 | 10.714 | 1.00 | 31.01 | E |
| ATOM | 3442 | CG | TRP | E | 412 | 12.357 | 75.111 | 10.520 | 1.00 | 29.88 | E |
| ATOM | 3443 | CD2 | TRP | E | 412 | 12.802 | 74.514 | 9.306 | 1.00 | 30.53 | E |
| ATOM | 3444 | CE2 | TRP | E | 412 | 14.074 | 73.964 | 9.554 | 1.00 | 29.64 | E |
| ATOM | 3445 | CE3 | TRP | E | 412 | 12.242 | 74.378 | 8.030 | 1.00 | 31.08 | E |
| ATOM | 3446 | CD1 | TRP | E | 412 | 13.356 | 74.909 | 11.431 | 1.00 | 29.83 | E |
| ATOM | 3447 | NE1 | TRP | E | 412 | 14.391 | 74.226 | 10.858 | 1.00 | 26.29 | E |
| ATOM | 3448 | CZ2 | TRP | E | 412 | 14.804 | 73.304 | 8.568 | 1.00 | 30.77 | E |
| ATOM | 3449 | CZ3 | TRP | E | 412 | 12.972 | 73.721 | 7.051 | 1.00 | 33.89 | E |
| ATOM | 3450 | CH2 | TRP | E | 412 | 14.235 | 73.184 | 7.328 | 1.00 | 34.12 | E |
| ATOM | 3451 | C | TRP | E | 412 | 9.470 | 76.872 | 12.287 | 1.00 | 53.01 | E |
| ATOM | 3452 | O | TRP | E | 412 | 9.598 | 78.044 | 11.975 | 1.00 | 59.24 | E |
| ATOM | 3453 | N | ASP | E | 413 | 8.334 | 76.356 | 12.749 | 1.00 | 60.21 | E |
| ATOM | 3454 | CA | ASP | E | 413 | 7.127 | 77.155 | 12.904 | 1.00 | 65.98 | E |
| ATOM | 3455 | CB | ASP | E | 413 | 7.357 | 78.308 | 13.886 | 1.00 | 65.12 | E |
| ATOM | 3456 | CG | ASP | E | 413 | 7.976 | 77.848 | 15.206 | 1.00 | 73.02 | E |
| ATOM | 3457 | OD1 | ASP | E | 413 | 7.517 | 76.827 | 15.778 | 1.00 | 69.22 | E |
| ATOM | 3458 | OD2 | ASP | E | 413 | 8.918 | 78.522 | 15.682 | 1.00 | 71.14 | E |
| ATOM | 3459 | C | ASP | E | 413 | 6.800 | 77.709 | 11.529 | 1.00 | 73.27 | E |
| ATOM | 3460 | O | ASP | E | 413 | 6.734 | 78.919 | 11.332 | 1.00 | 79.88 | E |
| ATOM | 3461 | N | VAL | E | 414 | 6.617 | 76.810 | 10.572 | 1.00 | 78.65 | E |
| ATOM | 3462 | CA | VAL | E | 414 | 6.313 | 77.195 | 9.201 | 1.00 | 83.57 | E |
| ATOM | 3463 | CB | VAL | E | 414 | 7.148 | 76.388 | 8.201 | 1.00 | 81.07 | E |
| ATOM | 3464 | CG1 | VAL | E | 414 | 6.874 | 76.876 | 6.782 | 1.00 | 78.91 | E |
| ATOM | 3465 | CG2 | VAL | E | 414 | 8.605 | 76.474 | 8.565 | 1.00 | 83.03 | E |
| ATOM | 3466 | C | VAL | E | 414 | 4.861 | 76.936 | 8.841 | 1.00 | 91.08 | E |
| ATOM | 3467 | O | VAL | E | 414 | 4.515 | 75.832 | 8.421 | 1.00 | 98.61 | E |
| ATOM | 3468 | N | VAL | E | 415 | 3.998 | 77.924 | 9.002 | 1.00 | 93.34 | E |
| ATOM | 3469 | CA | VAL | E | 415 | 2.611 | 77.707 | 8.614 | 1.00 | 94.81 | E |
| ATOM | 3470 | CB | VAL | E | 415 | 1.706 | 77.355 | 9.848 | 1.00 | 90.47 | E |
| ATOM | 3471 | CG1 | VAL | E | 415 | 1.624 | 75.834 | 10.020 | 1.00 | 78.74 | E |
| ATOM | 3472 | CG2 | VAL | E | 415 | 2.269 | 77.985 | 11.119 | 1.00 | 82.74 | E |
| ATOM | 3473 | C | VAL | E | 415 | 2.101 | 78.944 | 7.876 | 1.00 | 97.73 | E |
| ATOM | 3474 | O | VAL | E | 415 | 2.139 | 78.927 | 6.624 | 1.00 | 97.64 | E |
| ATOM | 3475 | OXT | VAL | E | 415 | 1.716 | 79.926 | 8.541 | 1.00 | 98.46 | E |
| ATOM | 3476 | O1 | PON | A | 1 | 21.880 | 64.675 | 1.836 | 1.00 | 39.29 | A |
| ATOM | 3477 | O2 | PON | A | 1 | 19.250 | 68.890 | 4.074 | 1.00 | 49.48 | A |
| ATOM | 3478 | O3 | PON | A | 1 | 24.588 | 66.480 | 7.635 | 1.00 | 41.10 | A |
| ATOM | 3479 | O4 | PON | A | 1 | 24.235 | 64.175 | 5.968 | 1.00 | 45.83 | A |
| ATOM | 3480 | O6 | PON | A | 1 | 18.231 | 74.846 | 1.917 | 1.00 | 33.99 | A |
| ATOM | 3481 | O7 | PON | A | 1 | 20.248 | 73.129 | 1.273 | 1.00 | 33.86 | A |
| ATOM | 3482 | C1 | PON | A | 1 | 24.365 | 67.114 | 5.289 | 1.00 | 35.43 | A |
| ATOM | 3483 | C2 | PON | A | 1 | 23.687 | 66.508 | 6.523 | 1.00 | 38.34 | A |
| ATOM | 3484 | C3 | PON | A | 1 | 23.150 | 65.064 | 6.265 | 1.00 | 36.40 | A |
| ATOM | 3485 | C4 | PON | A | 1 | 22.176 | 65.084 | 5.024 | 1.00 | 33.74 | A |
| ATOM | 3486 | C5 | PON | A | 1 | 22.927 | 65.679 | 3.758 | 1.00 | 31.18 | A |
| ATOM | 3487 | C6 | PON | A | 1 | 21.955 | 65.657 | 2.563 | 1.00 | 33.89 | A |
| ATOM | 3488 | C7 | PON | A | 1 | 21.097 | 66.795 | 2.300 | 1.00 | 28.82 | A |
| ATOM | 3489 | C8 | PON | A | 1 | 21.130 | 67.924 | 3.082 | 1.00 | 37.62 | A |
| ATOM | 3490 | C9 | PON | A | 1 | 22.125 | 68.074 | 4.307 | 1.00 | 31.36 | A |
| ATOM | 3491 | C10 | PON | A | 1 | 23.441 | 67.156 | 4.046 | 1.00 | 29.60 | A |
| ATOM | 3492 | C11 | PON | A | 1 | 22.560 | 69.547 | 4.592 | 1.00 | 32.32 | A |
| ATOM | 3493 | C12 | PON | A | 1 | 21.489 | 70.626 | 4.357 | 1.00 | 38.09 | A |
| ATOM | 3494 | C13 | PON | A | 1 | 20.762 | 70.529 | 2.973 | 1.00 | 36.47 | A |
| ATOM | 3495 | C14 | PON | A | 1 | 20.134 | 69.048 | 2.915 | 1.00 | 39.30 | A |
| ATOM | 3496 | C15 | PON | A | 1 | 19.155 | 69.066 | 1.737 | 1.00 | 40.89 | A |
| ATOM | 3497 | C16 | PON | A | 1 | 18.747 | 70.529 | 1.546 | 1.00 | 39.22 | A |
| ATOM | 3498 | C17 | PON | A | 1 | 19.416 | 71.300 | 2.743 | 1.00 | 35.47 | A |
| ATOM | 3499 | C18 | PON | A | 1 | 21.769 | 70.748 | 1.787 | 1.00 | 32.19 | A |
| ATOM | 3500 | C19 | PON | A | 1 | 24.255 | 67.664 | 2.827 | 1.00 | 24.46 | A |
| ATOM | 3501 | C20 | PON | A | 1 | 19.466 | 72.863 | 2.472 | 1.00 | 35.95 | A |
| ATOM | 3502 | C21 | PON | A | 1 | 20.120 | 73.624 | 3.669 | 1.00 | 36.02 | A |
| ATOM | 3503 | C22 | PON | A | 1 | 18.054 | 73.441 | 2.175 | 1.00 | 32.10 | A |
| ATOM | 3504 | C23 | PON | A | 1 | 16.966 | 73.306 | 3.300 | 1.00 | 27.82 | A |
| ATOM | 3505 | C24 | PON | A | 1 | 15.596 | 73.682 | 2.708 | 1.00 | 19.20 | A |
| ATOM | 3506 | C25 | PON | A | 1 | 14.375 | 73.327 | 3.427 | 1.00 | 28.59 | A |
| ATOM | 3507 | C26 | PON | A | 1 | 13.612 | 72.211 | 2.623 | 1.00 | 27.63 | A |
| ATOM | 3508 | C27 | PON | A | 1 | 13.497 | 74.515 | 3.656 | 1.00 | 26.34 | A |
| ATOM | 3509 | P | PO4 | | 1 | 20.235 | 86.852 | 20.495 | 1.00 | 30.27 | |
| ATOM | 3510 | O1 | PO4 | | 1 | 20.367 | 87.795 | 21.734 | 1.00 | 30.23 | |
| ATOM | 3511 | O2 | PO4 | | 1 | 21.496 | 86.086 | 20.199 | 1.00 | 24.49 | |
| ATOM | 3512 | O3 | PO4 | | 1 | 19.795 | 87.730 | 19.291 | 1.00 | 36.16 | |
| ATOM | 3513 | O4 | PO4 | | 1 | 19.068 | 85.895 | 20.750 | 1.00 | 32.97 | |
| ATOM | 3514 | P | PO4 | | 2 | 39.059 | 93.718 | 35.395 | 1.00 | 78.13 | |
| ATOM | 3515 | O1 | PO4 | | 2 | 40.205 | 94.770 | 35.302 | 1.00 | 80.90 | |
| ATOM | 3516 | O2 | PO4 | | 2 | 39.127 | 92.675 | 34.321 | 1.00 | 73.64 | |
| ATOM | 3517 | O3 | PO4 | | 2 | 37.709 | 94.500 | 35.366 | 1.00 | 80.87 | |
| ATOM | 3518 | O4 | PO4 | | 2 | 39.152 | 93.072 | 36.788 | 1.00 | 87.72 | |
| ATOM | 3519 | P | PO4 | | 3 | 15.547 | 99.875 | 0.242 | 1.00 | 66.29 | |
| ATOM | 3520 | O1 | PO4 | | 3 | 14.096 | 99.637 | −0.237 | 1.00 | 69.21 | |
| ATOM | 3521 | O2 | PO4 | | 3 | 16.172 | 98.596 | 0.756 | 1.00 | 58.29 | |
| ATOM | 3522 | O3 | PO4 | | 3 | 15.467 | 101.019 | 1.344 | 1.00 | 49.78 | |
| ATOM | 3523 | O4 | PO4 | | 3 | 16.339 | 100.404 | −1.015 | 1.00 | 64.10 | |
| ATOM | 3524 | P | PO4 | | 4 | 20.453 | 67.865 | −11.707 | 1.00 | 75.67 | |
| ATOM | 3525 | O1 | PO4 | | 4 | 20.348 | 66.382 | −12.180 | 1.00 | 76.23 | |
| ATOM | 3526 | O2 | PO4 | | 4 | 20.722 | 67.960 | −10.231 | 1.00 | 79.04 | |
| ATOM | 3527 | O3 | PO4 | | 4 | 19.108 | 68.571 | −12.112 | 1.00 | 66.80 | |
| ATOM | 3528 | O4 | PO4 | | 4 | 21.613 | 68.530 | −12.504 | 1.00 | 62.79 | |
| END | | | | | | | | | | | |

1. A crystalline composition comprising the BtEcR/BtUSP heterodimer ligand binding domain (LBD) or portion thereof of the ecdysone receptor from Bemisia tabaci.

2. A crystalline composition according to claim 1, wherein the LBD or portion thereof is co-crystallized with a ligand.

3. A method of selecting or designing a compound that interacts with an ecdysone receptor and modulates an activity mediated by the receptor, the method comprising the step of assessing the stereochemical complementarity between the compound and a topographic region of the BtEcR/BtUSP heterodimer LBD, wherein the heterodimer is characterised by:
(a) amino acids 179-415 of the BtEcR monomer and amino acids 300-492 of the BtUSP monomer positioned at atomic coordinates as shown in Appendix I, or structural coordinates wherein the backbone atoms of each monomer has a root mean square deviation from the backbone atoms of their corresponding partners in either amino acids 179-415 of the BtEcR monomer or amino acids 300-492 of the BtUSP monomer of not more than 1.5 Å; or
(b) one or more subsets of said amino acids related to the coordinates of the monomers shown in Appendix I by whole body translations and/or rotations.

4. The method according to claim 3, wherein the structural coordinates have a root mean square deviation from the backbone atoms of said amino acids of not more than 1.0 Å.

5. The method according to claim 3, wherein the structural coordinates have a root mean square deviation from the backbone atoms of said amino acids of not more than 0.7 Å.

6. The method according to claim 3, wherein the topographic region of the ecdysone receptor to which the compound, or a portion thereof has stereochemical complementarity is the ligand-binding pocket of the BtEcR subunit defined by amino acids F194, Q195, N196, Y198, E199, H200, P201, H226, I227, T228,1230, T231, L233, T234, L237, I238, F241, S242, V267, M268, M269, F270, R271, M272, R274, R275, I283, L284, F285, A286, Y296, M301, T304, L308, Y325, A326, T329, I333, M389, N390, T393, C394, L397, V404, P405, L408 and W412.

7. The method according to claim 3, wherein the topographic region of the ecdysone receptor to which the compound, or a portion thereof has stereochemical complementarity is the interface between the BtEcR and BtUSP subunits defined by BtEcR residues H314, M315, I331, S335, E336, R337, P338, E347, Q350, E351, I354, E355, K358, T370, T371, F373, A374, K375, L377, S378, L380, T381, E382, R384, T385 and N388 and BtUSP residues E342, R383, T386, E387, K391, E414, E425, E429, Y432, A433, E436, S447, G448, F450, A451, K452, L454, L455, R456, L457, P458, A459, R461, S462 and L465.

8. The method according to claim 3, wherein the topographic region of the ecdysone receptor to which the compound, or portion thereof has stereochemical complementarity is the co-activator/co-repressor binding groove formed by helices H3 and H4 of the surface of BtEcR defined by residues I232, V235, Q236, V239, E240, K243, F248, R253, E254, Q256, I257, L260, K261, S264, S265, M268, S406, F407, L408, E410, I411 and D413.

9. (canceled)

10. The method according to claim 6, wherein the method comprises selecting a compound which forms hydrogen bonds with at least one amino acid residue selected from the group consisting of E199, I227, T231, T234, R271, A286 Y296, T304, N390 and C394 of the ligand-binding pocket of the BtEcR LBD, wherein the compound is not a naturally-occurring ecdysteroid ligand of the ligand-binding pocket of the receptor.

11. The method according to claim 6, wherein the method comprises selecting a compound which forms hydrophobic contacts with the side chains of at least one amino acid residue selected from the group consisting of P201, I227, T228, I230, M268, M269, R271, M272, R275, I283, F285, A286, M301, L308, M389, L397, P405, L408 and W412 of the ligand-binding pocket of the BtEcR subunit, wherein the compound is not the natural ligand of the ligand-binding pocket of the receptor.

12. The method according to claim 3, wherein the compound is selected or designed to interact with the B. tabaci ecdysone receptor in a manner such as to interfere with the association of the BtEcR and BtUSP subunits by inhibiting the association of BtEcR residues H314, M315, I331, S335, E336, R337, P338, E347, Q350, E351, I354, E355, K358, T370, T371, F373, A374, K375, L377, S378, V379, L380, T381, E382, R384, T385 and N388 and BtUSP residues E342, R383, T386, E387, K391, I408, V409, E414, E425, R428, E429, Y432, A433, E436, S447, G448, F450, A451, K452, L454, L455, R456, L457, A459, R461, S462 and L465.

13. The method according to claim 3, wherein the compound is selected or designed to interfere with signalling of the receptor.

14. The method according to claim 3, wherein the compound is selected or designed based on the natural ligand of the B. tabaci ecdysone receptor, the compound being designed or selected such that it interacts with at least one amino acid selected from the group consisting of F194, Q195, N196, Y198, E199, H200, P201, H226, I227, T228, I230, T231, L233, T234, L237, I238, F241, S242, V267, M268, M269, F270, R271, M272, R274, R275, I283, L284, F285, A286, Y296, M301, T304, L308, Y325, A326, T329, I333, M389, N390, T393, C394, L397, V404, P405, L408 and W412.

15. The method according to claim 14, wherein the compound is selected or designed such that the interaction between the compound and the B. tabaci ecdysone receptor is preferred over the interaction of the natural ligand with the B. tabaci ecdysone receptor.

16. The method according to claim 15, wherein the compound is an agonist of B. tabaci ecdysone receptor activity.

17. The method according to claim 15, wherein the compound is an antagonist of B. tabaci ecdysone receptor activity.

18. The method according to claim 3, the method further comprising the step of obtaining a compound which possesses stereochemical complementarity to a topographic region of the BtEcR/BtUSP heterodimer LBD and testing the compound for insecticidal activity.

19. A computer-assisted method for identifying potential compounds able to interact with an ecdysone receptor and thereby modulate an activity mediated by the receptor, using a programmed computer comprising a processor, an input device, and an output device, comprising the steps of:
(a) inputting into the programmed computer, through the input device, data comprising the atomic coordinates of amino acids 179-415 of the BtEcR monomer and amino acids 300-492 of the BtUSP monomer and ponasterone A positioned at atomic coordinates as shown in Appendix I, or structural coordinates wherein the backbone atoms of each monomer has a root mean square deviation from the backbone atoms of their corresponding partners in either amino acids 179-415 of the BtEcR monomer or amino acids 300-492 of the BtUSP monomer of not more than 1.5 Å, or one or more subsets of said amino acids, or one or more subsets of said amino acids related to the coordinates shown in Appendix I by whole body translations and/or rotations;
(b) generating, using computer methods, a set of atomic coordinates of a structure that possesses stereochemical complementarity to the atomic coordinates of amino acids 179-415 of the BtEcR monomer and/or amino acids 300-492 of the BtUSP monomer positioned at atomic coordinates as shown in Appendix I, or structural coordinates having a root mean square deviation from the backbone atoms of their corresponding partners in either amino acids 179-415 of the BtEcR monomer or amino acids 300-492 of the BtUSP monomer of not more than 1.5 Å, or one or more subsets of said amino acids, or one or more subsets of said amino acids related to the coordinates shown in Appendix I by whole body translations and/or rotations, thereby generating a criteria data set;
(c) comparing, using the processor, the criteria data set to a computer database of chemical structures;
(d) selecting from the database, using computer methods, chemical structures which are similar to a portion of said criteria data set; and
(e) outputting, to the output device, the selected chemical structures which are complementary to or similar to a portion of the criteria data set.

20. The method according to claim 19, wherein the structural coordinates have a root mean square deviation from the backbone atoms of said amino acids of not more than 1.0 Å.

21. The method according to claim 19, wherein the structural coordinates have a root mean square deviation from the backbone atoms of said amino acids of not more than 0.7 Å.

22. The method according to claim 19, wherein the method further comprises the step of obtaining a compound with a chemical structure selected in steps (d) and (e) and testing the compound for insecticidal activity.

23. The method according claim 19, wherein the subset of amino acids is that defining the ligand-binding pocket of the BtEcR subunit, namely F194, Q195, N196, Y198, E199, H200, P201, H226, I227, T228, I230, T231, L233, T234, L237, I238, F241, S242, V267, M268, M269, F270, R271, M272, R274, R275, I283, L284, F285, A286, Y296, M301, T304, L308, Y325, A326, T329, I333, M389, N390, T393, C394, L397, V404, P405, L408 and W412.

24. The method according to claim 19, wherein the subset of amino acids is that defining the interface between the BtEcR and BtUSP subunits defined by BtEcR residues H314, M315, I331, S335, E336, R337, P338, E347, Q350, E351, I354, E355, K358, T370, T371, F373, A374, K375, L377, S378, L380, T381, E382, R384, T385 and N388 and BtUSP residues E342, R383, T386, E387, K391, E414, E425, E429, Y432, A433, E436, S447, G448, F450, A451, K452, L454, L455, R456, L457, P458, A459, R461, S462 and L465.

25. The method according to claim 19, wherein the subset of amino acids is that defining the co-activator/co-repressor binding groove formed by helices H3 and H4 on the surface of BtEcR defined by residues I232, V235, Q236, V239, E240, K243, F248, R253, E254, Q256, I257, L260, K261, S264, S265, M268, S406, F407, L408, E410, I411 and D413.

26. A method of screening a putative compound having the ability to modulate the activity of the B. tabaci ecdysone receptor (BtEcR/BtUSP) or a heterodimer comprising the BtEcR monomer (SEQ ID No:1) paired with another functional partner protein such as the retinoic X receptor (RXR), comprising the steps of identifying a putative compound according to claim 3 or claim 19, and testing the compound for activity.

27. The method according to claim 26, wherein the testing of the compound is carried out in vitro.

28. The method according to claim 27, wherein the in vitro test is a high throughput assay.

29. The method according to claim 26, wherein the testing of the compound is carried out in vivo employing cell-based or whole organism-based screens.

30-43. (canceled)

44. A method for evaluating the ability of a chemical entity to interact with the BtEcR/BtUSP heterodimer LBD, said method comprising the steps of:
(a) creating a computer model of at least one region of the BtEcR/BtUSP heterodimer LBD using structure coordinates wherein the root mean square deviation between the backbone atoms of the (i) the BtEcR component of the model and the corresponding structure coordinates of amino acids 179-415 of the BtEcR monomer or (ii) the BtUSP component of the model and the corresponding structure coordinates of amino acids 300-492 of the BtUSP monomer, as set forth in Appendix I, are not more than 1.5 Å;
(b) employing computational means to perform a fitting operation between the chemical entity and said computer model of at least one region of the monomers of the BtEcR/BtUSP heterodimer LBD; and
(c) analysing the results of said fitting operation to quantify the association between the chemical entity and at least one region of the BtEcR/BtUSP heterodimer LBD model.

45. The method according to claim 44, wherein the structural coordinates have a root mean square deviation from the backbone atoms of said amino acids of not more than 1.0 Å.

46. The method according to claim 44, wherein the structural coordinates have a root mean square deviation from the backbone atoms of said amino acids of not more than 0.7 Å.

47. The method according to claim 44, wherein the region is the ligand-binding pocket of the BtEcR subunit defined by amino acids F194, Q195, N196, Y198, E199, H200, P201, H226, I227, T228, I230, T231, L233, T234, L237, I238, F241, S242, V267, M268, M269, F270, R271, M272, R274, R275, I283, L284, F285, A286, Y296, M301, T304, L308, Y325, A326, T329, I333, M389, N390, T393, C394, L397, V404, P405, L408 and W412.

48. The method according to claim 44, wherein the region is the interface between the BtEcR and BtUSP subunits defined by BtEcR residues H314, M315, I331, S335, E336, R337, P338, E347, Q350, E351, I354, E355, K358, T370, T371, F373, A374, K375, L377, S378, L380, T381, E382, R384, T385 and N388 and BtUSP residues E342, R383, T386, E387, K391, E414, E425, E429, Y432, A433, E436, S447, G448, F450, A451, K452, L454, L455, R456, L457, P458, A459, R461, S462 and L465.

49. The method according to claim 44, wherein the region is the co-activator/co-repressor binding groove formed by helices H3 and H4 of the surface of BtEcR defined by residues I232, V235, Q236, V239, E240, K243, F248, R253, E254, Q256, I257, L260, K261, S264, S265, M268, S406, F407, L408, E410, I411 and D413.

50-53. (canceled)

54. An isolated nucleic acid molecule comprising a nucleotide sequence which encodes at least the LBD of BtEcR, wherein the nucleotide sequence is selected from the group consisting of:
(i) a nucleotide sequence comprising a sequence having at least 90% identity to the sequence from nucleotide 535 to nucleotide 1248 of SEQ ID No: 1 or the complementary nucleotide sequence;
(ii) a nucleotide sequence comprising a sequence that hybridises under high stringency conditions to the sequence from nucleotide 535 to nucleotide 1248 of SEQ ID No: 1 or the complementary nucleotide sequence; and
(iii) a nucleotide sequence which encodes a polypeptide comprising the sequence from amino acid P179 to amino acid S416 of SEQ ID No: 2.

55-59. (canceled)

60. A nucleic acid molecule according to claim 54, wherein the nucleic acid molecule comprises a nucleotide sequence which encodes the polypeptide of SEQ ID No:2.


SEQUENCE LISTING (2 sequences)

SEQ ID NO: 1 - DNA, 1248 bp, Bemisia tabaci
atgtcttcgggcagctacgacccttatagtcccaccaatggaagaatagggaaagaagagctttcgccggcgaatagtttgaacgggtacagcgtggatagctgcgatgcgtcgcggaagaagaagggaggcacgggtcggcagcaggaggagctgtgtctcgtctgcggggaccgcgcctccggctaccactacaacgccctcacctgcgaaggctgcaagggcttcttccgtcggagcatcaccaagaatgccgtctaccagtgtaaatatggaaataattgtgaaatcgacatgtacatgaggcgaaaatgccaggagtgtcgtctcaagaagtgtctcagcgttggcatgaggccagaatgtgtagttcccgaattccagtgtgctgtgaagcgaaaagagaaaaaagcgcaaaaggacaaagataaacctaactcaacgacgagttgttctccagatggaatcaaacaagagatagatcctcaaaggctggatacagattcgcagctattgtctgtaaatggagttaaacccattactccagagcaagaagagctcatccataggctagtttattttcaaaatgaatatgaacatccatccccagaggatatcaaaaggatagttaatgctgcaccagaagaagaaaatgtagctgaagaaaggtttaggcatattacagaaattacaattctcactgtacagttaattgtggaattttctaagcgattacctggttttgacaaactaattcgtgaagatcaaatagctttattaaaggcatgtagtagtgaagtaatgatgtttagaatggcaaggaggtatgatgctgaaacagattcgatattgtttgcaactaaccagccgtatacgagagaatcatacactgtagctggcatgggtgatactgtggaggatctgctccgattttgtcgacatatgtgtgccatgaaagtcgataacgcagaatatgctcttctcactgccattgtaattttttcagaacgaccatctctaagtgaaggctggaaggttgagaagattcaagaaatttacatagaagcattaaaagcatatgttgaaaatcgaaggaaaccatatgcaacaaccatttttgctaagttactatctgttttaactgaactacgaacattagggaatatgaattcagaaacatgcttctcattgaagctgaagaatagaaaggtgccatccttcctcgaggagatttgggatgttgtttca

SEQ ID NO: 2 - protein, 416 aa, Bemisia tabaci
MSSGSYDPYSPTNGRIGKEELSPANSLNGYSVDSCDASRKKKGGTGRQQEELCLVCGDRASGYHYNALTCEGCKGFFRRSITKNAVYQCKYGNNCEIDMYMRRKCQECRLKKCLSVGMRPECVVPEFQCAVKRKEKKAQKDKDKPNSTTSCSPDGIKQEIDPQRLDTDSQLLSVNGVKPITPEQEELIHRLVYFQNEYEHPSPEDIKRIVNAAPEEENVAEERFRHITEITILTVQLIVEFSKRLPGFDKLIREDQIALLKACSSEVMMFRMARRYDAETDSILFATNQPYTRESYTVAGMGDTVEDLLRFCRHMCAMKVDNAEYALLTAIVIFSERPSLSEGWKVEKIQEIYIEALKAYVENRRKPYATTIFAKLLSVLTELRTLGNMNSETCFSLKLKNRKVPSFLEEIWDVVS